(19) United States
(12) Patent Application Publication
(10) Pub. No.: US 2026/0255780 A1
(43) Pub. Date: Aug. 27, 2026

(54) ARYLAMINE COMPOUND, AND ORGANIC ELECTROLUMINESCENT DEVICE AND ELECTRONIC APPARATUS

(71) Applicant: SHAANXI LIGHTE OPTOELECTRONICS MATERIAL CO., LTD., Xi'an, Shaanxi (CN)

(72) Inventors: Xianbin XU, Xi'an, Shaanxi (CN); Lei YANG, Xi'an, Shaanxi (CN)

(21) Appl. No.: 19/161,603

(22) PCT Filed: Apr. 15, 2024
(86) PCT No.: PCT/CN2024/087821

§ 371 Date: Sep. 2, 2025

(30) Foreign Application Priority Data
Aug. 25, 2023 (CN) 202311084253.7

(51) Int. Cl. H10K 50/12; C07D 407/12; C09K 11/06; H10K 85/30; H10K 85/60

(52) U.S. Cl. CPC H10K 50/12; C07D 407/12; C09K 11/06; H10K 85/342; H10K 85/351; H10K 85/626; H10K 85/633; H10K 85/636; H10K 85/654; H10K 85/6572; H10K 85/6574; H10K 85/6576; C09K 2211/1088

(57) ABSTRACT

The present application relates to the technical field of organic electroluminescent materials, and provides an arylamine compound having a structure represented by formula 1, and an organic electroluminescent device and electronic device comprising same. When used as a hole transport material in a hybrid host material, the arylamine compound of the present application can improve the carrier balance in a light-emitting layer, broaden the carrier recombination area, improve the exciton generation and utilization efficiency, improve the light-emitting efficiency of a device and prolong the service life of the device.

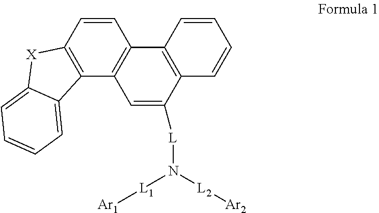

### CROSS-REFERENCE TO RELATED APPLICATIONS

The present application claims the priority of Chinese patent application No. 202311084253.7 filed on Aug. 25, 2023, which are incorporated herein by reference in its entirety as a part of this application.

### TECHNICAL FIELD

The present application relates to the technical field of organic electroluminescent materials, and in particular to an arylamine compound, and an organic electroluminescent device and an electronic apparatus comprising the same.

### BACKGROUND

With the development of electronic technology and the progress of material science, the application range of electronic components and devices used to realize electroluminescence or photoelectric conversion is increasingly extensive. An organic electroluminescent device (OLED) usually comprises: a cathode and an anode disposed opposite each other, and a functional layer disposed between the cathode and the anode. The functional layer is composed of multiple organic or inorganic film layers, and generally includes an organic light-emitting layer, a hole transport layer, an electron transport layer, etc. When a voltage is applied to the cathode and the anode, an electric field is generated between the two electrodes. Under the influence of the electric field, electrons on the cathode side move towards the electroluminescent layer, and holes on the anode side also move towards the luminescent layer. Electrons and holes combine in the electroluminescent layer to form excitons, which are in an excited state and release energy outward, thereby causing the electroluminescent layer to emit light externally.

In the existing organic electroluminescent devices, the primary issues are manifested in lifespan and efficiency. As displays become larger, driving voltages also increase. Research aimed at improving the performance of OLED devices includes: reducing the driving voltage of the device, enhancing the luminous efficiency of the device, and increasing operational lifespan of the device. In order to improve the device performance of OLEDs, a multilayer sandwich structure is usually adopted when designing the device structure, i.e., an anode, cathode and multiple organic functional layers jointly form a complete device. The host material in the light-emitting layer can be one or more materials. Host material is a material capable of accepting positively charged hole carriers and negatively charged electron carriers and combining them for effective energy transfer, which typically has a high first triplet energy level and is a crucial part of organic electroluminescent devices. There is still a need to carry out further research of novel host material in the light-emitting layer to further enhance the performance of organic electroluminescent devices.

### SUMMARY

Against the above problem in the prior art, the objective of the present application is to provide an arylamine compound, an organic electroluminescent device and an electronic apparatus comprising the same. The arylamine compound, when utilized in an organic electroluminescent device, can improve the performance of the device.

According to a first aspect of the present application, there is provided an arylamine compound having a structure represented by Formula 1:

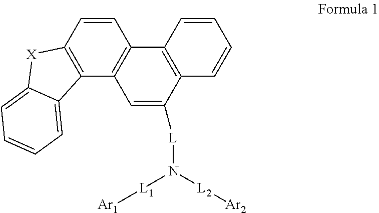

wherein, X is selected from O or S;
L, L₁ and L₂ are each independently selected from the group consisting of a single bond, a substituted or unsubstituted arylene having 6 to 30 carbon atoms, and a substituted or unsubstituted heteroarylene having 3 to 20 carbon atoms;
Ar₁ and Ar₂ are the same or different, and are each independently selected from the group consisting of a substituted or unsubstituted aryl having 6 to 30 carbon atoms, and a substituted or unsubstituted heteroaryl having 3 to 20 carbon atoms;
the substituents in L, L₁, L₂, Ar₁, and Ar₂ are the same or different, and are each independently selected from the group consisting of deuterium, cyano, halogen, alkyl having 1-10 carbon atoms, haloalkyl having 1-10 carbon atoms, deuteroalkyl having 1-10 carbon atoms, alkoxy having 1-10 carbon atoms, alkylthio having 1-10 carbon atoms, trialkylsilyl having 3-12 carbon atoms, aryl having 6-15 carbon atoms, heteroaryl having 3-12 carbon atoms, and cycloalkyl having 3-10 carbon atoms; optionally, any two adjacent substituents form a saturated or unsaturated 3-15 membered ring.

According to a second aspect of the present application, there is provided an organic electroluminescent device, comprising an anode and a cathode arranged opposite each other, and a functional layer disposed between the anode and the cathode; the functional layer contains the aforementioned arylamine compound.

According to a third aspect of the present application, there is provided an electronic apparatus, comprising the organic electroluminescent device described in the second aspect.

The compound of the present application comprises a core structure of a phenanthro[2,1-b]benzo five-membered heteroaryl ring, which is connected to an arylamine-like hole transporting fragment through a specific position

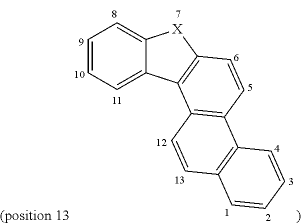

as a hole transport type light-emitting host material. On the one hand, the unique fusion pattern of phenanthrene and benzofive-membered heteroaryl ring ensures that the core of phenanthro[2,1-b]benzo five-membered heteroaryl ring possesses an appropriate first excited triplet energy level, which is suitable to be used as a fragment for light-emitting host materials; on the other hand, the core structure of phenanthro[2,1-b]benzo five-membered heteroaryl ring features an extended conjugated system. When the core is linked to an arylamine-like hole transporting fragment through position 13, the intermolecular interactions can be enhanced, which improves the hole mobility of the compound. The arylamine compound of the present application, when utilized as a hole transport host material in a hybrid light-emitting host material, can improve the carrier balance within the light-emitting layer, expanding the carrier recombination region, enhancing the efficiency of exciton generation and utilization, and increasing the luminous efficiency and operational lifespan of the devices.

### BRIEF DESCRIPTION OF THE DRAWINGS

The drawings are used for a further understanding of the present application and constitute a part of the specification and are used to explain the present application together with the following specific embodiments but do not constitute a limitation of the present application.

FIG. 1 is a schematic structural diagram of an organic electroluminescent device according to one embodiment of the present application.

FIG. 2 is a schematic structural diagram of an electronic apparatus according to one embodiment of the present application.

Reference Signs

| | | | |
|---|---|---|---|
| 100: Anode | 200: Cathode | 300: Functional layer | 310: Hole injection layer |
| 321: First hole | 322: Light-emitting | 330: Organic light- | 340: Electron transport |
| transport layer | adjustment layer | emitting layer | layer |
| 350: Electron injection | 320: Hole transport | 400: Electronic | |
| layer | layer | apparatus | |

### DETAILED DESCRIPTION OF THE EMBODIMENTS

Exemplary embodiments will now be described more comprehensively with reference to the accompanying drawings. The exemplary embodiments, however, can be implemented in a variety of forms and should not be interpreted as being limited to the examples set forth herein. On the contrary, these embodiments are provided to make the present application more comprehensive and complete, and to fully convey the concepts of these exemplary embodiments to those skilled in the art. Features, structures, or characteristics described herein can be combined in one or more embodiments in any suitable manner. In the following description, many specific details are provided to give a full understanding of the embodiments of the present application.

In a first aspect, the present application provides an arylamine compound having a structure represented by Formula 1:

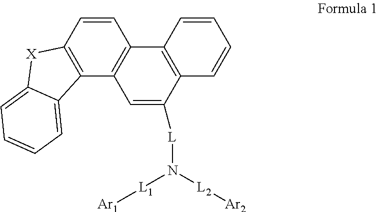

wherein, X is selected from O or S;
L, L₁ and L₂ are each independently selected from the group consisting of a single bond, a substituted or unsubstituted arylene having 6 to 30 carbon atoms, and a substituted or unsubstituted heteroarylene having 3 to 20 carbon atoms;
Ar₁ and Ar₂ are the same or different, and are each independently selected from the group consisting of a substituted or unsubstituted aryl having 6 to 30 carbon atoms, and a substituted or unsubstituted heteroaryl having 3 to 20 carbon atoms;
the substituents in L, L₁, L₂, Ar₁, and Ar₂ are the same or different, and are each independently selected from the group consisting of deuterium, cyano, halogen, alkyl having 1-10 carbon atoms, haloalkyl having 1-10 carbon atoms, deuterated alkyl having 1-10 carbon atoms, alkoxy having 1-10 carbon atoms, alkylthio having 1-10 carbon atoms, trialkylsilyl having 3-12 carbon atoms, aryl having 6-15 carbon atoms, heteroaryl having 3-12 carbon atoms, and cycloalkyl having 3-10 carbon atoms; optionally, any two adjacent substituents form a saturated or unsaturated 3-15 membered ring.

In the present application, the terms “optional” and “optionally” mean that the subsequently described event or circumstance may or may not occur. For example, “optionally, any two adjacent substituents form a saturated or unsaturated 3-15 membered ring” includes scenarios both where two adjacent substituents form a ring and where two adjacent substituents are independently present without forming a ring. “Any two adjacent substituents” may include having two substituents on the same atom, and may also include having one substituent on each of adjacent atoms; among them, when there are two substituents on the same atom, the two substituents can form a saturated or unsaturated spiro-ring with the atom they are jointly connected to; when two adjacent atoms each have a substituent, these two substituents can be fused into a ring.

In the present application, the terms “alternatively” and “preferably” have the same meaning as “in some embodiments”.

In the present application, the descriptive expressions “each . . . be independently” and “ . . . be respectively independently” and “ . . . be each independently” can be interchanged and all these expressions should be interpreted in a broad sense. They can both refer to that specific options expressed by the same symbol in different groups are mutually non-influential, and to that specific options expressed by the same symbols within the same group are mutually non-influential. For example,

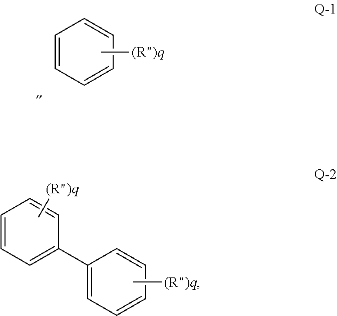

in which each q is independently 0, 1, 2, or 3, and each R″ is independently selected from the group consisting of hydrogen, deuterium, fluorine, and chlorine” means that Formula Q-1 represents that there are q substituents R″ on the benzene ring, and each R″ can be the same or different, with mutual non-influence between the options for each R″; Formula Q-2 represents that there are q substituents R″ on each benzene ring of biphenyl, and the number q of R″ substituents on the two benzene rings can be the same or different, with mutual non-influence between the options for each R″.

In the present application, the term “substituted or unsubstituted” means that the functional group defined by the term may or may not have a substituent (hereinafter referred to as Rc for ease of description). For example, “a substituted or unsubstituted aryl” refers to an aryl having a substituent Rc or an unsubstituted aryl. Among them, the above substituent, i.e., Rc, may be, for example, deuterium, fluorine, cyano, heteroaryl, aryl, deuterated aryl, trialkylsilyl, alkyl, haloalkyl, deuterated alkyl, cycloalkyl, etc. The number of substituents may be one or more.

In the present application, “more” refers to two or more, for example, 2, 3, 4, 5, 6, etc.

In the present disclosure, the number of carbon atoms of a substituted or unsubstituted functional group refers to the total number of carbon atoms of the group and all substituents thereon. For example, if L₁ is a substituted arylene having 12 carbon atoms, the total number of carbon atoms in the arylene and the substituents thereon is 12.

In the structure of the compound of the present disclosure, hydrogen atoms include various isotopic atoms of the hydrogen element, such as hydrogen (H), deuterium (D), or tritium (T).

The “D” in the structural formula of the compound of the present application indicates deuteration.

In the present disclosure, a saturated or unsaturated 3-15 membered ring refers to a cyclic group comprising 3-15 ring atoms; for example, but is not limited to cyclopentane, cyclohexane, benzene ring, fluorene ring, etc.

In the present application, aryl refers to an optional functional group or substituent derived from an aromatic carbon ring. Aryl may be a monocyclic aryl (e.g., phenyl) or a polycyclic aryl. In other words, aryl may be a monocyclic aryl, a fused-ring aryl, two or more monocyclic aryls linked by carbon-carbon bond, a monocyclic aryl and a fused-ring aryl linked by carbon-carbon bond, or two or more fused-ring aryls linked by carbon-carbon bond. That is, unless otherwise specified, two or more aromatic groups linked by carbon-carbon bond may also be regarded as the aryl in the present application. Among them, a fused-ring aryl may include, for example, a bicyclic fused aryl (e.g., naphthyl), a tricyclic fused aryl (e.g., phenanthryl, fluorenyl, and anthracenyl), etc. Aryl does not contain heteroatoms such as B, N, O, S, P, Se and Si. Examples of the aryl include, but are not limited to, phenyl, naphthyl, fluorenyl, spirobifluorenyl, anthracenyl, phenanthryl, biphenyl, terphenyl, quaterphenyl, quinquephenyl, triphenylene, perylenyl, benzo[9,10]phenanthryl, pyrenyl, benzofluoranthryl, chrysenyl, etc.

In the present application, “arylene” involved refers to a divalent or multivalent group formed by further removing one or more hydrogen atoms from aryl.

In the present application, terphenyl includes

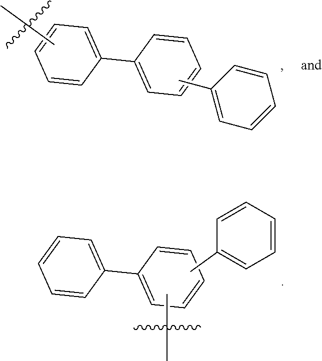

In the present application, the number of carbon atoms of a substituted or unsubstituted aryl (arylene) may be 6, 8, 10, 12, 13, 14, 15, 16, 17, 18, 19, 20, 21, 22, 23, 24, 25, or 30. In some embodiments, a substituted or unsubstituted aryl is a substituted or unsubstituted aryl having 6-30 carbon atoms; in other embodiments, a substituted or unsubstituted aryl is a substituted or unsubstituted aryl having 6-25 carbon atoms; in other embodiments, a substituted or unsubstituted aryl is a substituted or unsubstituted aryl having 6-18 carbon atoms; and in other embodiments, a substituted or unsubstituted aryl is a substituted or unsubstituted aryl having 6-15 carbon atoms.

In the present application, fluorenyl can be substituted by one or more substituents. In the case that the above-mentioned fluorenyl is substituted, the substituted fluorenyl may be:

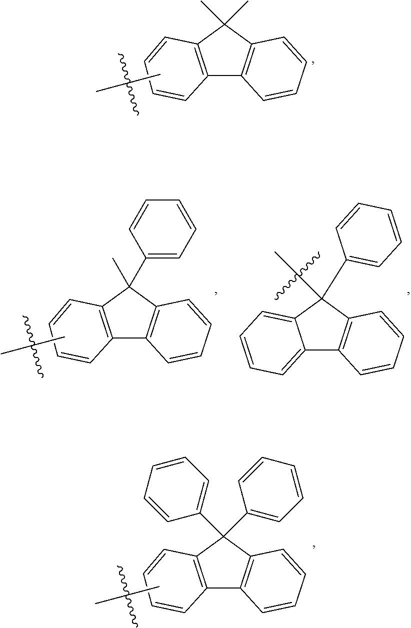

etc, but are not limited thereto.

In the present application, the aryl as the substituent of L, L₁, L₂, Ar₁, and Ar₂ is for example, but is not limited to, phenyl, naphthyl, phenanthryl, biphenyl, fluorenyl, dimethylfluorenyl, etc.

In the present application, “heteroaryl” refers to a monovalent aromatic ring containing 1, 2, 3, 4, 5, or 6 heteroatoms or derivative thereof. The heteroatoms may be one or more of B, O, N, P, Si, Se, and S. Heteroaryl may be a monocyclic heteroaryl or a polycyclic heteroaryl. In other words, heteroaryl may be a single aromatic ring system, or multiple aromatic ring systems linked by carbon-carbon bond, with any of the aromatic ring systems being an aromatic monocyclic ring or a fused aromatic ring. For example, heteroaryl may include, thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, N-phenylcarbazolyl, N-pyridylcarbazolyl, N-methylcarbazolyl, etc, but be not limited thereto.

In the present disclosure, heteroaryl as the substituent of L, L₁, L₂, Ar₁, and Ar₂ is for example, but is not limited to, dibenzothienyl, dibenzofuranyl, carbazolyl, etc.

In the present disclosure, “heteroarylene” involved refers to a divalent or multivalent group formed by further removing one or more hydrogen atoms from heteroaryl.

In the present disclosure, the number of carbon atoms of a substituted or unsubstituted aryl (arylene) may be selected from the group consisting of 3, 4, 5, 6, 7, 8, 9, 10, 11, 12, 13, 14, 15, 16, 17, 18, or 20. In some embodiments, a substituted or unsubstituted heteroaryl is a substituted or unsubstituted heteroaryl having 3-30 carbon atoms; in other embodiments, a substituted or unsubstituted heteroaryl is a substituted or unsubstituted heteroaryl having 3-18 carbon atoms; and in other embodiments, a substituted or unsubstituted heteroaryl is a substituted or unsubstituted heteroaryl having 12-18 carbon atoms.

In the present disclosure, alkyl having 1-10 carbon atoms may include a straight-chain alkyl having 1-10 carbon atoms, and a branched alkyl having 3-10 carbon atoms. The number of carbon atoms of the alkyl may be, for example, 1, 2, 3, 4, 5, 6, 7, 8, 9, or 10, and the specific examples of the alkyl include, but are not limited to, methyl, ethyl, n-propyl, isopropyl, n-butyl, isobutyl, tert-butyl, n-pentyl, isopentyl, neopentyl, n-hexyl, etc.

In the present application, halogen may be, for example, fluorine, chlorine, bromine, or iodine.

In the present application, the specific examples of trialkylsilyl include, but are not limited to, trimethylsilyl, triethylsilyl, etc.

In the present application, haloalkyl refers to alkyl substituted with halogen, the specific examples of haloalkyl include, but are not limited to, a trifluoromethyl.

In the present application, deuterated alky refers to alky substituted with one or more deuterium, the specific examples of deuterated alkyl include, but are not limited to, trideuteromethyl.

In the present application, the number of carbon atoms of cycloalkyl having 3-10 carbon atoms may be, for example, 3, 4, 5, 6, 7, 8, or 10. The specific examples of cycloalkyl include, but are not limited to, a cyclopentyl, a cyclohexyl, an adamantyl, etc.

In the present application, the number of carbon atoms of deuterated alkyl having 1-10 carbon atoms is, for example, 1, 2, 3, 4, 5, 6, 7, 8, or 10. The specific examples of deuterated alkyl include, but are not limited to, trideuteromethyl.

In the present application, the number of carbon atoms of haloalkyl having 1-10 carbon atoms is, for example, 1, 2, 3, 4, 5, 6, 7, 8, or 10. The specific examples of haloalkyl include, but are not limited to, trifluoromethyl.

In the present application,

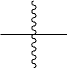

refers to a chemical bond interconnected with other groups.

In the present application, a non-positional connection bond involves a single bond

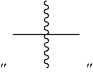

extending from the ring system, which represents that one end of the connection bond can connect to any position in the ring system through which the bond passes, and the other end connects to the rest of the compound molecule. For example, as shown in Formula (f) below, the naphthyl represented by Formula (f) is connected to other positions of the molecule through two non-positional connection bonds passing through the two rings, which indicates any of possible connection modes shown in Formulae (f-1) to (f-10):

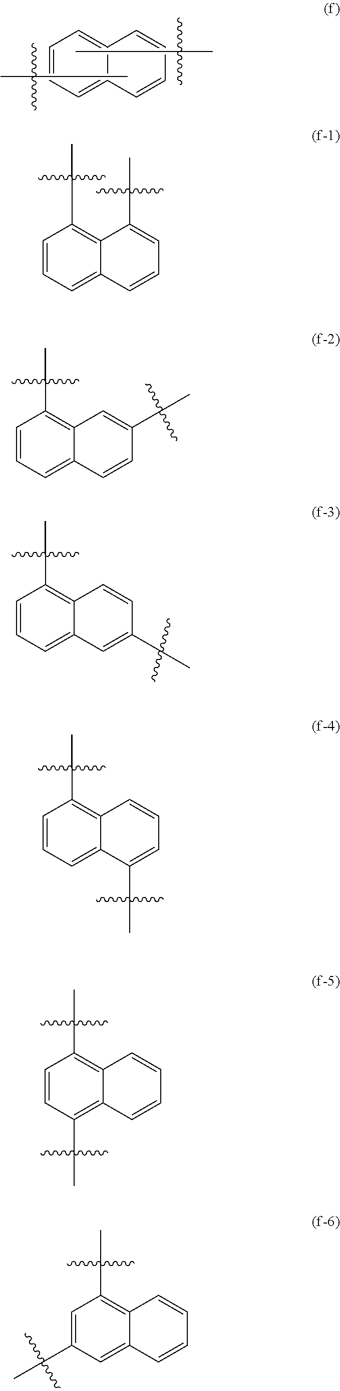

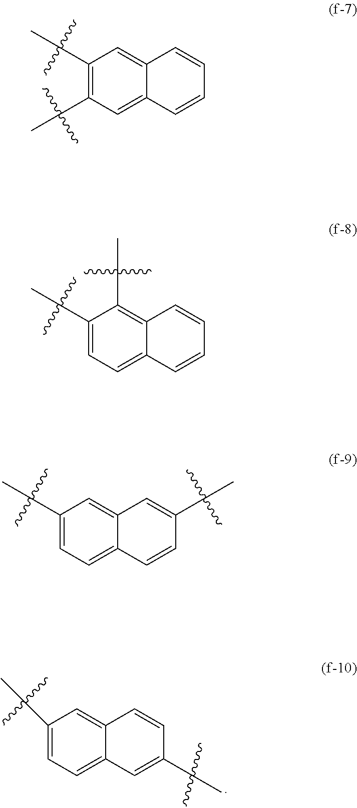 [SMILES: *c1cc2ccccc2cc1* ; *c1ccc2ccccc2c1* ; *c1ccc2ccc(*)cc2c1 ; *c1ccc2cc(*)ccc2c1]

As another example, as shown in Formula (X′) below, the dibenzofuranyl represented by Formula (X′) is connected to other positions of the molecule via a non-positional connection bond extending from the center of a benzene ring at one side, which indicates any of possible connection modes shown in Formulae (X′-1) to (X′-4):

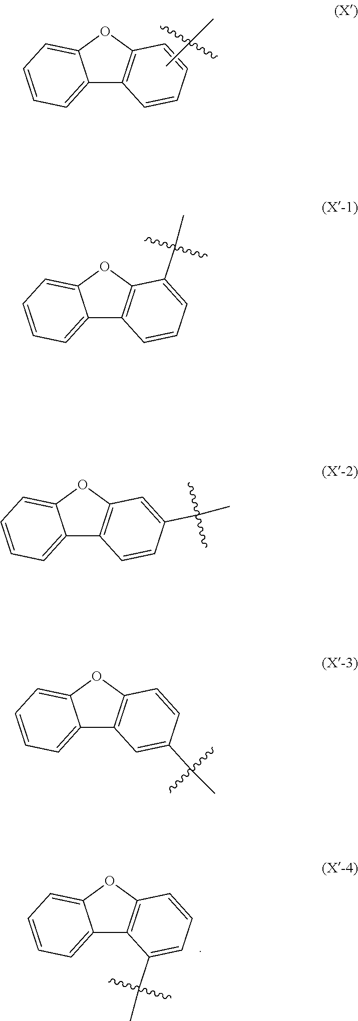

The non positioned substituent in the present application refers to a substituent connected by a single bond extending from the center of the ring system, indicating that the substituent can be connected to any possible position in the ring system. For example, as represented by Formula (Y) below, the substituent R′ represented by Formula (Y) is linked to a quinoline ring via a non-positional connection bond, which indicates any of possible connection mode shown in Formulae (Y-1) to (Y-7):

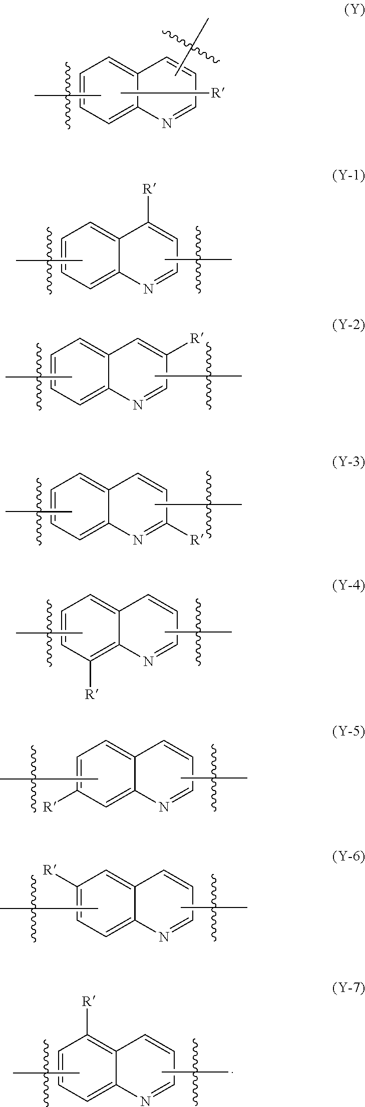

In some embodiments, L, L₁ and L₂ are the same or different, and are each independently selected from the group consisting of a single bond, a substituted or unsubstituted arylene having 6, 7, 8, 9, 10, 11, 12, 13, 14, or 15, a substituted or unsubstituted heteroarylene having 5, 6, 7, 8, 9, 10, 11, 12, 13, 14, 15, 16, 17, or 18.

Alternatively, the substituents in L, L₁ and L₂ are the same or different, and are each independently selected from the group consisting of deuterium, cyano, halogen, alkyl having 1-4 carbon atoms, haloalkyl having 1-4 carbon atoms, deuterated alkyl having 1-4 carbon atoms, trialkylsilyl having 3-7 carbon atoms, and phenyl.

In some embodiments, L, L₁ and L₂ are each independently selected from the group consisting of a single bond, a substituted or unsubstituted phenylene, a substituted or unsubstituted naphthylene, a substituted or unsubstituted biphenylene, a substituted or unsubstituted fluorenylene, a substituted or unsubstituted phenanthrylene, a substituted or unsubstituted dibenzothienylene, a substituted or unsubstituted dibenzofuranylene, and a substituted or unsubstituted carbazolylene.

Alternatively, the substituents in L, L₁ and L₂ are the same or different, and are each independently selected from the group consisting of deuterium, fluorine, cyano, methyl, ethyl, isopropyl, tert-butyl, trifluoromethyl, trideuteromethyl, trimethylsilyl, pentadeuterophenyl, or phenyl.

In some embodiments, L₁ and L₂ are each independently selected from the group consisting of a single bond and the following groups:

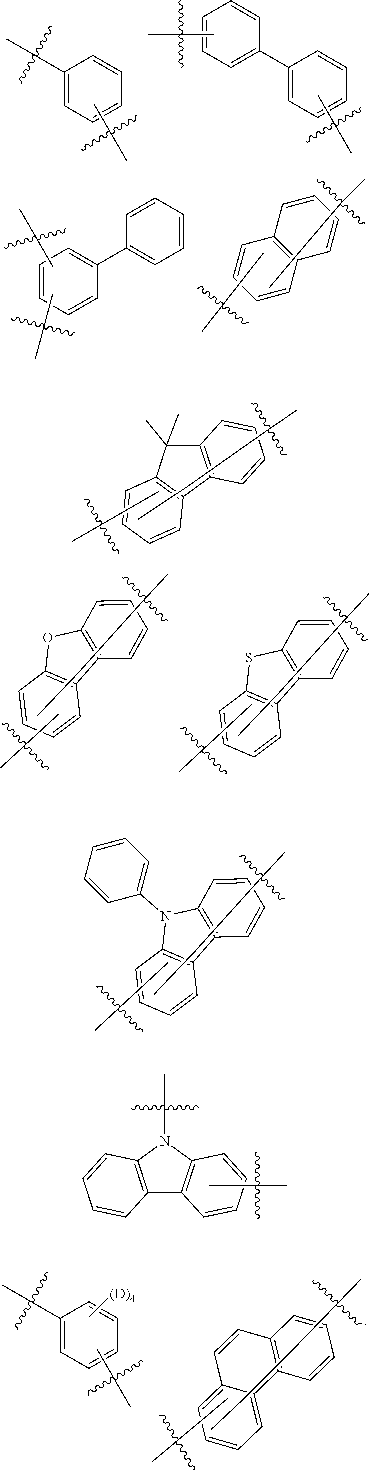

In some embodiments, L is selected from the group consisting of a single bond and the following groups:

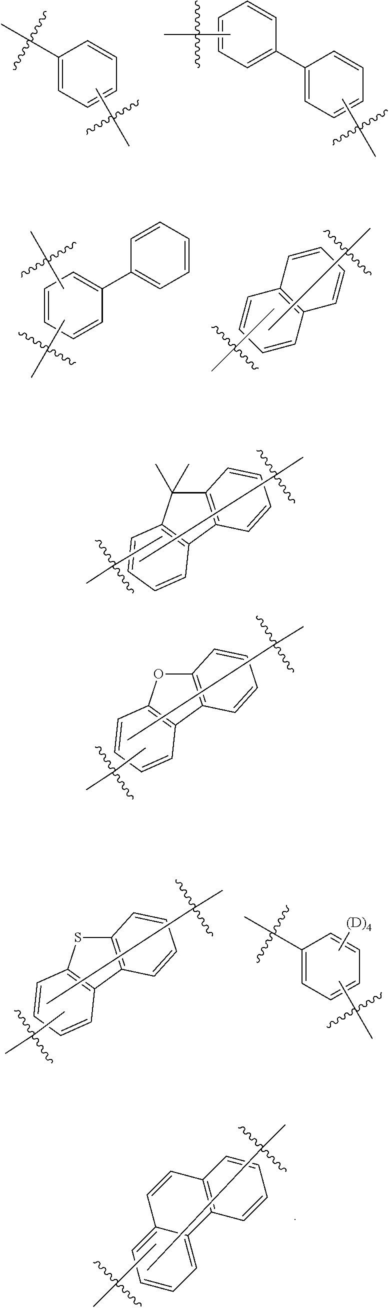

In some embodiments, L₁ and L₂ are the same or different, and are each independently selected from the group consisting of a single bond and the following groups:

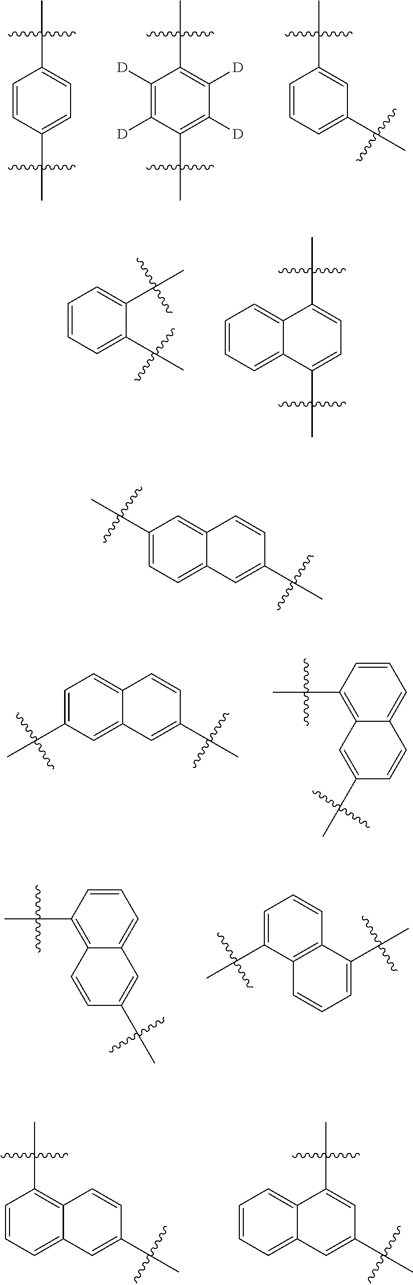

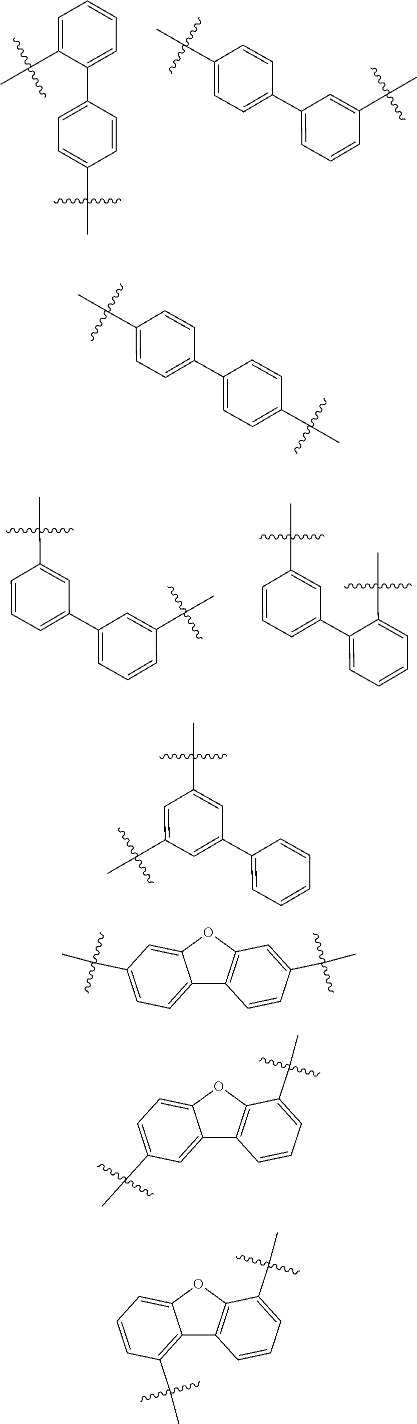

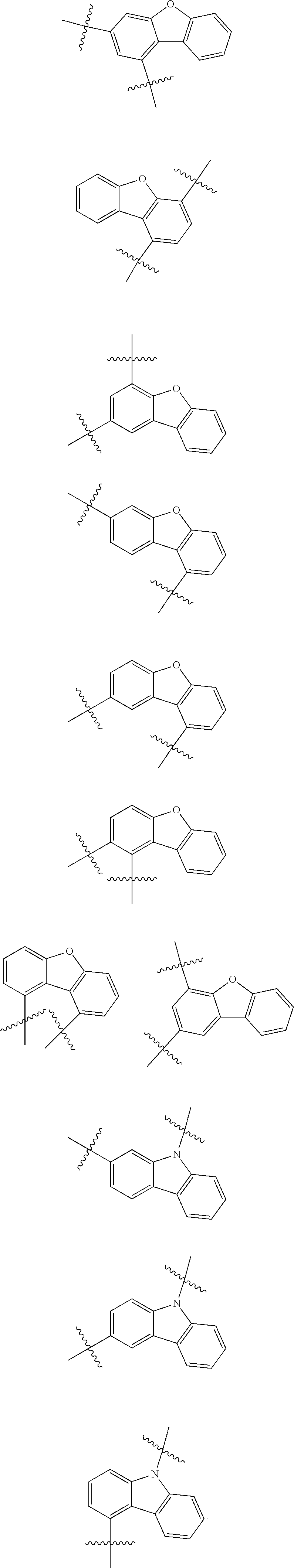

In some embodiments, L is selected from the group consisting of a single bond and the following groups:

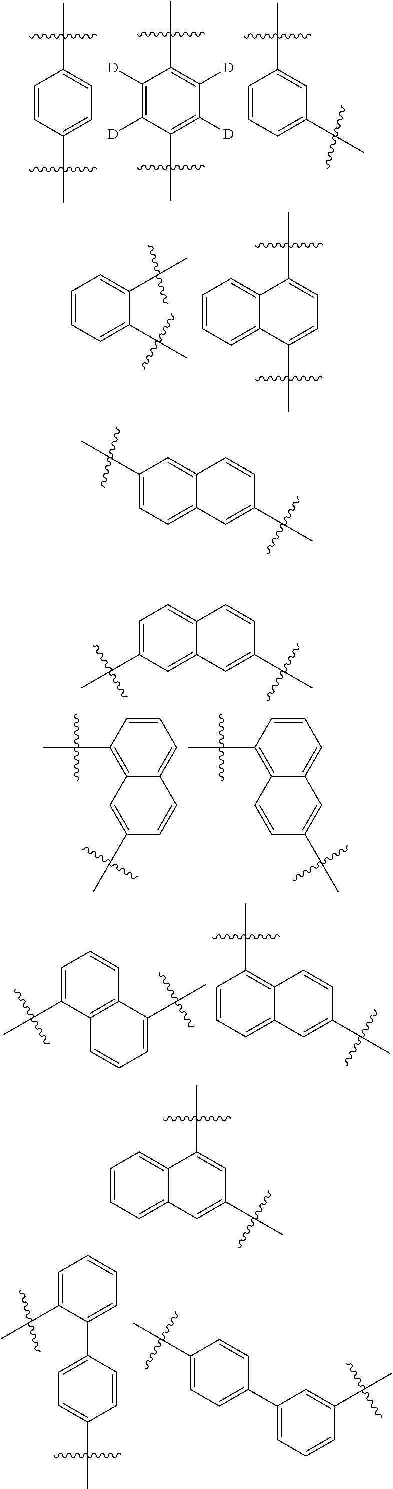

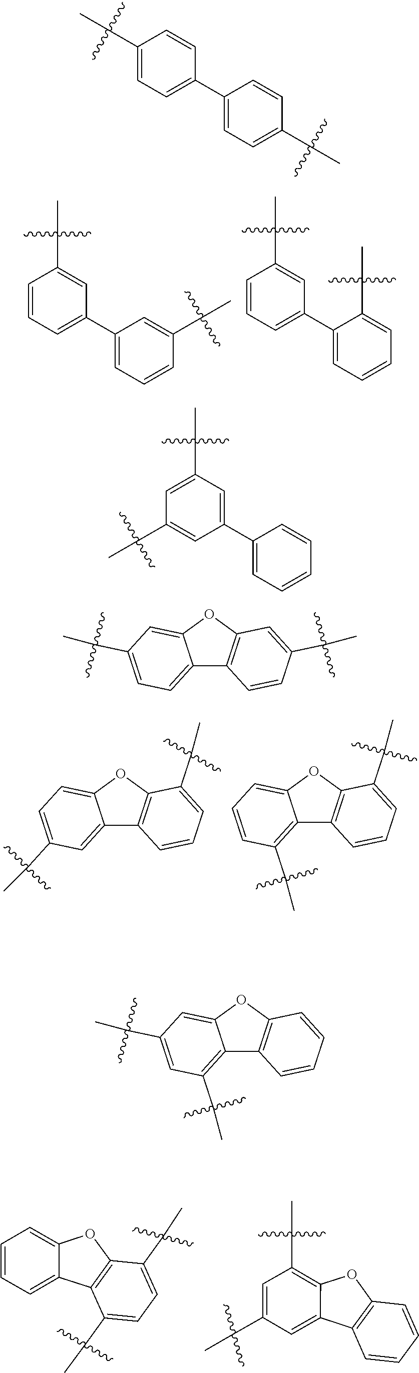

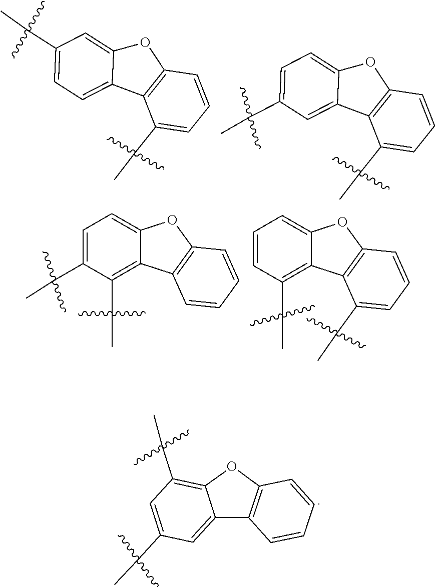

In some embodiments, Ar₁ and Ar₂ are each independently selected from the group consisting of a substituted or unsubstituted aryl having 6, 7, 8, 9, 10, 11, 12, 13, 14, 15, 16, 17, 18, 19, 20, 21, 22, 23, 24, 25, 26, 27, 28, 29, or 30 carbon atoms, and a substituted or unsubstituted heteroaryl having 5, 6, 7, 8, 9, 10, 11, 12, 13, 14, 15, 16, 17, or 18 carbon atoms.

In some embodiments, Ar₁ and Ar₂ are each independently selected from the group consisting of aryl having 6-25 carbon atoms, and heteroaryl having 12-18 carbon atoms.

Alternatively, the substituents in Ar₁ and Ar₂ are each independently selected from the group consisting of deuterium, halogen, cyano, haloalkyl having 1-4 carbon atoms, deuterated alkyl having 1-4 carbon atoms, alkyl having 1-4 carbon atoms, cycloalkyl having 5-10 carbon atoms, aryl having 6-15 carbon atoms, heteroaryl having 5-12 carbon atoms, trialkylsilyl having 3-7 carbon atoms, and deuterated aryl having 6-15 carbon atoms; optionally, any two adjacent substituents form a benzene ring or a fluorene ring.

In some embodiments, Ar₁ and Ar₂ are the same or different, and are each independently selected from the group consisting of a substituted or unsubstituted phenyl, a substituted or unsubstituted naphthyl, a substituted or unsubstituted biphenyl, a substituted or unsubstituted terphenyl, a substituted or unsubstituted fluorenyl, a substituted or unsubstituted phenanthryl, a substituted or unsubstituted anthracenyl, a substituted or unsubstituted pyrenyl, a substituted or unsubstituted triphenylene, a substituted or unsubstituted spirobifluorenyl, a substituted or unsubstituted dibenzothienyl, a substituted or unsubstituted dibenzofuranyl, and a substituted or unsubstituted carbazolyl.

Alternatively, the substituents in Ar₁ and Ar₂ are the same or different, and are each independently selected from the group consisting of deuterium, fluorine, cyano, methyl, ethyl, isopropyl, tert-butyl, trifluoromethyl, trideuteromethyl, trimethylsilyl, pentadeuterophenyl, phenyl, and naphthyl.

In some embodiments, Ar₁ and Ar₂ are the same or different, and are each independently selected from the group consisting of the following group:

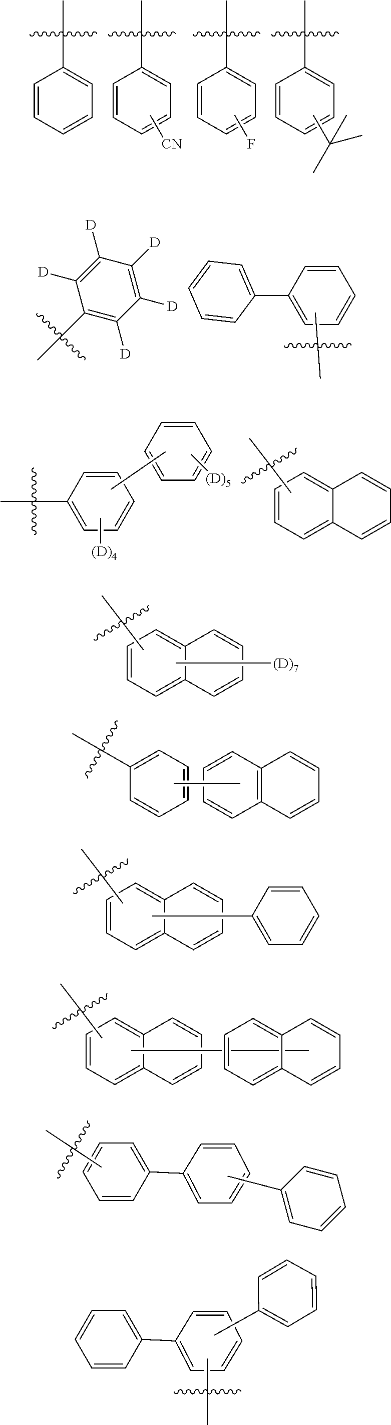

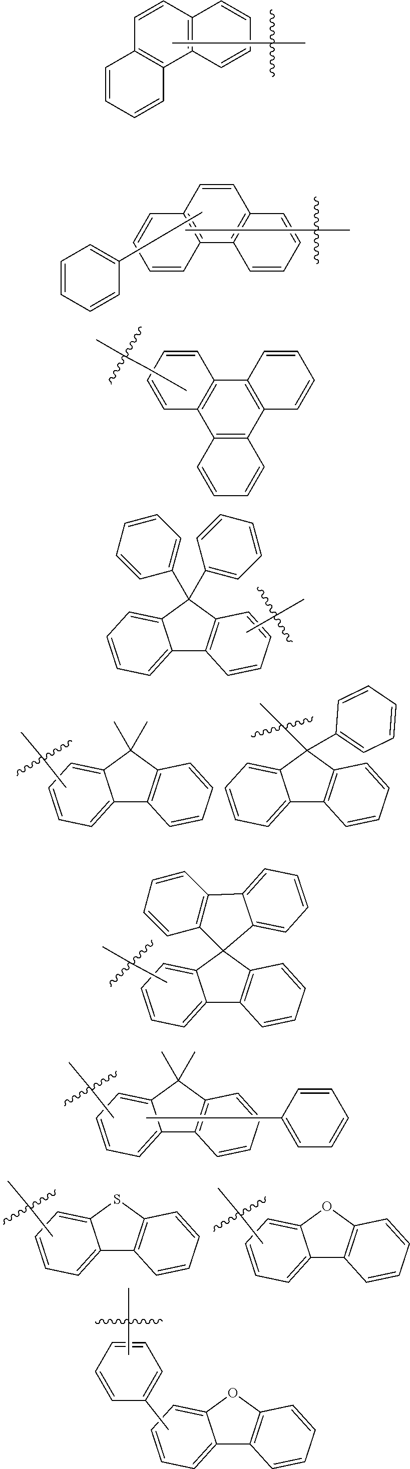

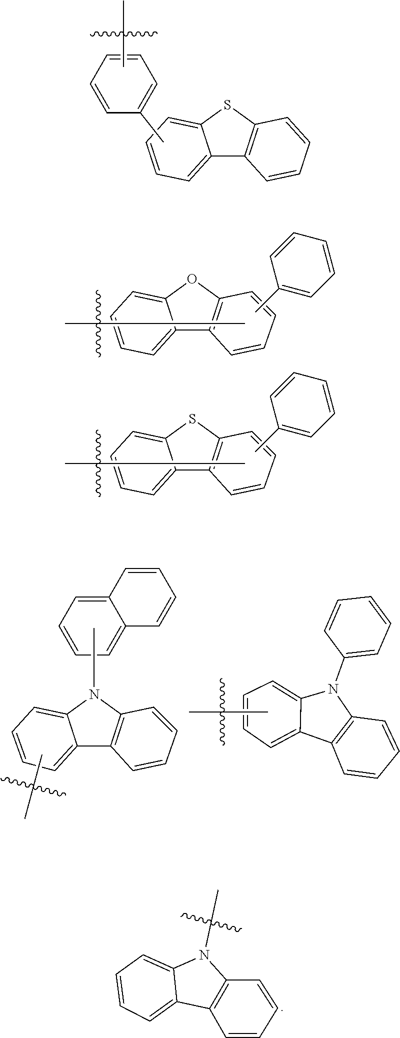

In some embodiments, Ar₁ and Ar₂ are the same or different, and are each independently selected from the group consisting of the following group:

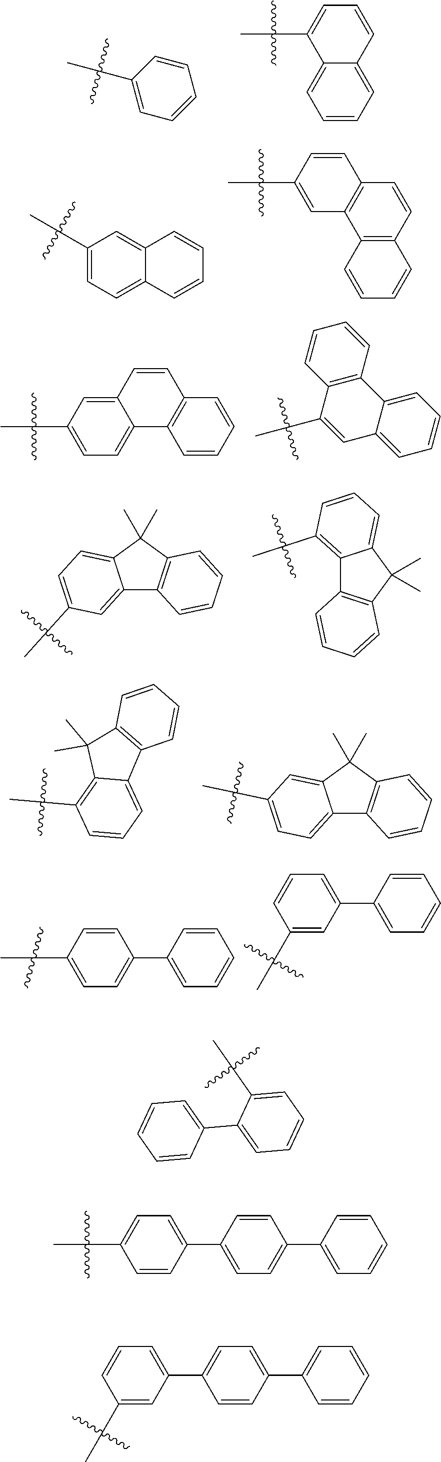

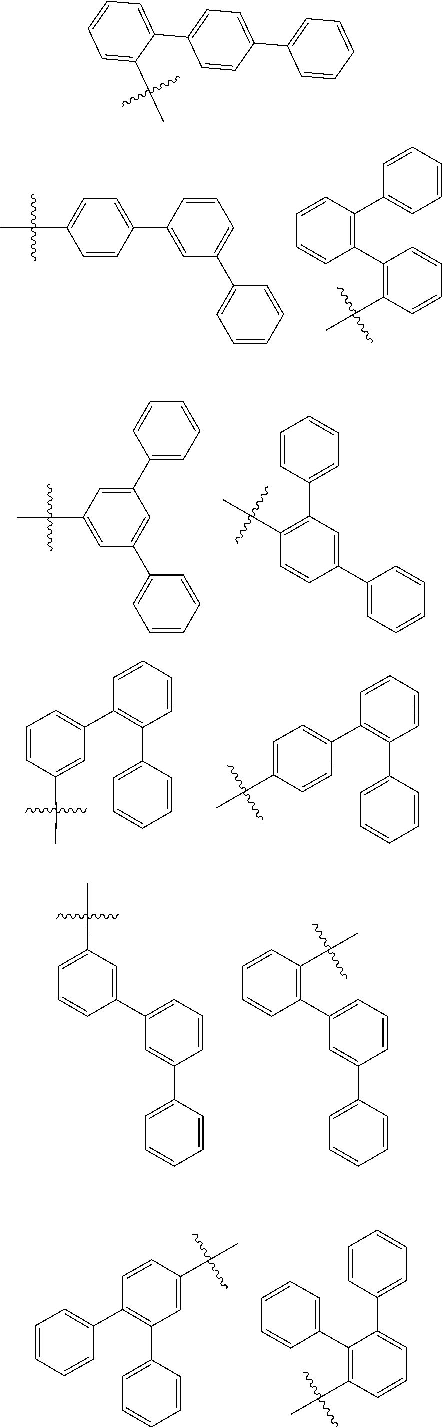

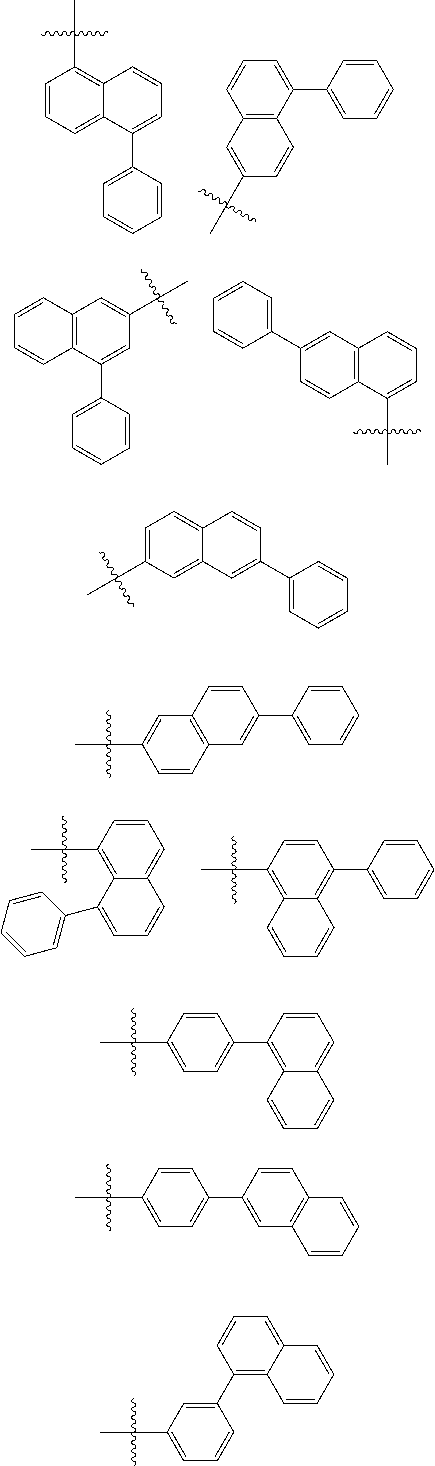

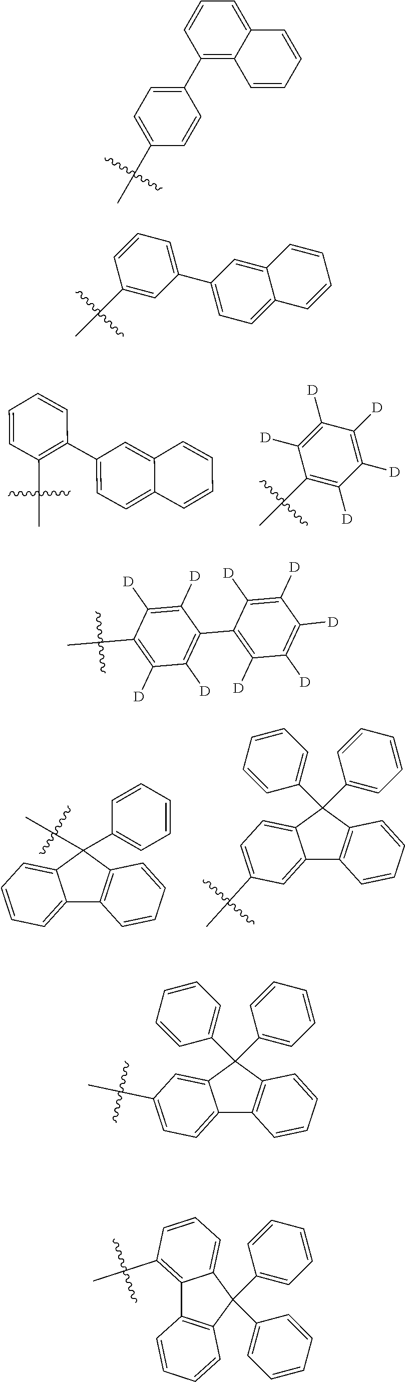

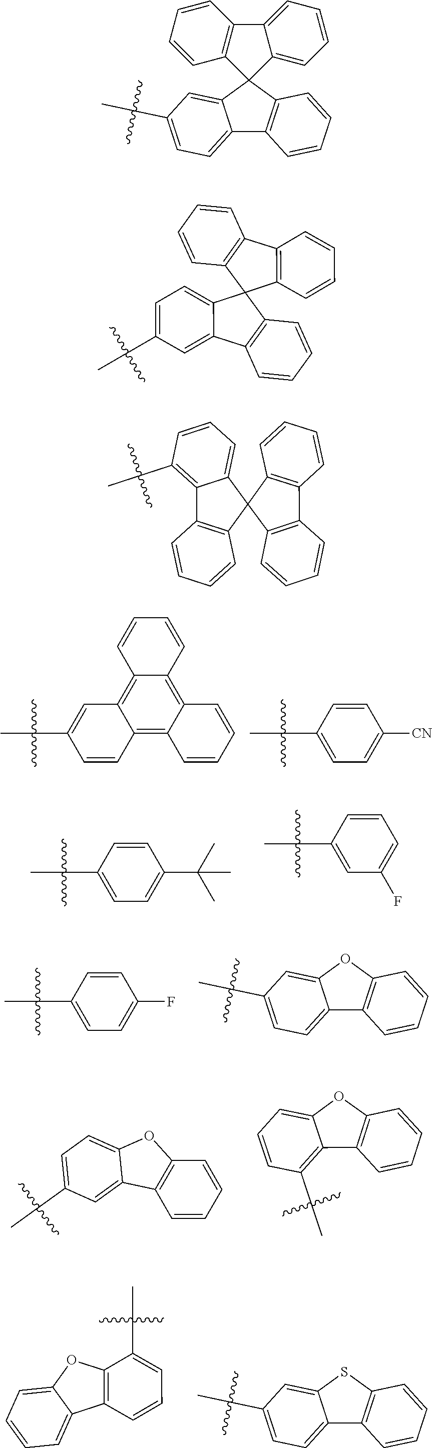

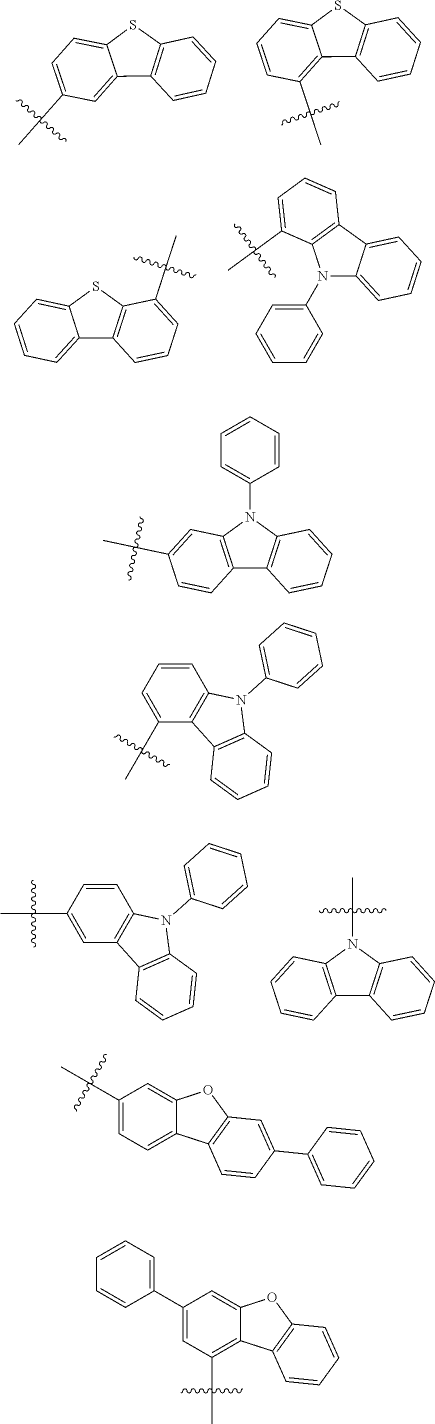

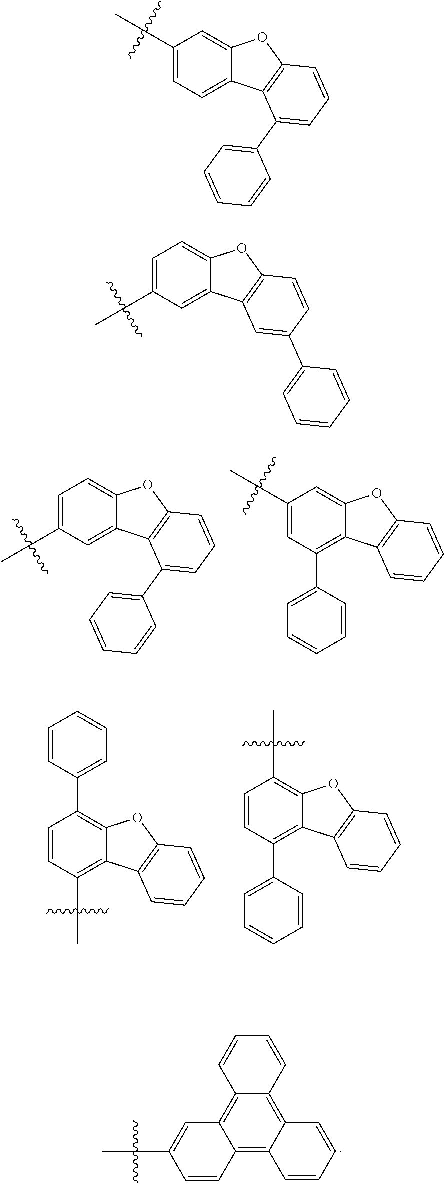

In some embodiments,

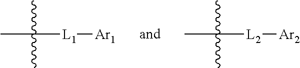

are the same or different, and are each independently selected from the group consisting of the following group:

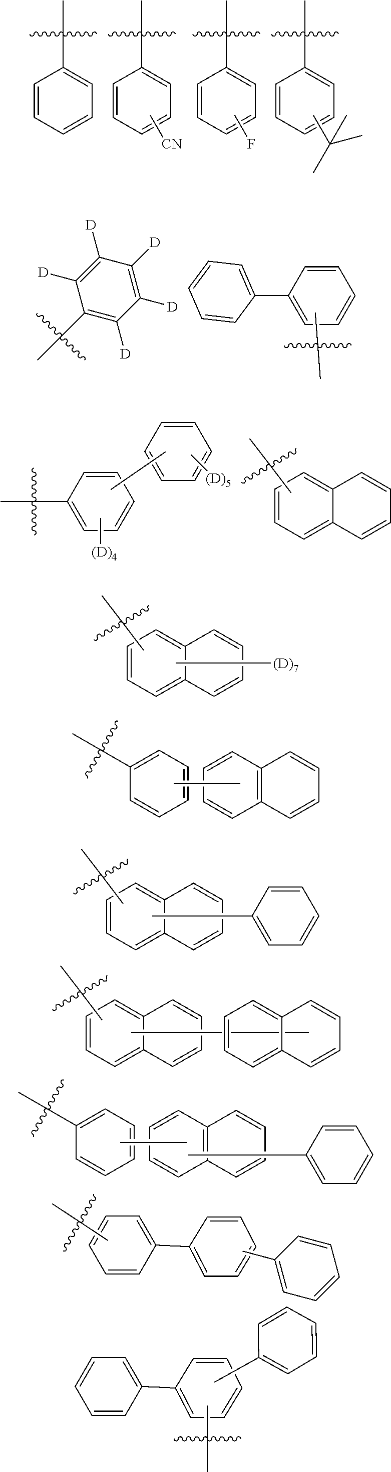

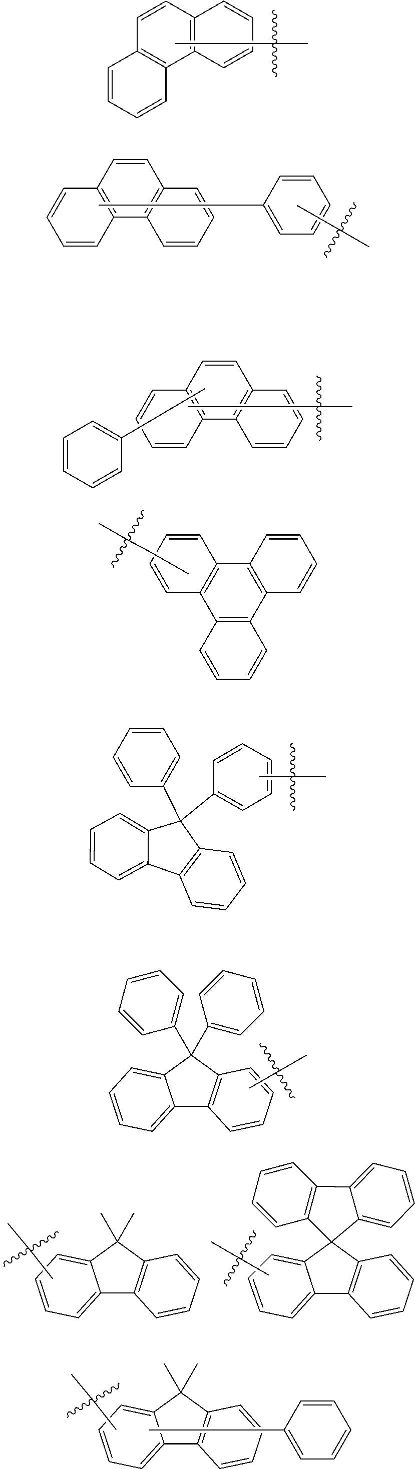

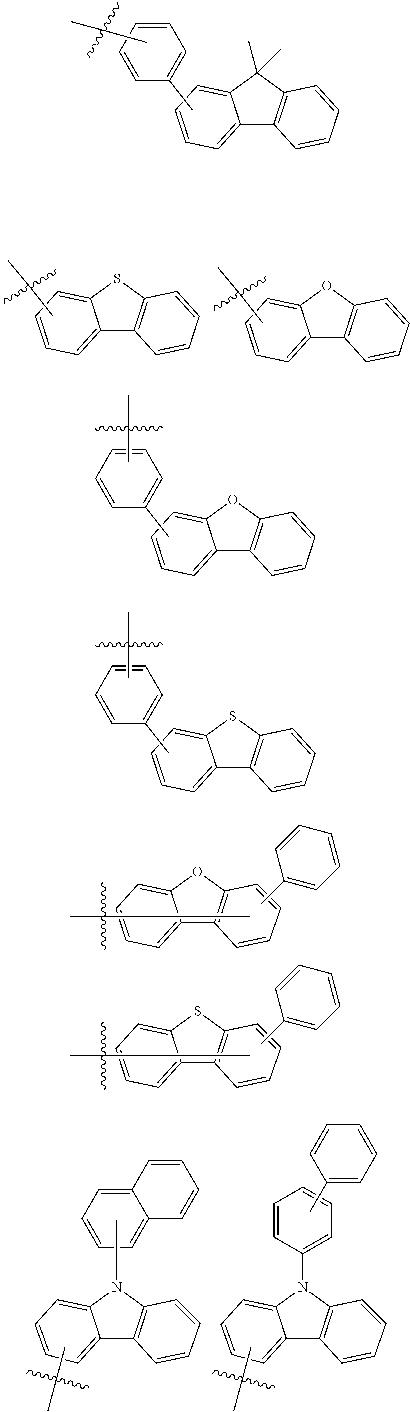

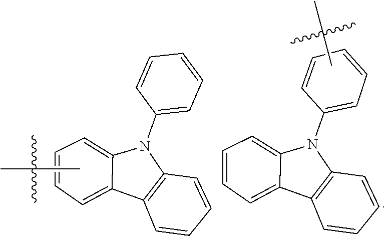

In some embodiments,

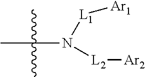

in Formula 1 is selected from the group consisting of the following groups:

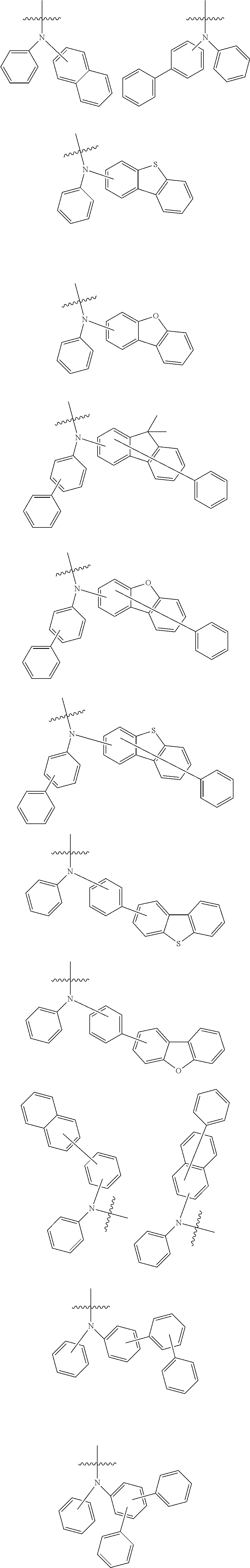

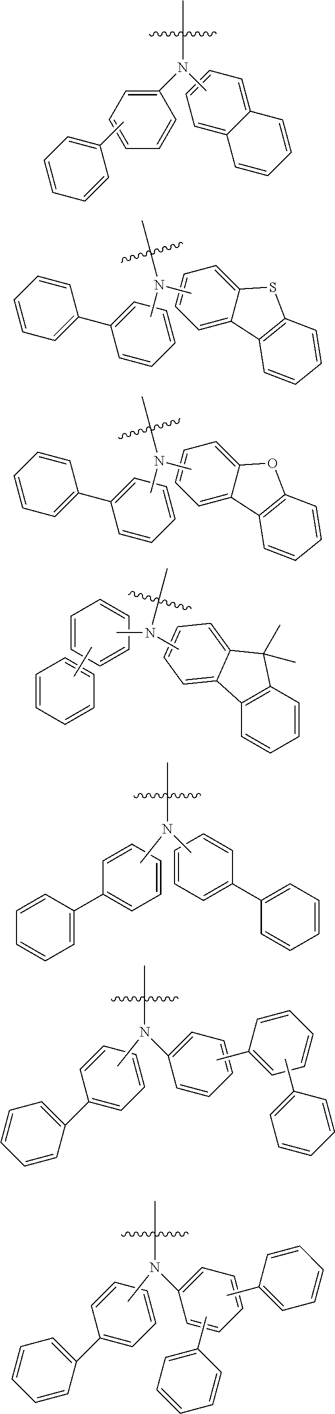

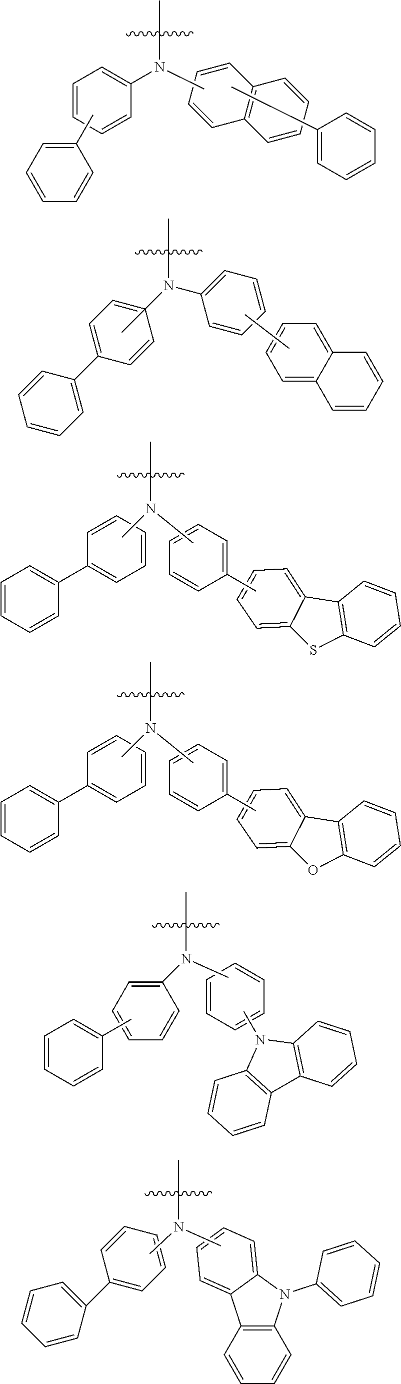

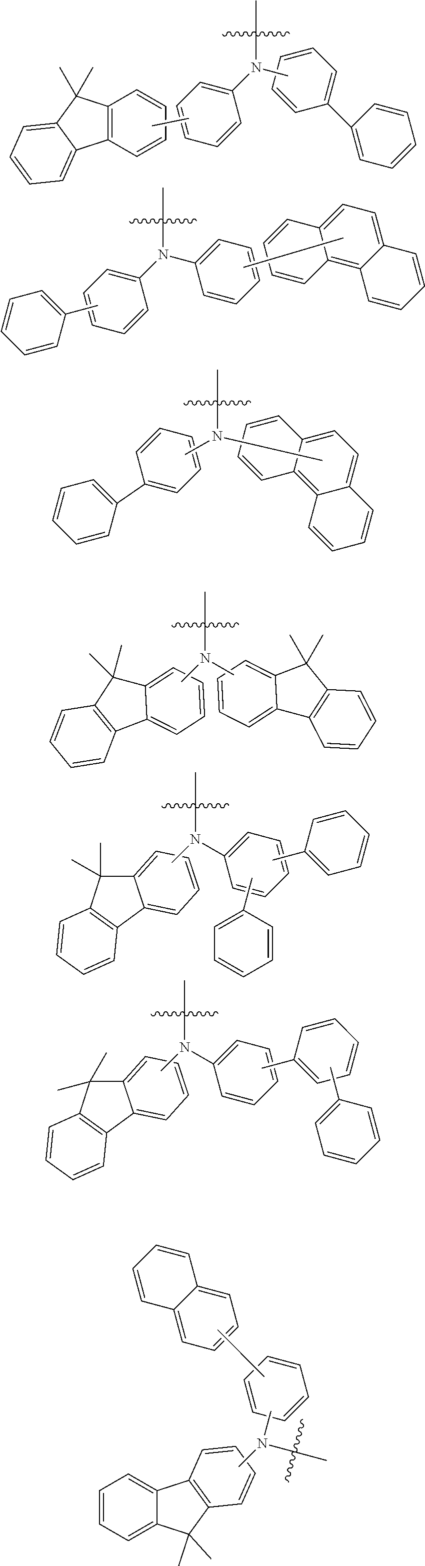

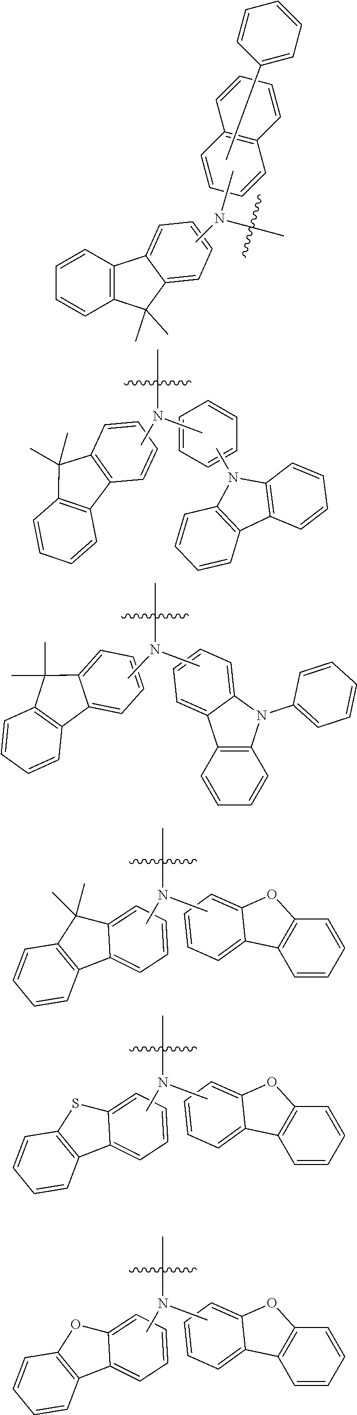

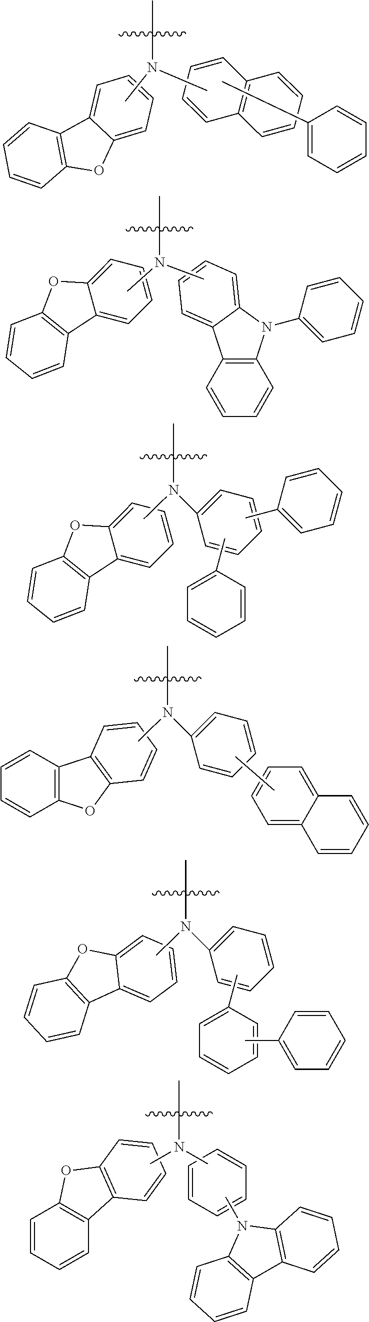

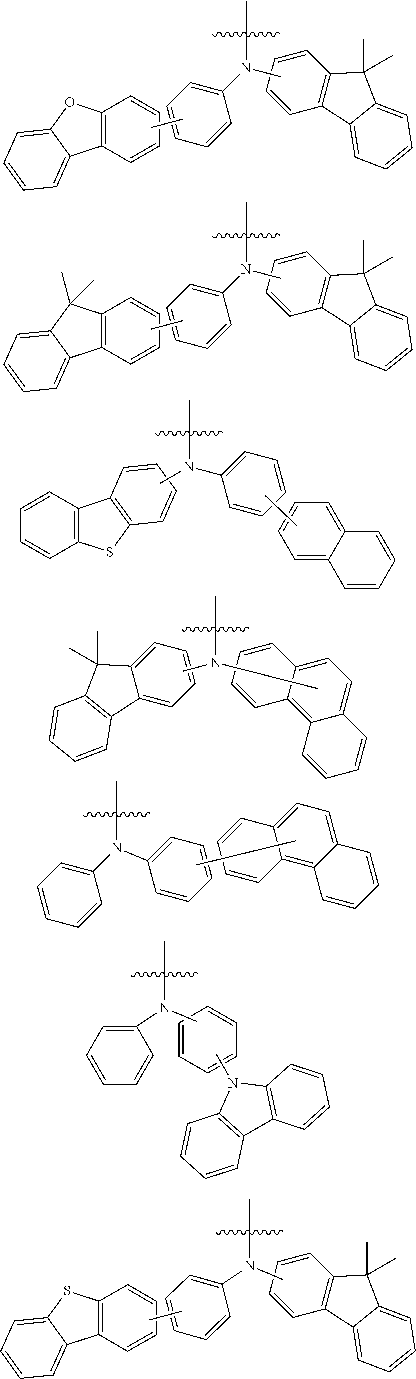

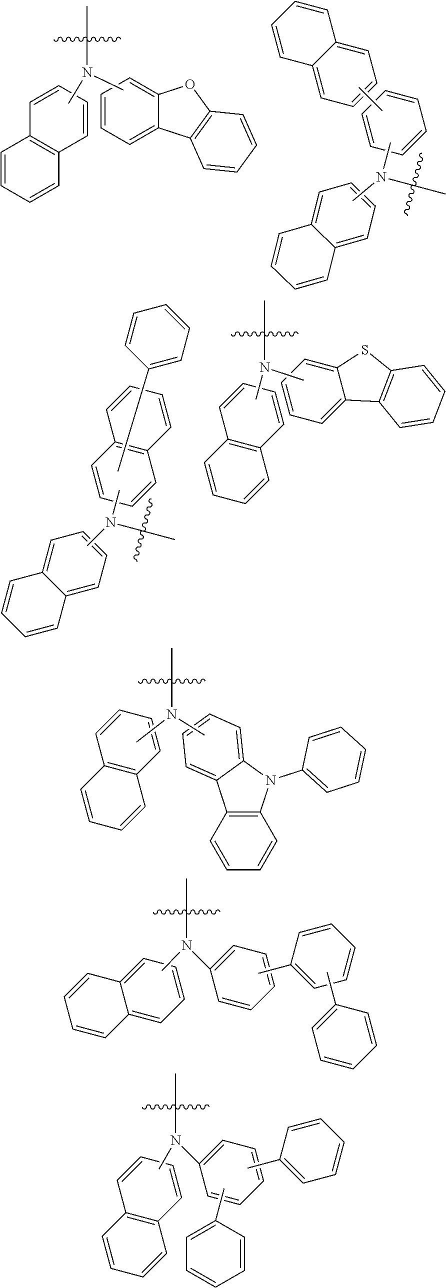

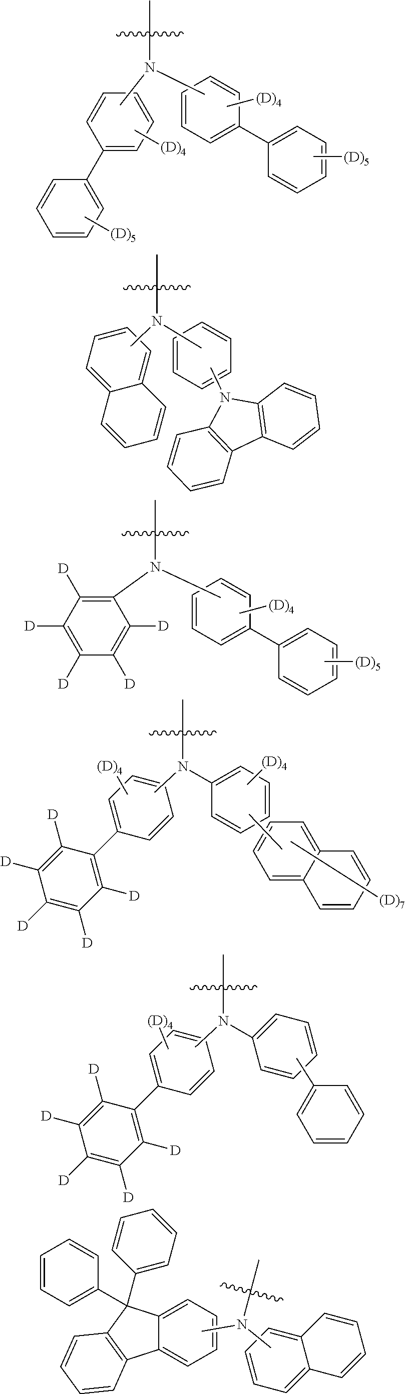

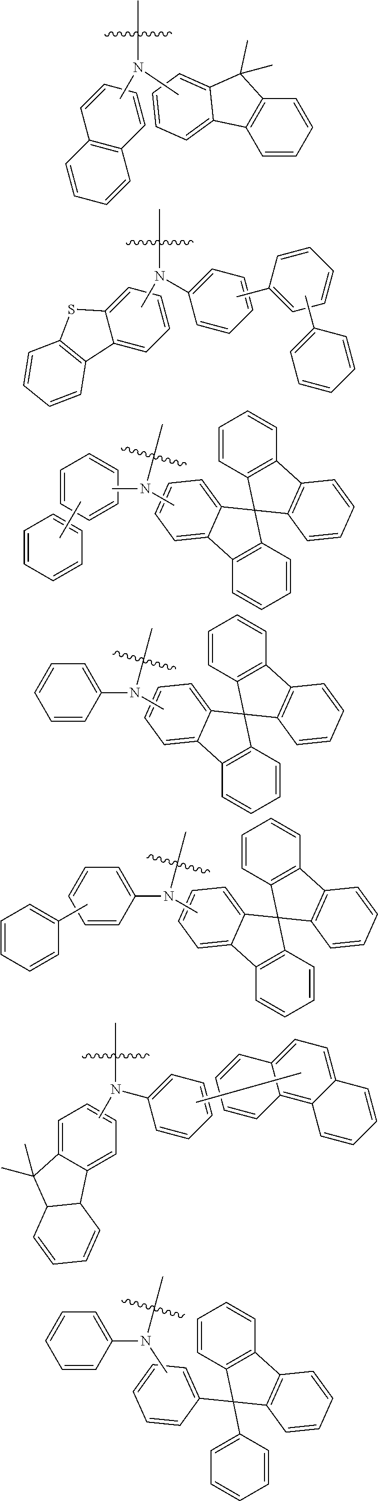

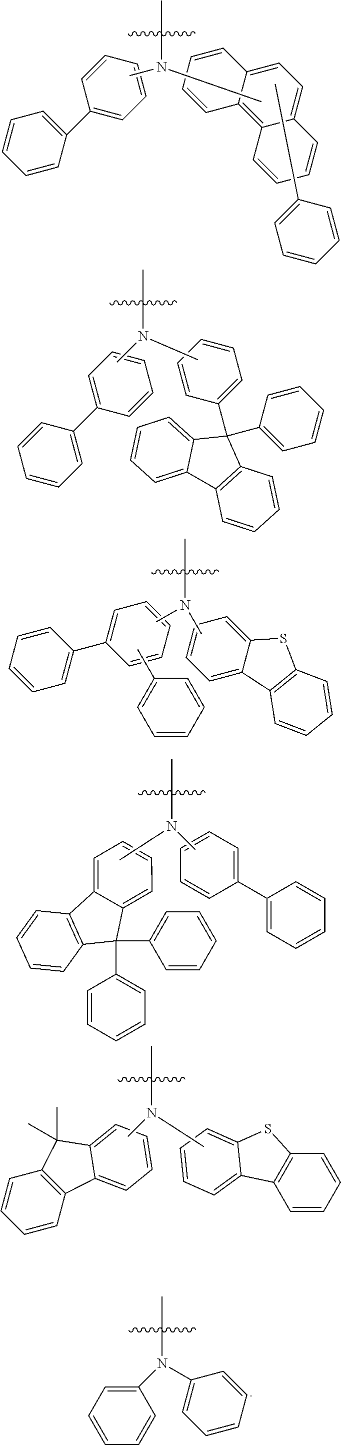

In some embodiments, the arylamine compound is selected from the group consisting of the following compounds:

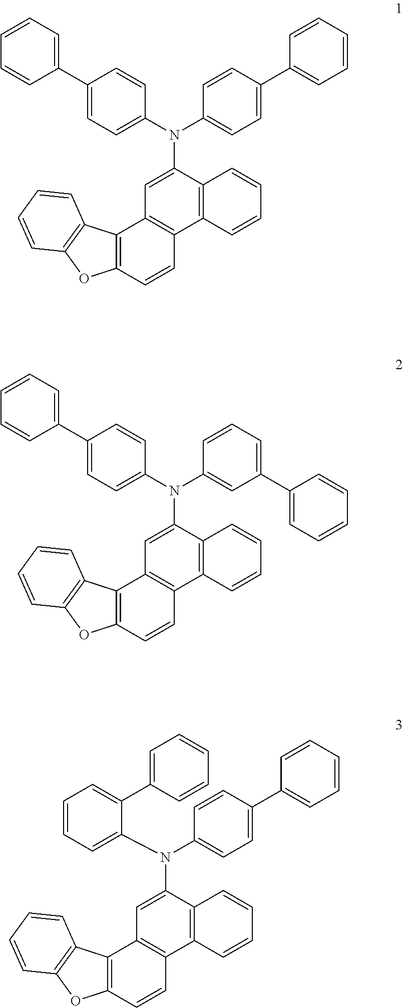 [SMILES: c1ccc(-c2ccc(N(c3ccc(-c4ccccc4)cc3)c3cc4c(ccc5oc6ccccc6c54)c4ccccc34)cc2)cc1 ; c1ccc(-c2ccc(N(c3cccc(-c4ccccc4)c3)c3cc4c(ccc5oc6ccccc6c54)c4ccccc34)cc2)cc1 ; c1ccc(-c2ccc(N(c3ccccc3-c3ccccc3)c3cc4c(ccc5oc6ccccc6c54)c4ccccc34)cc2)cc1]

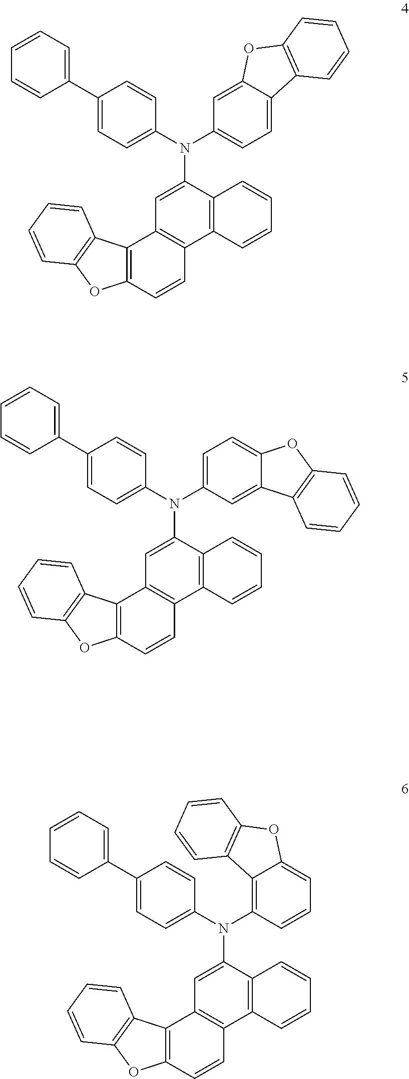 [SMILES: c1ccc(-c2ccc(N(c3ccc4c(c3)oc3ccccc34)c3cc4c(ccc5oc6ccccc6c54)c4ccccc34)cc2)cc1 ; c1ccc(-c2ccc(N(c3ccc4oc5ccccc5c4c3)c3cc4c(ccc5oc6ccccc6c54)c4ccccc34)cc2)cc1 ; c1ccc(-c2ccc(N(c3cc4c(ccc5oc6ccccc6c54)c4ccccc34)c3cccc4oc5ccccc5c34)cc2)cc1]

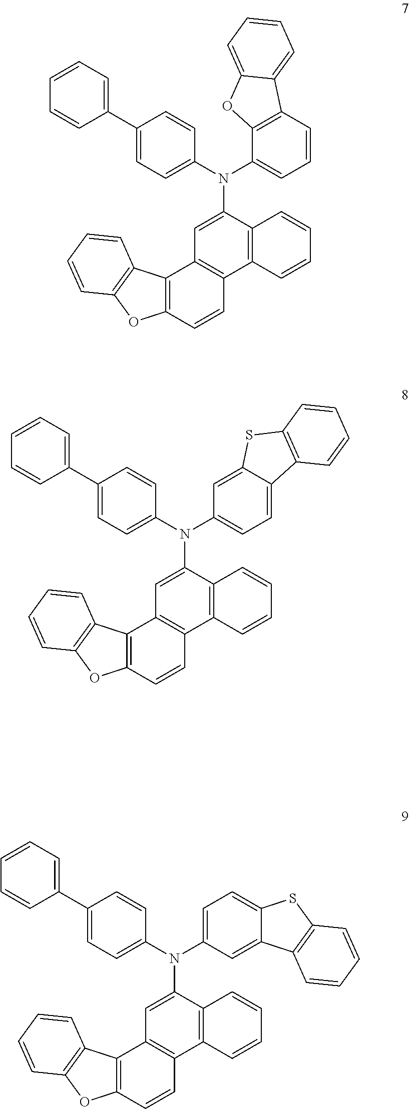 [SMILES: c1ccc(-c2ccc(N(c3cc4c(ccc5oc6ccccc6c54)c4ccccc34)c3cccc4c3oc3ccccc34)cc2)cc1 ; c1ccc(-c2ccc(N(c3ccc4c(c3)sc3ccccc34)c3cc4c(ccc5oc6ccccc6c54)c4ccccc34)cc2)cc1 ; c1ccc(-c2ccc(N(c3ccc4sc5ccccc5c4c3)c3cc4c(ccc5oc6ccccc6c54)c4ccccc34)cc2)cc1]

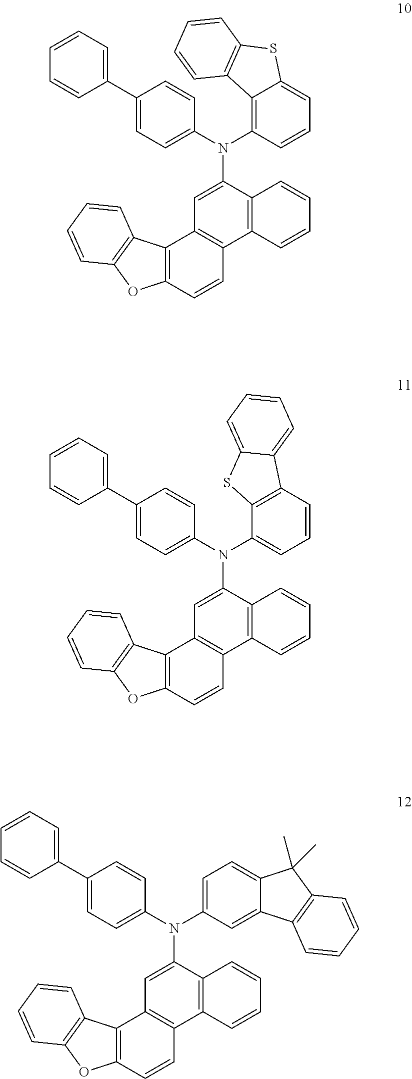 [SMILES: c1ccc(-c2ccc(N(c3cc4c(ccc5oc6ccccc6c54)c4ccccc34)c3cccc4sc5ccccc5c34)cc2)cc1 ; c1ccc(-c2ccc(N(c3cc4c(ccc5oc6ccccc6c54)c4ccccc34)c3cccc4c3sc3ccccc34)cc2)cc1 ; CC1(C)c2ccccc2-c2cc(N(c3ccc(-c4ccccc4)cc3)c3cc4c(ccc5oc6ccccc6c54)c4ccccc34)ccc21]

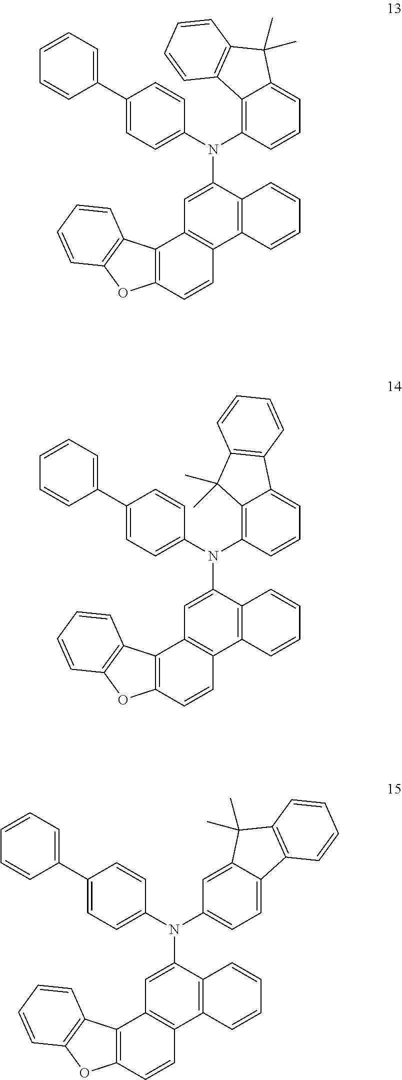 [SMILES: CC1(C)c2ccccc2-c2c(N(c3ccc(-c4ccccc4)cc3)c3cc4c(ccc5oc6ccccc6c54)c4ccccc34)cccc21 ; CC1(C)c2ccccc2-c2cccc(N(c3ccc(-c4ccccc4)cc3)c3cc4c(ccc5oc6ccccc6c54)c4ccccc34)c21 ; CC1(C)c2ccccc2-c2ccc(N(c3ccc(-c4ccccc4)cc3)c3cc4c(ccc5oc6ccccc6c54)c4ccccc34)cc21]

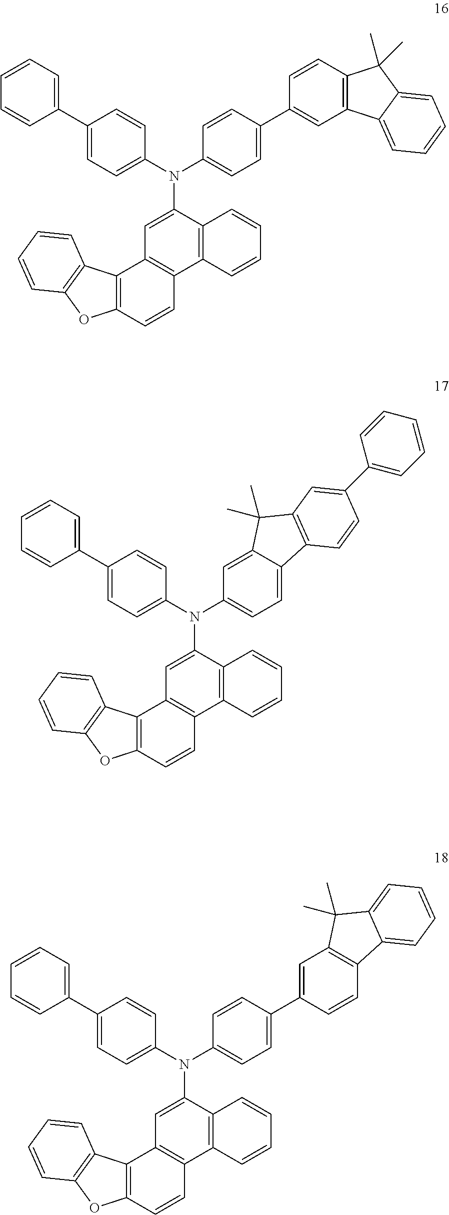 [SMILES: CC1(C)c2ccccc2-c2cc(-c3ccc(N(c4ccc(-c5ccccc5)cc4)c4cc5c(ccc6oc7ccccc7c65)c5ccccc45)cc3)ccc21 ; CC1(C)c2cc(-c3ccccc3)ccc2-c2ccc(N(c3ccc(-c4ccccc4)cc3)c3cc4c(ccc5oc6ccccc6c54)c4ccccc34)cc21 ; CC1(C)c2ccccc2-c2ccc(-c3ccc(N(c4ccc(-c5ccccc5)cc4)c4cc5c(ccc6oc7ccccc7c65)c5ccccc45)cc3)cc21]

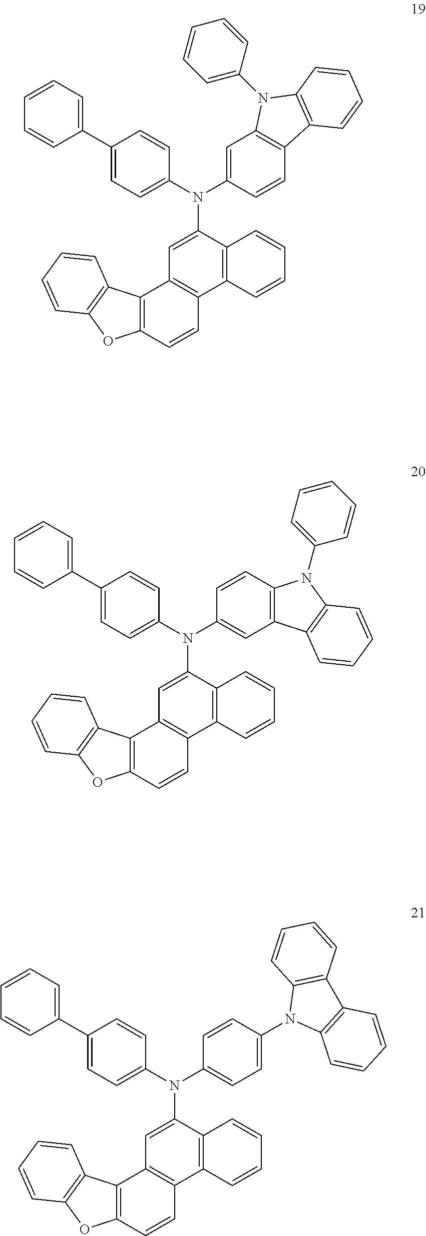 [SMILES: c1ccc(-c2ccc(N(c3ccc4c5ccccc5n(-c5ccccc5)c4c3)c3cc4c(ccc5oc6ccccc6c54)c4ccccc34)cc2)cc1 ; c1ccc(-c2ccc(N(c3ccc4c(c3)c3ccccc3n4-c3ccccc3)c3cc4c(ccc5oc6ccccc6c54)c4ccccc34)cc2)cc1 ; c1ccc(-c2ccc(N(c3ccc(-n4c5ccccc5c5ccccc54)cc3)c3cc4c(ccc5oc6ccccc6c54)c4ccccc34)cc2)cc1]

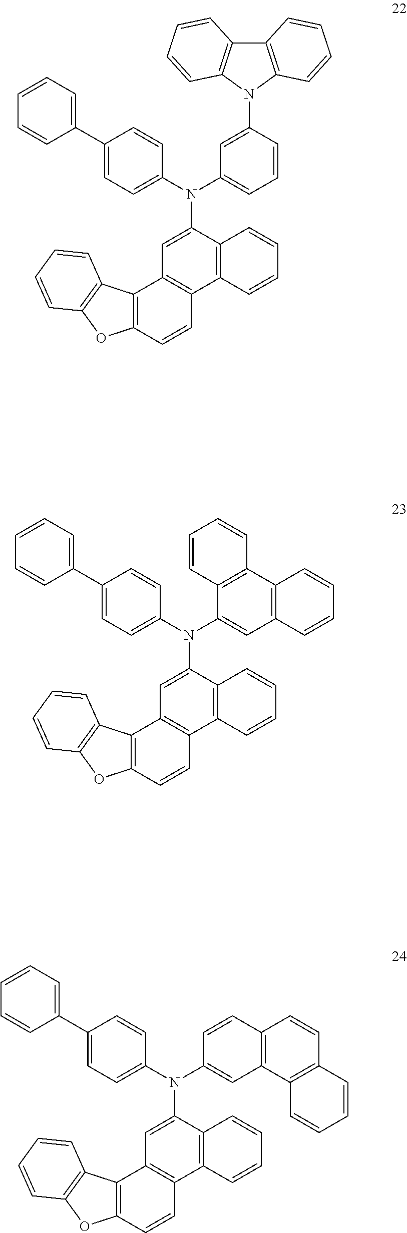 [SMILES: c1ccc(-c2ccc(N(c3cccc(-n4c5ccccc5c5ccccc54)c3)c3cc4c(ccc5oc6ccccc6c54)c4ccccc34)cc2)cc1 ; c1ccc(-c2ccc(N(c3cc4ccccc4c4ccccc34)c3cc4c(ccc5oc6ccccc6c54)c4ccccc34)cc2)cc1 ; c1ccc(-c2ccc(N(c3ccc4ccc5ccccc5c4c3)c3cc4c(ccc5oc6ccccc6c54)c4ccccc34)cc2)cc1]

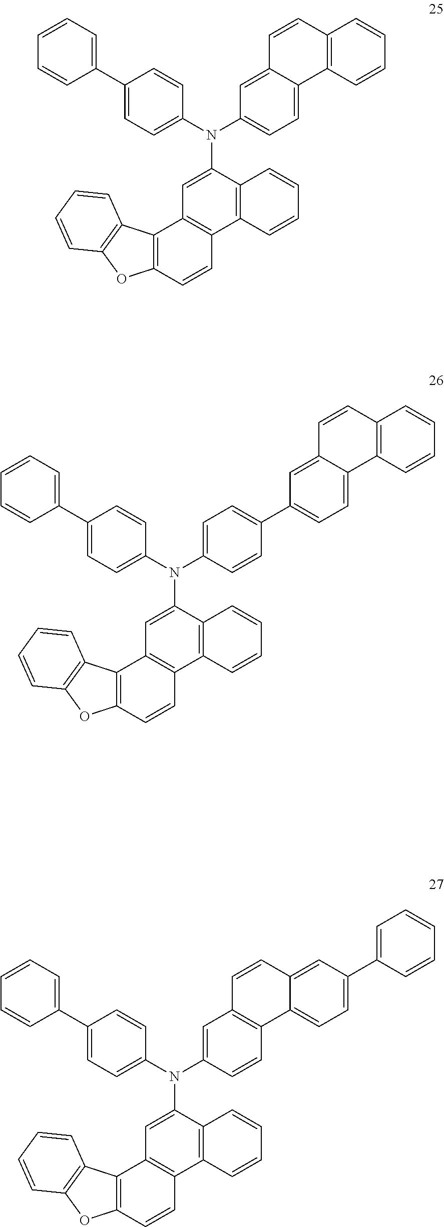 [SMILES: c1ccc(-c2ccc(N(c3ccc4c(ccc5ccccc54)c3)c3cc4c(ccc5oc6ccccc6c54)c4ccccc34)cc2)cc1 ; c1ccc(-c2ccc(N(c3ccc(-c4ccc5c(ccc6ccccc65)c4)cc3)c3cc4c(ccc5oc6ccccc6c54)c4ccccc34)cc2)cc1 ; c1ccc(-c2ccc(N(c3ccc4c(ccc5cc(-c6ccccc6)ccc54)c3)c3cc4c(ccc5oc6ccccc6c54)c4ccccc34)cc2)cc1]

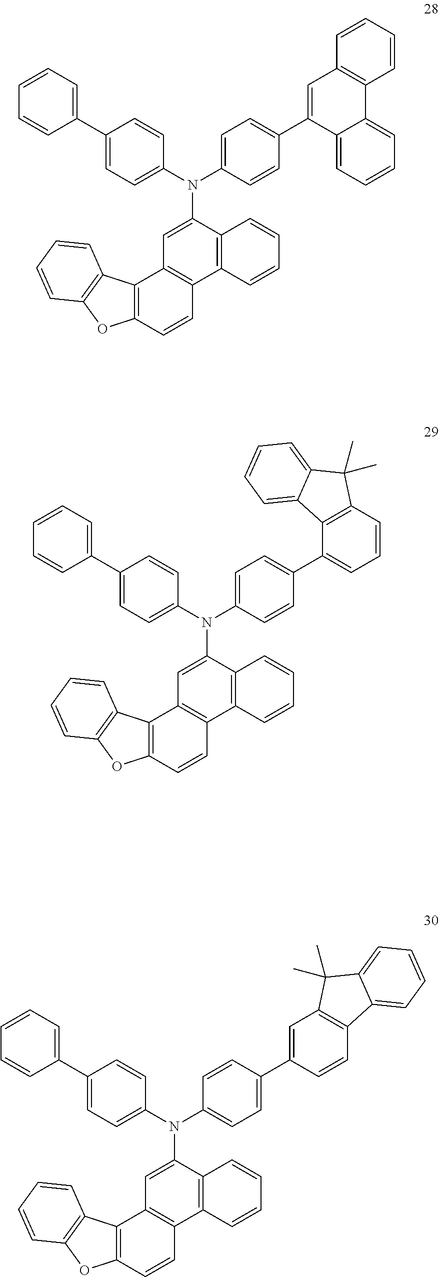 [SMILES: c1ccc(-c2ccc(N(c3ccc(-c4cc5ccccc5c5ccccc45)cc3)c3cc4c(ccc5oc6ccccc6c54)c4ccccc34)cc2)cc1 ; CC1(C)c2ccccc2-c2c(-c3ccc(N(c4ccc(-c5ccccc5)cc4)c4cc5c(ccc6oc7ccccc7c65)c5ccccc45)cc3)cccc21 ; CC1(C)c2ccccc2-c2ccc(-c3ccc(N(c4ccc(-c5ccccc5)cc4)c4cc5c(ccc6oc7ccccc7c65)c5ccccc45)cc3)cc21]

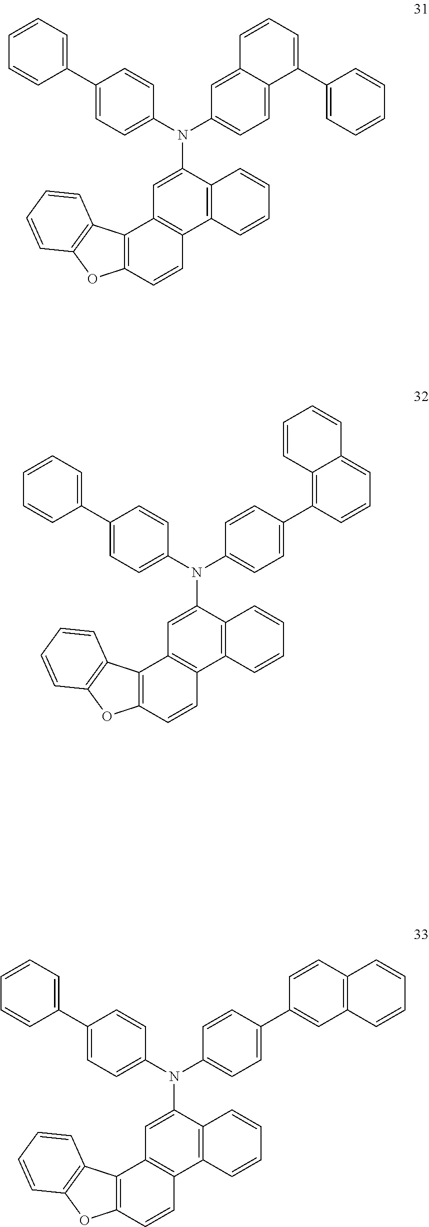 [SMILES: c1ccc(-c2ccc(N(c3ccc4c(-c5ccccc5)cccc4c3)c3cc4c(ccc5oc6ccccc6c54)c4ccccc34)cc2)cc1 ; c1ccc(-c2ccc(N(c3ccc(-c4cccc5ccccc45)cc3)c3cc4c(ccc5oc6ccccc6c54)c4ccccc34)cc2)cc1 ; c1ccc(-c2ccc(N(c3ccc(-c4ccc5ccccc5c4)cc3)c3cc4c(ccc5oc6ccccc6c54)c4ccccc34)cc2)cc1]

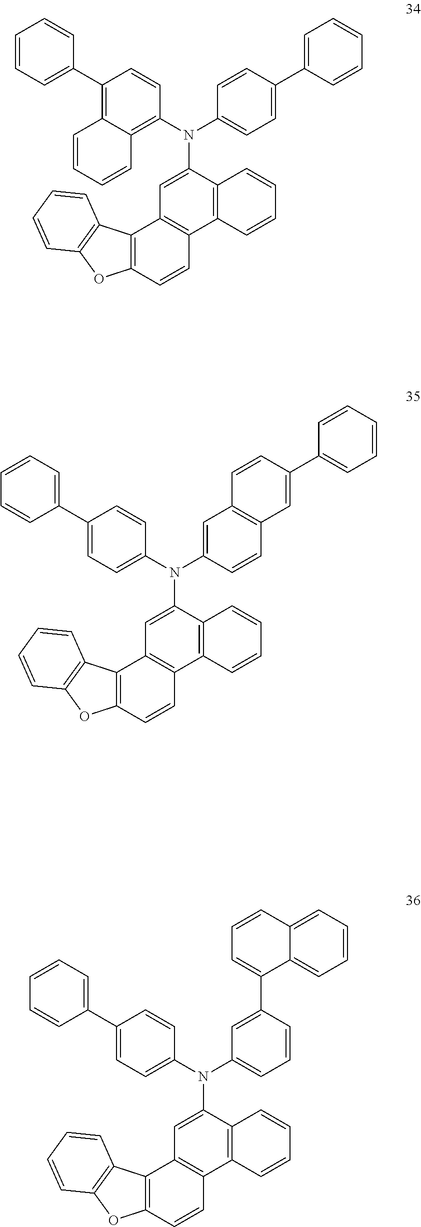 [SMILES: c1ccc(-c2ccc(N(c3ccc(-c4ccccc4)c4ccccc34)c3cc4c(ccc5oc6ccccc6c54)c4ccccc34)cc2)cc1 ; c1ccc(-c2ccc(N(c3ccc4cc(-c5ccccc5)ccc4c3)c3cc4c(ccc5oc6ccccc6c54)c4ccccc34)cc2)cc1 ; c1ccc(-c2ccc(N(c3cccc(-c4cccc5ccccc45)c3)c3cc4c(ccc5oc6ccccc6c54)c4ccccc34)cc2)cc1]

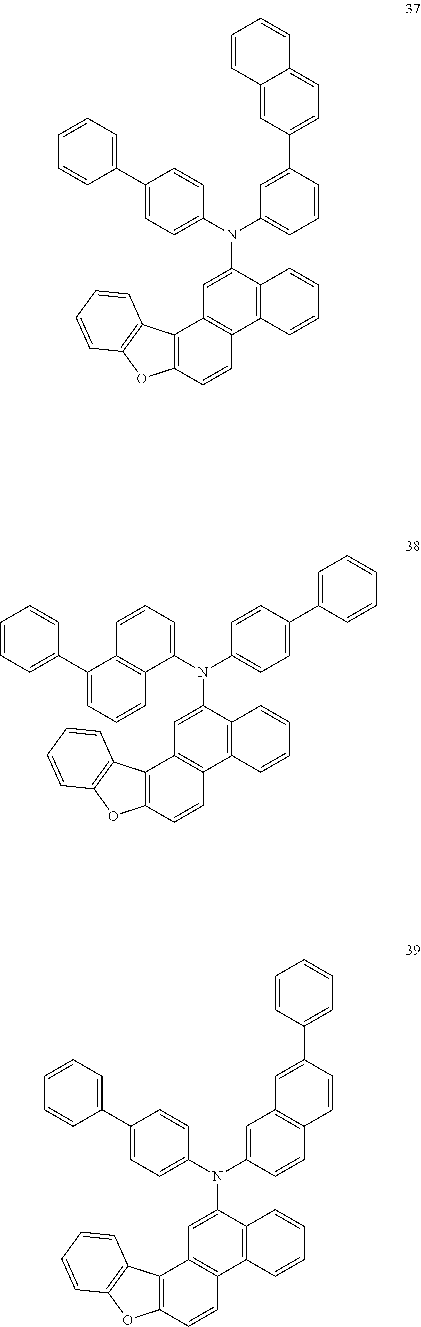 [SMILES: c1ccc(-c2ccc(N(c3cccc(-c4ccc5ccccc5c4)c3)c3cc4c(ccc5oc6ccccc6c54)c4ccccc34)cc2)cc1 ; c1ccc(-c2ccc(N(c3cccc4c(-c5ccccc5)cccc34)c3cc4c(ccc5oc6ccccc6c54)c4ccccc34)cc2)cc1 ; c1ccc(-c2ccc(N(c3ccc4ccc(-c5ccccc5)cc4c3)c3cc4c(ccc5oc6ccccc6c54)c4ccccc34)cc2)cc1]

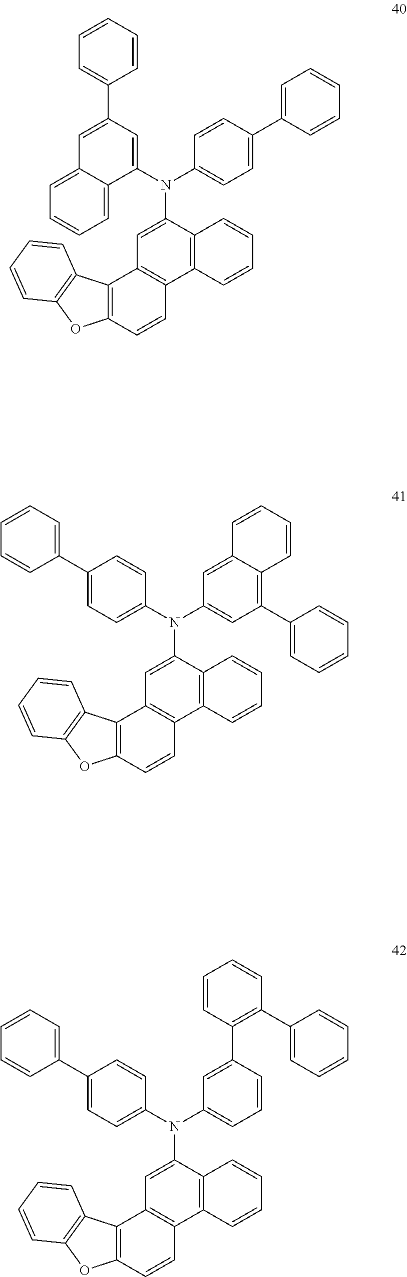 [SMILES: c1ccc(-c2ccc(N(c3cc(-c4ccccc4)cc4ccccc34)c3cc4c(ccc5oc6ccccc6c54)c4ccccc34)cc2)cc1 ; c1ccc(-c2ccc(N(c3cc(-c4ccccc4)c4ccccc4c3)c3cc4c(ccc5oc6ccccc6c54)c4ccccc34)cc2)cc1 ; c1ccc(-c2ccc(N(c3cccc(-c4ccccc4-c4ccccc4)c3)c3cc4c(ccc5oc6ccccc6c54)c4ccccc34)cc2)cc1]

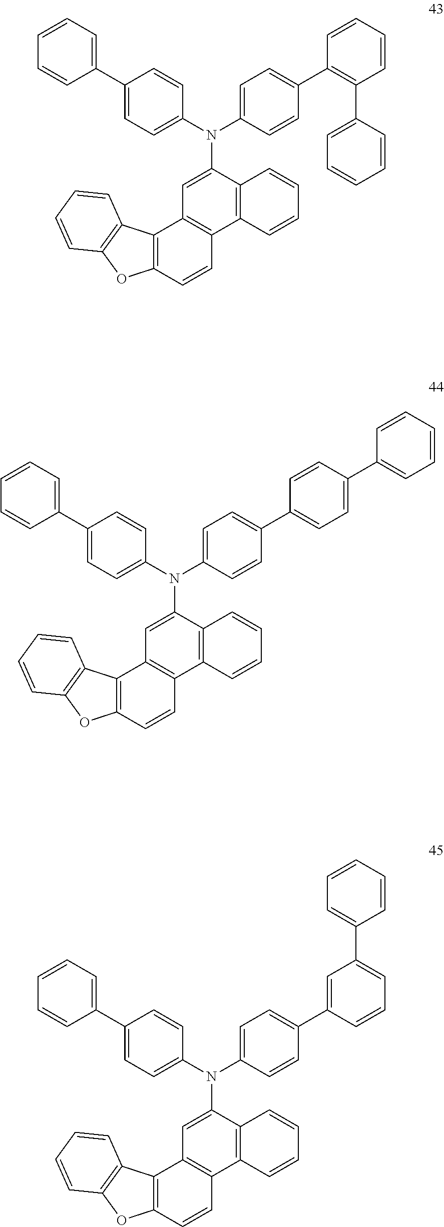 [SMILES: c1ccc(-c2ccc(N(c3ccc(-c4ccccc4-c4ccccc4)cc3)c3cc4c(ccc5oc6ccccc6c54)c4ccccc34)cc2)cc1 ; c1ccc(-c2ccc(-c3ccc(N(c4ccc(-c5ccccc5)cc4)c4cc5c(ccc6oc7ccccc7c65)c5ccccc45)cc3)cc2)cc1 ; c1ccc(-c2ccc(N(c3ccc(-c4cccc(-c5ccccc5)c4)cc3)c3cc4c(ccc5oc6ccccc6c54)c4ccccc34)cc2)cc1]

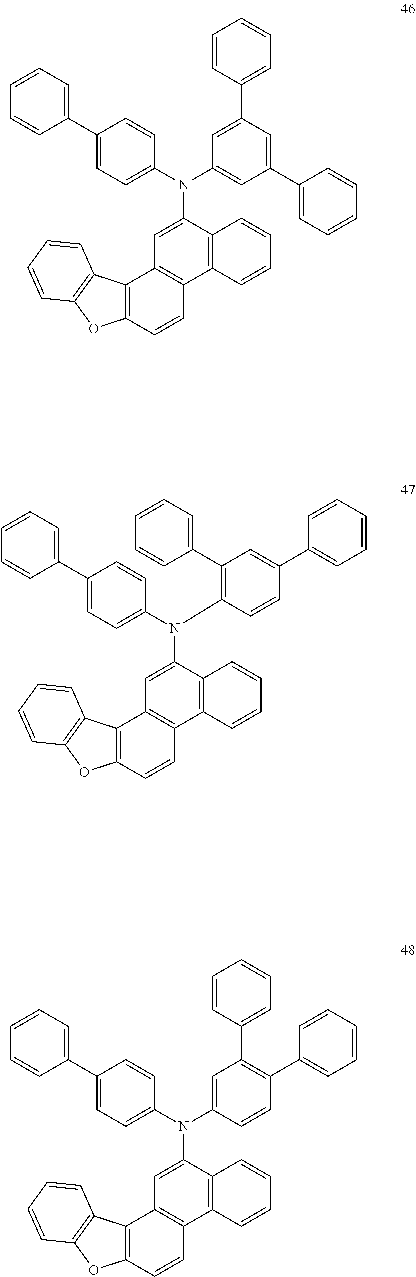 [SMILES: c1ccc(-c2ccc(N(c3cc(-c4ccccc4)cc(-c4ccccc4)c3)c3cc4c(ccc5oc6ccccc6c54)c4ccccc34)cc2)cc1 ; c1ccc(-c2ccc(N(c3ccc(-c4ccccc4)cc3-c3ccccc3)c3cc4c(ccc5oc6ccccc6c54)c4ccccc34)cc2)cc1 ; c1ccc(-c2ccc(N(c3ccc(-c4ccccc4)c(-c4ccccc4)c3)c3cc4c(ccc5oc6ccccc6c54)c4ccccc34)cc2)cc1]

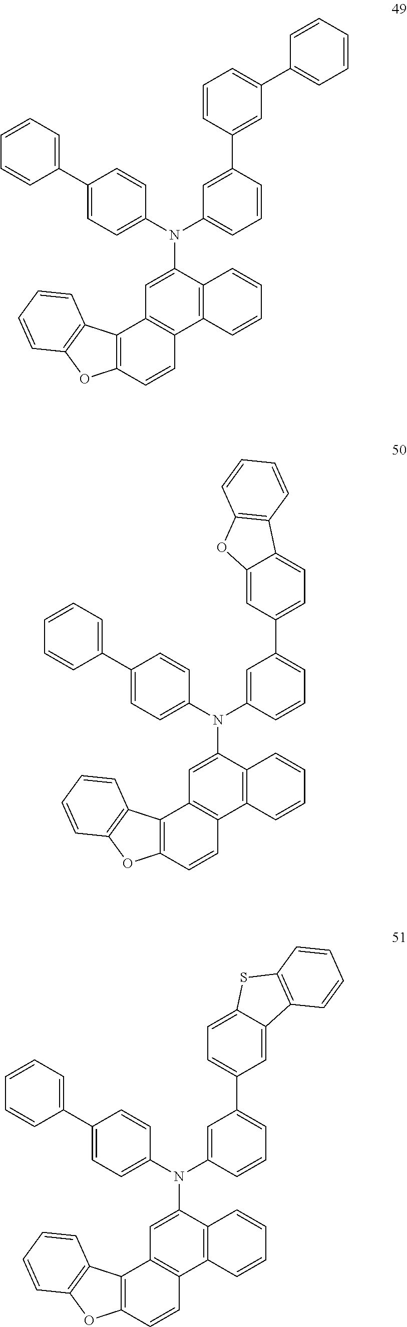 [SMILES: c1ccc(-c2ccc(N(c3cccc(-c4cccc(-c5ccccc5)c4)c3)c3cc4c(ccc5oc6ccccc6c54)c4ccccc34)cc2)cc1 ; c1ccc(-c2ccc(N(c3cccc(-c4ccc5c(c4)oc4ccccc45)c3)c3cc4c(ccc5oc6ccccc6c54)c4ccccc34)cc2)cc1 ; c1ccc(-c2ccc(N(c3cccc(-c4ccc5sc6ccccc6c5c4)c3)c3cc4c(ccc5oc6ccccc6c54)c4ccccc34)cc2)cc1]

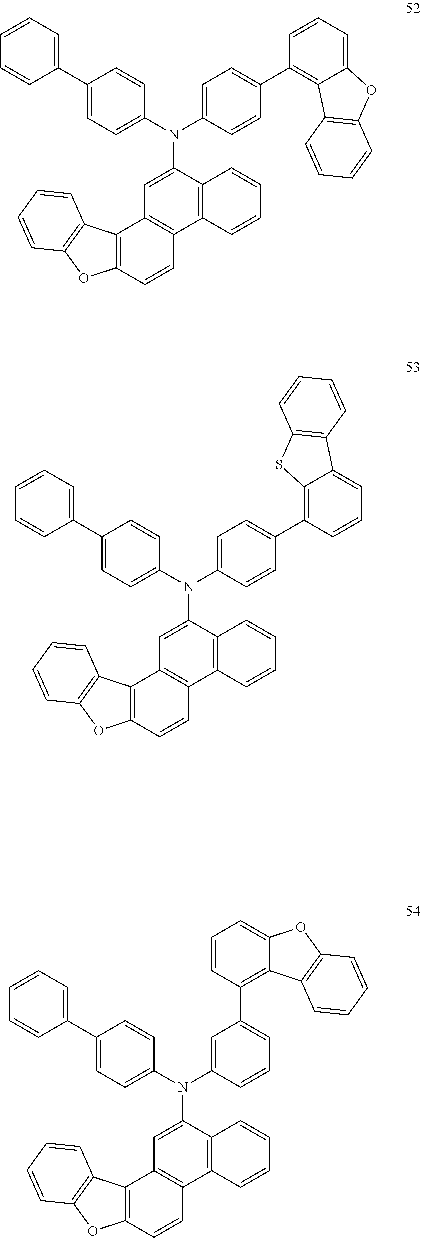 [SMILES: c1ccc(-c2ccc(N(c3ccc(-c4cccc5oc6ccccc6c45)cc3)c3cc4c(ccc5oc6ccccc6c54)c4ccccc34)cc2)cc1 ; c1ccc(-c2ccc(N(c3ccc(-c4cccc5c4sc4ccccc45)cc3)c3cc4c(ccc5oc6ccccc6c54)c4ccccc34)cc2)cc1 ; c1ccc(-c2ccc(N(c3cccc(-c4cccc5oc6ccccc6c45)c3)c3cc4c(ccc5oc6ccccc6c54)c4ccccc34)cc2)cc1]

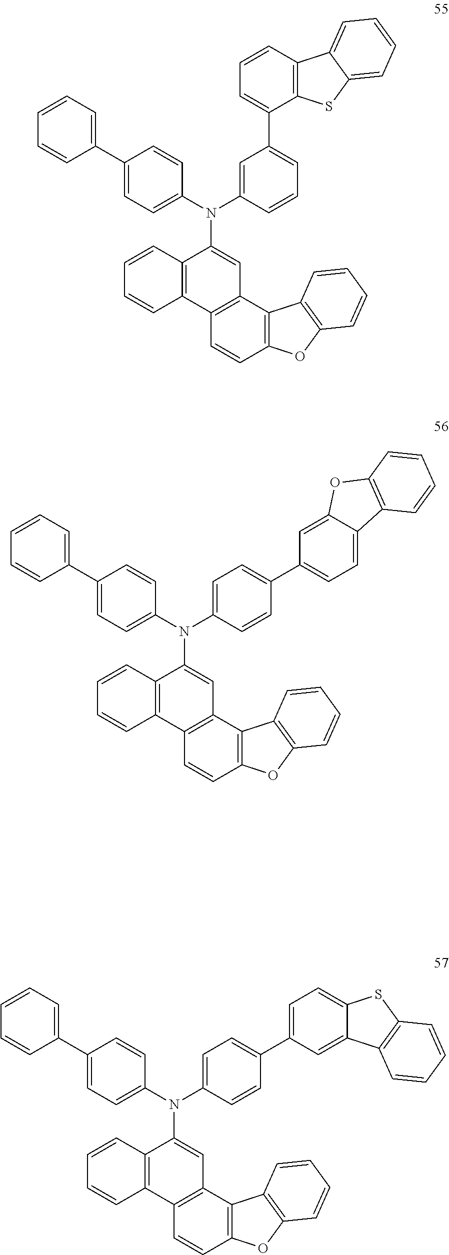 [SMILES: c1ccc(-c2ccc(N(c3cccc(-c4cccc5c4sc4ccccc45)c3)c3cc4c(ccc5oc6ccccc6c54)c4ccccc34)cc2)cc1 ; c1ccc(-c2ccc(N(c3ccc(-c4ccc5c(c4)oc4ccccc45)cc3)c3cc4c(ccc5oc6ccccc6c54)c4ccccc34)cc2)cc1 ; c1ccc(-c2ccc(N(c3ccc(-c4ccc5sc6ccccc6c5c4)cc3)c3cc4c(ccc5oc6ccccc6c54)c4ccccc34)cc2)cc1]

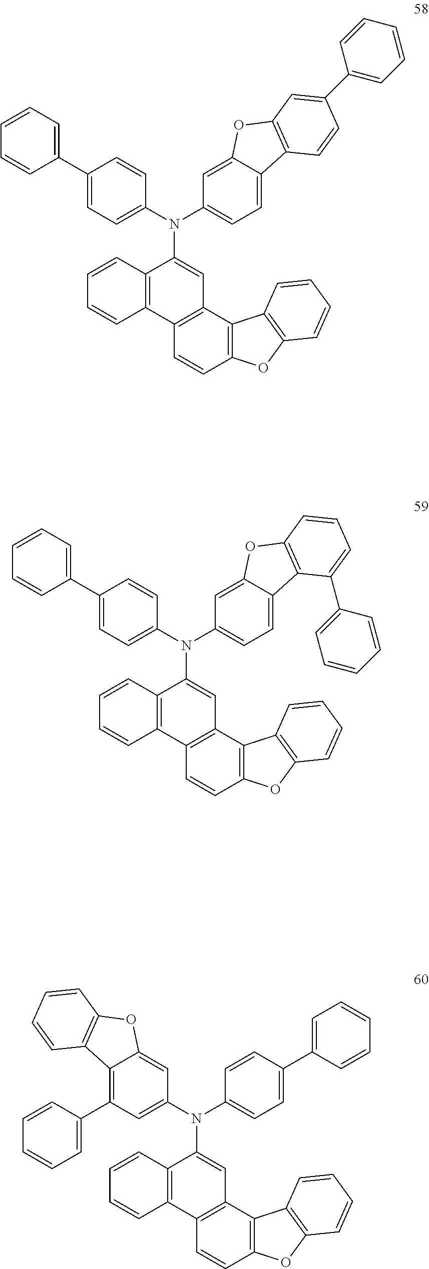 [SMILES: c1ccc(-c2ccc(N(c3ccc4c(c3)oc3cc(-c5ccccc5)ccc34)c3cc4c(ccc5oc6ccccc6c54)c4ccccc34)cc2)cc1 ; c1ccc(-c2ccc(N(c3ccc4c(c3)oc3cccc(-c5ccccc5)c34)c3cc4c(ccc5oc6ccccc6c54)c4ccccc34)cc2)cc1 ; c1ccc(-c2ccc(N(c3cc(-c4ccccc4)c4c(c3)oc3ccccc34)c3cc4c(ccc5oc6ccccc6c54)c4ccccc34)cc2)cc1]

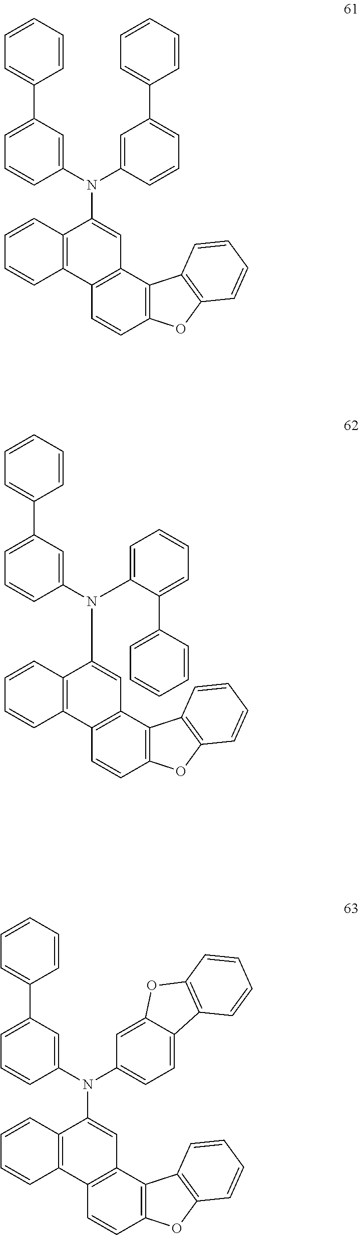 [SMILES: c1ccc(-c2cccc(N(c3cccc(-c4ccccc4)c3)c3cc4c(ccc5oc6ccccc6c54)c4ccccc34)c2)cc1 ; c1ccc(-c2cccc(N(c3ccccc3-c3ccccc3)c3cc4c(ccc5oc6ccccc6c54)c4ccccc34)c2)cc1 ; c1ccc(-c2cccc(N(c3ccc4c(c3)oc3ccccc34)c3cc4c(ccc5oc6ccccc6c54)c4ccccc34)c2)cc1]

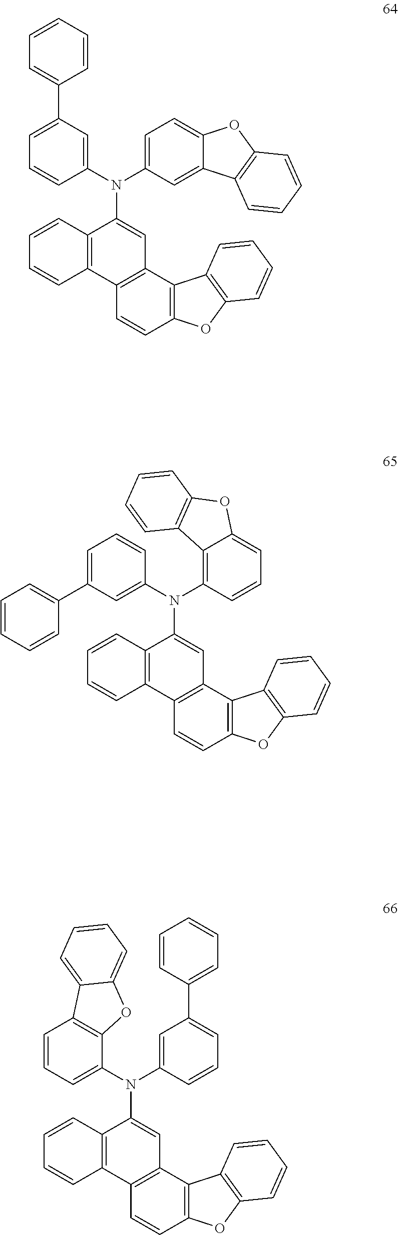 [SMILES: c1ccc(-c2cccc(N(c3ccc4oc5ccccc5c4c3)c3cc4c(ccc5oc6ccccc6c54)c4ccccc34)c2)cc1 ; c1ccc(-c2cccc(N(c3cc4c(ccc5oc6ccccc6c54)c4ccccc34)c3cccc4oc5ccccc5c34)c2)cc1 ; c1ccc(-c2cccc(N(c3cc4c(ccc5oc6ccccc6c54)c4ccccc34)c3cccc4c3oc3ccccc34)c2)cc1]

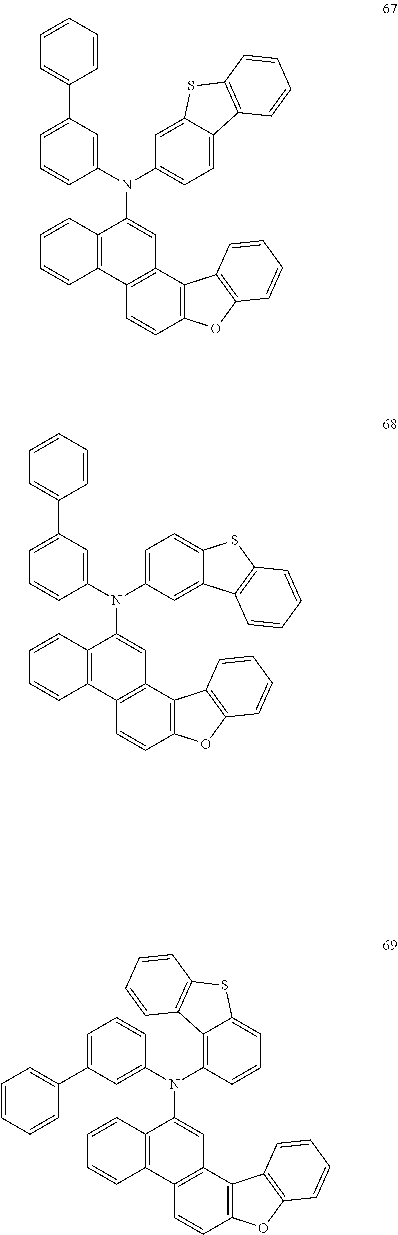 [SMILES: c1ccc(-c2cccc(N(c3ccc4c(c3)sc3ccccc34)c3cc4c(ccc5oc6ccccc6c54)c4ccccc34)c2)cc1 ; c1ccc(-c2cccc(N(c3ccc4sc5ccccc5c4c3)c3cc4c(ccc5oc6ccccc6c54)c4ccccc34)c2)cc1 ; c1ccc(-c2cccc(N(c3cc4c(ccc5oc6ccccc6c54)c4ccccc34)c3cccc4sc5ccccc5c34)c2)cc1]

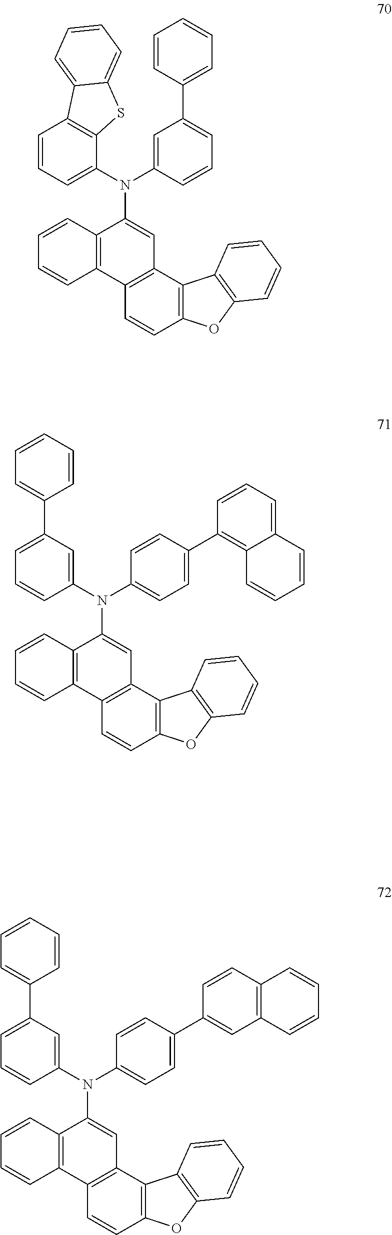 [SMILES: c1ccc(-c2cccc(N(c3cc4c(ccc5oc6ccccc6c54)c4ccccc34)c3cccc4c3sc3ccccc34)c2)cc1 ; c1ccc(-c2cccc(N(c3ccc(-c4cccc5ccccc45)cc3)c3cc4c(ccc5oc6ccccc6c54)c4ccccc34)c2)cc1 ; c1ccc(-c2cccc(N(c3ccc(-c4ccc5ccccc5c4)cc3)c3cc4c(ccc5oc6ccccc6c54)c4ccccc34)c2)cc1]

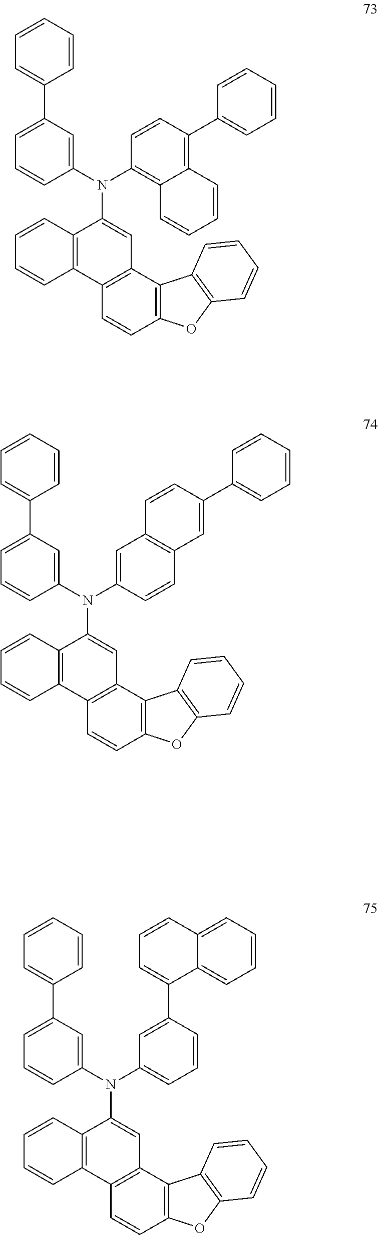 [SMILES: c1ccc(-c2cccc(N(c3ccc(-c4ccccc4)c4ccccc34)c3cc4c(ccc5oc6ccccc6c54)c4ccccc34)c2)cc1 ; c1ccc(-c2cccc(N(c3ccc4cc(-c5ccccc5)ccc4c3)c3cc4c(ccc5oc6ccccc6c54)c4ccccc34)c2)cc1 ; c1ccc(-c2cccc(N(c3cccc(-c4cccc5ccccc45)c3)c3cc4c(ccc5oc6ccccc6c54)c4ccccc34)c2)cc1]

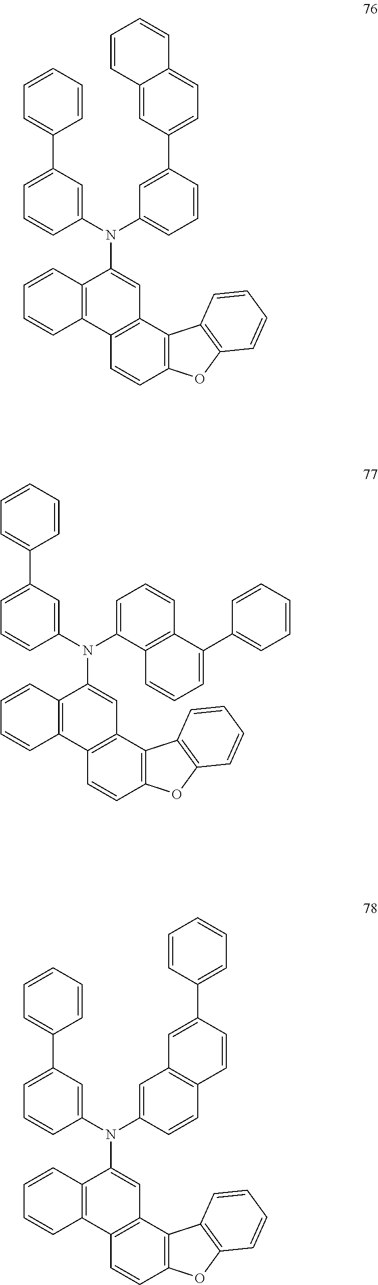 [SMILES: c1ccc(-c2cccc(N(c3cccc(-c4ccc5ccccc5c4)c3)c3cc4c(ccc5oc6ccccc6c54)c4ccccc34)c2)cc1 ; c1ccc(-c2cccc(N(c3cccc4c(-c5ccccc5)cccc34)c3cc4c(ccc5oc6ccccc6c54)c4ccccc34)c2)cc1 ; c1ccc(-c2cccc(N(c3ccc4ccc(-c5ccccc5)cc4c3)c3cc4c(ccc5oc6ccccc6c54)c4ccccc34)c2)cc1]

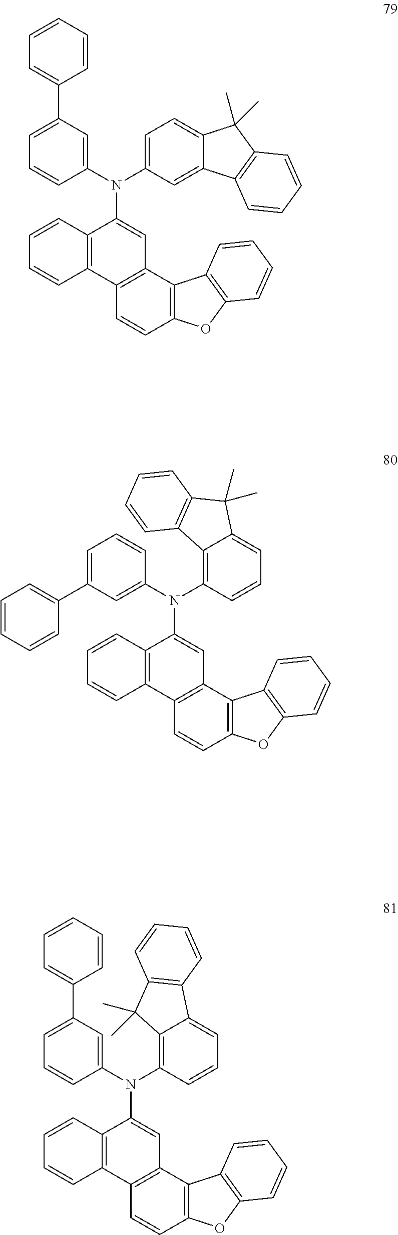 [SMILES: CC1(C)c2ccccc2-c2cc(N(c3cccc(-c4ccccc4)c3)c3cc4c(ccc5oc6ccccc6c54)c4ccccc34)ccc21 ; CC1(C)c2ccccc2-c2c(N(c3cccc(-c4ccccc4)c3)c3cc4c(ccc5oc6ccccc6c54)c4ccccc34)cccc21 ; CC1(C)c2ccccc2-c2cccc(N(c3cccc(-c4ccccc4)c3)c3cc4c(ccc5oc6ccccc6c54)c4ccccc34)c21]

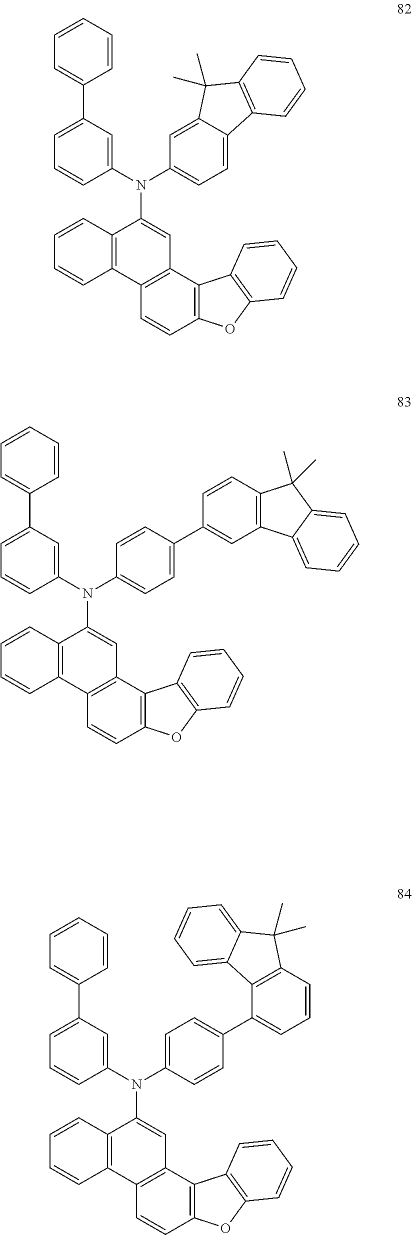 [SMILES: CC1(C)c2ccccc2-c2ccc(N(c3cccc(-c4ccccc4)c3)c3cc4c(ccc5oc6ccccc6c54)c4ccccc34)cc21 ; CC1(C)c2ccccc2-c2cc(-c3ccc(N(c4cccc(-c5ccccc5)c4)c4cc5c(ccc6oc7ccccc7c65)c5ccccc45)cc3)ccc21 ; CC1(C)c2ccccc2-c2c(-c3ccc(N(c4cccc(-c5ccccc5)c4)c4cc5c(ccc6oc7ccccc7c65)c5ccccc45)cc3)cccc21]

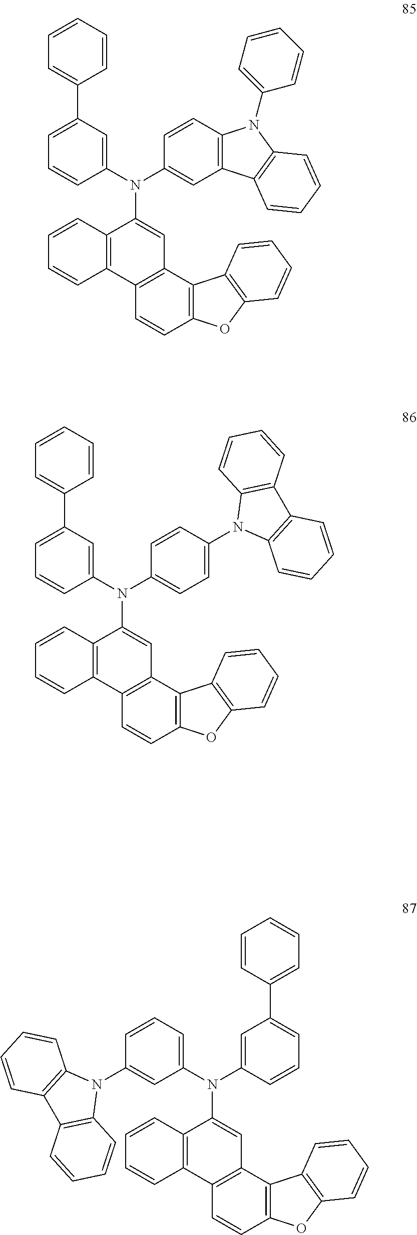 [SMILES: c1ccc(-c2cccc(N(c3ccc4c(c3)c3ccccc3n4-c3ccccc3)c3cc4c(ccc5oc6ccccc6c54)c4ccccc34)c2)cc1 ; c1ccc(-c2cccc(N(c3ccc(-n4c5ccccc5c5ccccc54)cc3)c3cc4c(ccc5oc6ccccc6c54)c4ccccc34)c2)cc1 ; c1ccc(-c2cccc(N(c3cccc(-n4c5ccccc5c5ccccc54)c3)c3cc4c(ccc5oc6ccccc6c54)c4ccccc34)c2)cc1]

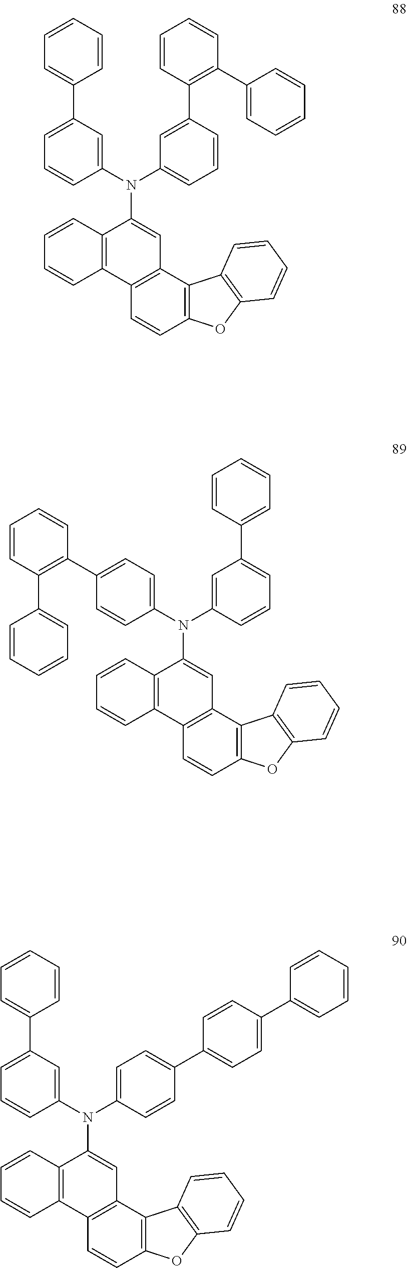 [SMILES: c1ccc(-c2cccc(N(c3cccc(-c4ccccc4-c4ccccc4)c3)c3cc4c(ccc5oc6ccccc6c54)c4ccccc34)c2)cc1 ; c1ccc(-c2cccc(N(c3ccc(-c4ccccc4-c4ccccc4)cc3)c3cc4c(ccc5oc6ccccc6c54)c4ccccc34)c2)cc1 ; c1ccc(-c2ccc(-c3ccc(N(c4cccc(-c5ccccc5)c4)c4cc5c(ccc6oc7ccccc7c65)c5ccccc45)cc3)cc2)cc1]

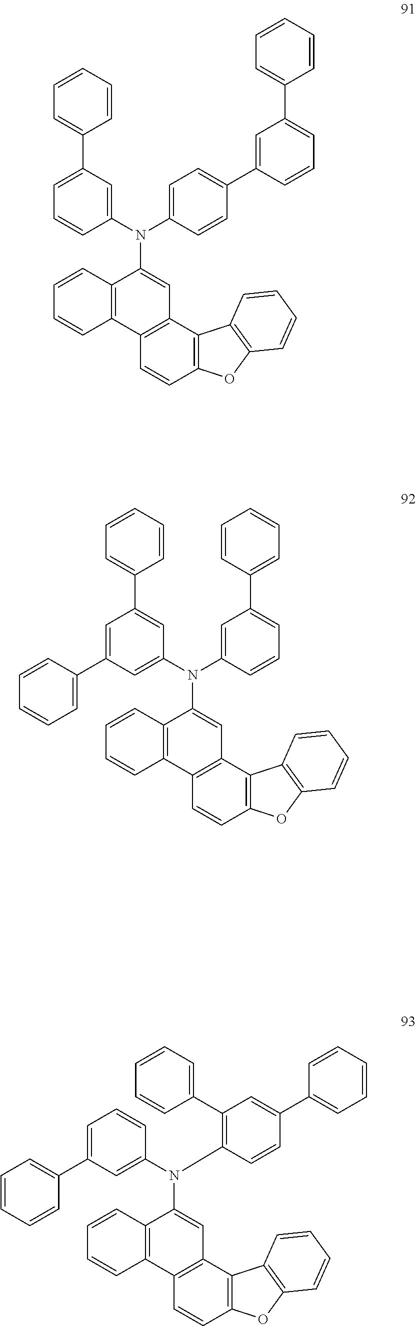 [SMILES: c1ccc(-c2cccc(-c3ccc(N(c4cccc(-c5ccccc5)c4)c4cc5c(ccc6oc7ccccc7c65)c5ccccc45)cc3)c2)cc1 ; c1ccc(-c2cccc(N(c3cc(-c4ccccc4)cc(-c4ccccc4)c3)c3cc4c(ccc5oc6ccccc6c54)c4ccccc34)c2)cc1 ; c1ccc(-c2cccc(N(c3ccc(-c4ccccc4)cc3-c3ccccc3)c3cc4c(ccc5oc6ccccc6c54)c4ccccc34)c2)cc1]

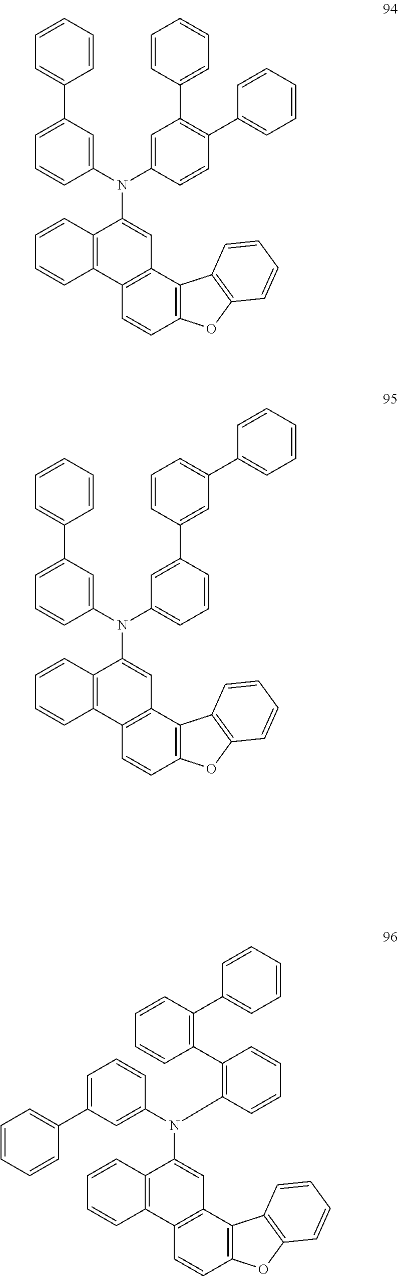 [SMILES: c1ccc(-c2cccc(N(c3ccc(-c4ccccc4)c(-c4ccccc4)c3)c3cc4c(ccc5oc6ccccc6c54)c4ccccc34)c2)cc1 ; c1ccc(-c2cccc(-c3cccc(N(c4cccc(-c5ccccc5)c4)c4cc5c(ccc6oc7ccccc7c65)c5ccccc45)c3)c2)cc1 ; c1ccc(-c2cccc(N(c3ccccc3-c3ccccc3-c3ccccc3)c3cc4c(ccc5oc6ccccc6c54)c4ccccc34)c2)cc1]

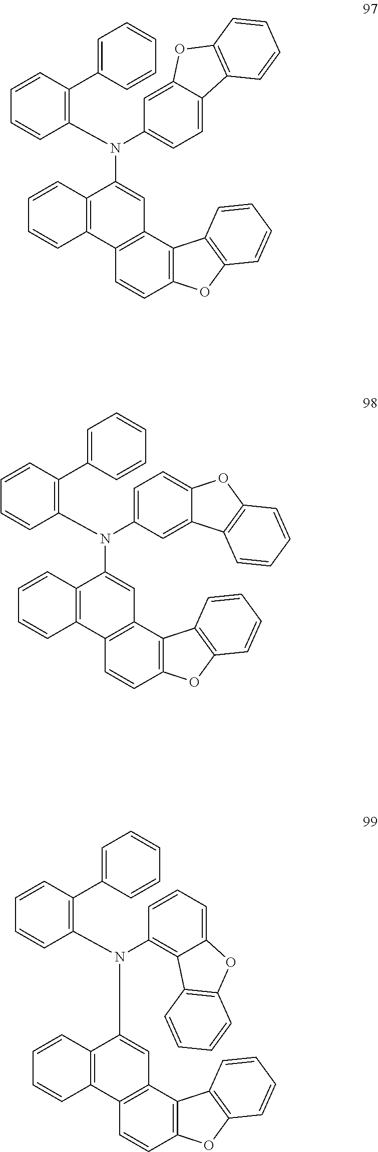 [SMILES: c1ccc(-c2ccccc2N(c2ccc3c(c2)oc2ccccc23)c2cc3c(ccc4oc5ccccc5c43)c3ccccc23)cc1 ; c1ccc(-c2ccccc2N(c2ccc3oc4ccccc4c3c2)c2cc3c(ccc4oc5ccccc5c43)c3ccccc23)cc1 ; c1ccc(-c2ccccc2N(c2cc3c(ccc4oc5ccccc5c43)c3ccccc23)c2cccc3oc4ccccc4c23)cc1]

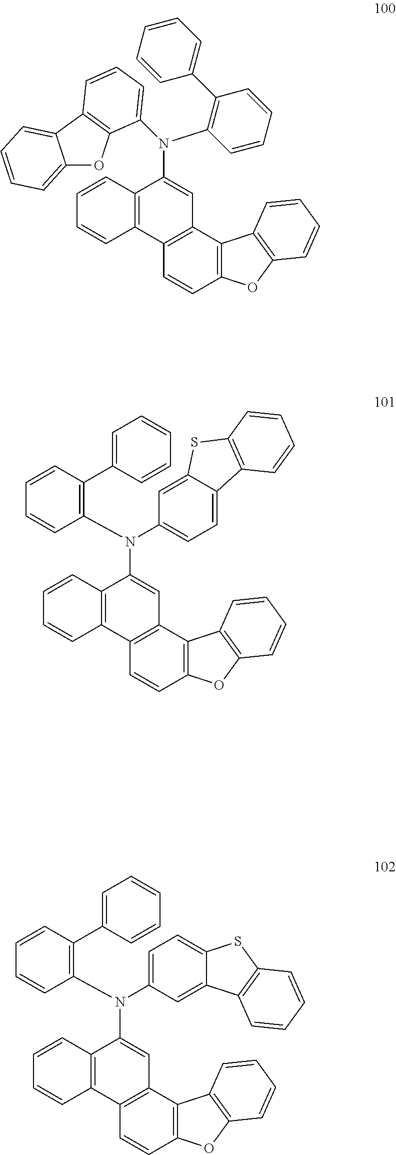 [SMILES: c1ccc(-c2ccccc2N(c2cc3c(ccc4oc5ccccc5c43)c3ccccc23)c2cccc3c2oc2ccccc23)cc1 ; c1ccc(-c2ccccc2N(c2ccc3c(c2)sc2ccccc23)c2cc3c(ccc4oc5ccccc5c43)c3ccccc23)cc1 ; c1ccc(-c2ccccc2N(c2ccc3sc4ccccc4c3c2)c2cc3c(ccc4oc5ccccc5c43)c3ccccc23)cc1]

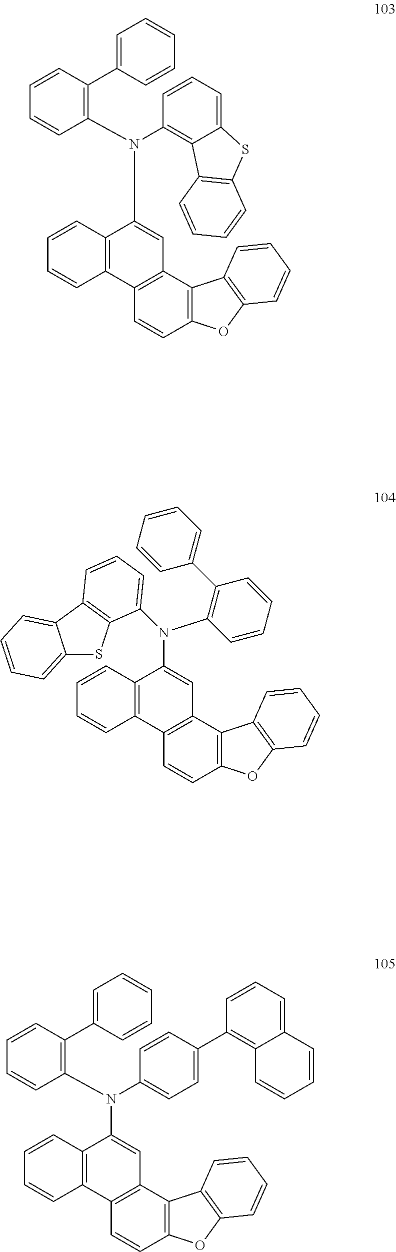 [SMILES: c1ccc(-c2ccccc2N(c2cc3c(ccc4oc5ccccc5c43)c3ccccc23)c2cccc3sc4ccccc4c23)cc1 ; c1ccc(-c2ccccc2N(c2cc3c(ccc4oc5ccccc5c43)c3ccccc23)c2cccc3c2sc2ccccc23)cc1 ; c1ccc(-c2ccccc2N(c2ccc(-c3cccc4ccccc34)cc2)c2cc3c(ccc4oc5ccccc5c43)c3ccccc23)cc1]

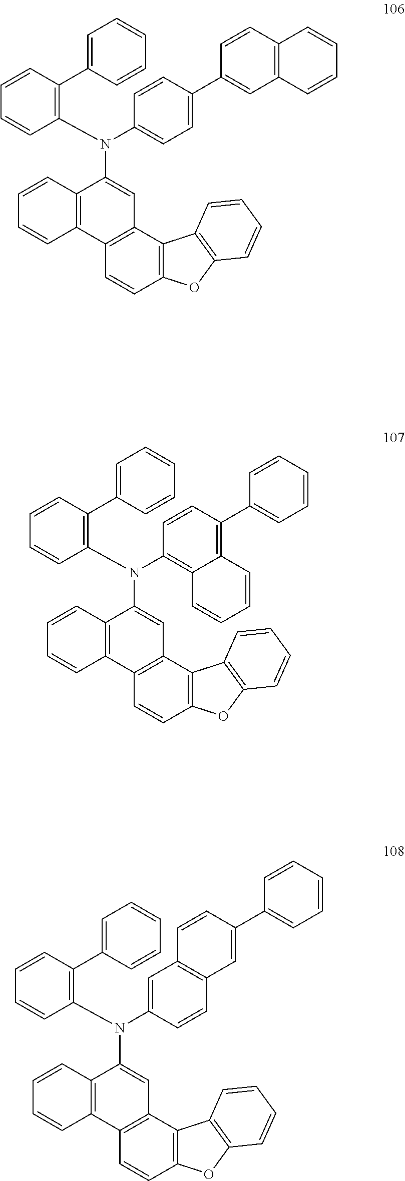 [SMILES: c1ccc(-c2ccccc2N(c2ccc(-c3ccc4ccccc4c3)cc2)c2cc3c(ccc4oc5ccccc5c43)c3ccccc23)cc1 ; c1ccc(-c2ccccc2N(c2ccc(-c3ccccc3)c3ccccc23)c2cc3c(ccc4oc5ccccc5c43)c3ccccc23)cc1 ; c1ccc(-c2ccc3cc(N(c4ccccc4-c4ccccc4)c4cc5c(ccc6oc7ccccc7c65)c5ccccc45)ccc3c2)cc1]

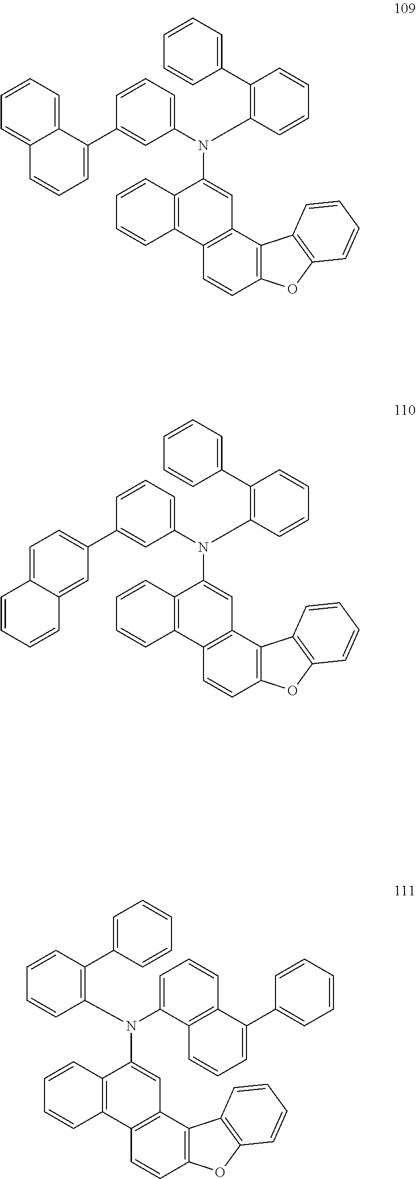 [SMILES: c1ccc(-c2ccccc2N(c2cccc(-c3cccc4ccccc34)c2)c2cc3c(ccc4oc5ccccc5c43)c3ccccc23)cc1 ; c1ccc(-c2ccccc2N(c2cccc(-c3ccc4ccccc4c3)c2)c2cc3c(ccc4oc5ccccc5c43)c3ccccc23)cc1 ; c1ccc(-c2ccccc2N(c2cccc3c(-c4ccccc4)cccc23)c2cc3c(ccc4oc5ccccc5c43)c3ccccc23)cc1]

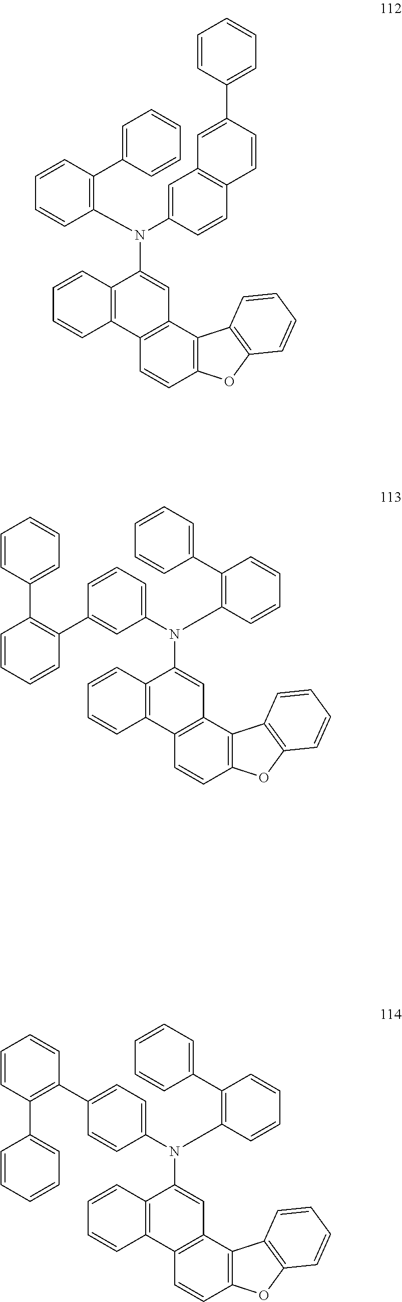 [SMILES: c1ccc(-c2ccc3ccc(N(c4ccccc4-c4ccccc4)c4cc5c(ccc6oc7ccccc7c65)c5ccccc45)cc3c2)cc1 ; c1ccc(-c2ccccc2-c2cccc(N(c3ccccc3-c3ccccc3)c3cc4c(ccc5oc6ccccc6c54)c4ccccc34)c2)cc1 ; c1ccc(-c2ccccc2-c2ccc(N(c3ccccc3-c3ccccc3)c3cc4c(ccc5oc6ccccc6c54)c4ccccc34)cc2)cc1]

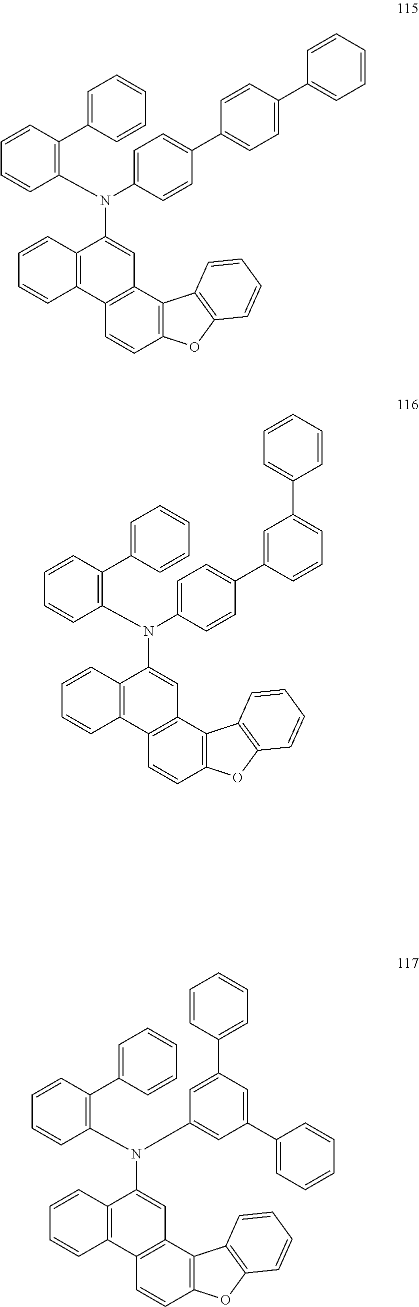 [SMILES: c1ccc(-c2ccc(-c3ccc(N(c4ccccc4-c4ccccc4)c4cc5c(ccc6oc7ccccc7c65)c5ccccc45)cc3)cc2)cc1 ; c1ccc(-c2cccc(-c3ccc(N(c4ccccc4-c4ccccc4)c4cc5c(ccc6oc7ccccc7c65)c5ccccc45)cc3)c2)cc1 ; c1ccc(-c2cc(-c3ccccc3)cc(N(c3ccccc3-c3ccccc3)c3cc4c(ccc5oc6ccccc6c54)c4ccccc34)c2)cc1]

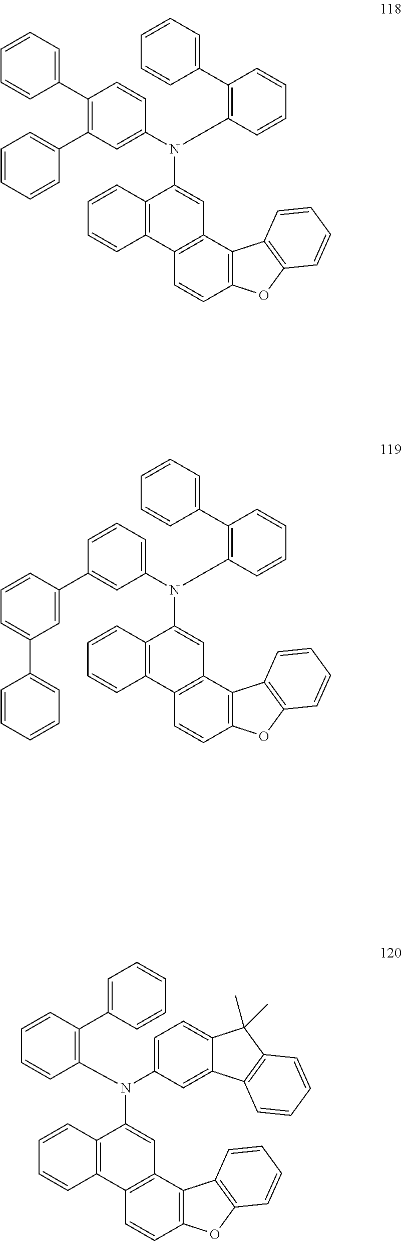 [SMILES: c1ccc(-c2ccc(N(c3ccccc3-c3ccccc3)c3cc4c(ccc5oc6ccccc6c54)c4ccccc34)cc2-c2ccccc2)cc1 ; c1ccc(-c2cccc(-c3cccc(N(c4ccccc4-c4ccccc4)c4cc5c(ccc6oc7ccccc7c65)c5ccccc45)c3)c2)cc1 ; CC1(C)c2ccccc2-c2cc(N(c3ccccc3-c3ccccc3)c3cc4c(ccc5oc6ccccc6c54)c4ccccc34)ccc21]

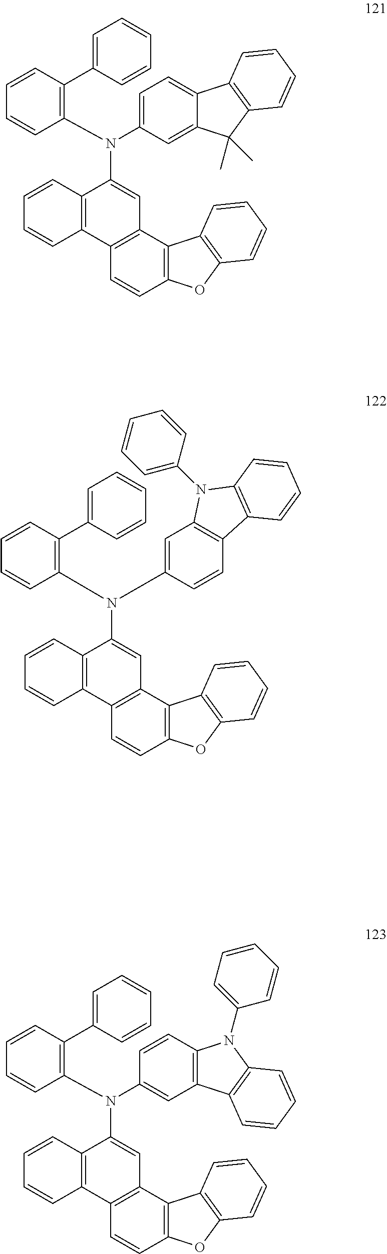 [SMILES: CC1(C)c2ccccc2-c2ccc(N(c3ccccc3-c3ccccc3)c3cc4c(ccc5oc6ccccc6c54)c4ccccc34)cc21 ; c1ccc(-c2ccccc2N(c2ccc3c4ccccc4n(-c4ccccc4)c3c2)c2cc3c(ccc4oc5ccccc5c43)c3ccccc23)cc1 ; c1ccc(-c2ccccc2N(c2ccc3c(c2)c2ccccc2n3-c2ccccc2)c2cc3c(ccc4oc5ccccc5c43)c3ccccc23)cc1]

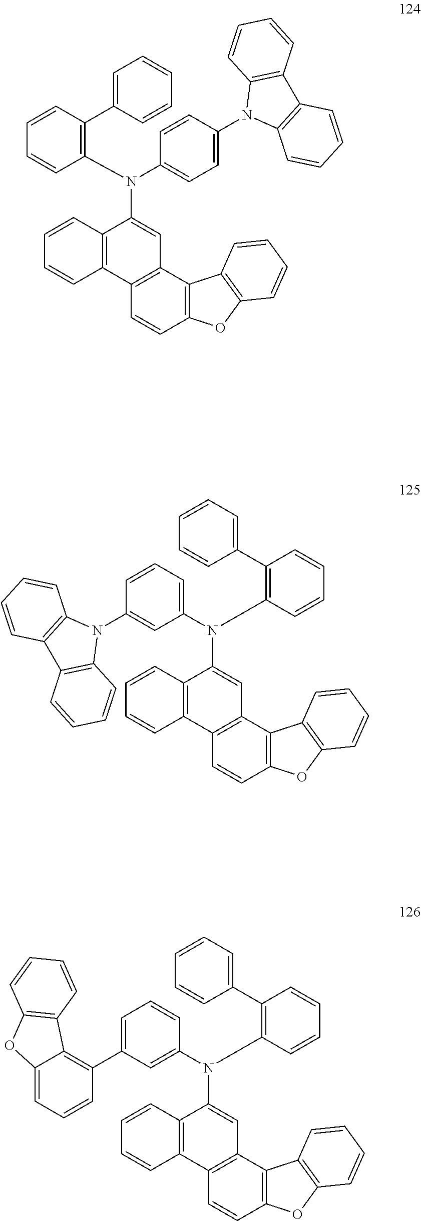 [SMILES: c1ccc(-c2ccccc2N(c2ccc(-n3c4ccccc4c4ccccc43)cc2)c2cc3c(ccc4oc5ccccc5c43)c3ccccc23)cc1 ; c1ccc(-c2ccccc2N(c2cccc(-n3c4ccccc4c4ccccc43)c2)c2cc3c(ccc4oc5ccccc5c43)c3ccccc23)cc1 ; c1ccc(-c2ccccc2N(c2cccc(-c3cccc4oc5ccccc5c34)c2)c2cc3c(ccc4oc5ccccc5c43)c3ccccc23)cc1]

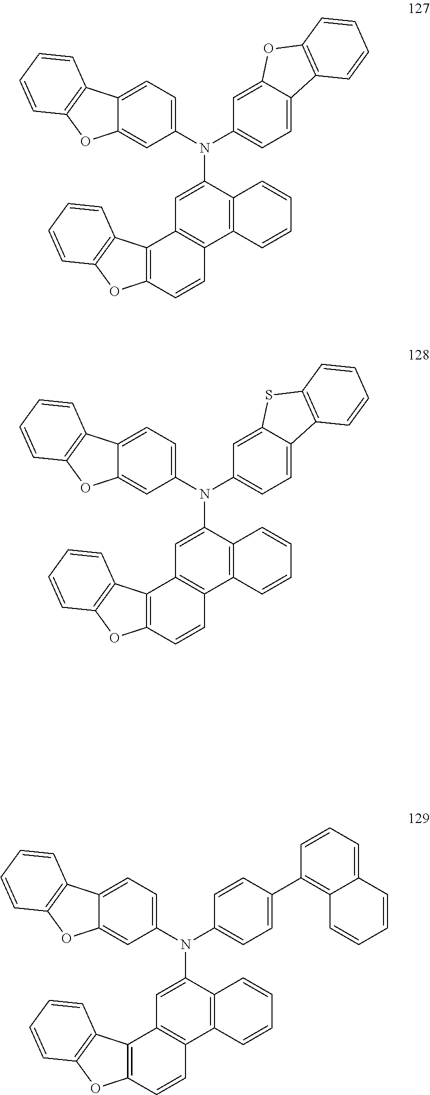 [SMILES: c1ccc2c(c1)oc1cc(N(c3ccc4c(c3)oc3ccccc34)c3cc4c(ccc5oc6ccccc6c54)c4ccccc34)ccc12 ; c1ccc2c(c1)oc1cc(N(c3ccc4c(c3)sc3ccccc34)c3cc4c(ccc5oc6ccccc6c54)c4ccccc34)ccc12 ; c1ccc2c(-c3ccc(N(c4ccc5c(c4)oc4ccccc45)c4cc5c(ccc6oc7ccccc7c65)c5ccccc45)cc3)cccc2c1]

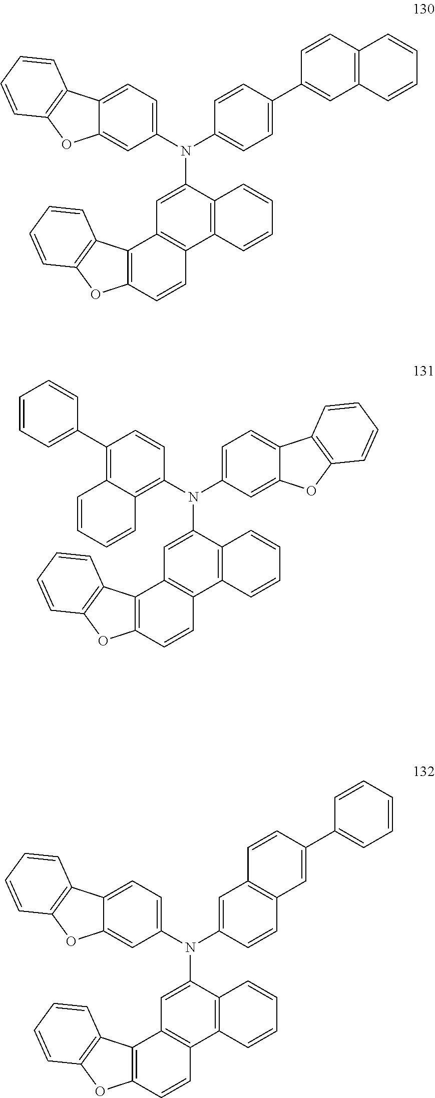 [SMILES: c1ccc2cc(-c3ccc(N(c4ccc5c(c4)oc4ccccc45)c4cc5c(ccc6oc7ccccc7c65)c5ccccc45)cc3)ccc2c1 ; c1ccc(-c2ccc(N(c3ccc4c(c3)oc3ccccc34)c3cc4c(ccc5oc6ccccc6c54)c4ccccc34)c3ccccc23)cc1 ; c1ccc(-c2ccc3cc(N(c4ccc5c(c4)oc4ccccc45)c4cc5c(ccc6oc7ccccc7c65)c5ccccc45)ccc3c2)cc1]

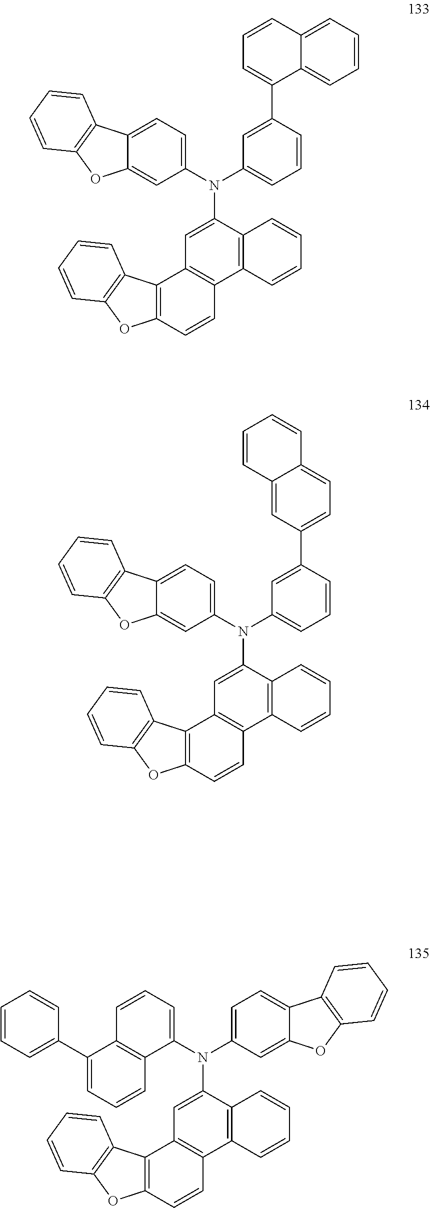 [SMILES: c1cc(-c2cccc3ccccc23)cc(N(c2ccc3c(c2)oc2ccccc23)c2cc3c(ccc4oc5ccccc5c43)c3ccccc23)c1 ; c1cc(-c2ccc3ccccc3c2)cc(N(c2ccc3c(c2)oc2ccccc23)c2cc3c(ccc4oc5ccccc5c43)c3ccccc23)c1 ; c1ccc(-c2cccc3c(N(c4ccc5c(c4)oc4ccccc45)c4cc5c(ccc6oc7ccccc7c65)c5ccccc45)cccc23)cc1]

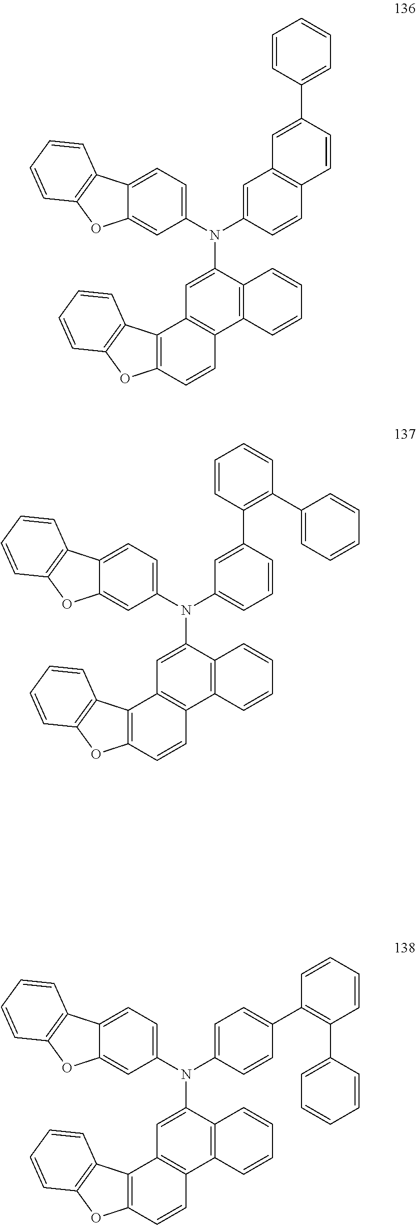 [SMILES: c1ccc(-c2ccc3ccc(N(c4ccc5c(c4)oc4ccccc45)c4cc5c(ccc6oc7ccccc7c65)c5ccccc45)cc3c2)cc1 ; c1ccc(-c2ccccc2-c2cccc(N(c3ccc4c(c3)oc3ccccc34)c3cc4c(ccc5oc6ccccc6c54)c4ccccc34)c2)cc1 ; c1ccc(-c2ccccc2-c2ccc(N(c3ccc4c(c3)oc3ccccc34)c3cc4c(ccc5oc6ccccc6c54)c4ccccc34)cc2)cc1]

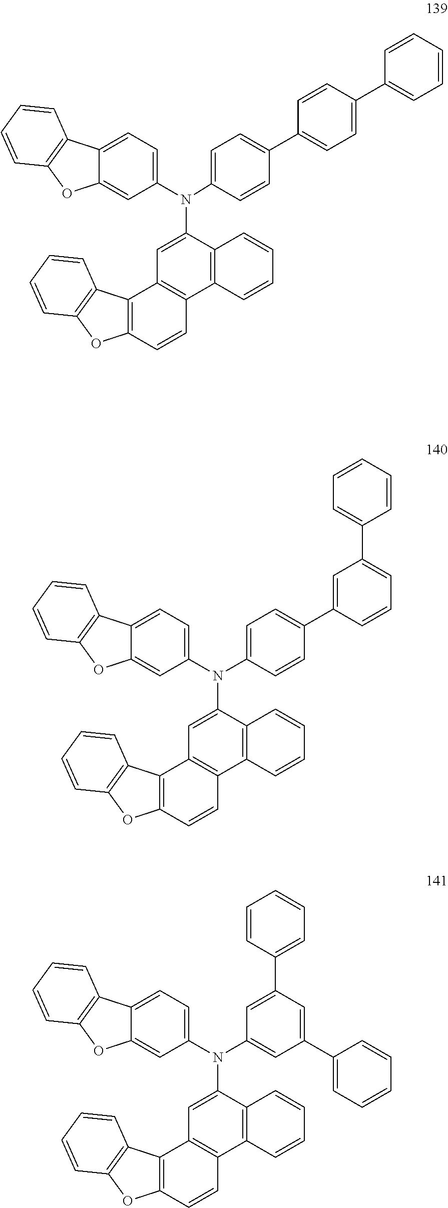 [SMILES: c1ccc(-c2ccc(-c3ccc(N(c4ccc5c(c4)oc4ccccc45)c4cc5c(ccc6oc7ccccc7c65)c5ccccc45)cc3)cc2)cc1 ; c1ccc(-c2cccc(-c3ccc(N(c4ccc5c(c4)oc4ccccc45)c4cc5c(ccc6oc7ccccc7c65)c5ccccc45)cc3)c2)cc1 ; c1ccc(-c2cc(-c3ccccc3)cc(N(c3ccc4c(c3)oc3ccccc34)c3cc4c(ccc5oc6ccccc6c54)c4ccccc34)c2)cc1]

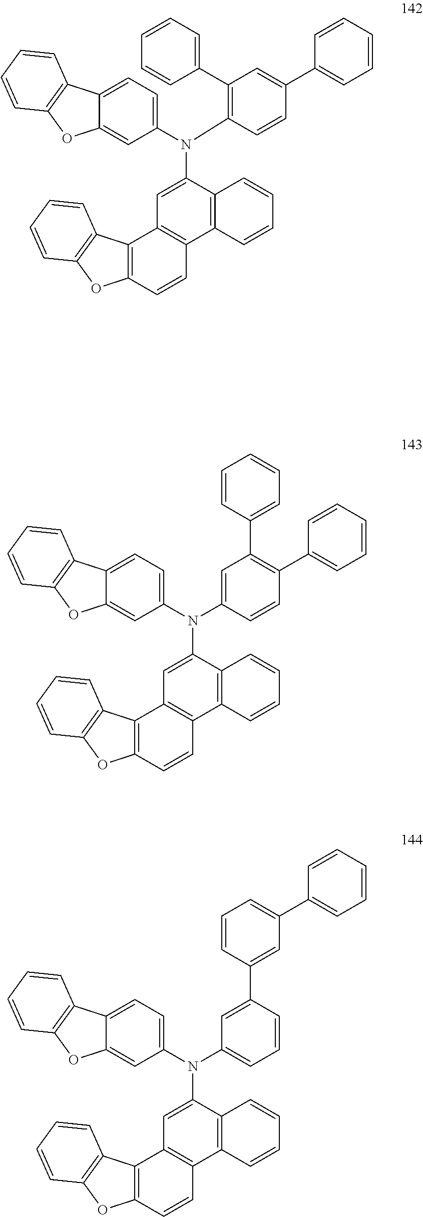 [SMILES: c1ccc(-c2ccc(N(c3ccc4c(c3)oc3ccccc34)c3cc4c(ccc5oc6ccccc6c54)c4ccccc34)c(-c3ccccc3)c2)cc1 ; c1ccc(-c2ccc(N(c3ccc4c(c3)oc3ccccc34)c3cc4c(ccc5oc6ccccc6c54)c4ccccc34)cc2-c2ccccc2)cc1 ; c1ccc(-c2cccc(-c3cccc(N(c4ccc5c(c4)oc4ccccc45)c4cc5c(ccc6oc7ccccc7c65)c5ccccc45)c3)c2)cc1]

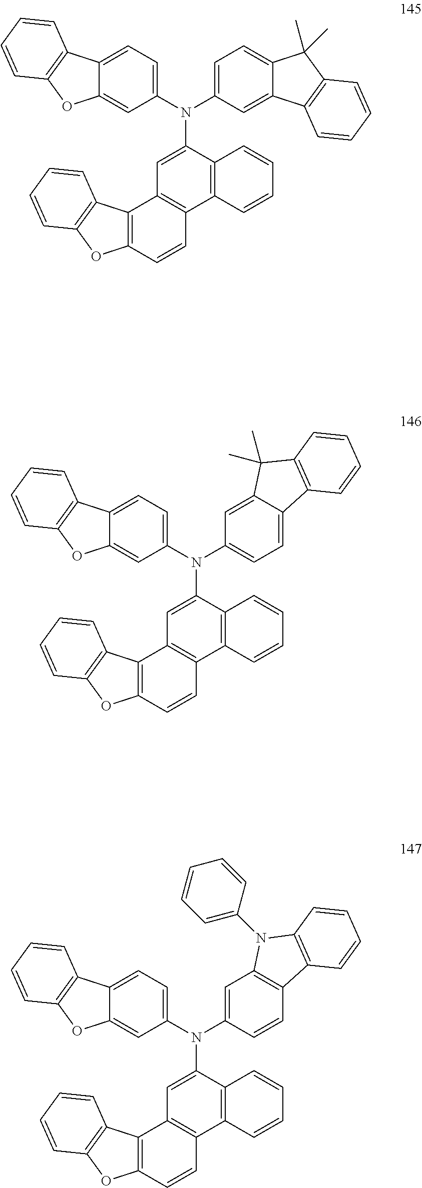 [SMILES: CC1(C)c2ccccc2-c2cc(N(c3ccc4c(c3)oc3ccccc34)c3cc4c(ccc5oc6ccccc6c54)c4ccccc34)ccc21 ; CC1(C)c2ccccc2-c2ccc(N(c3ccc4c(c3)oc3ccccc34)c3cc4c(ccc5oc6ccccc6c54)c4ccccc34)cc21 ; c1ccc(-n2c3ccccc3c3ccc(N(c4ccc5c(c4)oc4ccccc45)c4cc5c(ccc6oc7ccccc7c65)c5ccccc45)cc32)cc1]

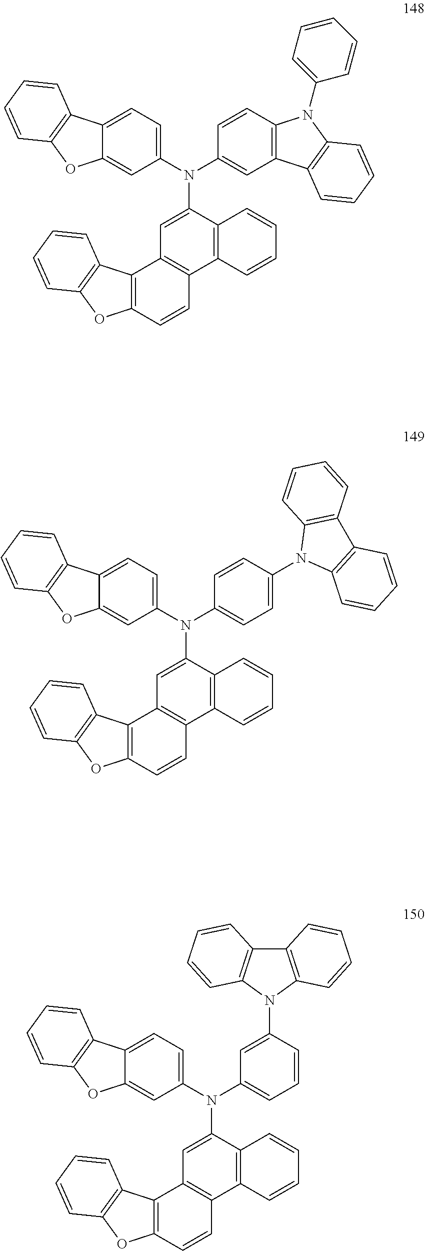 [SMILES: c1ccc(-n2c3ccccc3c3cc(N(c4ccc5c(c4)oc4ccccc45)c4cc5c(ccc6oc7ccccc7c65)c5ccccc45)ccc32)cc1 ; c1ccc2c(c1)oc1cc(N(c3ccc(-n4c5ccccc5c5ccccc54)cc3)c3cc4c(ccc5oc6ccccc6c54)c4ccccc34)ccc12 ; c1cc(N(c2ccc3c(c2)oc2ccccc23)c2cc3c(ccc4oc5ccccc5c43)c3ccccc23)cc(-n2c3ccccc3c3ccccc32)c1]

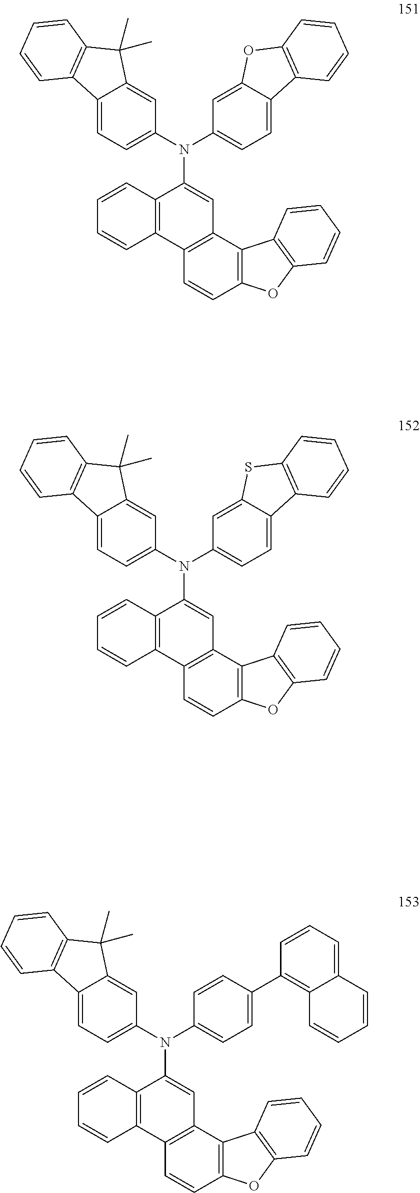 [SMILES: CC1(C)c2ccccc2-c2ccc(N(c3ccc4c(c3)oc3ccccc34)c3cc4c(ccc5oc6ccccc6c54)c4ccccc34)cc21 ; CC1(C)c2ccccc2-c2ccc(N(c3ccc4c(c3)sc3ccccc34)c3cc4c(ccc5oc6ccccc6c54)c4ccccc34)cc21 ; CC1(C)c2ccccc2-c2ccc(N(c3ccc(-c4cccc5ccccc45)cc3)c3cc4c(ccc5oc6ccccc6c54)c4ccccc34)cc21]

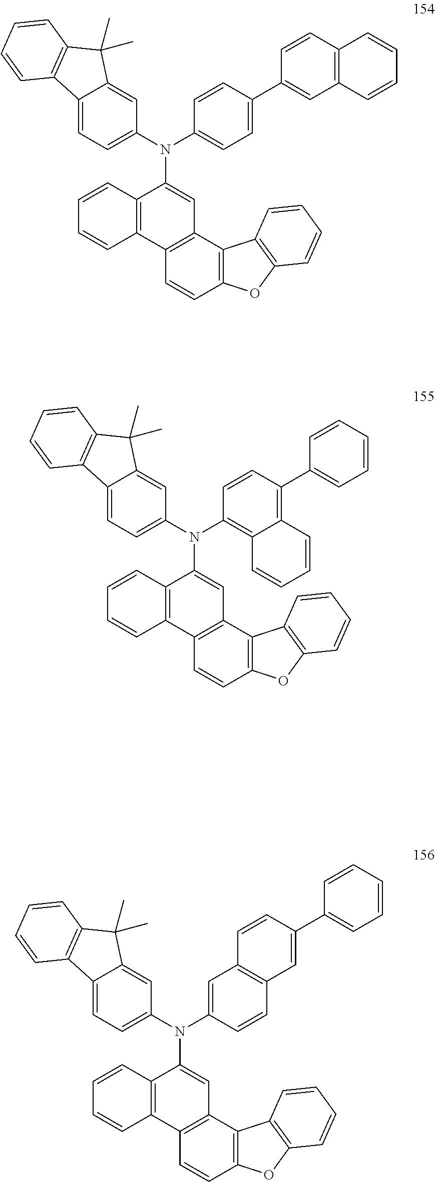 [SMILES: CC1(C)c2ccccc2-c2ccc(N(c3ccc(-c4ccc5ccccc5c4)cc3)c3cc4c(ccc5oc6ccccc6c54)c4ccccc34)cc21 ; CC1(C)c2ccccc2-c2ccc(N(c3ccc(-c4ccccc4)c4ccccc34)c3cc4c(ccc5oc6ccccc6c54)c4ccccc34)cc21 ; CC1(C)c2ccccc2-c2ccc(N(c3ccc4cc(-c5ccccc5)ccc4c3)c3cc4c(ccc5oc6ccccc6c54)c4ccccc34)cc21]

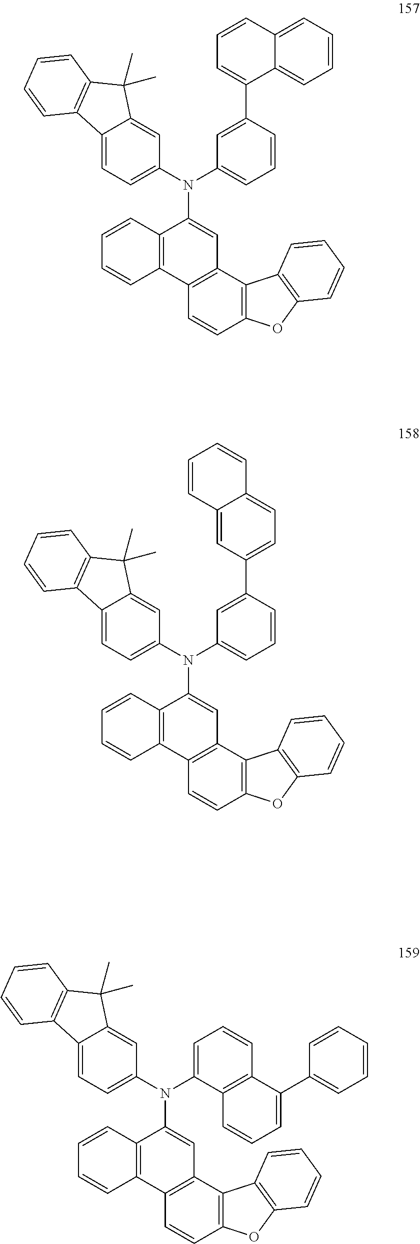 [SMILES: CC1(C)c2ccccc2-c2ccc(N(c3cccc(-c4cccc5ccccc45)c3)c3cc4c(ccc5oc6ccccc6c54)c4ccccc34)cc21 ; CC1(C)c2ccccc2-c2ccc(N(c3cccc(-c4ccc5ccccc5c4)c3)c3cc4c(ccc5oc6ccccc6c54)c4ccccc34)cc21 ; CC1(C)c2ccccc2-c2ccc(N(c3cccc4c(-c5ccccc5)cccc34)c3cc4c(ccc5oc6ccccc6c54)c4ccccc34)cc21]

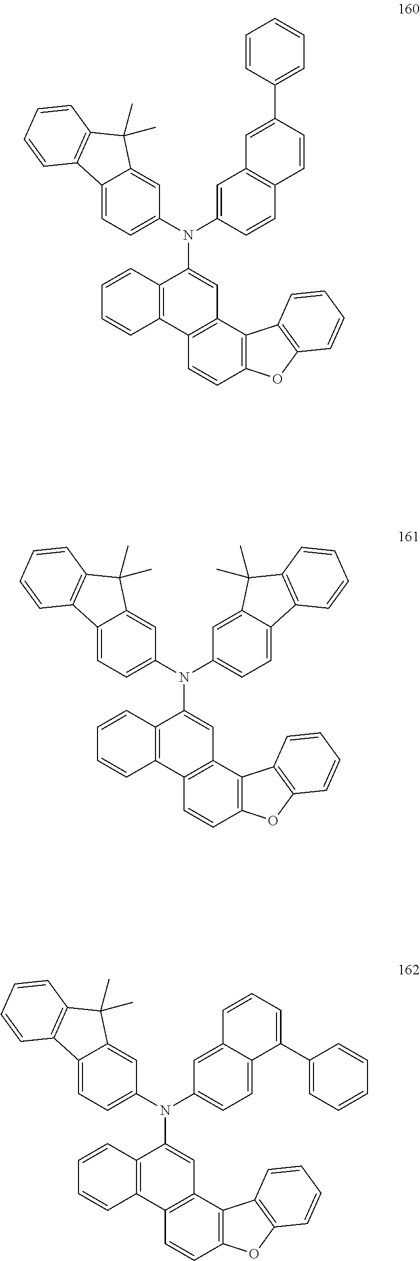 [SMILES: CC1(C)c2ccccc2-c2ccc(N(c3ccc4ccc(-c5ccccc5)cc4c3)c3cc4c(ccc5oc6ccccc6c54)c4ccccc34)cc21 ; CC1(C)c2ccccc2-c2ccc(N(c3ccc4c(c3)C(C)(C)c3ccccc3-4)c3cc4c(ccc5oc6ccccc6c54)c4ccccc34)cc21 ; CC1(C)c2ccccc2-c2ccc(N(c3ccc4c(-c5ccccc5)cccc4c3)c3cc4c(ccc5oc6ccccc6c54)c4ccccc34)cc21]

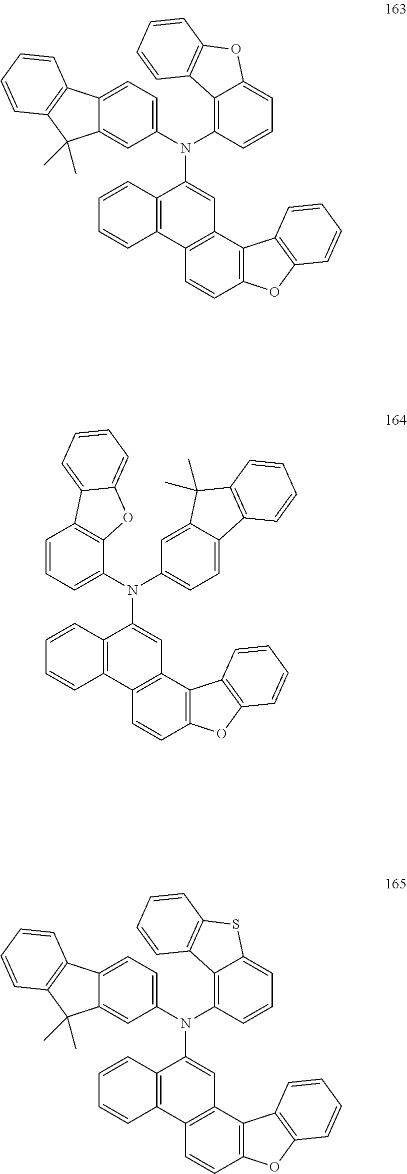 [SMILES: CC1(C)c2ccccc2-c2ccc(N(c3cc4c(ccc5oc6ccccc6c54)c4ccccc34)c3cccc4oc5ccccc5c34)cc21 ; CC1(C)c2ccccc2-c2ccc(N(c3cc4c(ccc5oc6ccccc6c54)c4ccccc34)c3cccc4c3oc3ccccc34)cc21 ; CC1(C)c2ccccc2-c2ccc(N(c3cc4c(ccc5oc6ccccc6c54)c4ccccc34)c3cccc4sc5ccccc5c34)cc21]

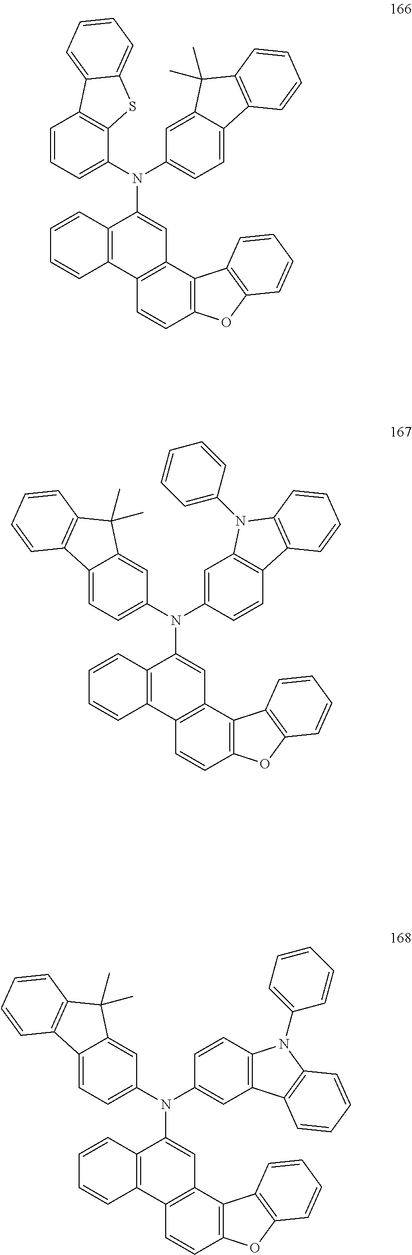 [SMILES: CC1(C)c2ccccc2-c2ccc(N(c3cc4c(ccc5oc6ccccc6c54)c4ccccc34)c3cccc4c3sc3ccccc34)cc21 ; CC1(C)c2ccccc2-c2ccc(N(c3ccc4c5ccccc5n(-c5ccccc5)c4c3)c3cc4c(ccc5oc6ccccc6c54)c4ccccc34)cc21 ; CC1(C)c2ccccc2-c2ccc(N(c3ccc4c(c3)c3ccccc3n4-c3ccccc3)c3cc4c(ccc5oc6ccccc6c54)c4ccccc34)cc21]

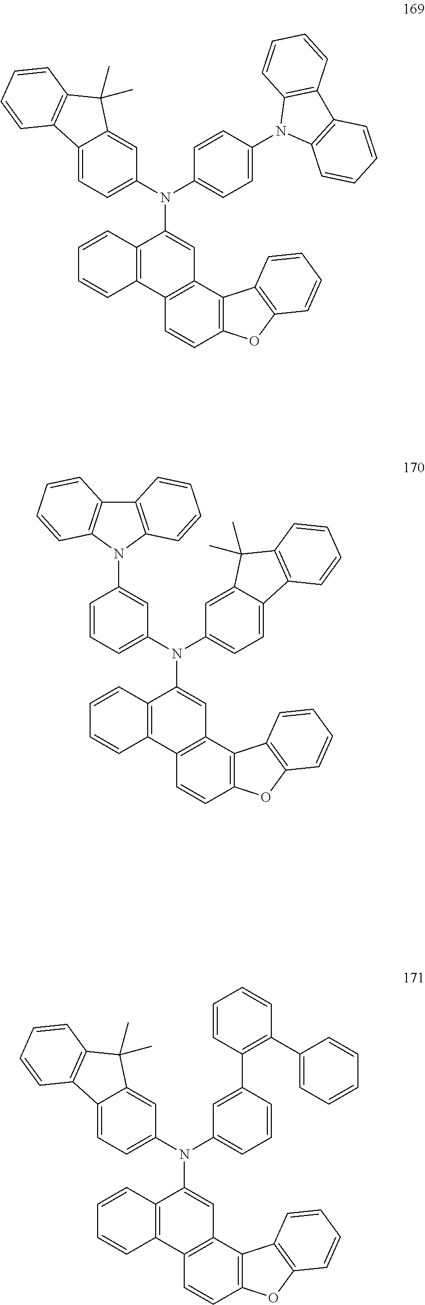 [SMILES: CC1(C)c2ccccc2-c2ccc(N(c3ccc(-n4c5ccccc5c5ccccc54)cc3)c3cc4c(ccc5oc6ccccc6c54)c4ccccc34)cc21 ; CC1(C)c2ccccc2-c2ccc(N(c3cccc(-n4c5ccccc5c5ccccc54)c3)c3cc4c(ccc5oc6ccccc6c54)c4ccccc34)cc21 ; CC1(C)c2ccccc2-c2ccc(N(c3cccc(-c4ccccc4-c4ccccc4)c3)c3cc4c(ccc5oc6ccccc6c54)c4ccccc34)cc21]

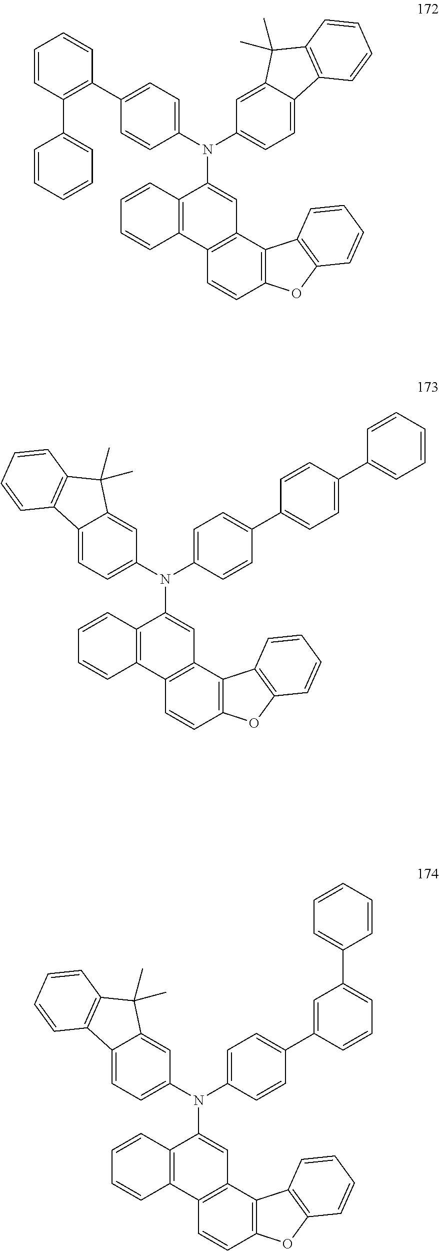 [SMILES: CC1(C)c2ccccc2-c2ccc(N(c3ccc(-c4ccccc4-c4ccccc4)cc3)c3cc4c(ccc5oc6ccccc6c54)c4ccccc34)cc21 ; CC1(C)c2ccccc2-c2ccc(N(c3ccc(-c4ccc(-c5ccccc5)cc4)cc3)c3cc4c(ccc5oc6ccccc6c54)c4ccccc34)cc21 ; CC1(C)c2ccccc2-c2ccc(N(c3ccc(-c4cccc(-c5ccccc5)c4)cc3)c3cc4c(ccc5oc6ccccc6c54)c4ccccc34)cc21]

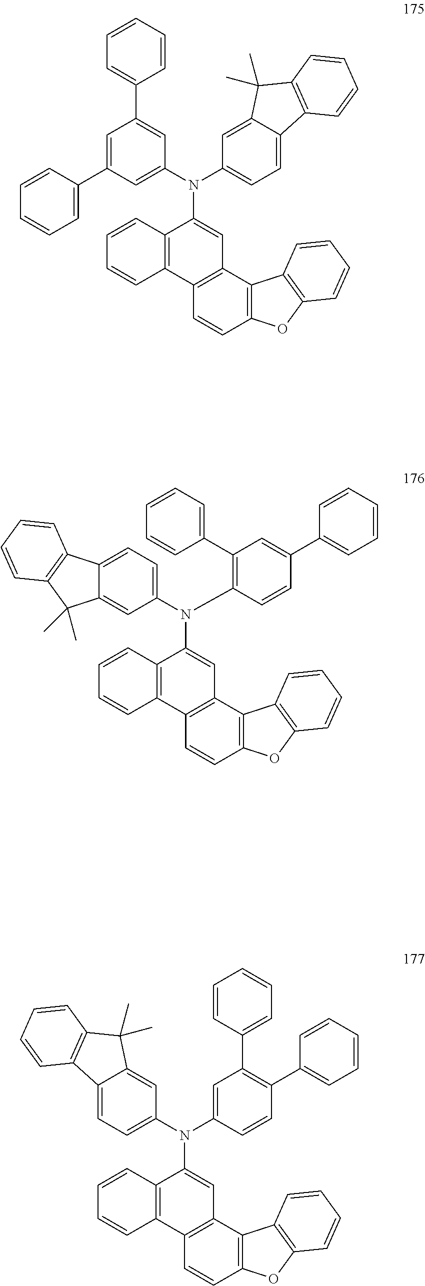 [SMILES: CC1(C)c2ccccc2-c2ccc(N(c3cc(-c4ccccc4)cc(-c4ccccc4)c3)c3cc4c(ccc5oc6ccccc6c54)c4ccccc34)cc21 ; CC1(C)c2ccccc2-c2ccc(N(c3ccc(-c4ccccc4)cc3-c3ccccc3)c3cc4c(ccc5oc6ccccc6c54)c4ccccc34)cc21 ; CC1(C)c2ccccc2-c2ccc(N(c3ccc(-c4ccccc4)c(-c4ccccc4)c3)c3cc4c(ccc5oc6ccccc6c54)c4ccccc34)cc21]

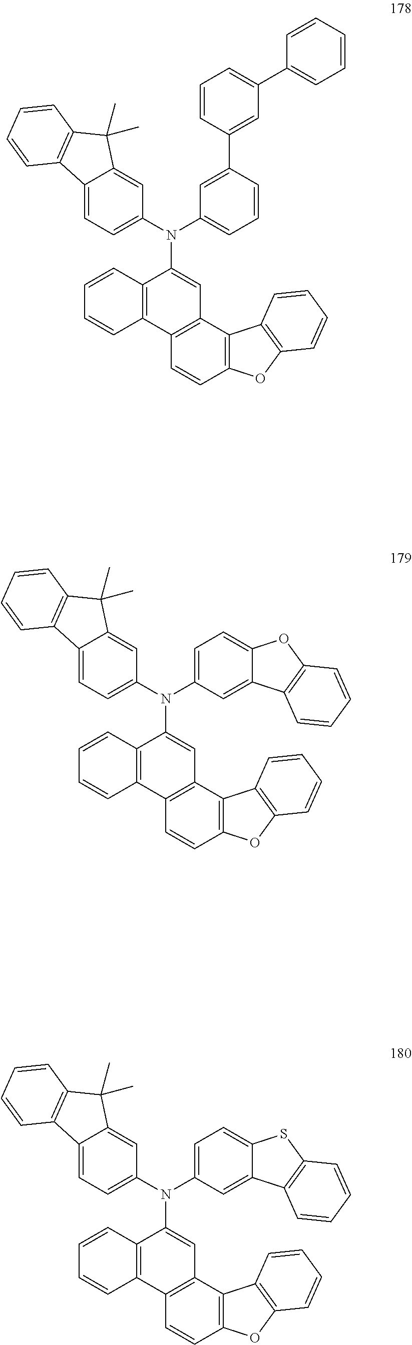 [SMILES: CC1(C)c2ccccc2-c2ccc(N(c3cccc(-c4cccc(-c5ccccc5)c4)c3)c3cc4c(ccc5oc6ccccc6c54)c4ccccc34)cc21 ; CC1(C)c2ccccc2-c2ccc(N(c3ccc4oc5ccccc5c4c3)c3cc4c(ccc5oc6ccccc6c54)c4ccccc34)cc21 ; CC1(C)c2ccccc2-c2ccc(N(c3ccc4sc5ccccc5c4c3)c3cc4c(ccc5oc6ccccc6c54)c4ccccc34)cc21]

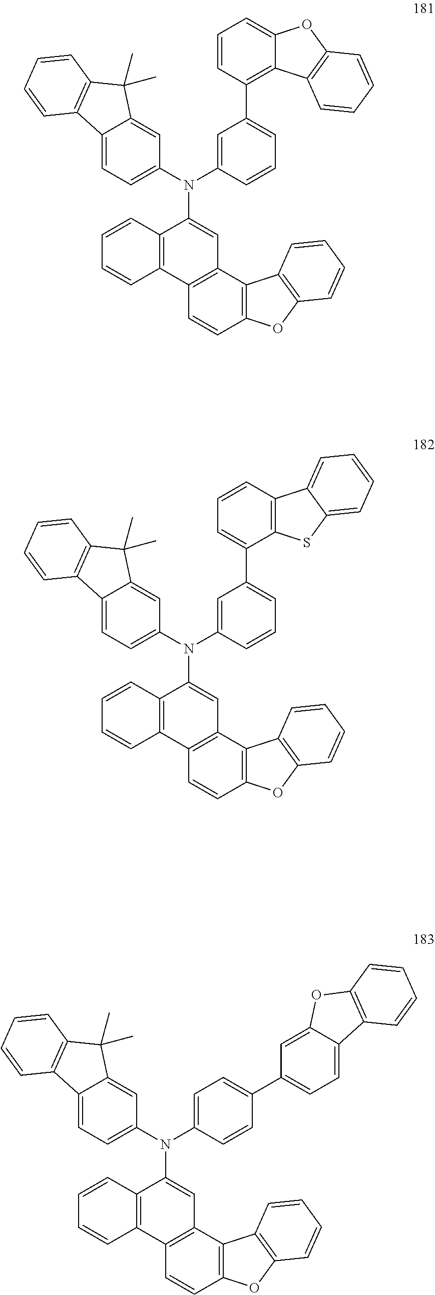 [SMILES: CC1(C)c2ccccc2-c2ccc(N(c3cccc(-c4cccc5oc6ccccc6c45)c3)c3cc4c(ccc5oc6ccccc6c54)c4ccccc34)cc21 ; CC1(C)c2ccccc2-c2ccc(N(c3cccc(-c4cccc5c4sc4ccccc45)c3)c3cc4c(ccc5oc6ccccc6c54)c4ccccc34)cc21 ; CC1(C)c2ccccc2-c2ccc(N(c3ccc(-c4ccc5c(c4)oc4ccccc45)cc3)c3cc4c(ccc5oc6ccccc6c54)c4ccccc34)cc21]

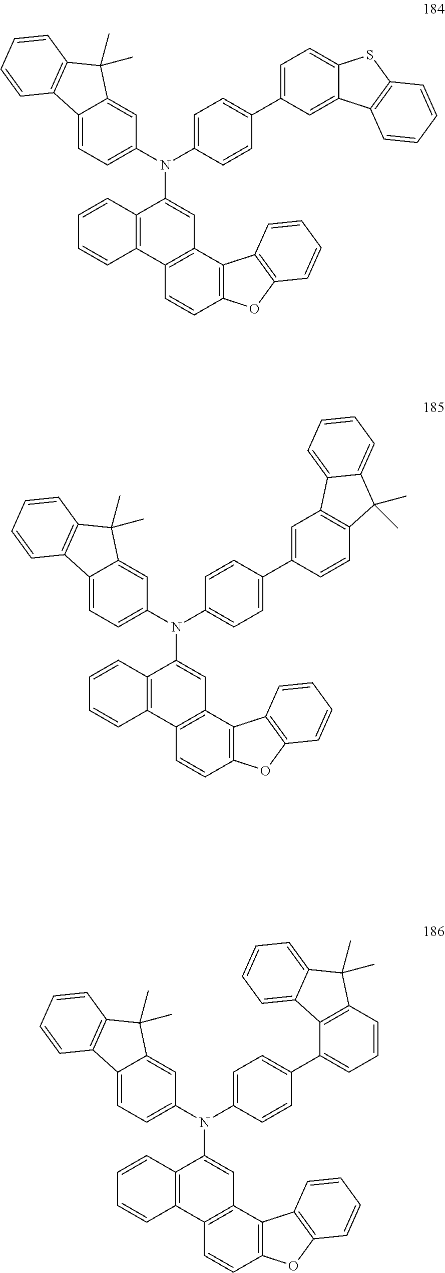 [SMILES: CC1(C)c2ccccc2-c2ccc(N(c3ccc(-c4ccc5sc6ccccc6c5c4)cc3)c3cc4c(ccc5oc6ccccc6c54)c4ccccc34)cc21 ; CC1(C)c2ccccc2-c2cc(-c3ccc(N(c4ccc5c(c4)C(C)(C)c4ccccc4-5)c4cc5c(ccc6oc7ccccc7c65)c5ccccc45)cc3)ccc21 ; CC1(C)c2ccccc2-c2ccc(N(c3ccc(-c4cccc5c4-c4ccccc4C5(C)C)cc3)c3cc4c(ccc5oc6ccccc6c54)c4ccccc34)cc21]

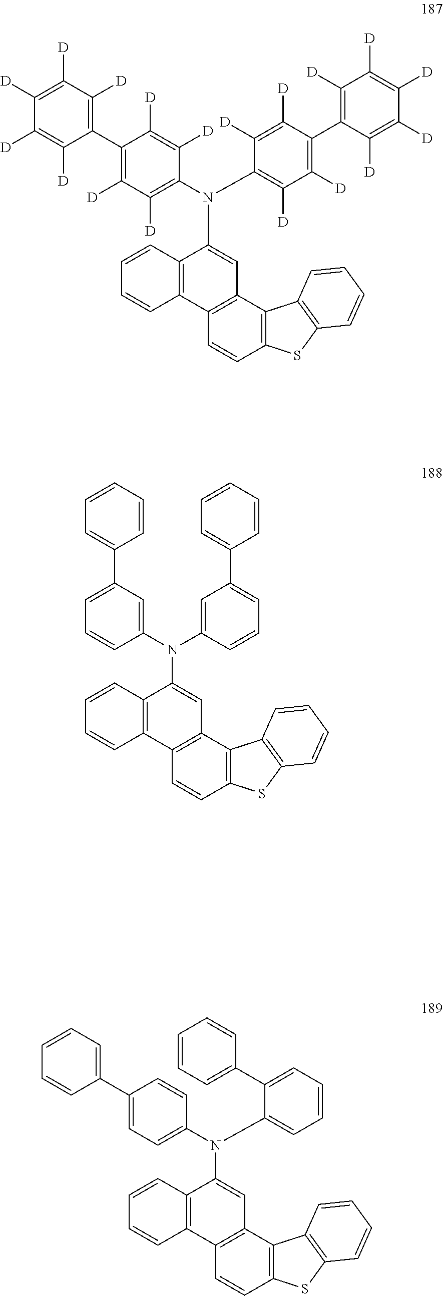 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3cc4c(ccc5sc6ccccc6c54)c4ccccc34)c([2H])c2[2H])c([2H])c1[2H] ; c1ccc(-c2cccc(N(c3cccc(-c4ccccc4)c3)c3cc4c(ccc5sc6ccccc6c54)c4ccccc34)c2)cc1 ; c1ccc(-c2ccc(N(c3ccccc3-c3ccccc3)c3cc4c(ccc5sc6ccccc6c54)c4ccccc34)cc2)cc1]

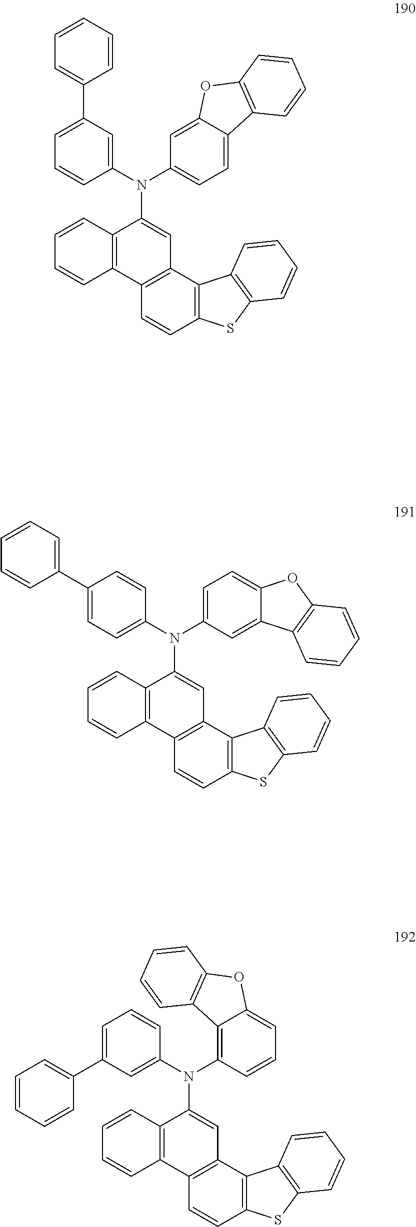 [SMILES: c1ccc(-c2cccc(N(c3ccc4c(c3)oc3ccccc34)c3cc4c(ccc5sc6ccccc6c54)c4ccccc34)c2)cc1 ; c1ccc(-c2ccc(N(c3ccc4oc5ccccc5c4c3)c3cc4c(ccc5sc6ccccc6c54)c4ccccc34)cc2)cc1 ; c1ccc(-c2cccc(N(c3cc4c(ccc5sc6ccccc6c54)c4ccccc34)c3cccc4oc5ccccc5c34)c2)cc1]

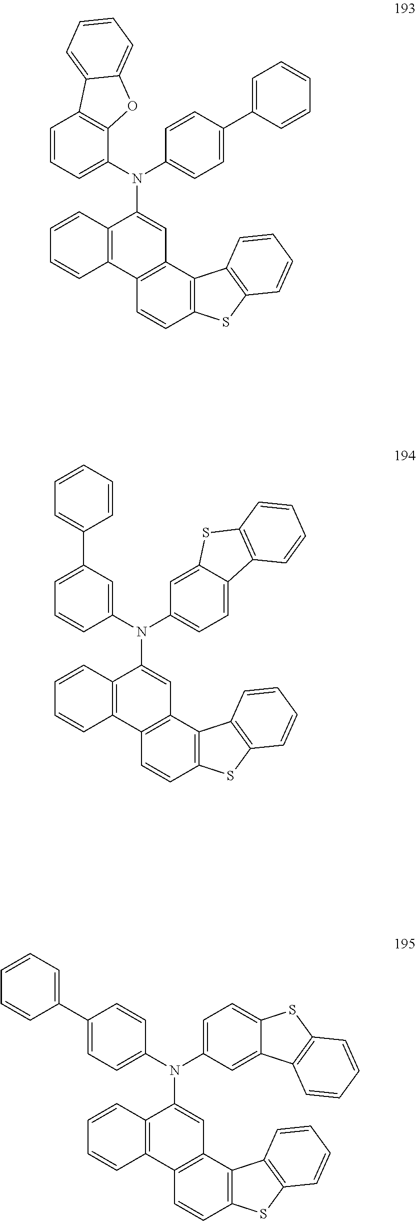 [SMILES: c1ccc(-c2ccc(N(c3cc4c(ccc5sc6ccccc6c54)c4ccccc34)c3cccc4c3oc3ccccc34)cc2)cc1 ; c1ccc(-c2cccc(N(c3ccc4c(c3)sc3ccccc34)c3cc4c(ccc5sc6ccccc6c54)c4ccccc34)c2)cc1 ; c1ccc(-c2ccc(N(c3ccc4sc5ccccc5c4c3)c3cc4c(ccc5sc6ccccc6c54)c4ccccc34)cc2)cc1]

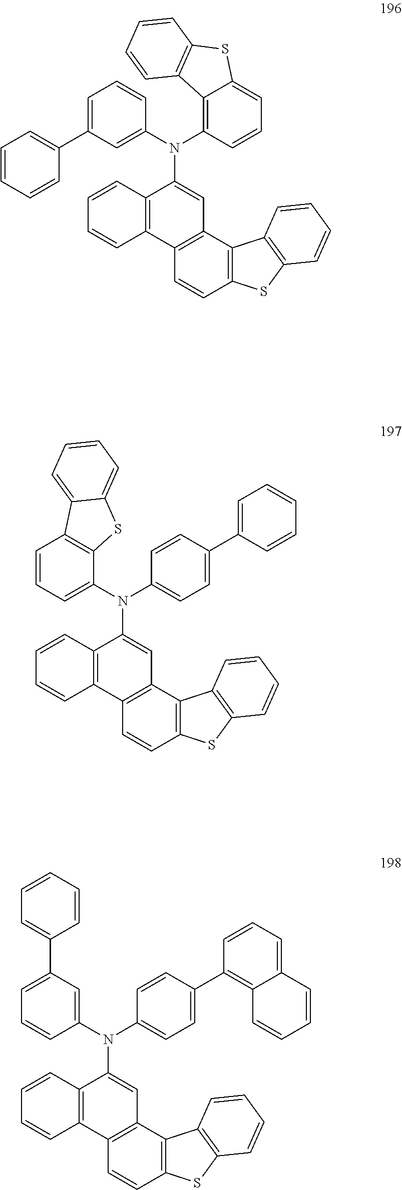 [SMILES: c1ccc(-c2cccc(N(c3cc4c(ccc5sc6ccccc6c54)c4ccccc34)c3cccc4sc5ccccc5c34)c2)cc1 ; c1ccc(-c2ccc(N(c3cc4c(ccc5sc6ccccc6c54)c4ccccc34)c3cccc4c3sc3ccccc34)cc2)cc1 ; c1ccc(-c2cccc(N(c3ccc(-c4cccc5ccccc45)cc3)c3cc4c(ccc5sc6ccccc6c54)c4ccccc34)c2)cc1]

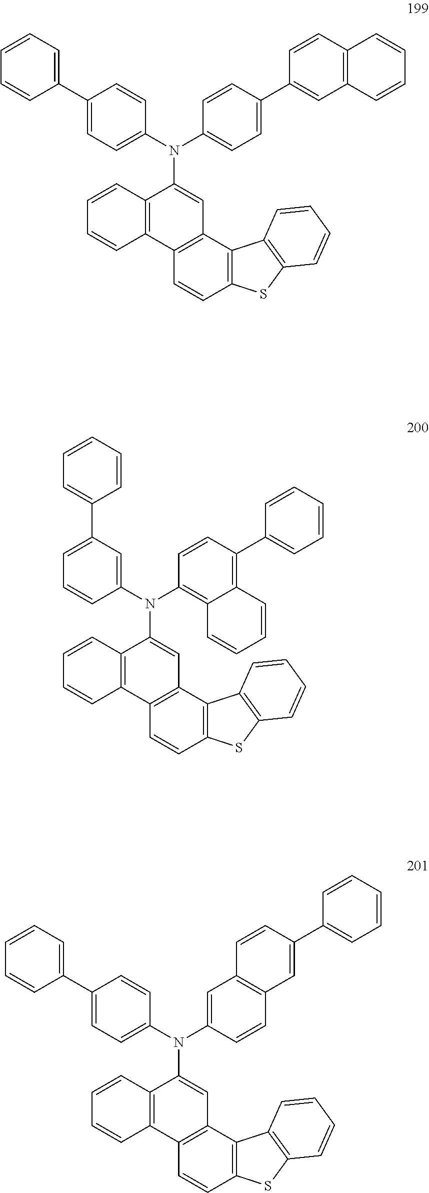 [SMILES: c1ccc(-c2ccc(N(c3ccc(-c4ccc5ccccc5c4)cc3)c3cc4c(ccc5sc6ccccc6c54)c4ccccc34)cc2)cc1 ; c1ccc(-c2cccc(N(c3ccc(-c4ccccc4)c4ccccc34)c3cc4c(ccc5sc6ccccc6c54)c4ccccc34)c2)cc1 ; c1ccc(-c2ccc(N(c3ccc4cc(-c5ccccc5)ccc4c3)c3cc4c(ccc5sc6ccccc6c54)c4ccccc34)cc2)cc1]

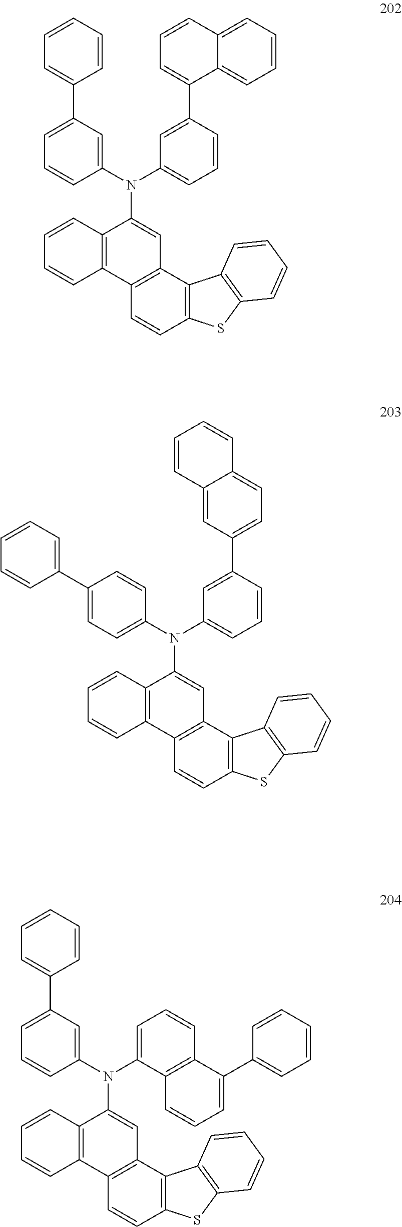 [SMILES: c1ccc(-c2cccc(N(c3cccc(-c4cccc5ccccc45)c3)c3cc4c(ccc5sc6ccccc6c54)c4ccccc34)c2)cc1 ; c1ccc(-c2ccc(N(c3cccc(-c4ccc5ccccc5c4)c3)c3cc4c(ccc5sc6ccccc6c54)c4ccccc34)cc2)cc1 ; c1ccc(-c2cccc(N(c3cccc4c(-c5ccccc5)cccc34)c3cc4c(ccc5sc6ccccc6c54)c4ccccc34)c2)cc1]

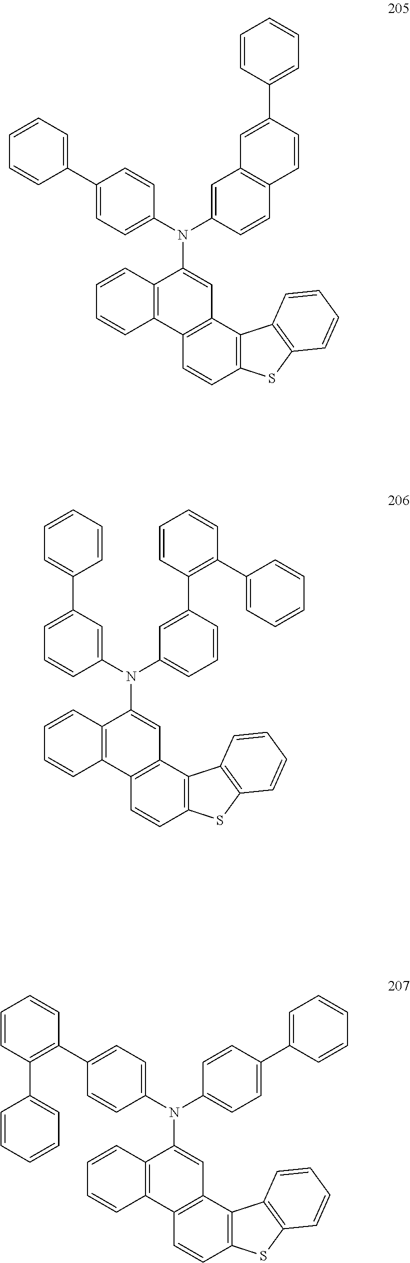 [SMILES: c1ccc(-c2ccc(N(c3ccc4ccc(-c5ccccc5)cc4c3)c3cc4c(ccc5sc6ccccc6c54)c4ccccc34)cc2)cc1 ; c1ccc(-c2cccc(N(c3cccc(-c4ccccc4-c4ccccc4)c3)c3cc4c(ccc5sc6ccccc6c54)c4ccccc34)c2)cc1 ; c1ccc(-c2ccc(N(c3ccc(-c4ccccc4-c4ccccc4)cc3)c3cc4c(ccc5sc6ccccc6c54)c4ccccc34)cc2)cc1]

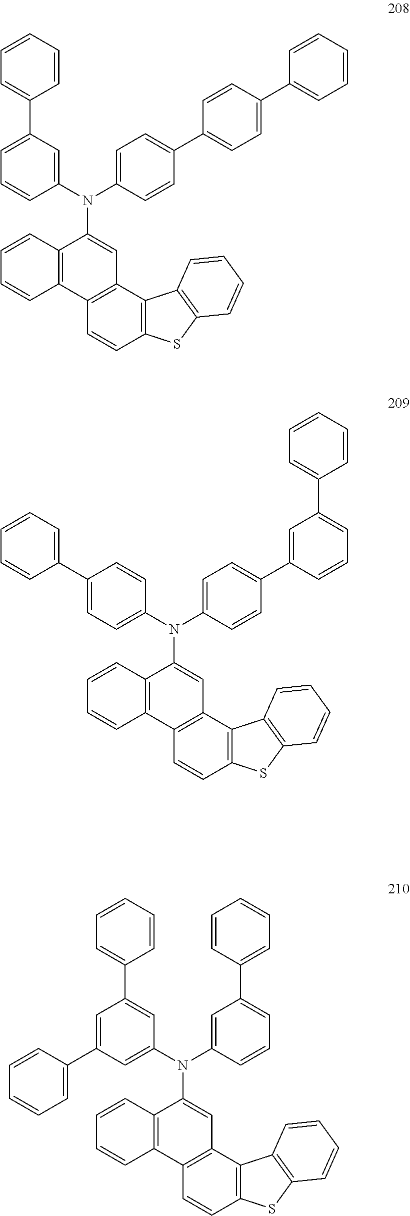 [SMILES: c1ccc(-c2ccc(-c3ccc(N(c4cccc(-c5ccccc5)c4)c4cc5c(ccc6sc7ccccc7c65)c5ccccc45)cc3)cc2)cc1 ; c1ccc(-c2ccc(N(c3ccc(-c4cccc(-c5ccccc5)c4)cc3)c3cc4c(ccc5sc6ccccc6c54)c4ccccc34)cc2)cc1 ; c1ccc(-c2cccc(N(c3cc(-c4ccccc4)cc(-c4ccccc4)c3)c3cc4c(ccc5sc6ccccc6c54)c4ccccc34)c2)cc1]

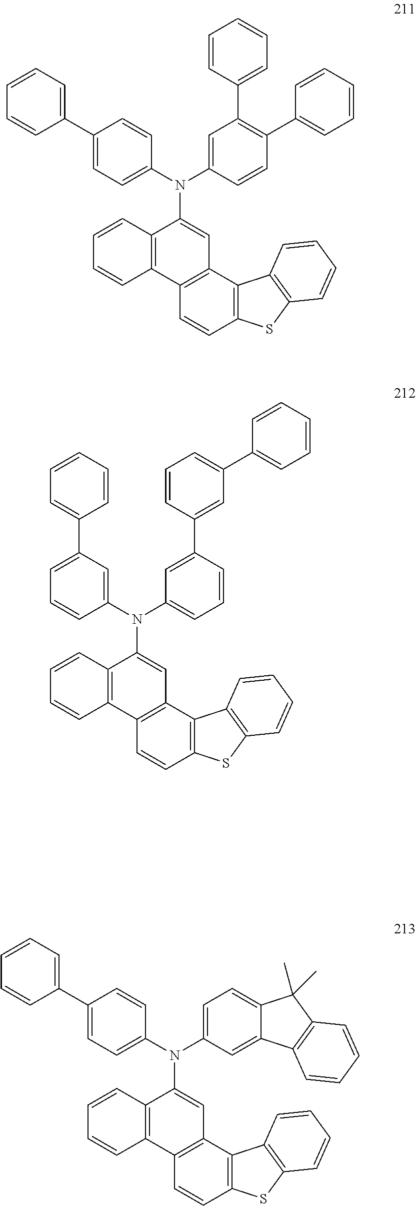 [SMILES: c1ccc(-c2ccc(N(c3ccc(-c4ccccc4)c(-c4ccccc4)c3)c3cc4c(ccc5sc6ccccc6c54)c4ccccc34)cc2)cc1 ; c1ccc(-c2cccc(-c3cccc(N(c4cccc(-c5ccccc5)c4)c4cc5c(ccc6sc7ccccc7c65)c5ccccc45)c3)c2)cc1 ; CC1(C)c2ccccc2-c2cc(N(c3ccc(-c4ccccc4)cc3)c3cc4c(ccc5sc6ccccc6c54)c4ccccc34)ccc21]

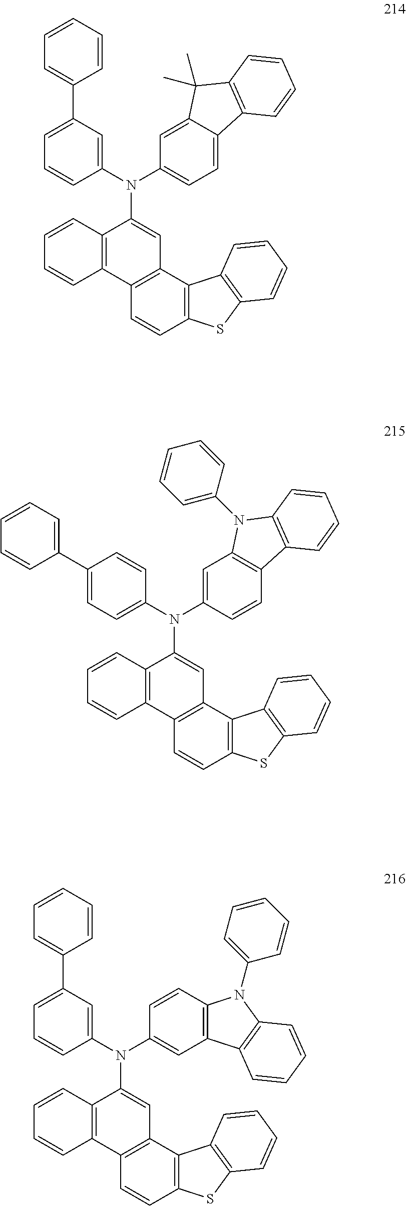 [SMILES: CC1(C)c2ccccc2-c2ccc(N(c3cccc(-c4ccccc4)c3)c3cc4c(ccc5sc6ccccc6c54)c4ccccc34)cc21 ; c1ccc(-c2ccc(N(c3ccc4c5ccccc5n(-c5ccccc5)c4c3)c3cc4c(ccc5sc6ccccc6c54)c4ccccc34)cc2)cc1 ; c1ccc(-c2cccc(N(c3ccc4c(c3)c3ccccc3n4-c3ccccc3)c3cc4c(ccc5sc6ccccc6c54)c4ccccc34)c2)cc1]

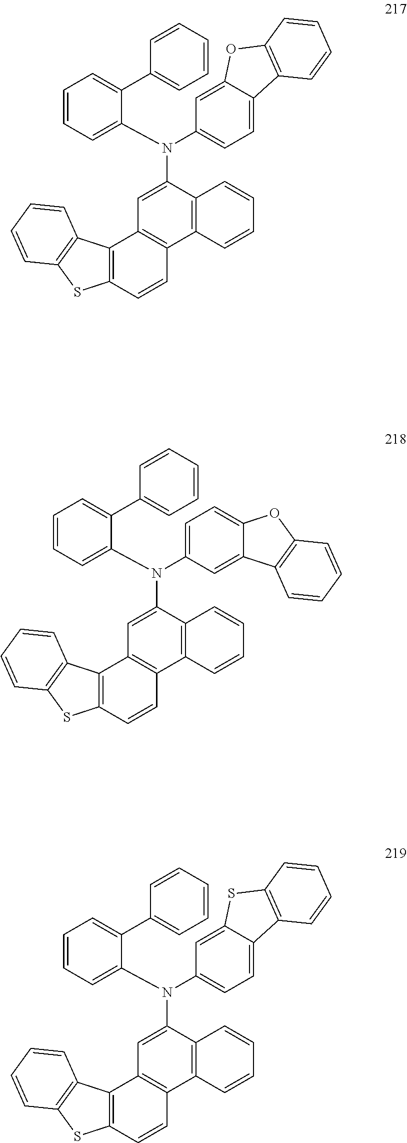 [SMILES: c1ccc(-c2ccccc2N(c2ccc3c(c2)oc2ccccc23)c2cc3c(ccc4sc5ccccc5c43)c3ccccc23)cc1 ; c1ccc(-c2ccccc2N(c2ccc3oc4ccccc4c3c2)c2cc3c(ccc4sc5ccccc5c43)c3ccccc23)cc1 ; c1ccc(-c2ccccc2N(c2ccc3c(c2)sc2ccccc23)c2cc3c(ccc4sc5ccccc5c43)c3ccccc23)cc1]

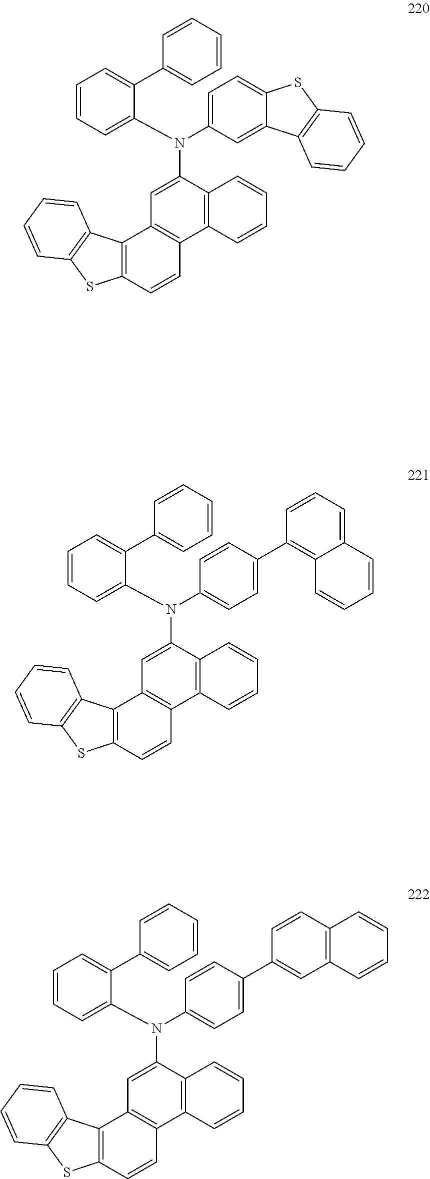 [SMILES: c1ccc(-c2ccccc2N(c2ccc3sc4ccccc4c3c2)c2cc3c(ccc4sc5ccccc5c43)c3ccccc23)cc1 ; c1ccc(-c2ccccc2N(c2ccc(-c3cccc4ccccc34)cc2)c2cc3c(ccc4sc5ccccc5c43)c3ccccc23)cc1 ; c1ccc(-c2ccccc2N(c2ccc(-c3ccc4ccccc4c3)cc2)c2cc3c(ccc4sc5ccccc5c43)c3ccccc23)cc1]

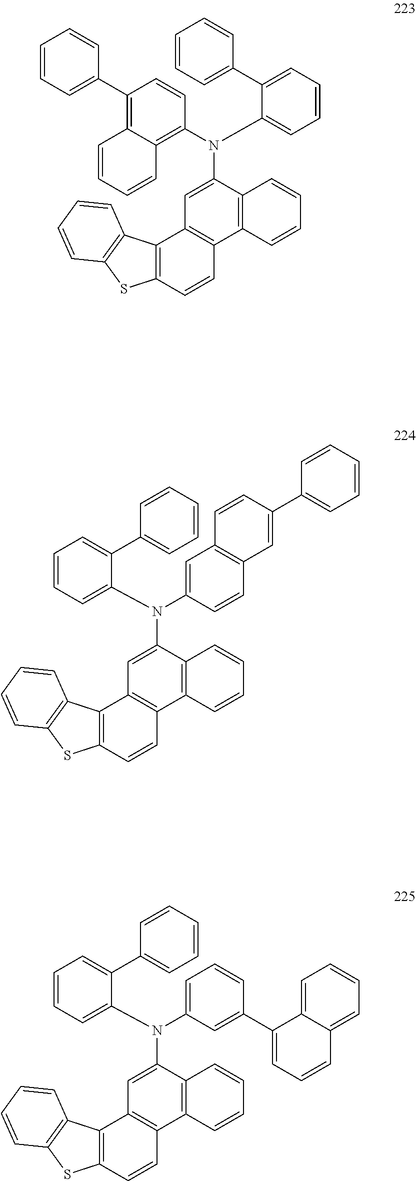 [SMILES: c1ccc(-c2ccccc2N(c2ccc(-c3ccccc3)c3ccccc23)c2cc3c(ccc4sc5ccccc5c43)c3ccccc23)cc1 ; c1ccc(-c2ccc3cc(N(c4ccccc4-c4ccccc4)c4cc5c(ccc6sc7ccccc7c65)c5ccccc45)ccc3c2)cc1 ; c1ccc(-c2ccccc2N(c2cccc(-c3cccc4ccccc34)c2)c2cc3c(ccc4sc5ccccc5c43)c3ccccc23)cc1]

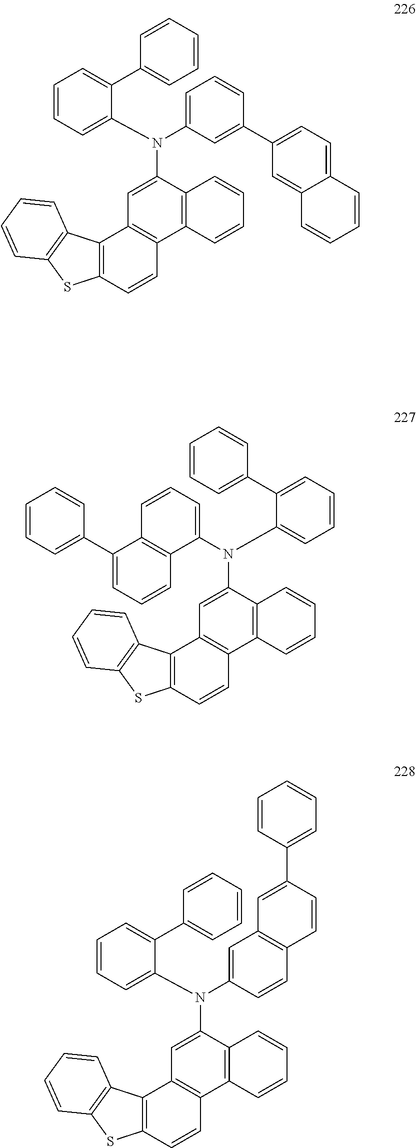 [SMILES: c1ccc(-c2ccccc2N(c2cccc(-c3ccc4ccccc4c3)c2)c2cc3c(ccc4sc5ccccc5c43)c3ccccc23)cc1 ; c1ccc(-c2ccccc2N(c2cccc3c(-c4ccccc4)cccc23)c2cc3c(ccc4sc5ccccc5c43)c3ccccc23)cc1 ; c1ccc(-c2ccc3ccc(N(c4ccccc4-c4ccccc4)c4cc5c(ccc6sc7ccccc7c65)c5ccccc45)cc3c2)cc1]

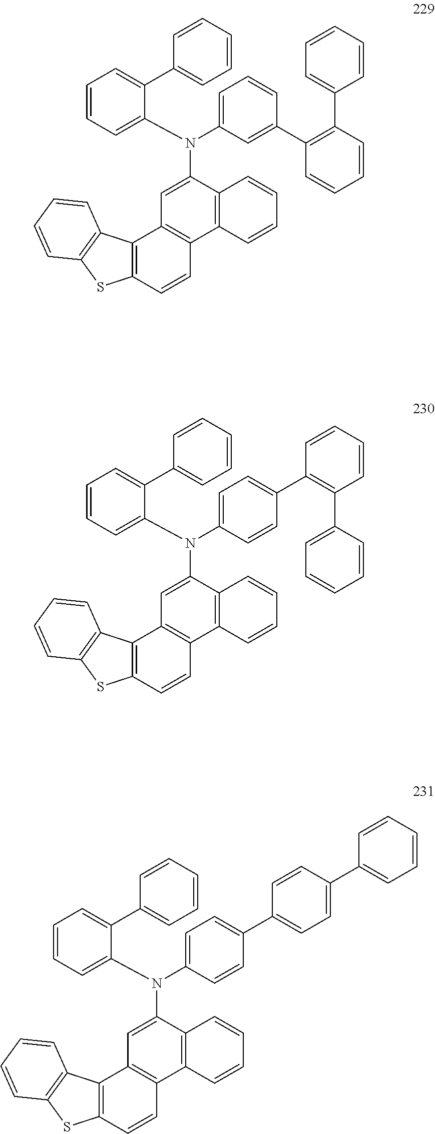 [SMILES: c1ccc(-c2ccccc2-c2cccc(N(c3ccccc3-c3ccccc3)c3cc4c(ccc5sc6ccccc6c54)c4ccccc34)c2)cc1 ; c1ccc(-c2ccccc2-c2ccc(N(c3ccccc3-c3ccccc3)c3cc4c(ccc5sc6ccccc6c54)c4ccccc34)cc2)cc1 ; c1ccc(-c2ccc(-c3ccc(N(c4ccccc4-c4ccccc4)c4cc5c(ccc6sc7ccccc7c65)c5ccccc45)cc3)cc2)cc1]

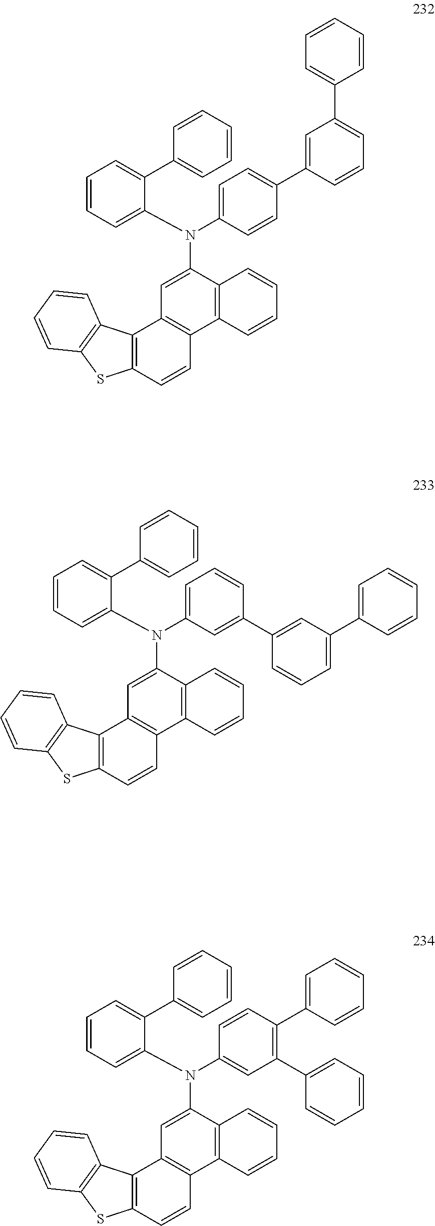 [SMILES: c1ccc(-c2cccc(-c3ccc(N(c4ccccc4-c4ccccc4)c4cc5c(ccc6sc7ccccc7c65)c5ccccc45)cc3)c2)cc1 ; c1ccc(-c2cccc(-c3cccc(N(c4ccccc4-c4ccccc4)c4cc5c(ccc6sc7ccccc7c65)c5ccccc45)c3)c2)cc1 ; c1ccc(-c2ccc(N(c3ccccc3-c3ccccc3)c3cc4c(ccc5sc6ccccc6c54)c4ccccc34)cc2-c2ccccc2)cc1]

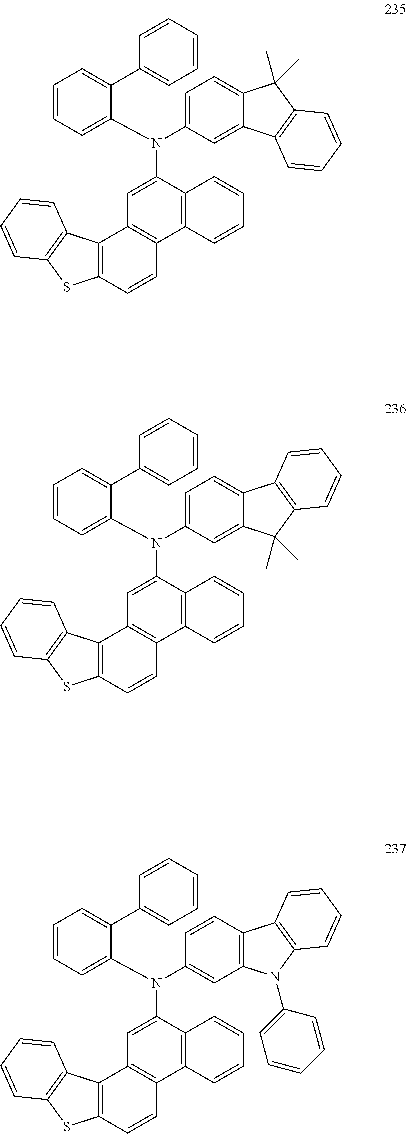 [SMILES: CC1(C)c2ccccc2-c2cc(N(c3ccccc3-c3ccccc3)c3cc4c(ccc5sc6ccccc6c54)c4ccccc34)ccc21 ; CC1(C)c2ccccc2-c2ccc(N(c3ccccc3-c3ccccc3)c3cc4c(ccc5sc6ccccc6c54)c4ccccc34)cc21 ; c1ccc(-c2ccccc2N(c2ccc3c4ccccc4n(-c4ccccc4)c3c2)c2cc3c(ccc4sc5ccccc5c43)c3ccccc23)cc1]

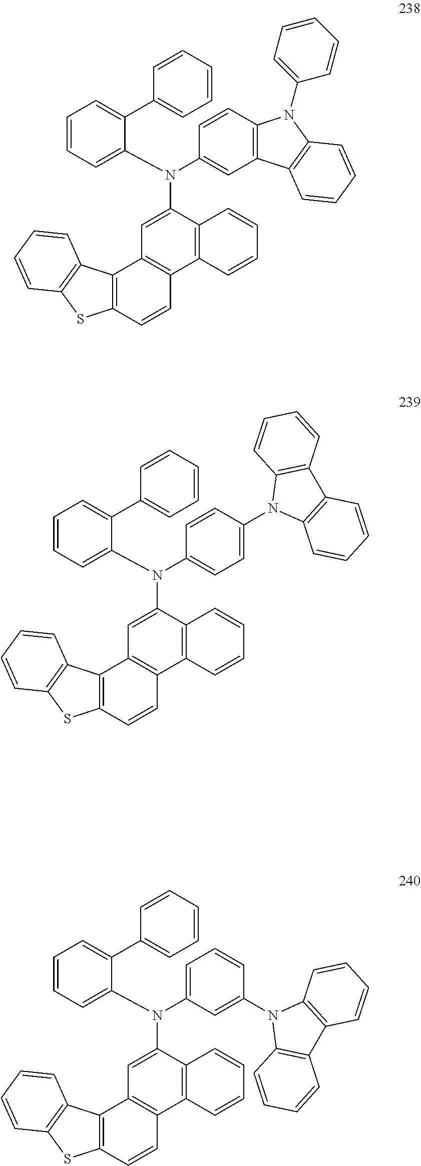 [SMILES: c1ccc(-c2ccccc2N(c2ccc3c(c2)c2ccccc2n3-c2ccccc2)c2cc3c(ccc4sc5ccccc5c43)c3ccccc23)cc1 ; c1ccc(-c2ccccc2N(c2ccc(-n3c4ccccc4c4ccccc43)cc2)c2cc3c(ccc4sc5ccccc5c43)c3ccccc23)cc1 ; c1ccc(-c2ccccc2N(c2cccc(-n3c4ccccc4c4ccccc43)c2)c2cc3c(ccc4sc5ccccc5c43)c3ccccc23)cc1]

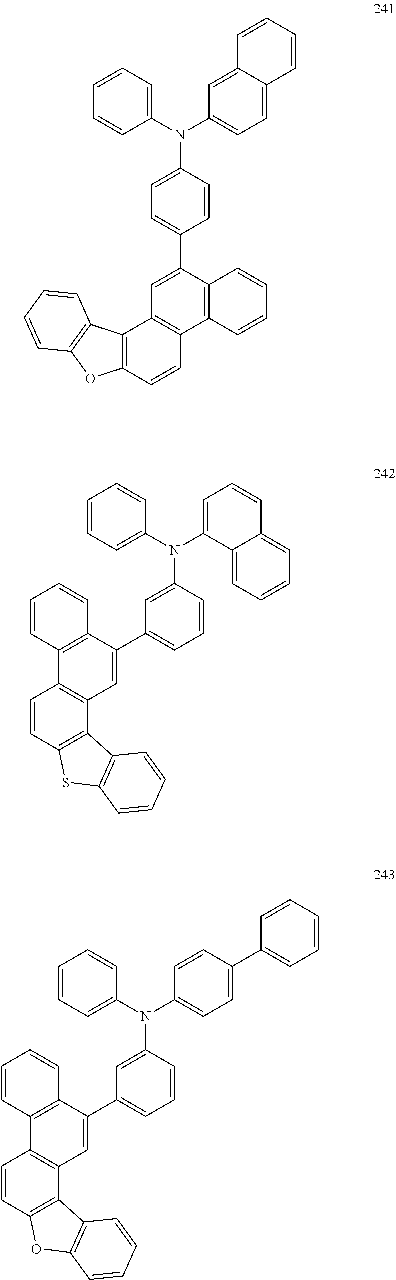 [SMILES: c1ccc(N(c2ccc(-c3cc4c(ccc5oc6ccccc6c54)c4ccccc34)cc2)c2ccc3ccccc3c2)cc1 ; c1ccc(N(c2cccc(-c3cc4c(ccc5sc6ccccc6c54)c4ccccc34)c2)c2cccc3ccccc23)cc1 ; c1ccc(-c2ccc(N(c3ccccc3)c3cccc(-c4cc5c(ccc6oc7ccccc7c65)c5ccccc45)c3)cc2)cc1]

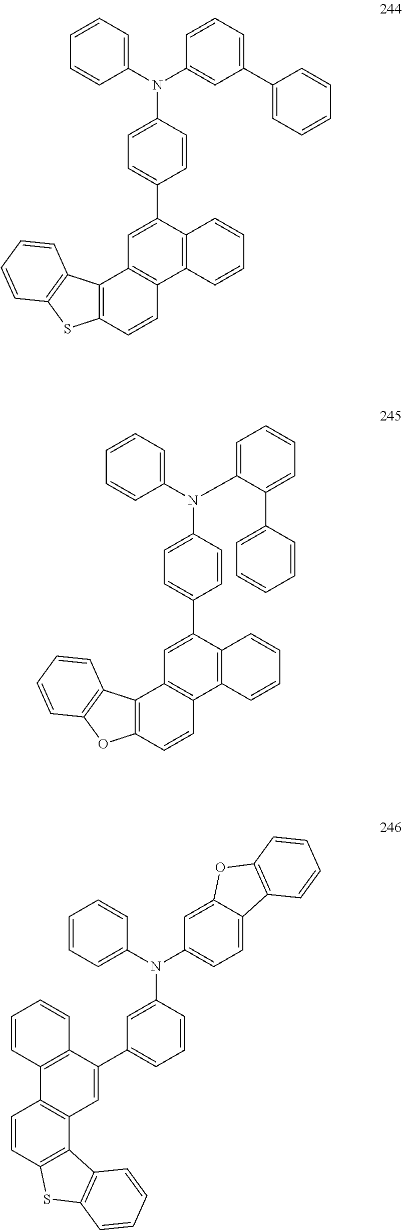 [SMILES: c1ccc(-c2cccc(N(c3ccccc3)c3ccc(-c4cc5c(ccc6sc7ccccc7c65)c5ccccc45)cc3)c2)cc1 ; c1ccc(-c2ccccc2N(c2ccccc2)c2ccc(-c3cc4c(ccc5oc6ccccc6c54)c4ccccc34)cc2)cc1 ; c1ccc(N(c2cccc(-c3cc4c(ccc5sc6ccccc6c54)c4ccccc34)c2)c2ccc3c(c2)oc2ccccc23)cc1]

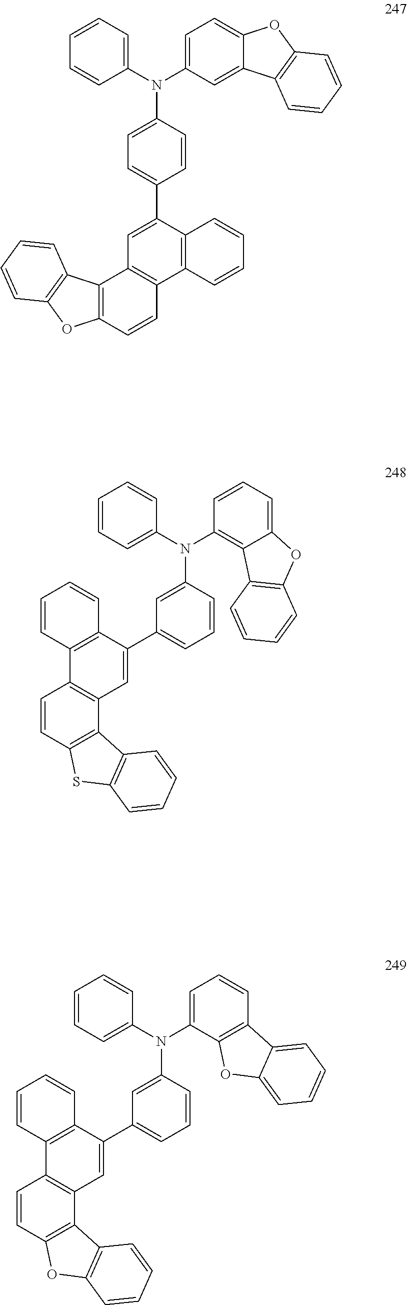 [SMILES: c1ccc(N(c2ccc(-c3cc4c(ccc5oc6ccccc6c54)c4ccccc34)cc2)c2ccc3oc4ccccc4c3c2)cc1 ; c1ccc(N(c2cccc(-c3cc4c(ccc5sc6ccccc6c54)c4ccccc34)c2)c2cccc3oc4ccccc4c23)cc1 ; c1ccc(N(c2cccc(-c3cc4c(ccc5oc6ccccc6c54)c4ccccc34)c2)c2cccc3c2oc2ccccc23)cc1]

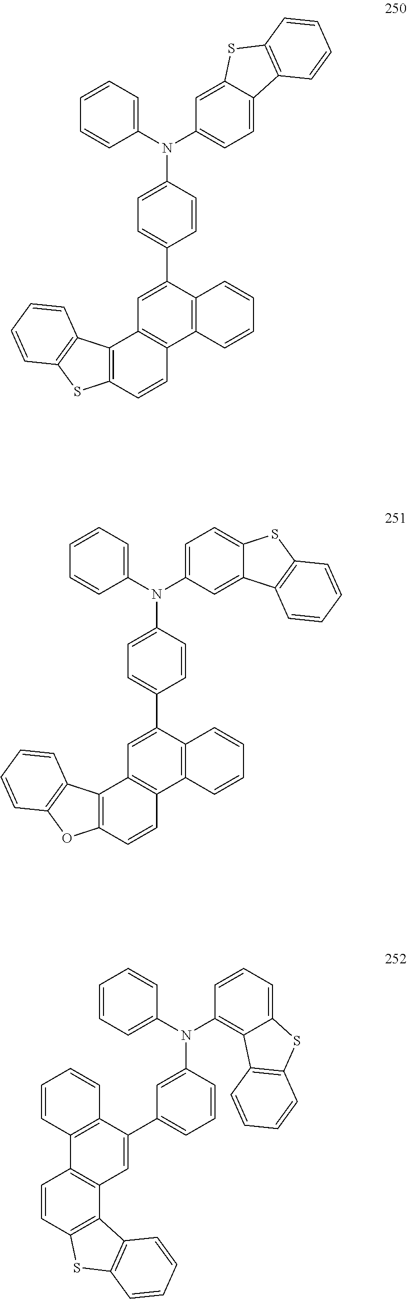 [SMILES: c1ccc(N(c2ccc(-c3cc4c(ccc5sc6ccccc6c54)c4ccccc34)cc2)c2ccc3c(c2)sc2ccccc23)cc1 ; c1ccc(N(c2ccc(-c3cc4c(ccc5oc6ccccc6c54)c4ccccc34)cc2)c2ccc3sc4ccccc4c3c2)cc1 ; c1ccc(N(c2cccc(-c3cc4c(ccc5sc6ccccc6c54)c4ccccc34)c2)c2cccc3sc4ccccc4c23)cc1]

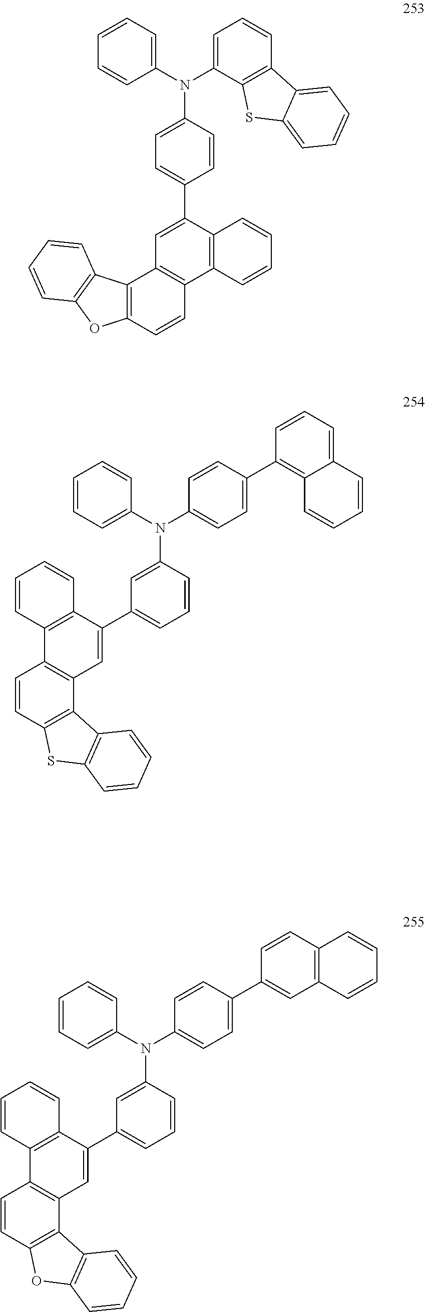 [SMILES: c1ccc(N(c2ccc(-c3cc4c(ccc5oc6ccccc6c54)c4ccccc34)cc2)c2cccc3c2sc2ccccc23)cc1 ; c1ccc(N(c2ccc(-c3cccc4ccccc34)cc2)c2cccc(-c3cc4c(ccc5sc6ccccc6c54)c4ccccc34)c2)cc1 ; c1ccc(N(c2ccc(-c3ccc4ccccc4c3)cc2)c2cccc(-c3cc4c(ccc5oc6ccccc6c54)c4ccccc34)c2)cc1]

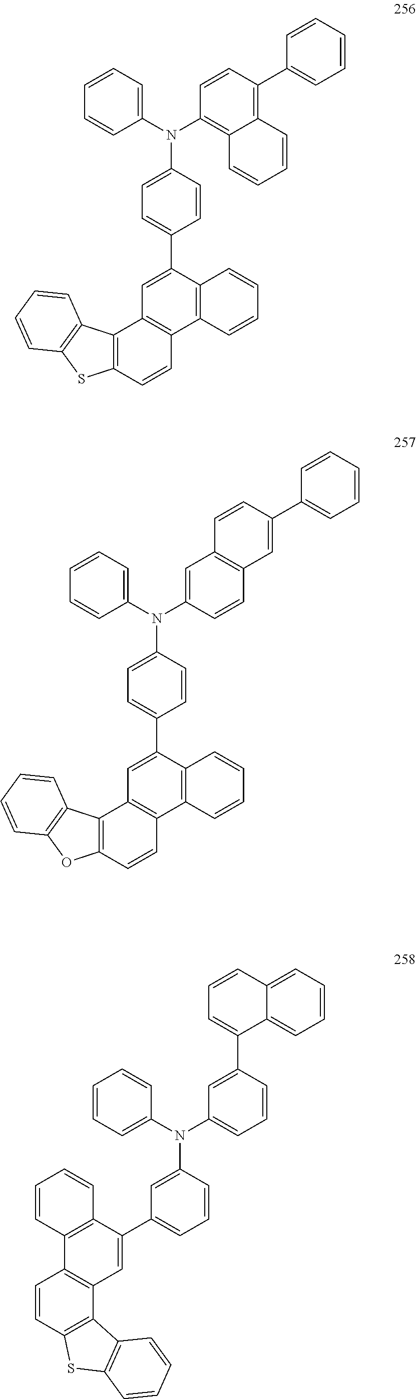 [SMILES: c1ccc(-c2ccc(N(c3ccccc3)c3ccc(-c4cc5c(ccc6sc7ccccc7c65)c5ccccc45)cc3)c3ccccc23)cc1 ; c1ccc(-c2ccc3cc(N(c4ccccc4)c4ccc(-c5cc6c(ccc7oc8ccccc8c76)c6ccccc56)cc4)ccc3c2)cc1 ; c1ccc(N(c2cccc(-c3cccc4ccccc34)c2)c2cccc(-c3cc4c(ccc5sc6ccccc6c54)c4ccccc34)c2)cc1]

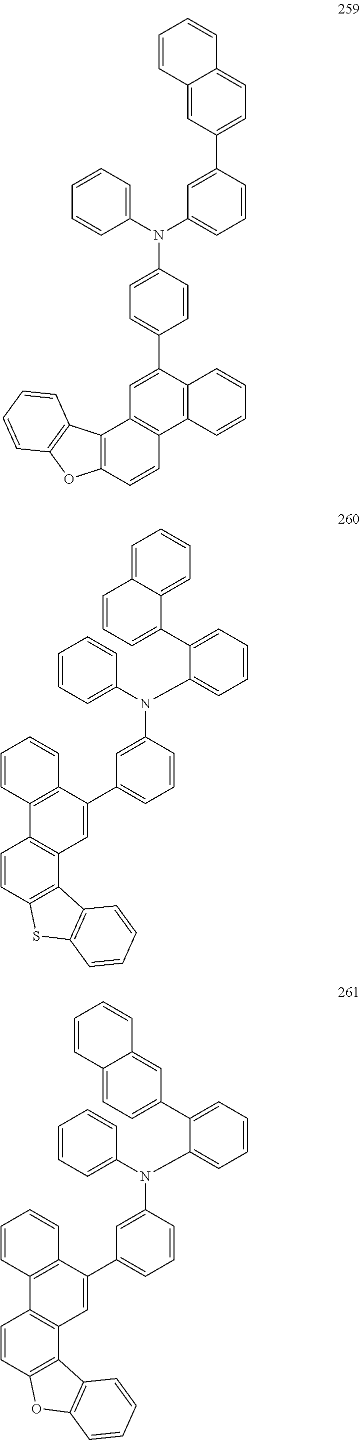

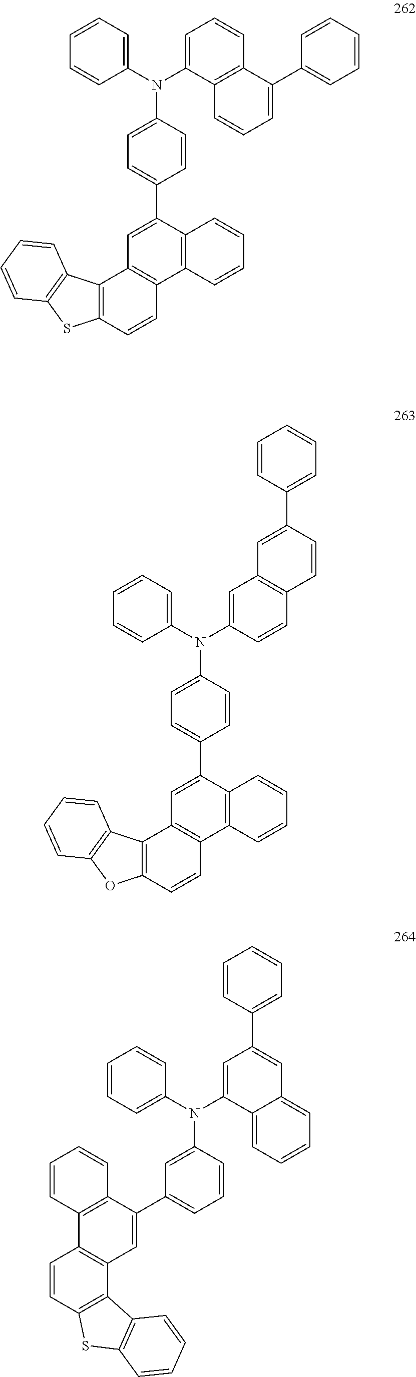 [SMILES: c1ccc(-c2cccc3c(N(c4ccccc4)c4ccc(-c5cc6c(ccc7sc8ccccc8c76)c6ccccc56)cc4)cccc23)cc1 ; c1ccc(-c2ccc3ccc(N(c4ccccc4)c4ccc(-c5cc6c(ccc7oc8ccccc8c76)c6ccccc56)cc4)cc3c2)cc1 ; c1ccc(-c2cc(N(c3ccccc3)c3cccc(-c4cc5c(ccc6sc7ccccc7c65)c5ccccc45)c3)c3ccccc3c2)cc1]

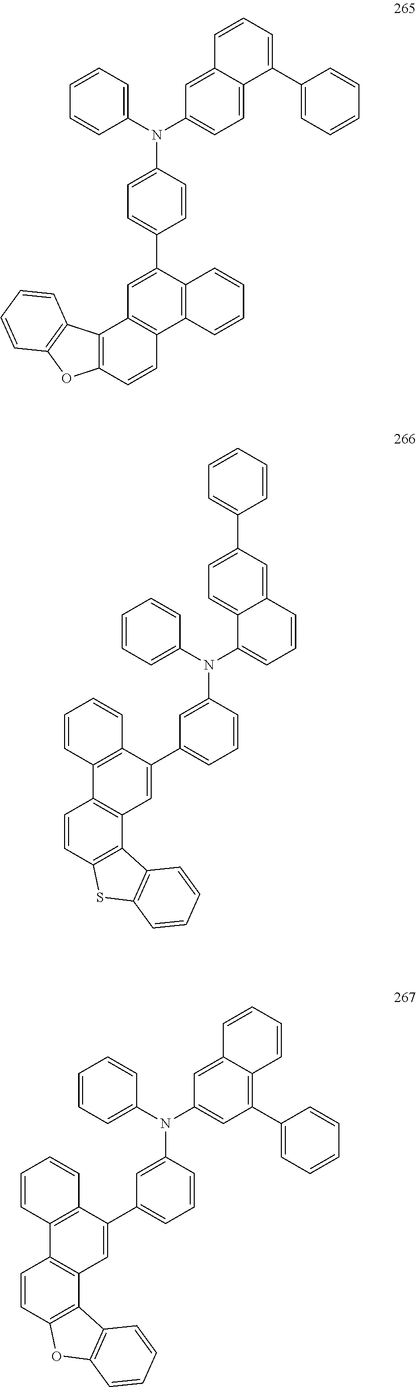 [SMILES: c1ccc(-c2cccc3cc(N(c4ccccc4)c4ccc(-c5cc6c(ccc7oc8ccccc8c76)c6ccccc56)cc4)ccc23)cc1 ; c1ccc(-c2ccc3c(N(c4ccccc4)c4cccc(-c5cc6c(ccc7sc8ccccc8c76)c6ccccc56)c4)cccc3c2)cc1 ; c1ccc(-c2cc(N(c3ccccc3)c3cccc(-c4cc5c(ccc6oc7ccccc7c65)c5ccccc45)c3)cc3ccccc23)cc1]

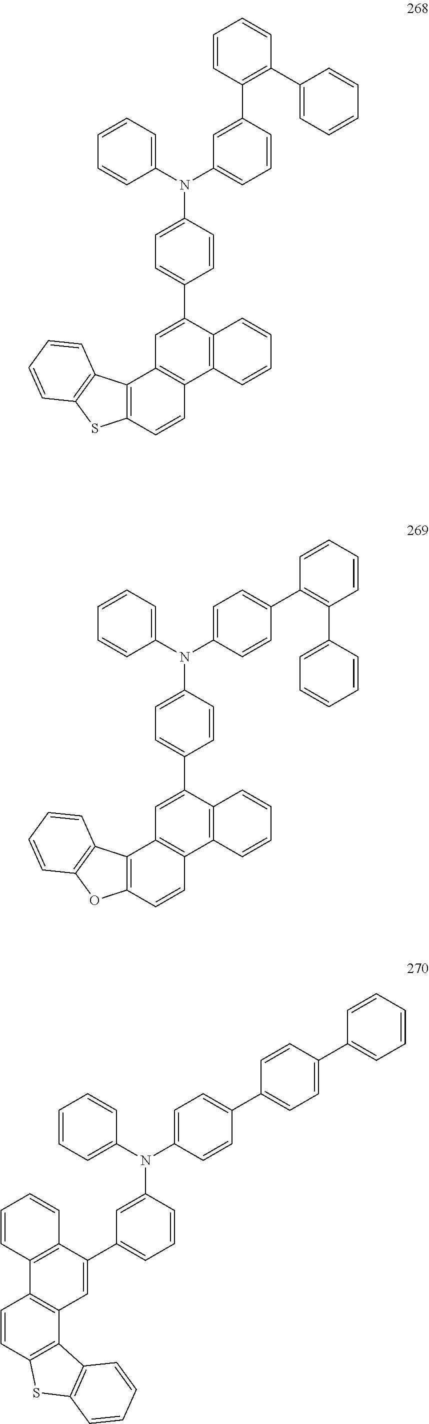 [SMILES: c1ccc(-c2ccccc2-c2cccc(N(c3ccccc3)c3ccc(-c4cc5c(ccc6sc7ccccc7c65)c5ccccc45)cc3)c2)cc1 ; c1ccc(-c2ccccc2-c2ccc(N(c3ccccc3)c3ccc(-c4cc5c(ccc6oc7ccccc7c65)c5ccccc45)cc3)cc2)cc1 ; c1ccc(-c2ccc(-c3ccc(N(c4ccccc4)c4cccc(-c5cc6c(ccc7sc8ccccc8c76)c6ccccc56)c4)cc3)cc2)cc1]

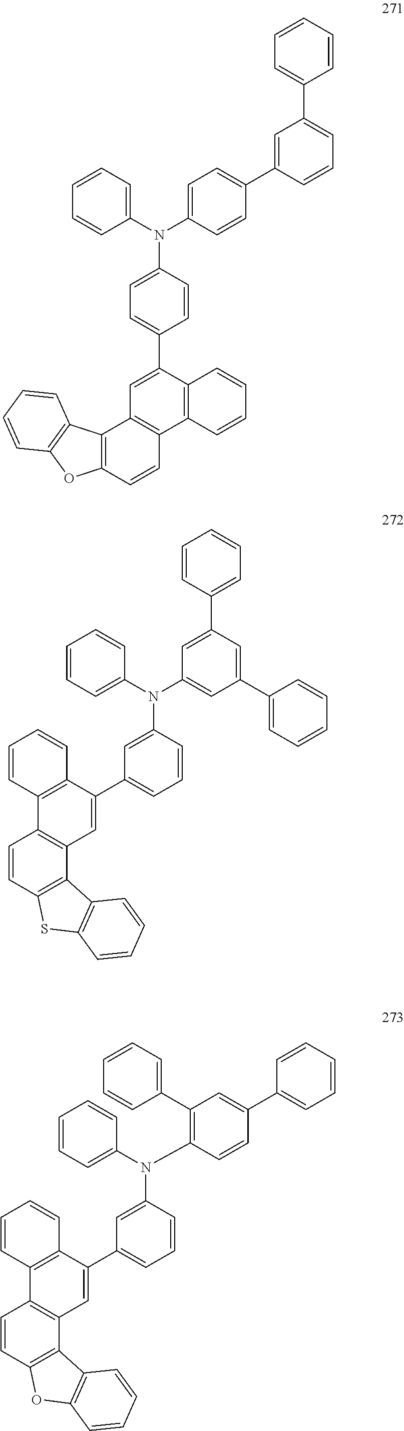 [SMILES: c1ccc(-c2cccc(-c3ccc(N(c4ccccc4)c4ccc(-c5cc6c(ccc7oc8ccccc8c76)c6ccccc56)cc4)cc3)c2)cc1 ; c1ccc(-c2cc(-c3ccccc3)cc(N(c3ccccc3)c3cccc(-c4cc5c(ccc6sc7ccccc7c65)c5ccccc45)c3)c2)cc1 ; c1ccc(-c2ccc(N(c3ccccc3)c3cccc(-c4cc5c(ccc6oc7ccccc7c65)c5ccccc45)c3)c(-c3ccccc3)c2)cc1]

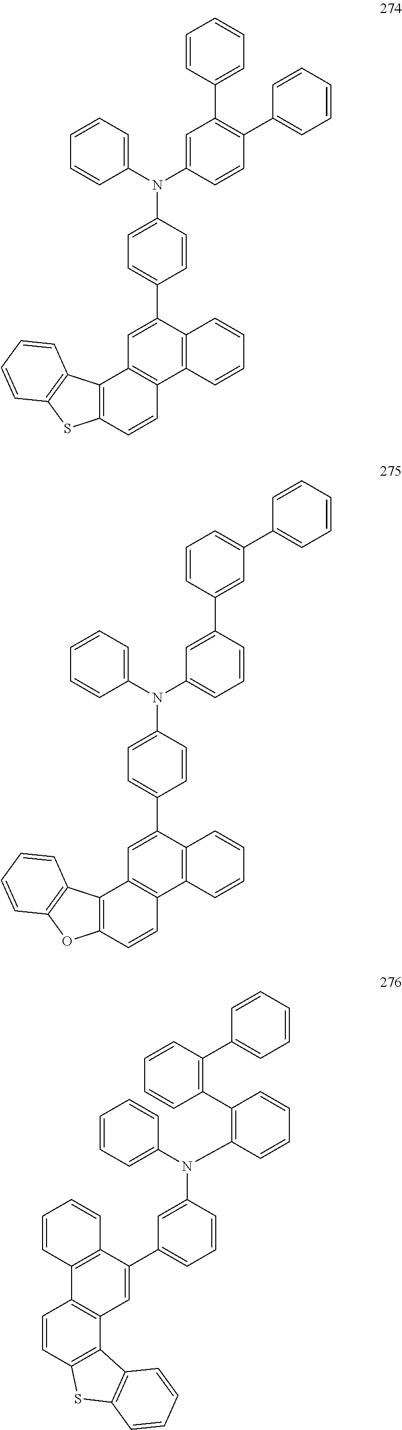 [SMILES: c1ccc(-c2ccc(N(c3ccccc3)c3ccc(-c4cc5c(ccc6sc7ccccc7c65)c5ccccc45)cc3)cc2-c2ccccc2)cc1 ; c1ccc(-c2cccc(-c3cccc(N(c4ccccc4)c4ccc(-c5cc6c(ccc7oc8ccccc8c76)c6ccccc56)cc4)c3)c2)cc1 ; c1ccc(-c2ccccc2-c2ccccc2N(c2ccccc2)c2cccc(-c3cc4c(ccc5sc6ccccc6c54)c4ccccc34)c2)cc1]

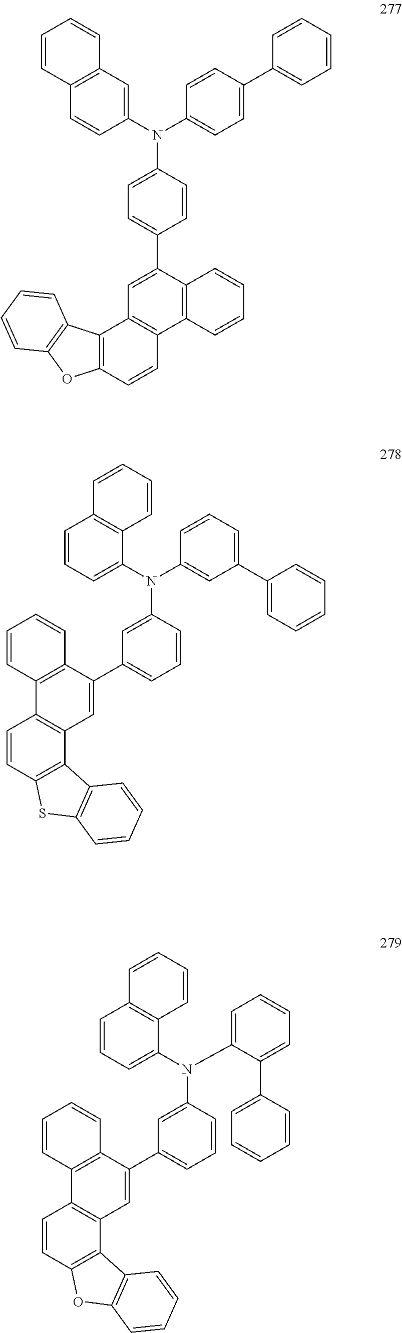 [SMILES: c1ccc(-c2ccc(N(c3ccc(-c4cc5c(ccc6oc7ccccc7c65)c5ccccc45)cc3)c3ccc4ccccc4c3)cc2)cc1 ; c1ccc(-c2cccc(N(c3cccc(-c4cc5c(ccc6sc7ccccc7c65)c5ccccc45)c3)c3cccc4ccccc34)c2)cc1 ; c1ccc(-c2ccccc2N(c2cccc(-c3cc4c(ccc5oc6ccccc6c54)c4ccccc34)c2)c2cccc3ccccc23)cc1]

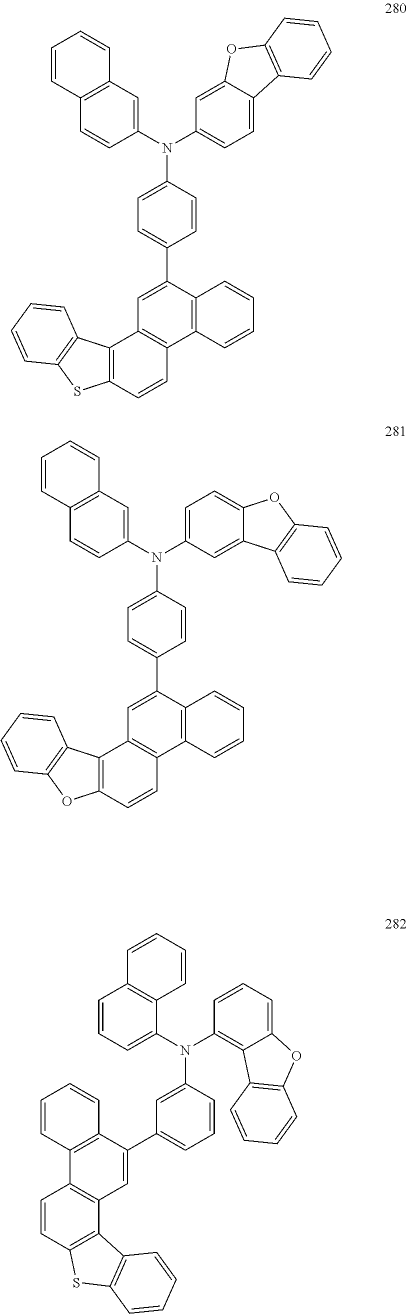 [SMILES: c1ccc2cc(N(c3ccc(-c4cc5c(ccc6sc7ccccc7c65)c5ccccc45)cc3)c3ccc4c(c3)oc3ccccc34)ccc2c1 ; c1ccc2cc(N(c3ccc(-c4cc5c(ccc6oc7ccccc7c65)c5ccccc45)cc3)c3ccc4oc5ccccc5c4c3)ccc2c1 ; c1cc(-c2cc3c(ccc4sc5ccccc5c43)c3ccccc23)cc(N(c2cccc3ccccc23)c2cccc3oc4ccccc4c23)c1]

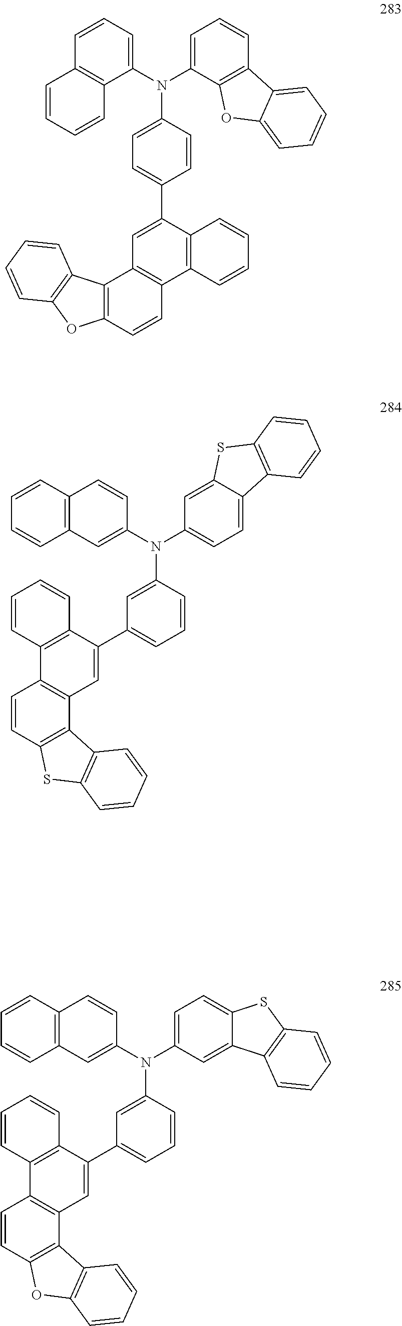 [SMILES: c1ccc2c(N(c3ccc(-c4cc5c(ccc6oc7ccccc7c65)c5ccccc45)cc3)c3cccc4c3oc3ccccc34)cccc2c1 ; c1cc(-c2cc3c(ccc4sc5ccccc5c43)c3ccccc23)cc(N(c2ccc3ccccc3c2)c2ccc3c(c2)sc2ccccc23)c1 ; c1cc(-c2cc3c(ccc4oc5ccccc5c43)c3ccccc23)cc(N(c2ccc3ccccc3c2)c2ccc3sc4ccccc4c3c2)c1]

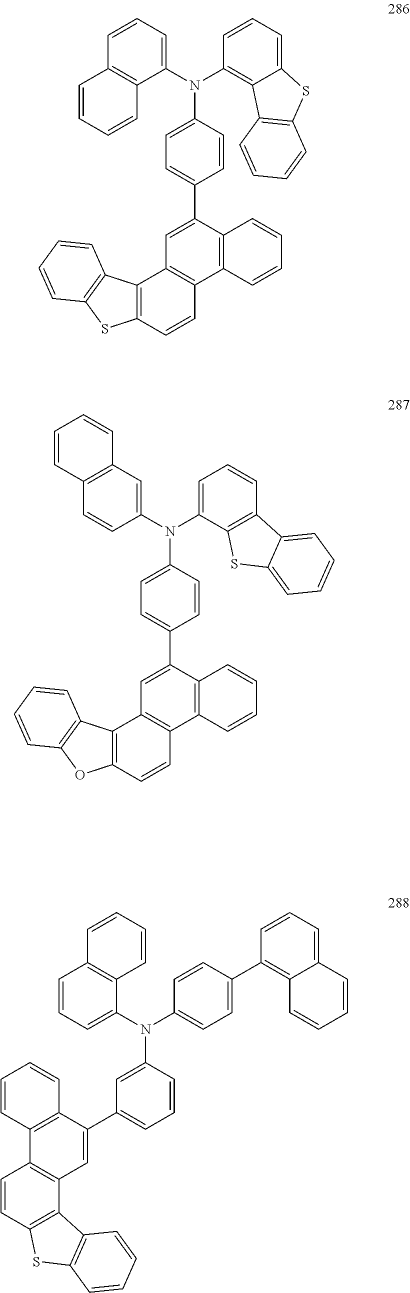 [SMILES: c1ccc2c(N(c3ccc(-c4cc5c(ccc6sc7ccccc7c65)c5ccccc45)cc3)c3cccc4sc5ccccc5c34)cccc2c1 ; c1ccc2cc(N(c3ccc(-c4cc5c(ccc6oc7ccccc7c65)c5ccccc45)cc3)c3cccc4c3sc3ccccc34)ccc2c1 ; c1cc(-c2cc3c(ccc4sc5ccccc5c43)c3ccccc23)cc(N(c2ccc(-c3cccc4ccccc34)cc2)c2cccc3ccccc23)c1]

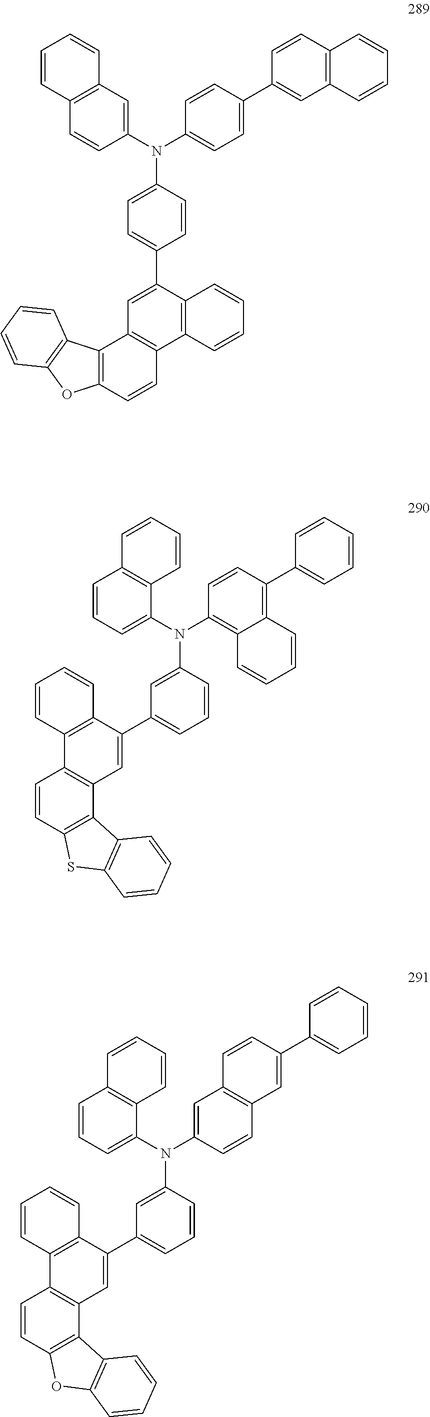 [SMILES: c1ccc2cc(-c3ccc(N(c4ccc(-c5cc6c(ccc7oc8ccccc8c76)c6ccccc56)cc4)c4ccc5ccccc5c4)cc3)ccc2c1 ; c1ccc(-c2ccc(N(c3cccc(-c4cc5c(ccc6sc7ccccc7c65)c5ccccc45)c3)c3cccc4ccccc34)c3ccccc23)cc1 ; c1ccc(-c2ccc3cc(N(c4cccc(-c5cc6c(ccc7oc8ccccc8c76)c6ccccc56)c4)c4cccc5ccccc45)ccc3c2)cc1]

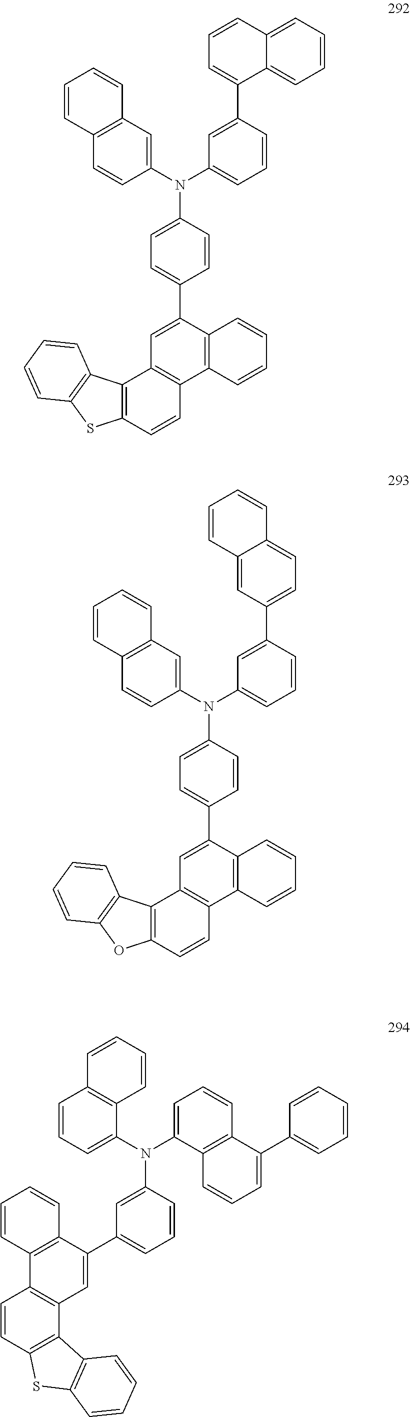 [SMILES: c1cc(-c2cccc3ccccc23)cc(N(c2ccc(-c3cc4c(ccc5sc6ccccc6c54)c4ccccc34)cc2)c2ccc3ccccc3c2)c1 ; c1cc(-c2ccc3ccccc3c2)cc(N(c2ccc(-c3cc4c(ccc5oc6ccccc6c54)c4ccccc34)cc2)c2ccc3ccccc3c2)c1 ; c1ccc(-c2cccc3c(N(c4cccc(-c5cc6c(ccc7sc8ccccc8c76)c6ccccc56)c4)c4cccc5ccccc45)cccc23)cc1]

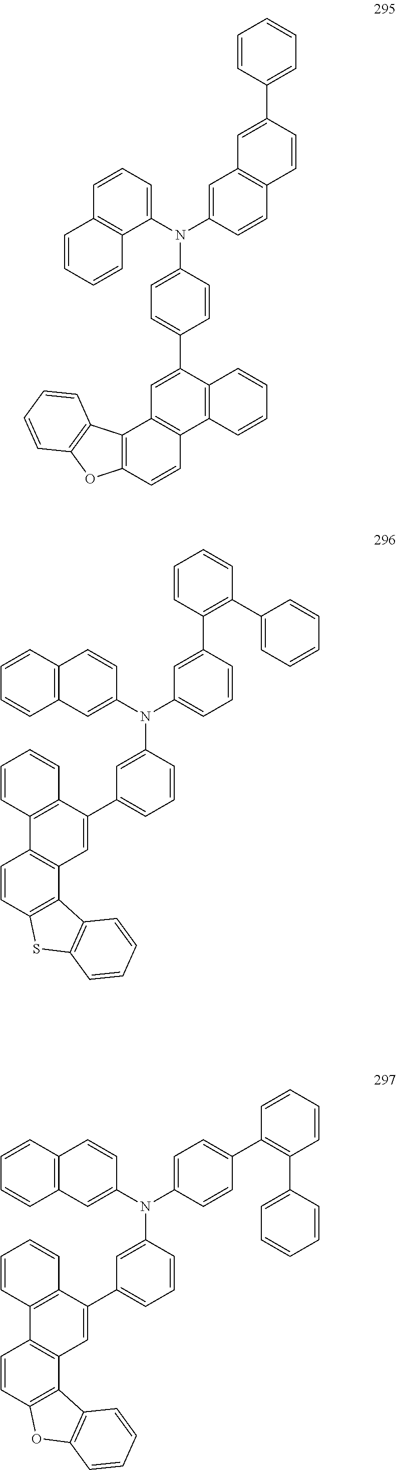 [SMILES: c1ccc(-c2ccc3ccc(N(c4ccc(-c5cc6c(ccc7oc8ccccc8c76)c6ccccc56)cc4)c4cccc5ccccc45)cc3c2)cc1 ; c1ccc(-c2ccccc2-c2cccc(N(c3cccc(-c4cc5c(ccc6sc7ccccc7c65)c5ccccc45)c3)c3ccc4ccccc4c3)c2)cc1 ; c1ccc(-c2ccccc2-c2ccc(N(c3cccc(-c4cc5c(ccc6oc7ccccc7c65)c5ccccc45)c3)c3ccc4ccccc4c3)cc2)cc1]

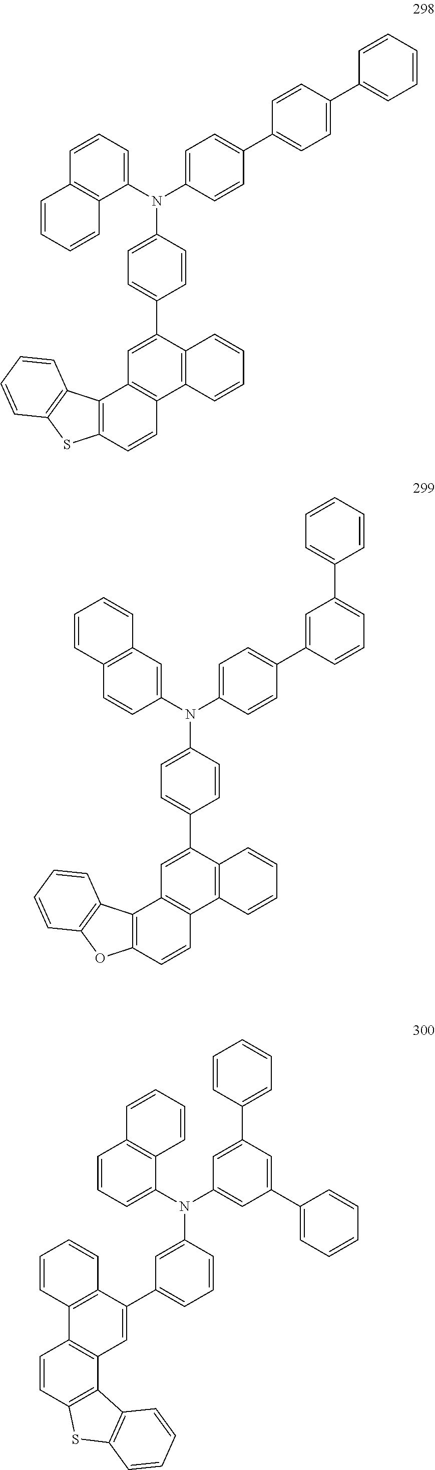 [SMILES: c1ccc(-c2ccc(-c3ccc(N(c4ccc(-c5cc6c(ccc7sc8ccccc8c76)c6ccccc56)cc4)c4cccc5ccccc45)cc3)cc2)cc1 ; c1ccc(-c2cccc(-c3ccc(N(c4ccc(-c5cc6c(ccc7oc8ccccc8c76)c6ccccc56)cc4)c4ccc5ccccc5c4)cc3)c2)cc1 ; c1ccc(-c2cc(-c3ccccc3)cc(N(c3cccc(-c4cc5c(ccc6sc7ccccc7c65)c5ccccc45)c3)c3cccc4ccccc34)c2)cc1]

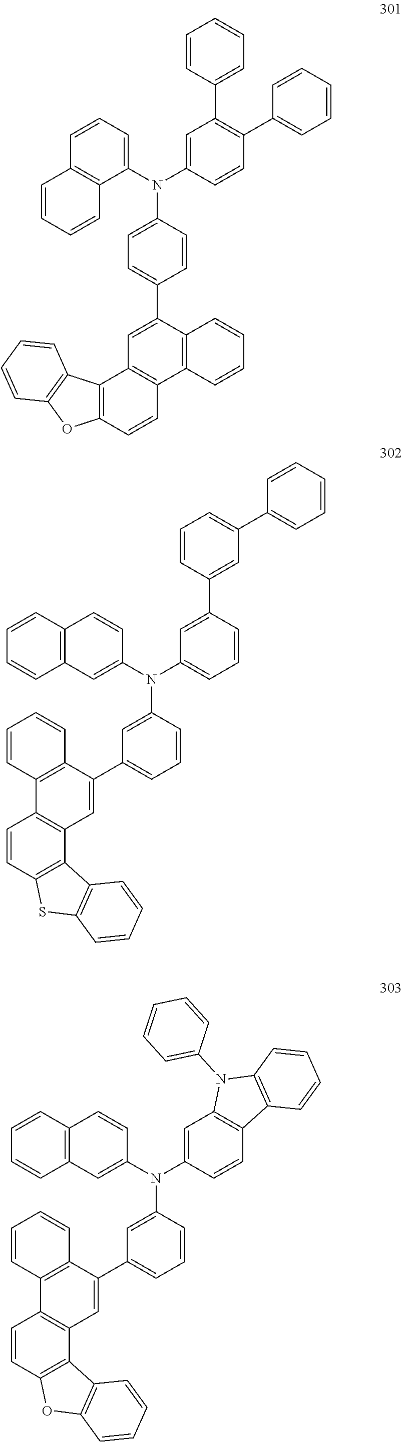 [SMILES: c1ccc(-c2ccc(N(c3ccc(-c4cc5c(ccc6oc7ccccc7c65)c5ccccc45)cc3)c3cccc4ccccc34)cc2-c2ccccc2)cc1 ; c1ccc(-c2cccc(-c3cccc(N(c4cccc(-c5cc6c(ccc7sc8ccccc8c76)c6ccccc56)c4)c4ccc5ccccc5c4)c3)c2)cc1 ; c1ccc(-n2c3ccccc3c3ccc(N(c4cccc(-c5cc6c(ccc7oc8ccccc8c76)c6ccccc56)c4)c4ccc5ccccc5c4)cc32)cc1]

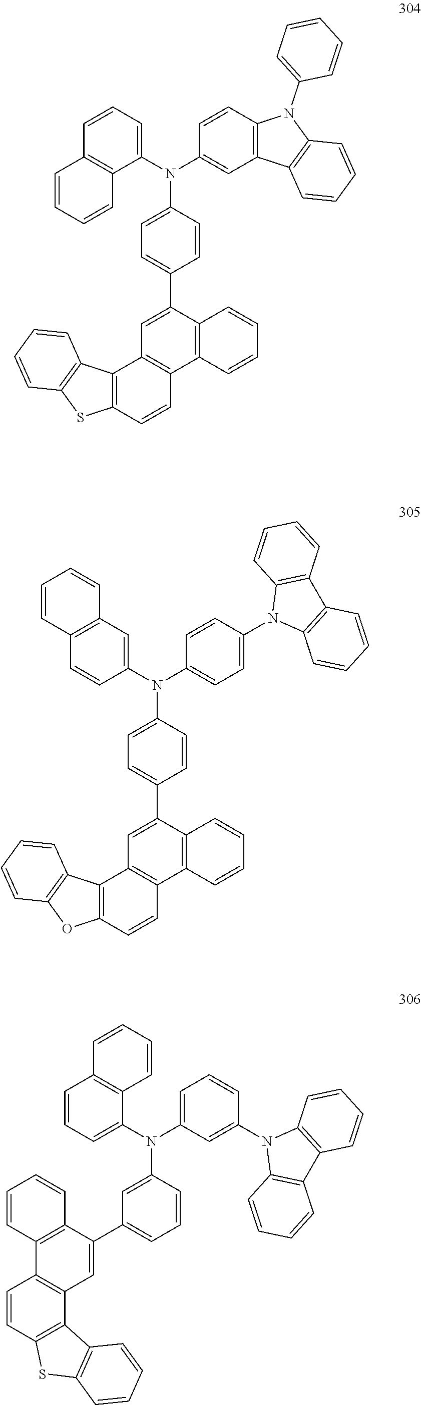 [SMILES: c1ccc(-n2c3ccccc3c3cc(N(c4ccc(-c5cc6c(ccc7sc8ccccc8c76)c6ccccc56)cc4)c4cccc5ccccc45)ccc32)cc1 ; c1ccc2cc(N(c3ccc(-c4cc5c(ccc6oc7ccccc7c65)c5ccccc45)cc3)c3ccc(-n4c5ccccc5c5ccccc54)cc3)ccc2c1 ; c1cc(-c2cc3c(ccc4sc5ccccc5c43)c3ccccc23)cc(N(c2cccc(-n3c4ccccc4c4ccccc43)c2)c2cccc3ccccc23)c1]

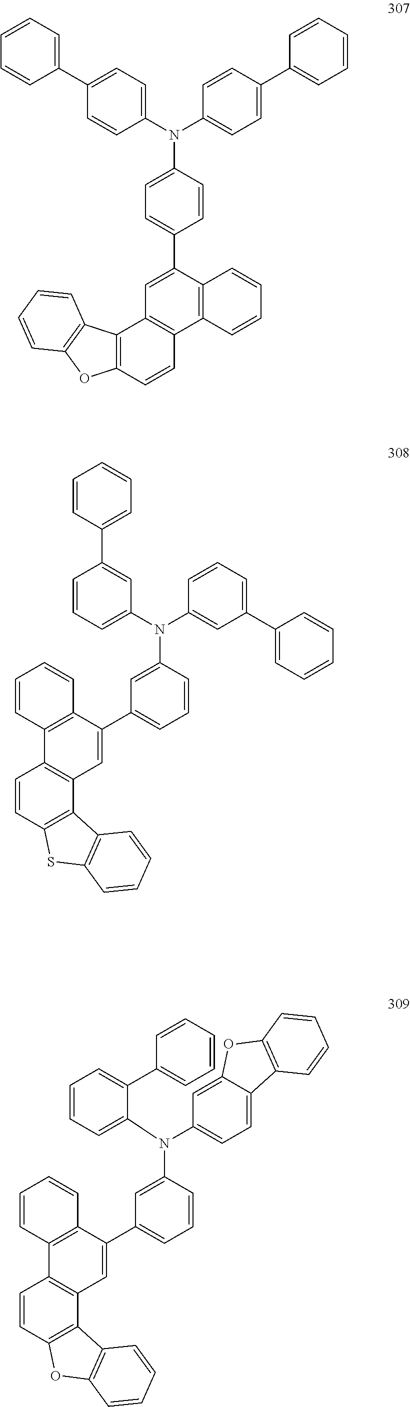 [SMILES: c1ccc(-c2ccc(N(c3ccc(-c4ccccc4)cc3)c3ccc(-c4cc5c(ccc6oc7ccccc7c65)c5ccccc45)cc3)cc2)cc1 ; c1ccc(-c2cccc(N(c3cccc(-c4ccccc4)c3)c3cccc(-c4cc5c(ccc6sc7ccccc7c65)c5ccccc45)c3)c2)cc1 ; c1ccc(-c2ccccc2N(c2cccc(-c3cc4c(ccc5oc6ccccc6c54)c4ccccc34)c2)c2ccc3c(c2)oc2ccccc23)cc1]

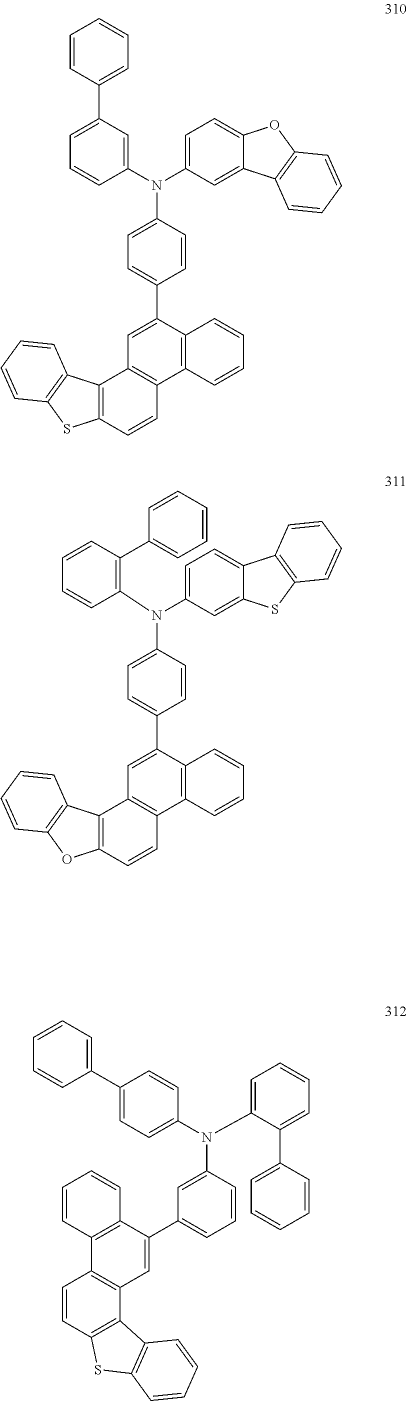 [SMILES: c1ccc(-c2cccc(N(c3ccc(-c4cc5c(ccc6sc7ccccc7c65)c5ccccc45)cc3)c3ccc4oc5ccccc5c4c3)c2)cc1 ; c1ccc(-c2ccccc2N(c2ccc(-c3cc4c(ccc5oc6ccccc6c54)c4ccccc34)cc2)c2ccc3c(c2)sc2ccccc23)cc1 ; c1ccc(-c2ccc(N(c3cccc(-c4cc5c(ccc6sc7ccccc7c65)c5ccccc45)c3)c3ccccc3-c3ccccc3)cc2)cc1]

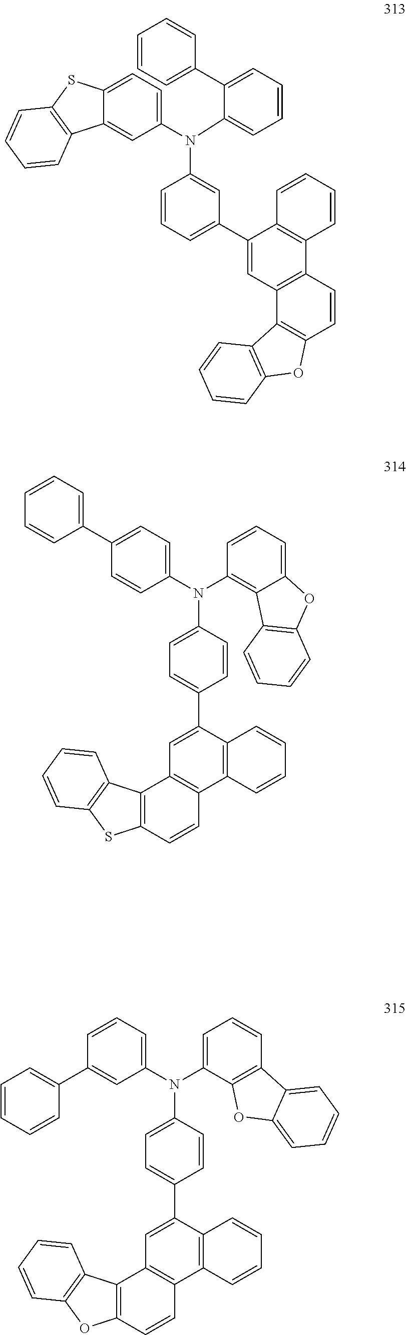 [SMILES: c1ccc(-c2ccccc2N(c2cccc(-c3cc4c(ccc5oc6ccccc6c54)c4ccccc34)c2)c2ccc3sc4ccccc4c3c2)cc1 ; c1ccc(-c2ccc(N(c3ccc(-c4cc5c(ccc6sc7ccccc7c65)c5ccccc45)cc3)c3cccc4oc5ccccc5c34)cc2)cc1 ; c1ccc(-c2cccc(N(c3ccc(-c4cc5c(ccc6oc7ccccc7c65)c5ccccc45)cc3)c3cccc4c3oc3ccccc34)c2)cc1]

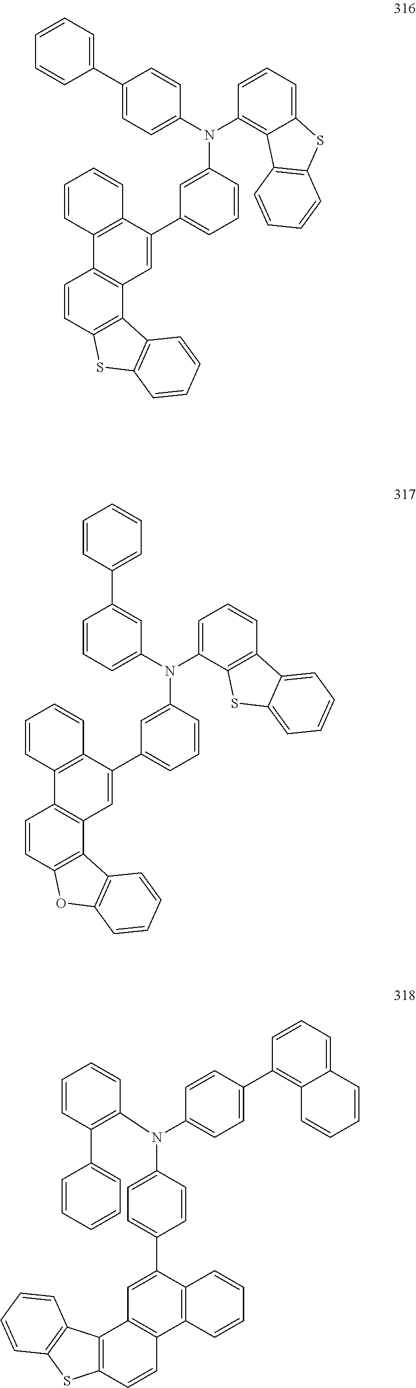 [SMILES: c1ccc(-c2ccc(N(c3cccc(-c4cc5c(ccc6sc7ccccc7c65)c5ccccc45)c3)c3cccc4sc5ccccc5c34)cc2)cc1 ; c1ccc(-c2cccc(N(c3cccc(-c4cc5c(ccc6oc7ccccc7c65)c5ccccc45)c3)c3cccc4c3sc3ccccc34)c2)cc1 ; c1ccc(-c2ccccc2N(c2ccc(-c3cccc4ccccc34)cc2)c2ccc(-c3cc4c(ccc5sc6ccccc6c54)c4ccccc34)cc2)cc1]

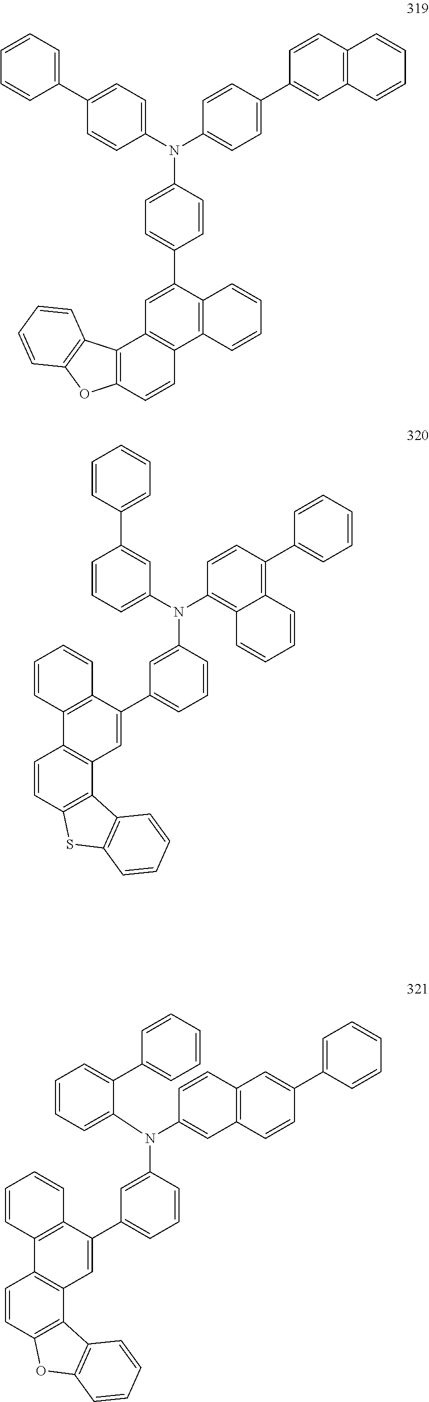 [SMILES: c1ccc(-c2ccc(N(c3ccc(-c4ccc5ccccc5c4)cc3)c3ccc(-c4cc5c(ccc6oc7ccccc7c65)c5ccccc45)cc3)cc2)cc1 ; c1ccc(-c2cccc(N(c3cccc(-c4cc5c(ccc6sc7ccccc7c65)c5ccccc45)c3)c3ccc(-c4ccccc4)c4ccccc34)c2)cc1 ; c1ccc(-c2ccc3cc(N(c4cccc(-c5cc6c(ccc7oc8ccccc8c76)c6ccccc56)c4)c4ccccc4-c4ccccc4)ccc3c2)cc1]

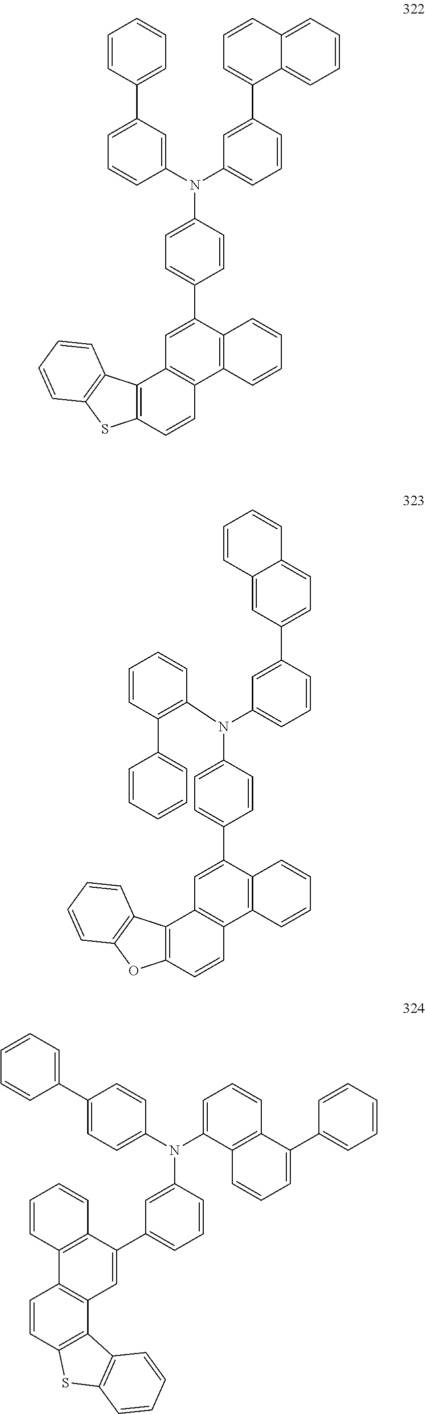 [SMILES: c1ccc(-c2cccc(N(c3ccc(-c4cc5c(ccc6sc7ccccc7c65)c5ccccc45)cc3)c3cccc(-c4cccc5ccccc45)c3)c2)cc1 ; c1ccc(-c2ccccc2N(c2ccc(-c3cc4c(ccc5oc6ccccc6c54)c4ccccc34)cc2)c2cccc(-c3ccc4ccccc4c3)c2)cc1 ; c1ccc(-c2ccc(N(c3cccc(-c4cc5c(ccc6sc7ccccc7c65)c5ccccc45)c3)c3cccc4c(-c5ccccc5)cccc34)cc2)cc1]

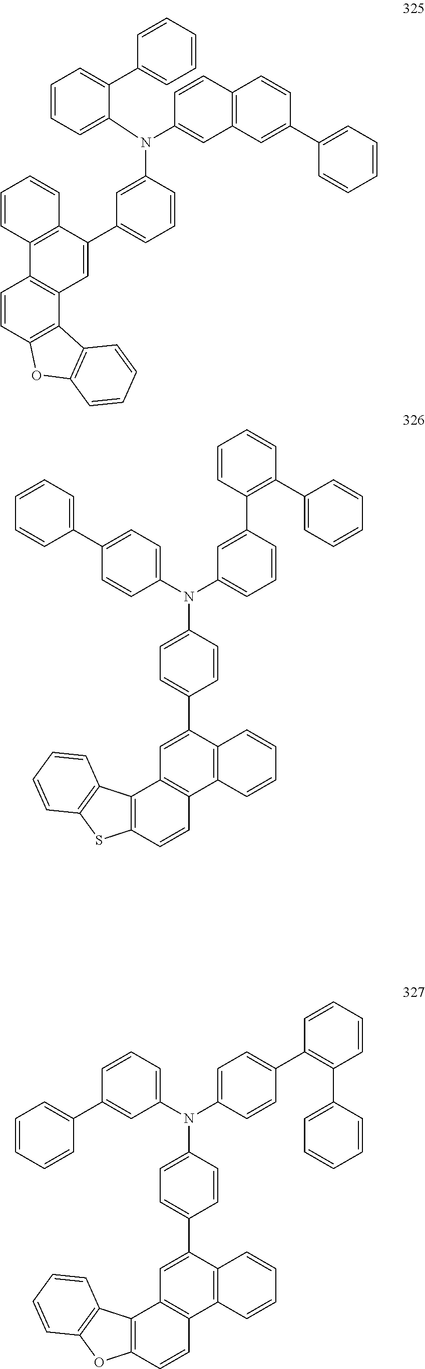 [SMILES: c1ccc(-c2ccc3ccc(N(c4cccc(-c5cc6c(ccc7oc8ccccc8c76)c6ccccc56)c4)c4ccccc4-c4ccccc4)cc3c2)cc1 ; c1ccc(-c2ccc(N(c3ccc(-c4cc5c(ccc6sc7ccccc7c65)c5ccccc45)cc3)c3cccc(-c4ccccc4-c4ccccc4)c3)cc2)cc1 ; c1ccc(-c2cccc(N(c3ccc(-c4ccccc4-c4ccccc4)cc3)c3ccc(-c4cc5c(ccc6oc7ccccc7c65)c5ccccc45)cc3)c2)cc1]

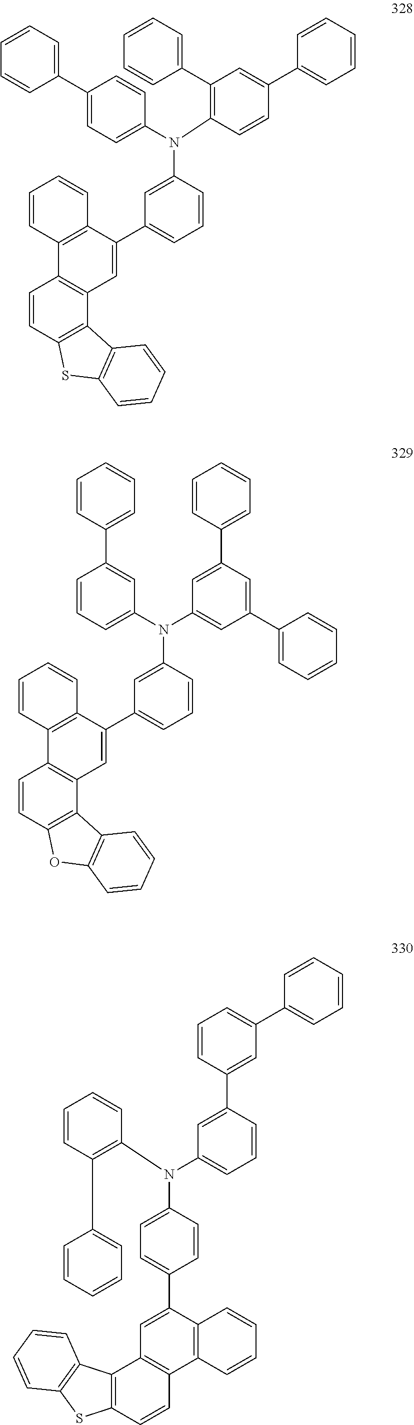 [SMILES: c1ccc(-c2ccc(N(c3cccc(-c4cc5c(ccc6sc7ccccc7c65)c5ccccc45)c3)c3ccc(-c4ccccc4)cc3-c3ccccc3)cc2)cc1 ; c1ccc(-c2cccc(N(c3cc(-c4ccccc4)cc(-c4ccccc4)c3)c3cccc(-c4cc5c(ccc6oc7ccccc7c65)c5ccccc45)c3)c2)cc1 ; c1ccc(-c2cccc(-c3cccc(N(c4ccc(-c5cc6c(ccc7sc8ccccc8c76)c6ccccc56)cc4)c4ccccc4-c4ccccc4)c3)c2)cc1]

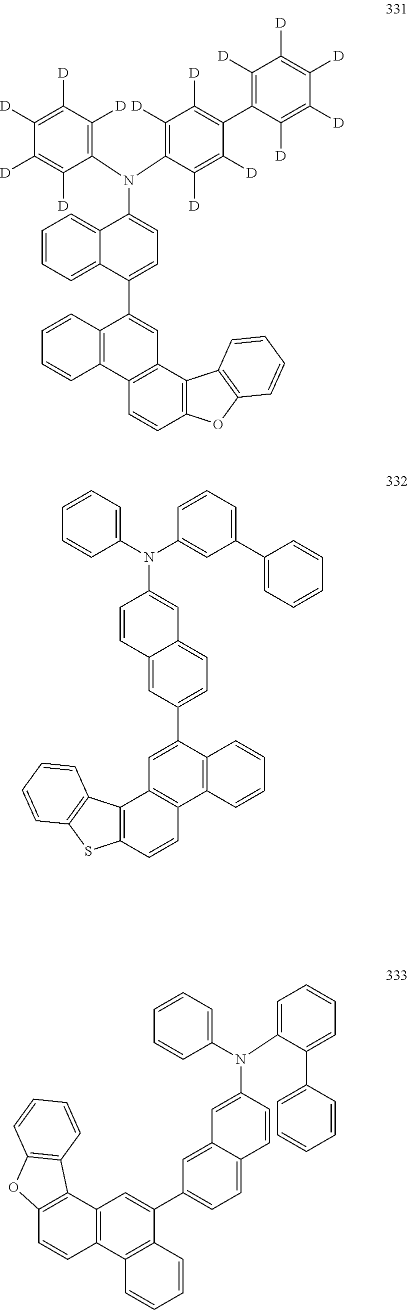 [SMILES: [2H]c1c([2H])c([2H])c(-c2c([2H])c([2H])c(N(c3c([2H])c([2H])c([2H])c([2H])c3[2H])c3ccc(-c4cc5c(ccc6oc7ccccc7c65)c5ccccc45)c4ccccc34)c([2H])c2[2H])c([2H])c1[2H] ; c1ccc(-c2cccc(N(c3ccccc3)c3ccc4cc(-c5cc6c(ccc7sc8ccccc8c76)c6ccccc56)ccc4c3)c2)cc1 ; c1ccc(-c2ccccc2N(c2ccccc2)c2ccc3ccc(-c4cc5c(ccc6oc7ccccc7c65)c5ccccc45)cc3c2)cc1]

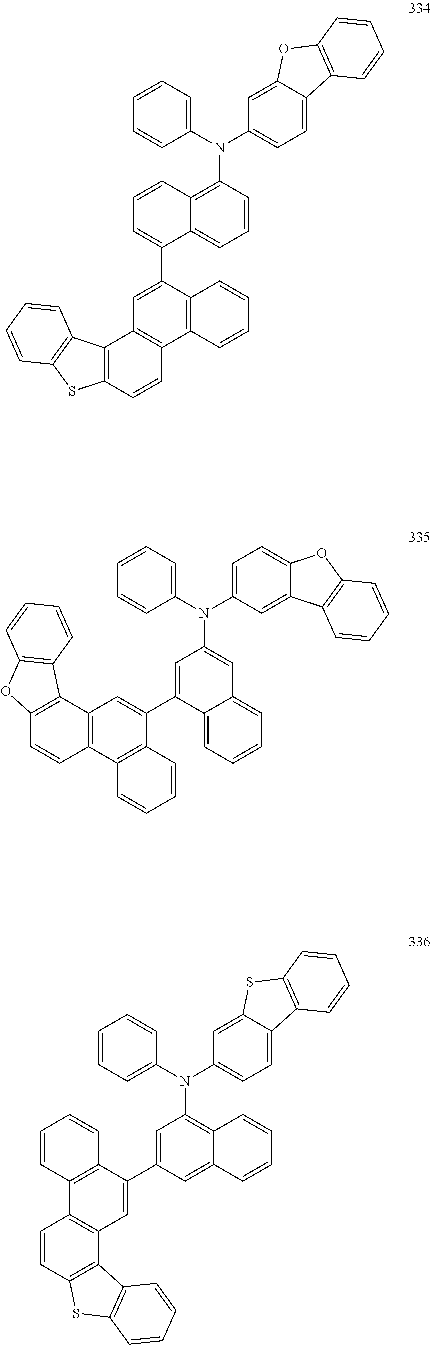 [SMILES: c1ccc(N(c2ccc3c(c2)oc2ccccc23)c2cccc3c(-c4cc5c(ccc6sc7ccccc7c65)c5ccccc45)cccc23)cc1 ; c1ccc(N(c2cc(-c3cc4c(ccc5oc6ccccc6c54)c4ccccc34)c3ccccc3c2)c2ccc3oc4ccccc4c3c2)cc1 ; c1ccc(N(c2ccc3c(c2)sc2ccccc23)c2cc(-c3cc4c(ccc5sc6ccccc6c54)c4ccccc34)cc3ccccc23)cc1]

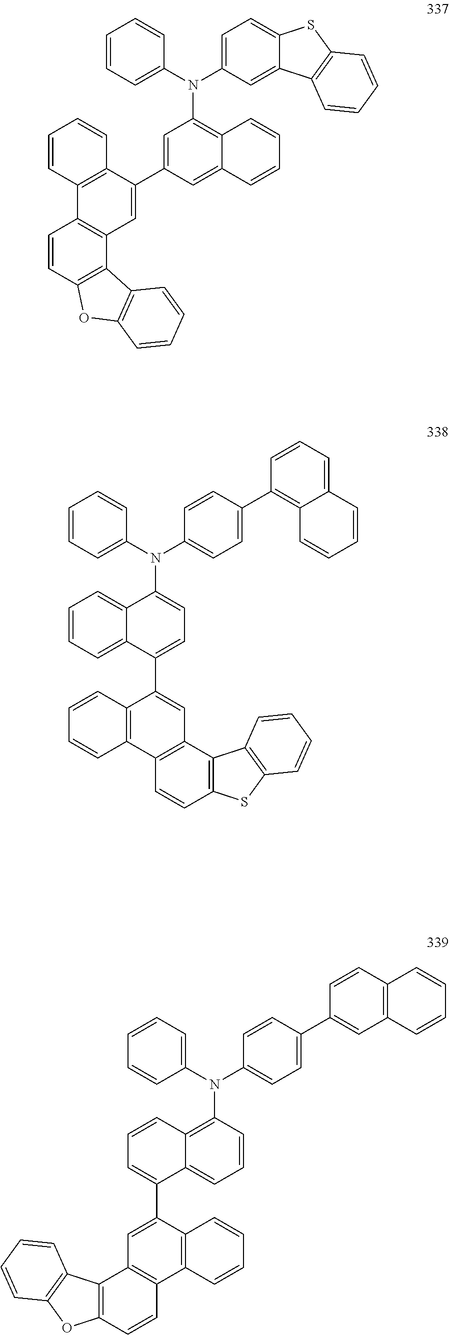 [SMILES: c1ccc(N(c2ccc3sc4ccccc4c3c2)c2cc(-c3cc4c(ccc5oc6ccccc6c54)c4ccccc34)cc3ccccc23)cc1 ; c1ccc(N(c2ccc(-c3cccc4ccccc34)cc2)c2ccc(-c3cc4c(ccc5sc6ccccc6c54)c4ccccc34)c3ccccc23)cc1 ; c1ccc(N(c2ccc(-c3ccc4ccccc4c3)cc2)c2cccc3c(-c4cc5c(ccc6oc7ccccc7c65)c5ccccc45)cccc23)cc1]

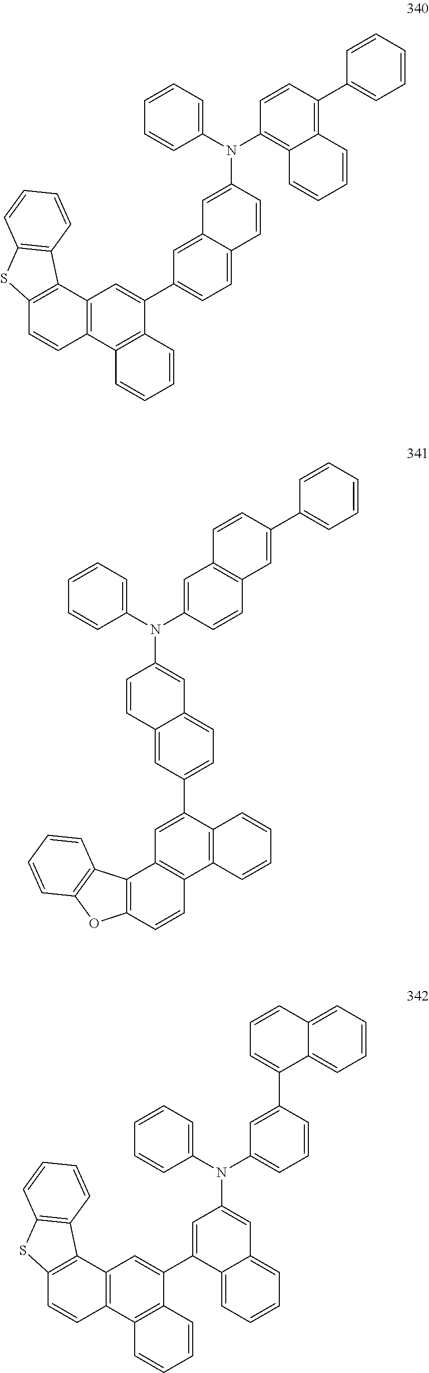 [SMILES: c1ccc(-c2ccc(N(c3ccccc3)c3ccc4ccc(-c5cc6c(ccc7sc8ccccc8c76)c6ccccc56)cc4c3)c3ccccc23)cc1 ; c1ccc(-c2ccc3cc(N(c4ccccc4)c4ccc5cc(-c6cc7c(ccc8oc9ccccc9c87)c7ccccc67)ccc5c4)ccc3c2)cc1 ; c1ccc(N(c2cccc(-c3cccc4ccccc34)c2)c2cc(-c3cc4c(ccc5sc6ccccc6c54)c4ccccc34)c3ccccc3c2)cc1]

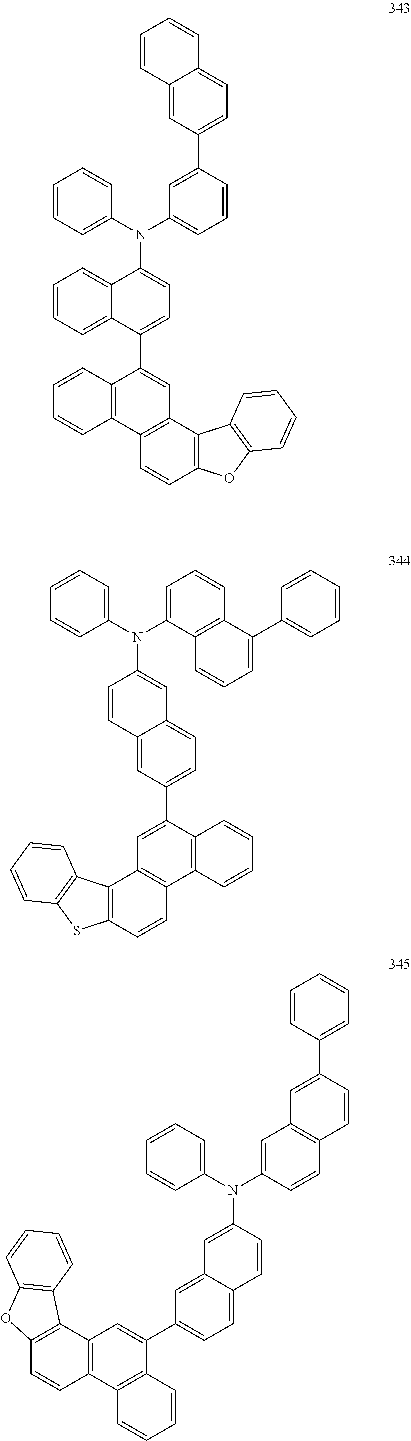 [SMILES: c1ccc(N(c2cccc(-c3ccc4ccccc4c3)c2)c2ccc(-c3cc4c(ccc5oc6ccccc6c54)c4ccccc34)c3ccccc23)cc1 ; c1ccc(-c2cccc3c(N(c4ccccc4)c4ccc5cc(-c6cc7c(ccc8sc9ccccc9c87)c7ccccc67)ccc5c4)cccc23)cc1 ; c1ccc(-c2ccc3ccc(N(c4ccccc4)c4ccc5ccc(-c6cc7c(ccc8oc9ccccc9c87)c7ccccc67)cc5c4)cc3c2)cc1]

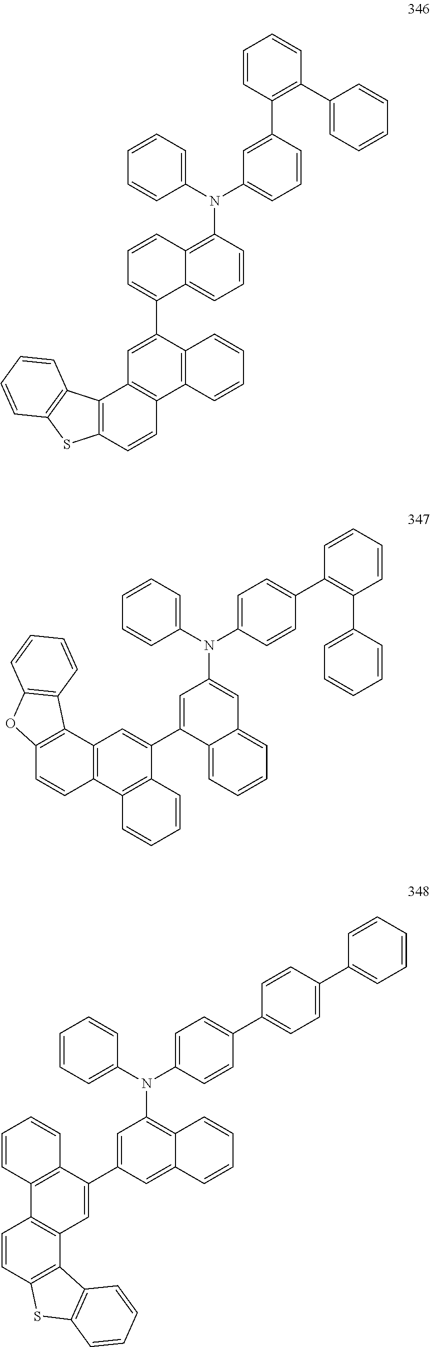 [SMILES: c1ccc(-c2ccccc2-c2cccc(N(c3ccccc3)c3cccc4c(-c5cc6c(ccc7sc8ccccc8c76)c6ccccc56)cccc34)c2)cc1 ; c1ccc(-c2ccccc2-c2ccc(N(c3ccccc3)c3cc(-c4cc5c(ccc6oc7ccccc7c65)c5ccccc45)c4ccccc4c3)cc2)cc1 ; c1ccc(-c2ccc(-c3ccc(N(c4ccccc4)c4cc(-c5cc6c(ccc7sc8ccccc8c76)c6ccccc56)cc5ccccc45)cc3)cc2)cc1]

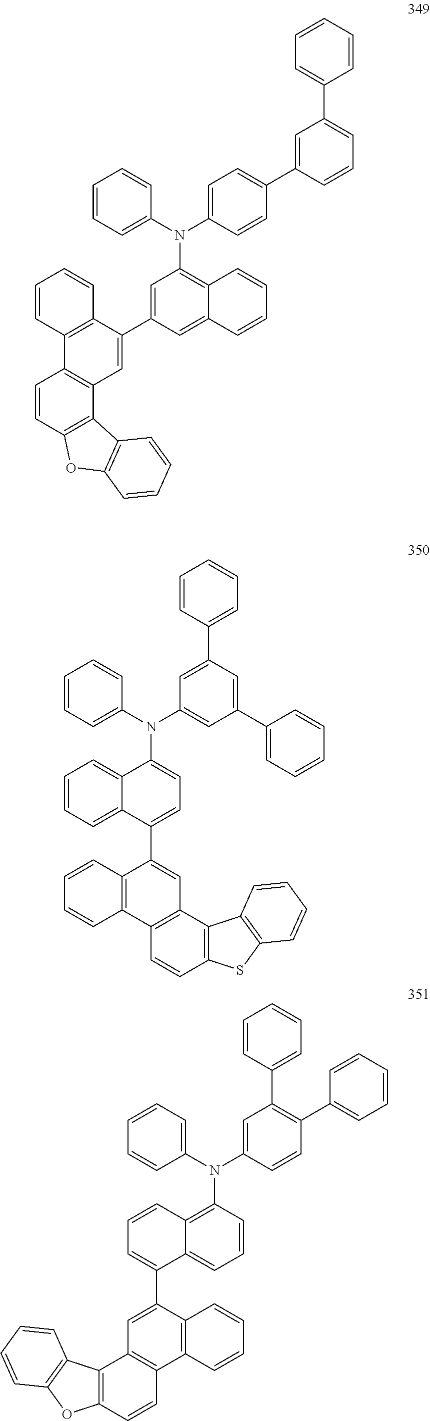

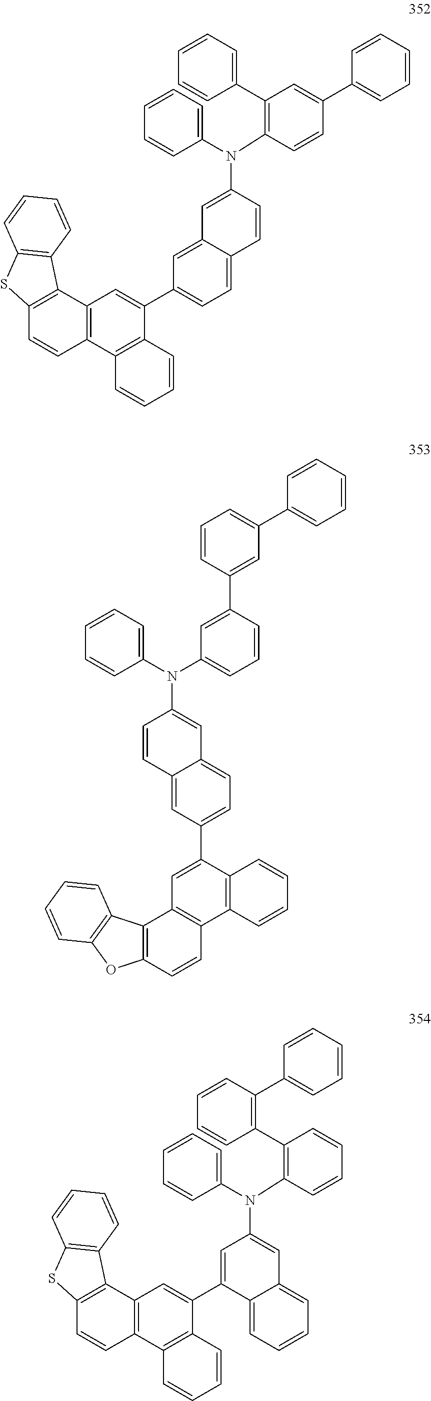 [SMILES: c1ccc(-c2ccc(N(c3ccccc3)c3ccc4ccc(-c5cc6c(ccc7sc8ccccc8c76)c6ccccc56)cc4c3)c(-c3ccccc3)c2)cc1 ; c1ccc(-c2cccc(-c3cccc(N(c4ccccc4)c4ccc5cc(-c6cc7c(ccc8oc9ccccc9c87)c7ccccc67)ccc5c4)c3)c2)cc1 ; c1ccc(-c2ccccc2-c2ccccc2N(c2ccccc2)c2cc(-c3cc4c(ccc5sc6ccccc6c54)c4ccccc34)c3ccccc3c2)cc1]

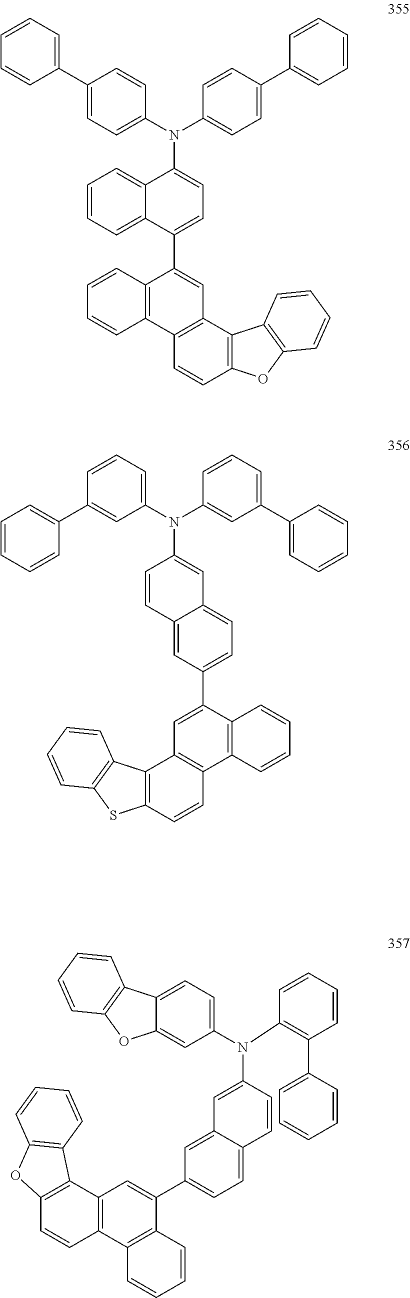 [SMILES: c1ccc(-c2ccc(N(c3ccc(-c4ccccc4)cc3)c3ccc(-c4cc5c(ccc6oc7ccccc7c65)c5ccccc45)c4ccccc34)cc2)cc1 ; c1ccc(-c2cccc(N(c3cccc(-c4ccccc4)c3)c3ccc4cc(-c5cc6c(ccc7sc8ccccc8c76)c6ccccc56)ccc4c3)c2)cc1 ; c1ccc(-c2ccccc2N(c2ccc3ccc(-c4cc5c(ccc6oc7ccccc7c65)c5ccccc45)cc3c2)c2ccc3c(c2)oc2ccccc23)cc1]

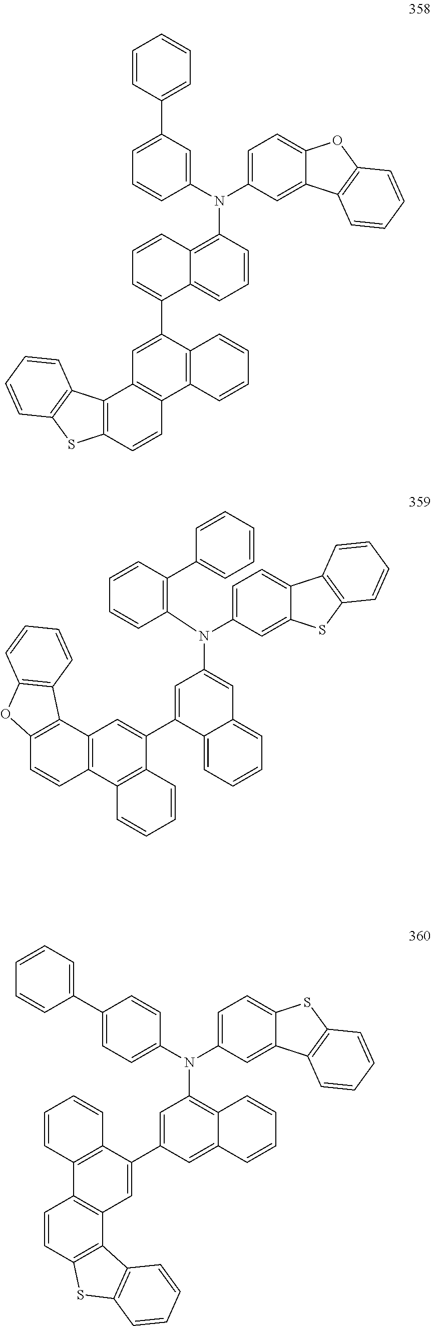 [SMILES: c1ccc(-c2cccc(N(c3ccc4oc5ccccc5c4c3)c3cccc4c(-c5cc6c(ccc7sc8ccccc8c76)c6ccccc56)cccc34)c2)cc1 ; c1ccc(-c2ccccc2N(c2cc(-c3cc4c(ccc5oc6ccccc6c54)c4ccccc34)c3ccccc3c2)c2ccc3c(c2)sc2ccccc23)cc1 ; c1ccc(-c2ccc(N(c3ccc4sc5ccccc5c4c3)c3cc(-c4cc5c(ccc6sc7ccccc7c65)c5ccccc45)cc4ccccc34)cc2)cc1]

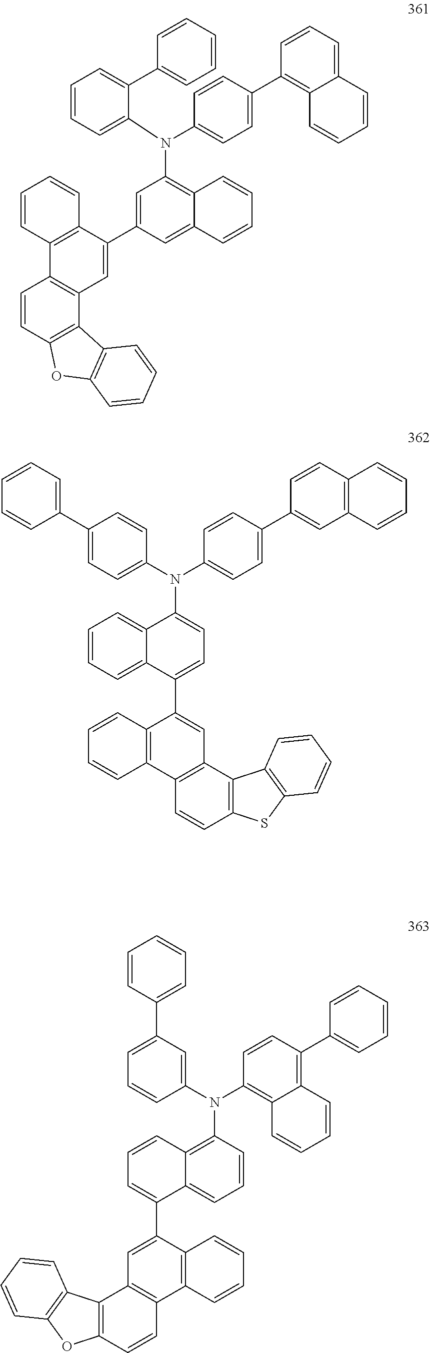 [SMILES: c1ccc(-c2ccccc2N(c2ccc(-c3cccc4ccccc34)cc2)c2cc(-c3cc4c(ccc5oc6ccccc6c54)c4ccccc34)cc3ccccc23)cc1 ; c1ccc(-c2ccc(N(c3ccc(-c4ccc5ccccc5c4)cc3)c3ccc(-c4cc5c(ccc6sc7ccccc7c65)c5ccccc45)c4ccccc34)cc2)cc1 ; c1ccc(-c2cccc(N(c3ccc(-c4ccccc4)c4ccccc34)c3cccc4c(-c5cc6c(ccc7oc8ccccc8c76)c6ccccc56)cccc34)c2)cc1]

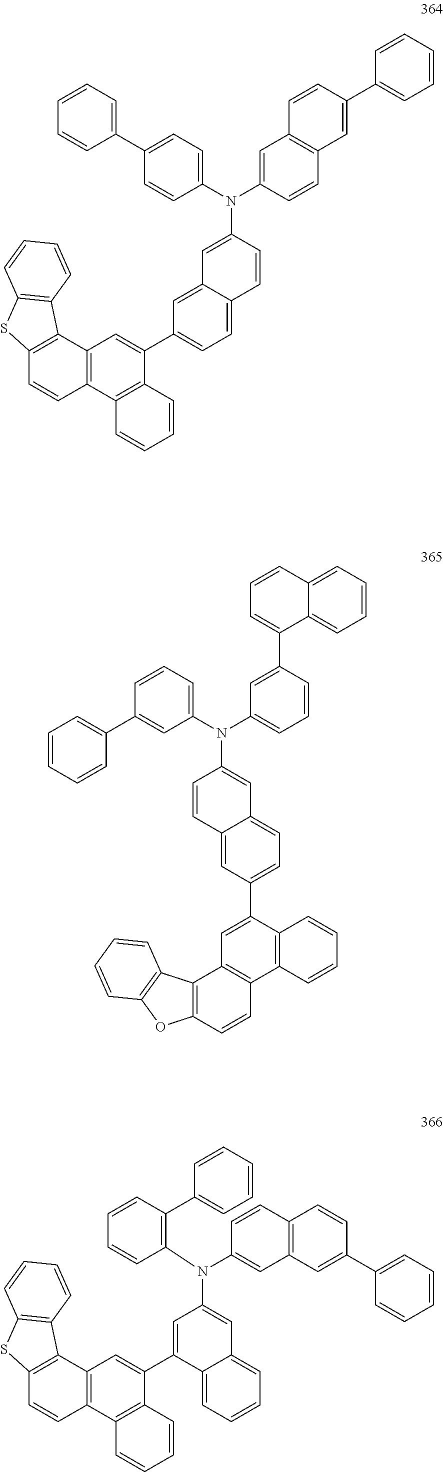 [SMILES: c1ccc(-c2ccc(N(c3ccc4cc(-c5ccccc5)ccc4c3)c3ccc4ccc(-c5cc6c(ccc7sc8ccccc8c76)c6ccccc56)cc4c3)cc2)cc1 ; c1ccc(-c2cccc(N(c3cccc(-c4cccc5ccccc45)c3)c3ccc4cc(-c5cc6c(ccc7oc8ccccc8c76)c6ccccc56)ccc4c3)c2)cc1 ; c1ccc(-c2ccc3ccc(N(c4cc(-c5cc6c(ccc7sc8ccccc8c76)c6ccccc56)c5ccccc5c4)c4ccccc4-c4ccccc4)cc3c2)cc1]

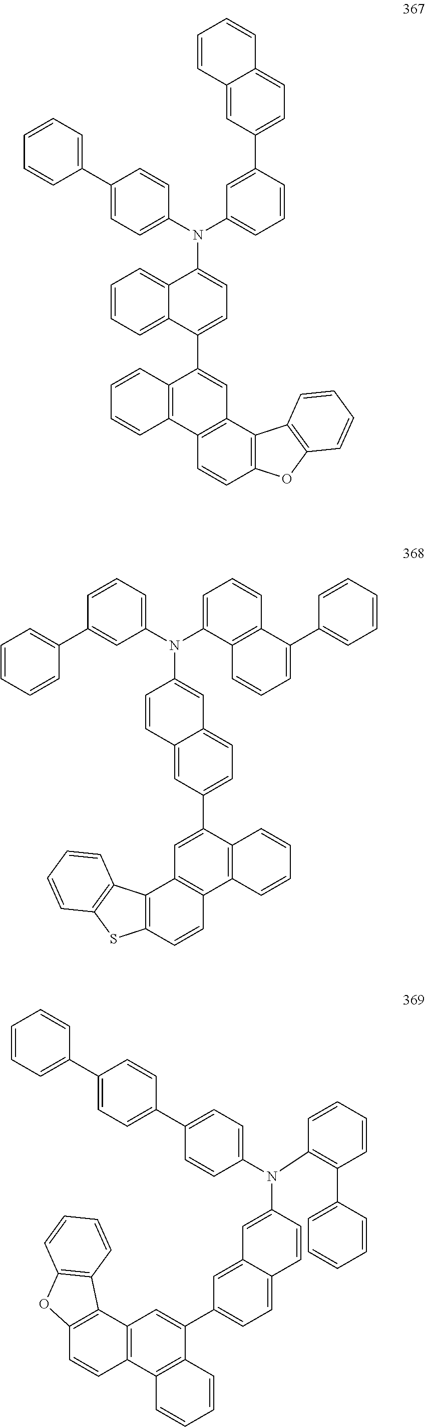 [SMILES: c1ccc(-c2ccc(N(c3cccc(-c4ccc5ccccc5c4)c3)c3ccc(-c4cc5c(ccc6oc7ccccc7c65)c5ccccc45)c4ccccc34)cc2)cc1 ; c1ccc(-c2cccc(N(c3ccc4cc(-c5cc6c(ccc7sc8ccccc8c76)c6ccccc56)ccc4c3)c3cccc4c(-c5ccccc5)cccc34)c2)cc1 ; c1ccc(-c2ccc(-c3ccc(N(c4ccc5ccc(-c6cc7c(ccc8oc9ccccc9c87)c7ccccc67)cc5c4)c4ccccc4-c4ccccc4)cc3)cc2)cc1]

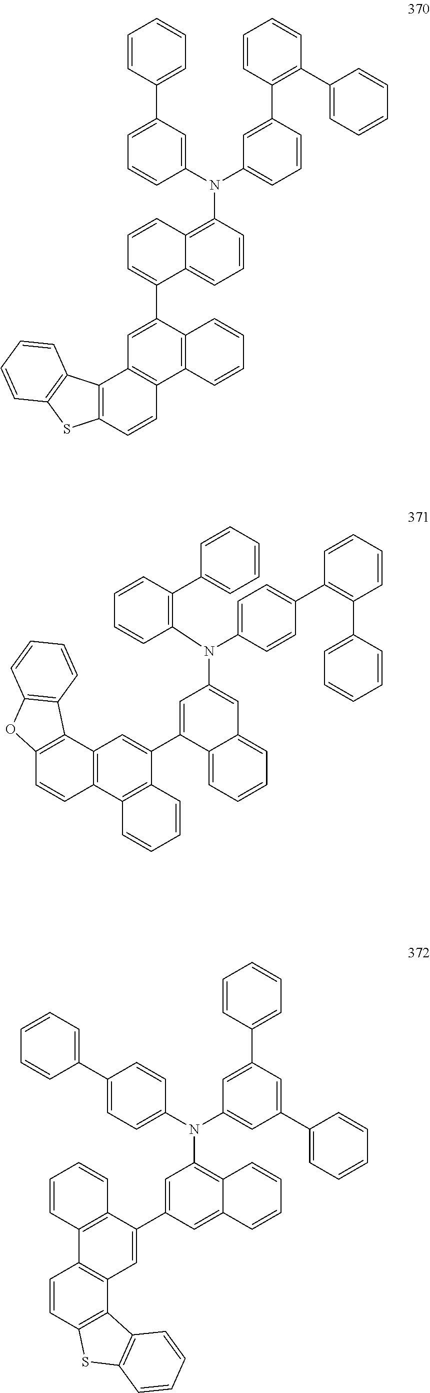 [SMILES: c1ccc(-c2cccc(N(c3cccc(-c4ccccc4-c4ccccc4)c3)c3cccc4c(-c5cc6c(ccc7sc8ccccc8c76)c6ccccc56)cccc34)c2)cc1 ; c1ccc(-c2ccccc2-c2ccc(N(c3cc(-c4cc5c(ccc6oc7ccccc7c65)c5ccccc45)c4ccccc4c3)c3ccccc3-c3ccccc3)cc2)cc1 ; c1ccc(-c2ccc(N(c3cc(-c4ccccc4)cc(-c4ccccc4)c3)c3cc(-c4cc5c(ccc6sc7ccccc7c65)c5ccccc45)cc4ccccc34)cc2)cc1]

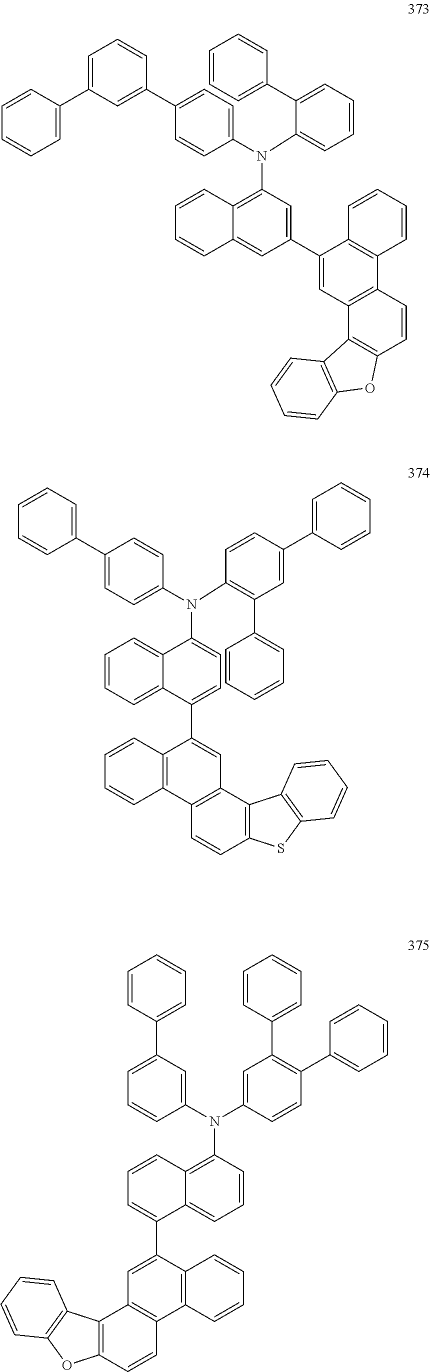 [SMILES: c1ccc(-c2cccc(-c3ccc(N(c4ccccc4-c4ccccc4)c4cc(-c5cc6c(ccc7oc8ccccc8c76)c6ccccc56)cc5ccccc45)cc3)c2)cc1 ; c1ccc(-c2ccc(N(c3ccc(-c4ccccc4)cc3-c3ccccc3)c3ccc(-c4cc5c(ccc6sc7ccccc7c65)c5ccccc45)c4ccccc34)cc2)cc1 ; c1ccc(-c2cccc(N(c3ccc(-c4ccccc4)c(-c4ccccc4)c3)c3cccc4c(-c5cc6c(ccc7oc8ccccc8c76)c6ccccc56)cccc34)c2)cc1]

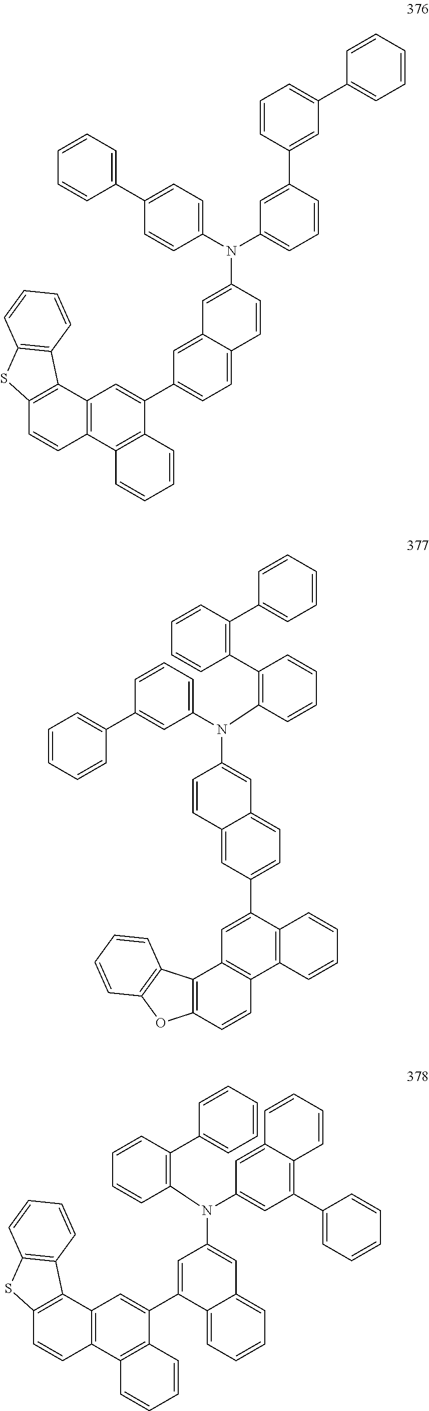 [SMILES: c1ccc(-c2ccc(N(c3cccc(-c4cccc(-c5ccccc5)c4)c3)c3ccc4ccc(-c5cc6c(ccc7sc8ccccc8c76)c6ccccc56)cc4c3)cc2)cc1 ; c1ccc(-c2cccc(N(c3ccc4cc(-c5cc6c(ccc7oc8ccccc8c76)c6ccccc56)ccc4c3)c3ccccc3-c3ccccc3-c3ccccc3)c2)cc1 ; c1ccc(-c2ccccc2N(c2cc(-c3ccccc3)c3ccccc3c2)c2cc(-c3cc4c(ccc5sc6ccccc6c54)c4ccccc34)c3ccccc3c2)cc1]

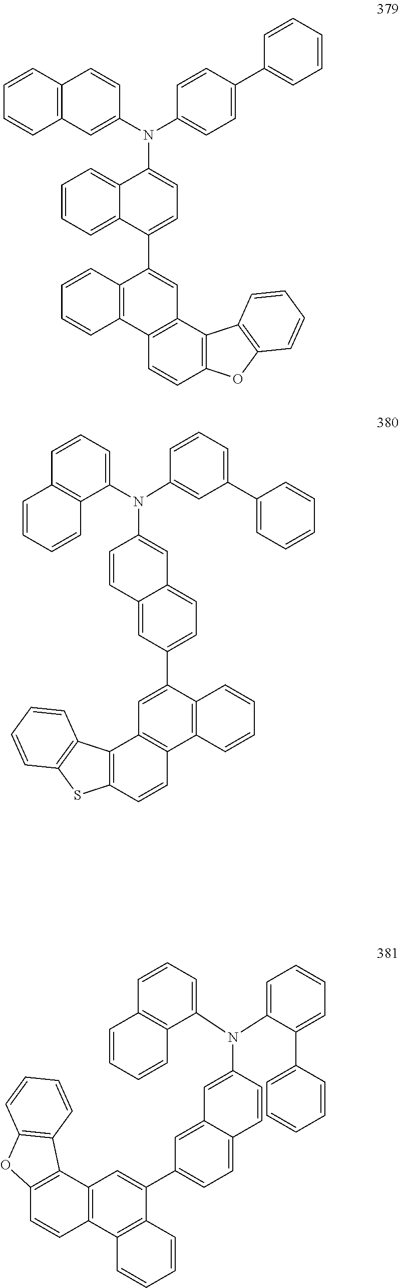 [SMILES: c1ccc(-c2ccc(N(c3ccc4ccccc4c3)c3ccc(-c4cc5c(ccc6oc7ccccc7c65)c5ccccc45)c4ccccc34)cc2)cc1 ; c1ccc(-c2cccc(N(c3ccc4cc(-c5cc6c(ccc7sc8ccccc8c76)c6ccccc56)ccc4c3)c3cccc4ccccc34)c2)cc1 ; c1ccc(-c2ccccc2N(c2ccc3ccc(-c4cc5c(ccc6oc7ccccc7c65)c5ccccc45)cc3c2)c2cccc3ccccc23)cc1]

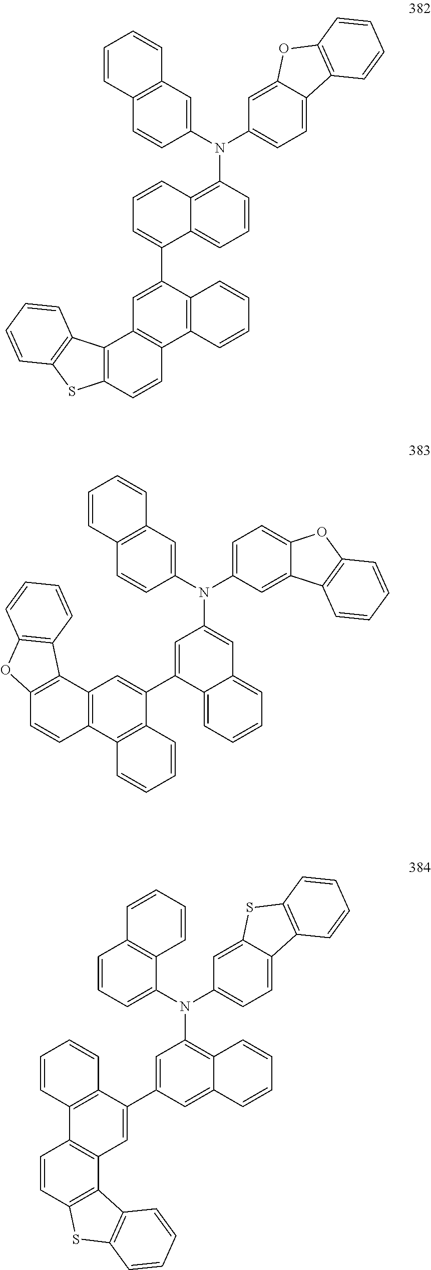 [SMILES: c1ccc2cc(N(c3ccc4c(c3)oc3ccccc34)c3cccc4c(-c5cc6c(ccc7sc8ccccc8c76)c6ccccc56)cccc34)ccc2c1 ; c1ccc2cc(N(c3cc(-c4cc5c(ccc6oc7ccccc7c65)c5ccccc45)c4ccccc4c3)c3ccc4oc5ccccc5c4c3)ccc2c1 ; c1ccc2c(N(c3ccc4c(c3)sc3ccccc34)c3cc(-c4cc5c(ccc6sc7ccccc7c65)c5ccccc45)cc4ccccc34)cccc2c1]

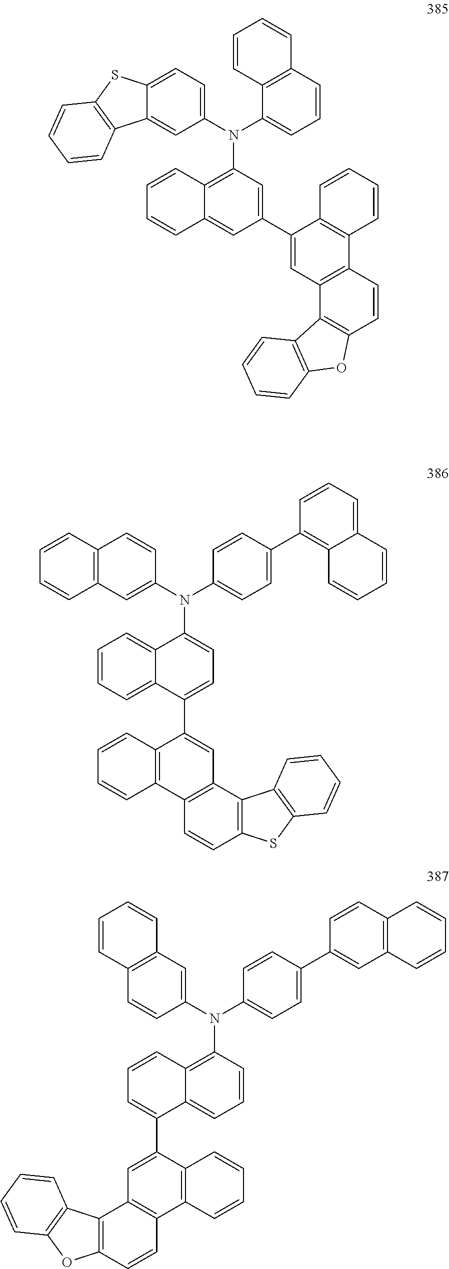 [SMILES: c1ccc2c(N(c3ccc4sc5ccccc5c4c3)c3cc(-c4cc5c(ccc6oc7ccccc7c65)c5ccccc45)cc4ccccc34)cccc2c1 ; c1ccc2cc(N(c3ccc(-c4cccc5ccccc45)cc3)c3ccc(-c4cc5c(ccc6sc7ccccc7c65)c5ccccc45)c4ccccc34)ccc2c1 ; c1ccc2cc(-c3ccc(N(c4ccc5ccccc5c4)c4cccc5c(-c6cc7c(ccc8oc9ccccc9c87)c7ccccc67)cccc45)cc3)ccc2c1]

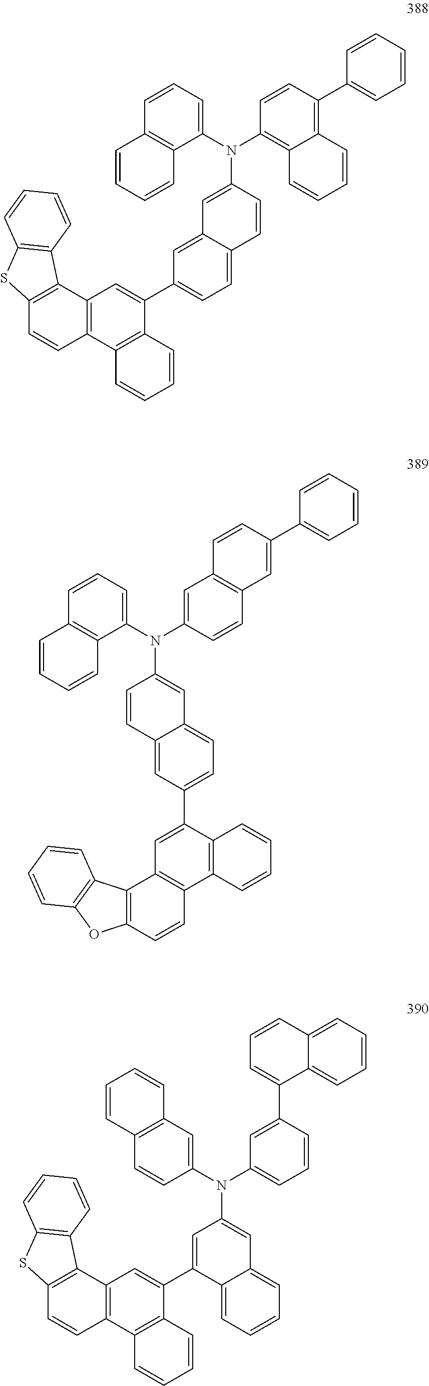 [SMILES: c1ccc(-c2ccc(N(c3ccc4ccc(-c5cc6c(ccc7sc8ccccc8c76)c6ccccc56)cc4c3)c3cccc4ccccc34)c3ccccc23)cc1 ; c1ccc(-c2ccc3cc(N(c4ccc5cc(-c6cc7c(ccc8oc9ccccc9c87)c7ccccc67)ccc5c4)c4cccc5ccccc45)ccc3c2)cc1 ; c1cc(-c2cccc3ccccc23)cc(N(c2ccc3ccccc3c2)c2cc(-c3cc4c(ccc5sc6ccccc6c54)c4ccccc34)c3ccccc3c2)c1]

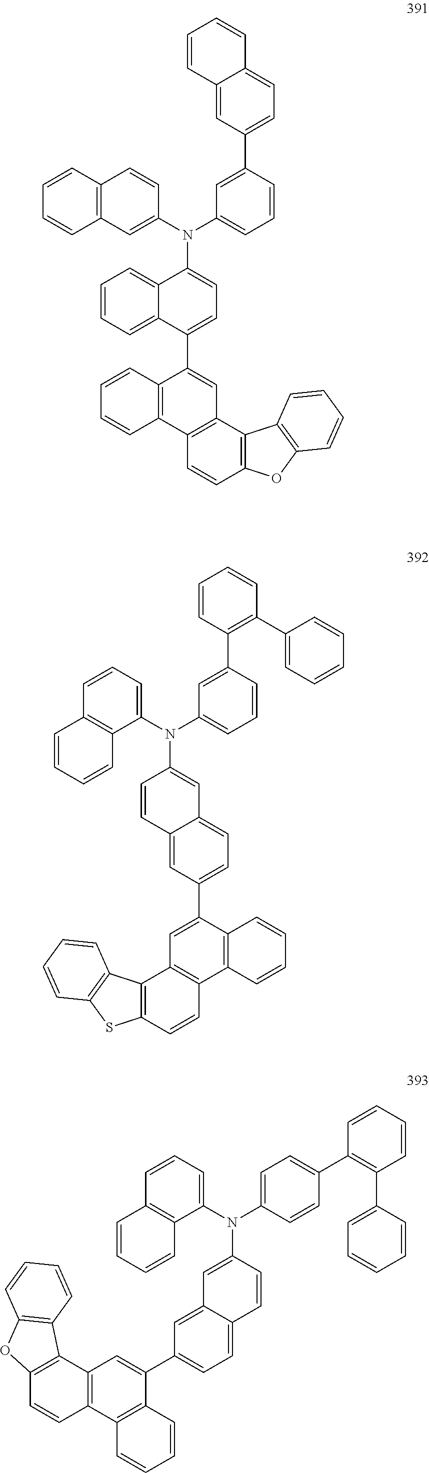 [SMILES: c1cc(-c2ccc3ccccc3c2)cc(N(c2ccc3ccccc3c2)c2ccc(-c3cc4c(ccc5oc6ccccc6c54)c4ccccc34)c3ccccc23)c1 ; c1ccc(-c2ccccc2-c2cccc(N(c3ccc4cc(-c5cc6c(ccc7sc8ccccc8c76)c6ccccc56)ccc4c3)c3cccc4ccccc34)c2)cc1 ; c1ccc(-c2ccccc2-c2ccc(N(c3ccc4ccc(-c5cc6c(ccc7oc8ccccc8c76)c6ccccc56)cc4c3)c3cccc4ccccc34)cc2)cc1]

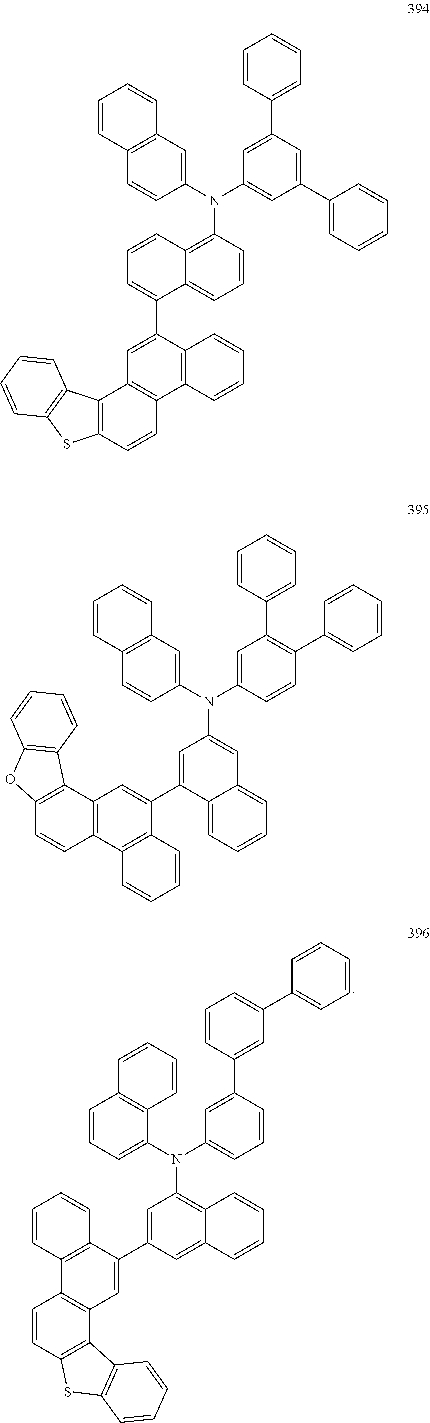 [SMILES: c1ccc(-c2cc(-c3ccccc3)cc(N(c3ccc4ccccc4c3)c3cccc4c(-c5cc6c(ccc7sc8ccccc8c76)c6ccccc56)cccc34)c2)cc1 ; c1ccc(-c2ccc(N(c3ccc4ccccc4c3)c3cc(-c4cc5c(ccc6oc7ccccc7c65)c5ccccc45)c4ccccc4c3)cc2-c2ccccc2)cc1 ; c1ccc(-c2cccc(-c3cccc(N(c4cccc5ccccc45)c4cc(-c5cc6c(ccc7sc8ccccc8c76)c6ccccc56)cc5ccccc45)c3)c2)cc1]

In a second aspect, the present application provides an organic electroluminescent device, comprising an anode, a cathode, and a functional layer disposed between the anode and the cathode; wherein the functional layer contains the arylamine compound of the first aspect of the present application.

The arylamine compounds provided in the present application can be utilized for the formation of at least one organic film layer in the functional layer, in order to improve the luminous efficiency and the lifespan and the other characteristics of organic electroluminescent devices.

Alternatively, the functional layer further comprises a hole transport region comprising a hole transport layer (also known as a first hole transport layer) and a light-emitting adjustment layer (also known as a second hole transport layer or hole auxiliary layer), the hole transport layer being disposed between the anode and the organic light-emitting layer, and the light-emitting adjustment layer being disposed between the first hole transport layer and the organic light-emitting layer.

Alternatively, the functional layer further comprises a light-emitting layer, comprising the host material of a light-emitting layer and a doping material, wherein the host material of the light-emitting layer comprises the arylamine compound of the present application.

In some embodiments, the host material of the light-emitting layer is jointly composed of the arylamine compound of the present application and the other materials.

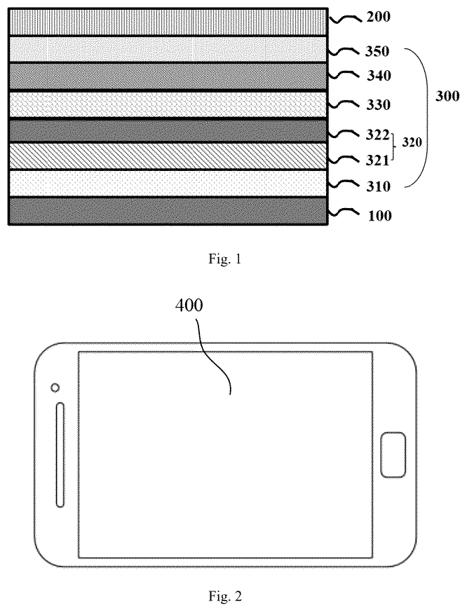

According to one specific embodiment, the organic electroluminescent device is as shown in FIG. 1, and comprises an anode 100, a hole injection layer 310, a first hole transport layer 321, a light-emitting adjustment layer 322, an organic light-emitting layer 330, an electron transport layer 340, an electron injection layer 350, and a cathode 200 that are stacked sequentially.

In the present application, the anode 100 comprises anode materials, which are preferably a high work function material contributing to injection of holes into the functional layer. The specific examples of the anode material include: metals such as nickel, platinum, vanadium, chromium, copper, zinc and gold, or alloys thereof; metal oxides such as zinc oxide, indium oxide, indium tin oxide (ITO), and indium zinc oxide (IZO); combinations of metals and oxides, such as ZnO:Al or SnO₂:Sb; or conductive polymers such as poly(3-methylthiophene), poly[3,4-(ethylene-1,2-dioxy)thiophene] (PEDT), polypyrrole, and polyaniline, but are not limited thereto. Preferably, a transparent electrode comprising indium tin oxide (ITO) as the anode is included.

In the present application, a first hole transport layer and a light-emitting adjustment layer may respectively include one or more hole transport materials. The hole transport materials may be selected from the group consisting of carbazole multimers, carbazole-connected triarylamine based compounds, and other types of compounds. Specifically, the hole transport materials may be selected from the group consisting of the following compounds or their combination:

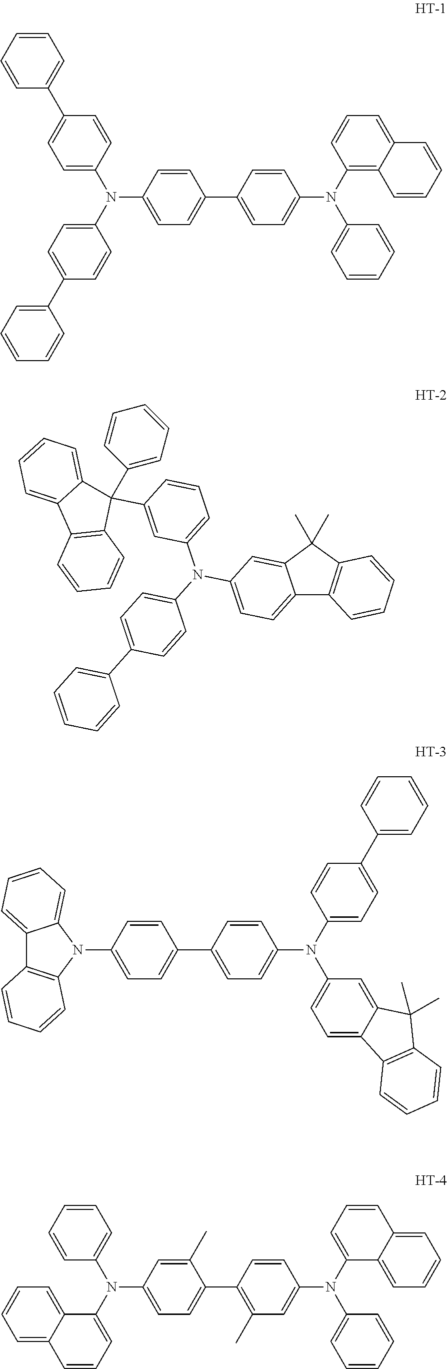 [SMILES: c1ccc(-c2ccc(N(c3ccc(-c4ccccc4)cc3)c3ccc(-c4ccc(N(c5ccccc5)c5cccc6ccccc56)cc4)cc3)cc2)cc1 ; CC1(C)c2ccccc2-c2ccc(N(c3ccc(-c4ccccc4)cc3)c3cccc(C4(c5ccccc5)c5ccccc5-c5ccccc54)c3)cc21 ; CC1(C)c2ccccc2-c2ccc(N(c3ccc(-c4ccccc4)cc3)c3ccc(-c4ccc(-n5c6ccccc6c6ccccc65)cc4)cc3)cc21 ; Cc1cc(N(c2ccccc2)c2cccc3ccccc23)ccc1-c1ccc(N(c2ccccc2)c2cccc3ccccc23)cc1C]

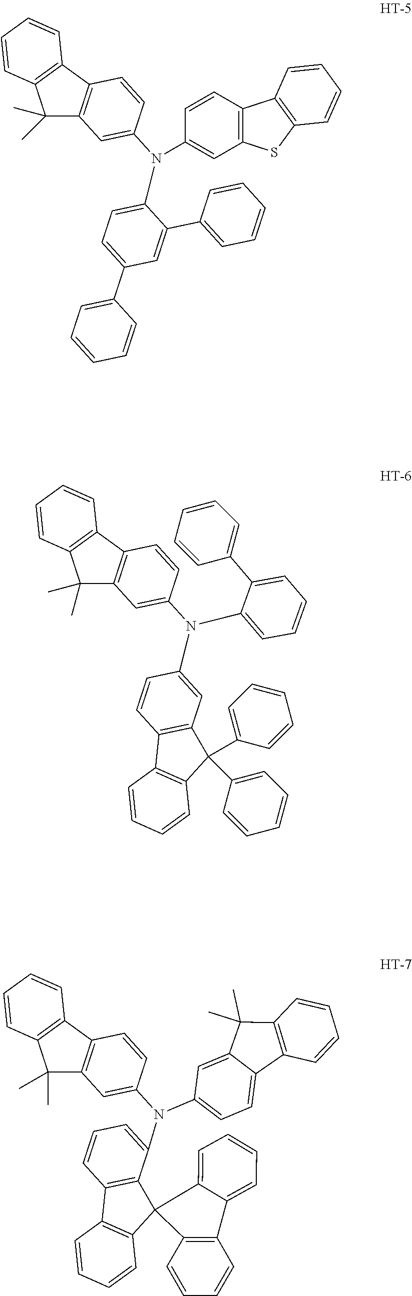 [SMILES: CC1(C)c2ccccc2-c2ccc(N(c3ccc4c(c3)sc3ccccc34)c3ccc(-c4ccccc4)cc3-c3ccccc3)cc21 ; CC1(C)c2ccccc2-c2ccc(N(c3ccc4c(c3)C(c3ccccc3)(c3ccccc3)c3ccccc3-4)c3ccccc3-c3ccccc3)cc21 ; CC1(C)c2ccccc2-c2ccc(N(c3ccc4c(c3)C(C)(C)c3ccccc3-4)c3cccc4c3C3(c5ccccc5-c5ccccc53)c3ccccc3-4)cc21]

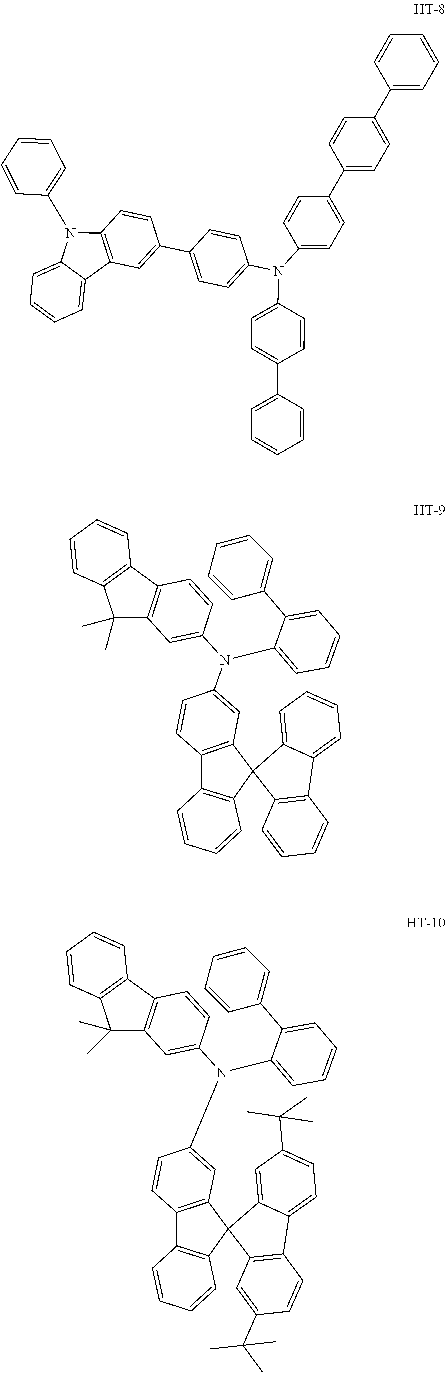 [SMILES: c1ccc(-c2ccc(-c3ccc(N(c4ccc(-c5ccccc5)cc4)c4ccc(-c5ccc6c(c5)c5ccccc5n6-c5ccccc5)cc4)cc3)cc2)cc1 ; CC1(C)c2ccccc2-c2ccc(N(c3ccc4c(c3)C3(c5ccccc5-c5ccccc53)c3ccccc3-4)c3ccccc3-c3ccccc3)cc21 ; CC(C)(C)c1ccc2c(c1)C1(c3ccccc3-c3ccc(N(c4ccc5c(c4)C(C)(C)c4ccccc4-5)c4ccccc4-c4ccccc4)cc31)c1cc(C(C)(C)C)ccc1-2]

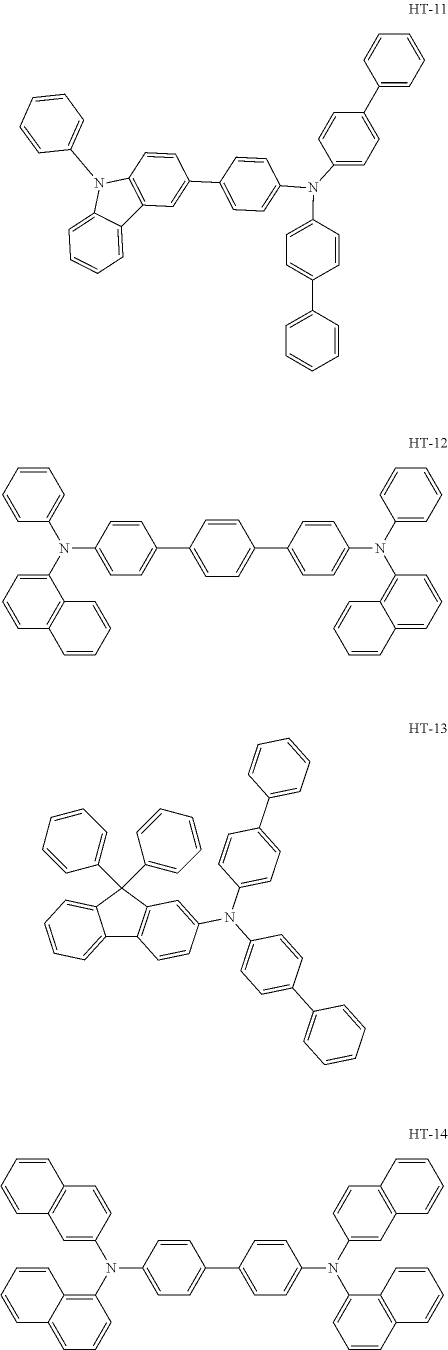 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; c1ccc(N(c2ccc(-c3ccc(-c4ccc(N(c5ccccc5)c5cccc6ccccc56)cc4)cc3)cc2)c2cccc3ccccc23)cc1 ; c1ccc(-c2ccc(N(c3ccc(-c4ccccc4)cc3)c3ccc4c(c3)C(c3ccccc3)(c3ccccc3)c3ccccc3-4)cc2)cc1 ; c1ccc2cc(N(c3ccc(-c4ccc(N(c5ccc6ccccc6c5)c5cccc6ccccc56)cc4)cc3)c3cccc4ccccc34)ccc2c1]

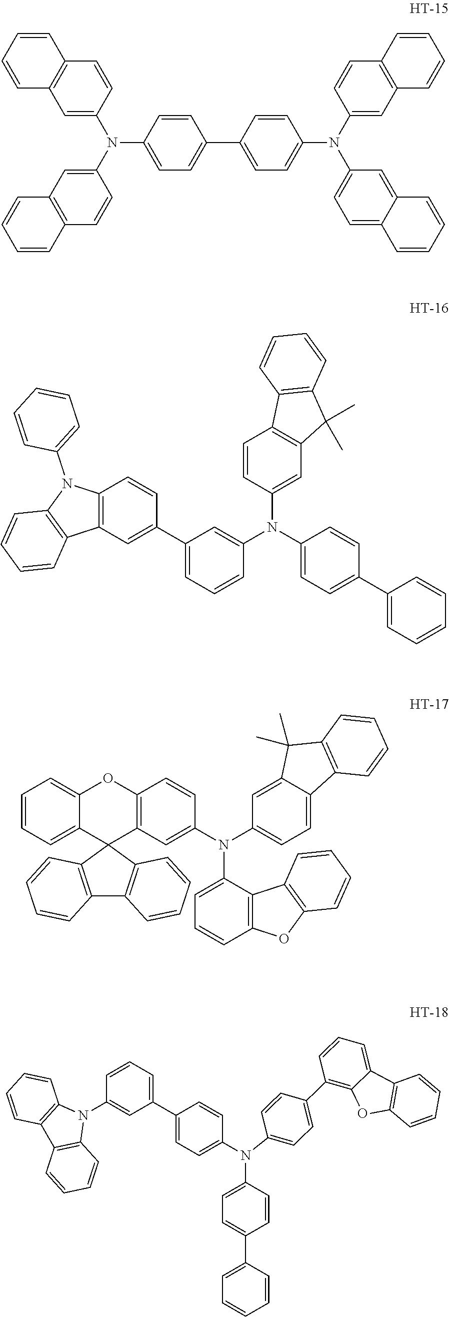 [SMILES: c1ccc2cc(N(c3ccc(-c4ccc(N(c5ccc6ccccc6c5)c5ccc6ccccc6c5)cc4)cc3)c3ccc4ccccc4c3)ccc2c1 ; CC1(C)c2ccccc2-c2ccc(N(c3ccc(-c4ccccc4)cc3)c3cccc(-c4ccc5c(c4)c4ccccc4n5-c4ccccc4)c3)cc21 ; CC1(C)c2ccccc2-c2ccc(N(c3ccc4c(c3)C3(c5ccccc5O4)c4ccccc4-c4ccccc43)c3cccc4oc5ccccc5c34)cc21 ; c1ccc(-c2ccc(N(c3ccc(-c4cccc(-n5c6ccccc6c6ccccc65)c4)cc3)c3ccc(-c4cccc5c4oc4ccccc45)cc3)cc2)cc1]

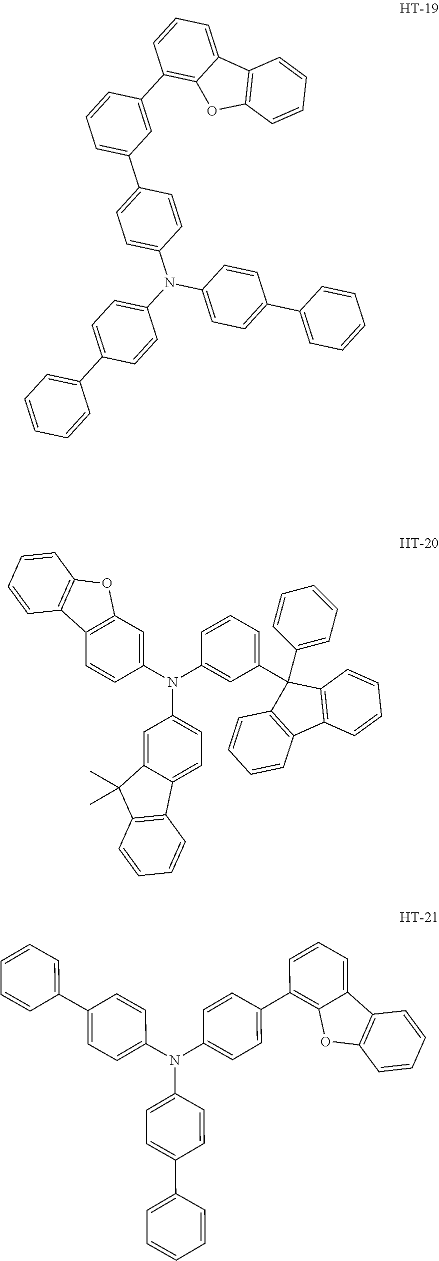 [SMILES: c1ccc(-c2ccc(N(c3ccc(-c4ccccc4)cc3)c3ccc(-c4cccc(-c5cccc6c5oc5ccccc56)c4)cc3)cc2)cc1 ; CC1(C)c2ccccc2-c2ccc(N(c3cccc(C4(c5ccccc5)c5ccccc5-c5ccccc54)c3)c3ccc4c(c3)oc3ccccc34)cc21 ; c1ccc(-c2ccc(N(c3ccc(-c4ccccc4)cc3)c3ccc(-c4cccc5c4oc4ccccc45)cc3)cc2)cc1]

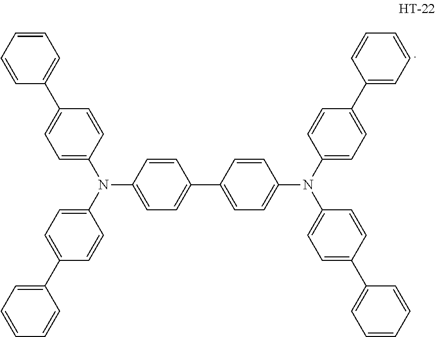 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

In one embodiment, the first hole transport layer 321 is composed of HT-1.

In one embodiment, the light-emitting adjustment layer 322 is composed of HT-2.

Alternatively, a hole injection layer 310 may be further provided between the anode 100 and the first hole transport layer 321 to enhance the ability to inject holes into the first hole transport layer 321. The hole injection layer 310 may choose to use a benzidine derivative, a starburst arylamine-based compound, a phthalocyanine derivative or other materials. It is not particularly limited in the present application. The material of the hole injection layer 310 is selected, for example, from the following compounds or any combination thereof:

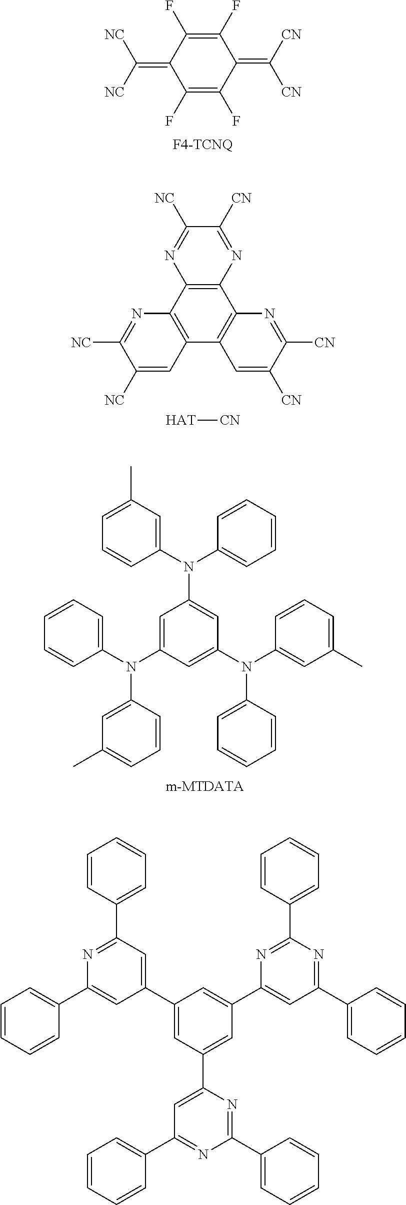

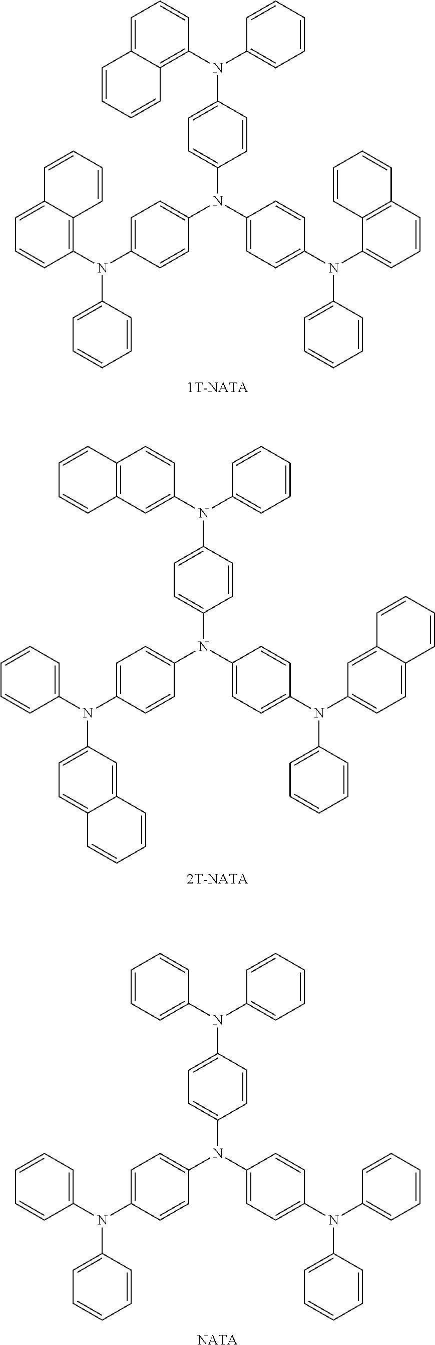

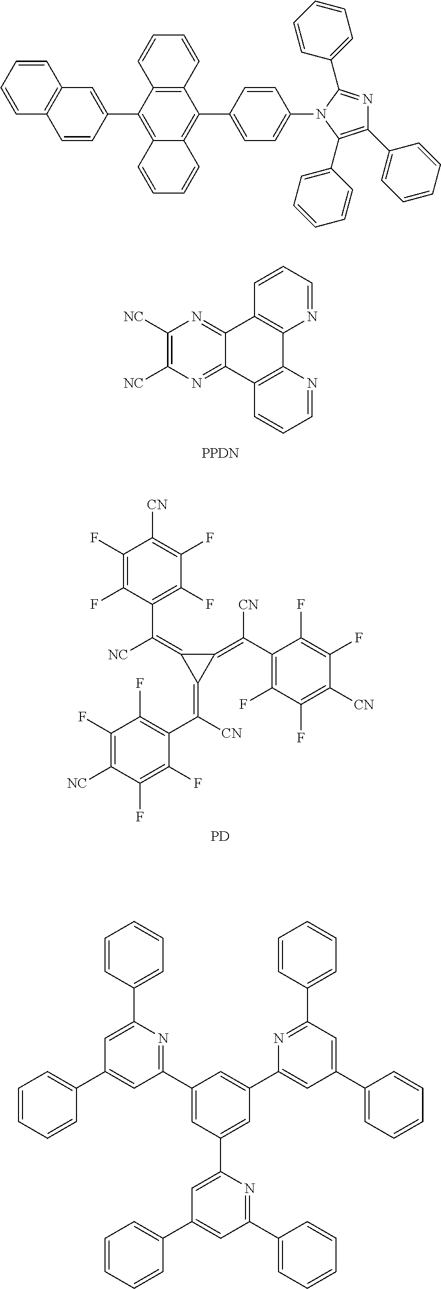 [SMILES: c1ccc(-c2nc(-c3ccccc3)n(-c3ccc(-c4c5ccccc5c(-c5ccc6ccccc6c5)c5ccccc45)cc3)c2-c2ccccc2)cc1 ; N#Cc1nc2c3cccnc3c3ncccc3c2nc1C#N ; "PPDN" ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; "PD" ; c1ccc(-c2cc(-c3ccccc3)nc(-c3cc(-c4cc(-c5ccccc5)cc(-c5ccccc5)n4)cc(-c4cc(-c5ccccc5)cc(-c5ccccc5)n4)c3)c2)cc1]

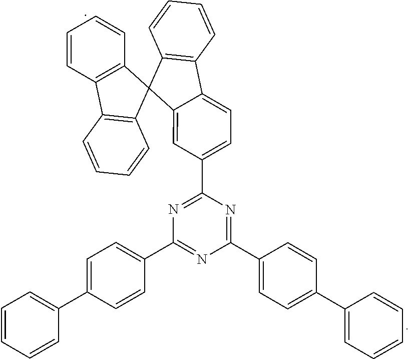 [SMILES: c1ccc(-c2ccc(-c3nc(-c4ccc(-c5ccccc5)cc4)nc(-c4ccc5c(c4)C4(c6ccccc6-c6ccccc64)c4ccccc4-5)n3)cc2)cc1]

In one embodiment of the present application, the hole injection layer 310 is composed of PD and HT-1.

Alternatively, the organic light-emitting layer 330 may be composed of a single luminescent material or may comprise a host material and a guest material. Alternatively, the organic light-emitting layer 330 is composed of a host material and a guest material. The holes injected into the organic light-emitting layer 330 and the electrons injected into the organic light-emitting layer 330 can recombine in the organic light-emitting layer 330 to form excitons. The excitons transmit energy to the host material, and the host material transmits energy to the guest material, thereby enabling the guest material to emit light.

The host material of the organic light-emitting layer 330 may include a metal chelating compound, a stilbene-based derivative, an aromatic amine derivative, a dibenzofuran derivative, or other types of materials. The host material of the organic light-emitting layer 330 may be a single compound, a combination of two or more compounds. Alternatively, the host material comprises the arylamine compounds of the present application.

The guest material of the organic light-emitting layer 330 may be a compound with a condensed aryl ring or derivative thereof, a compound with a heteroaryl ring or derivative thereof, an aromatic amine derivative, or other materials. It is not particularly limited in the present application. The guest material is also known as a doping material or dopant. The dopant can be categorized into fluorescent and phosphorescent dopants dependent on their luminescence mechanisms. For example, the specific examples of the phosphorescent dopant include but are not limited to,

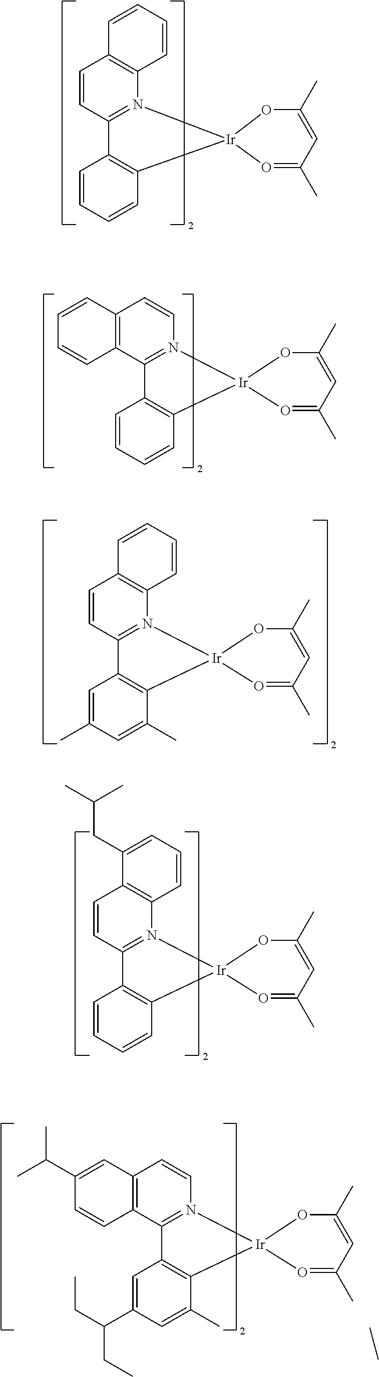

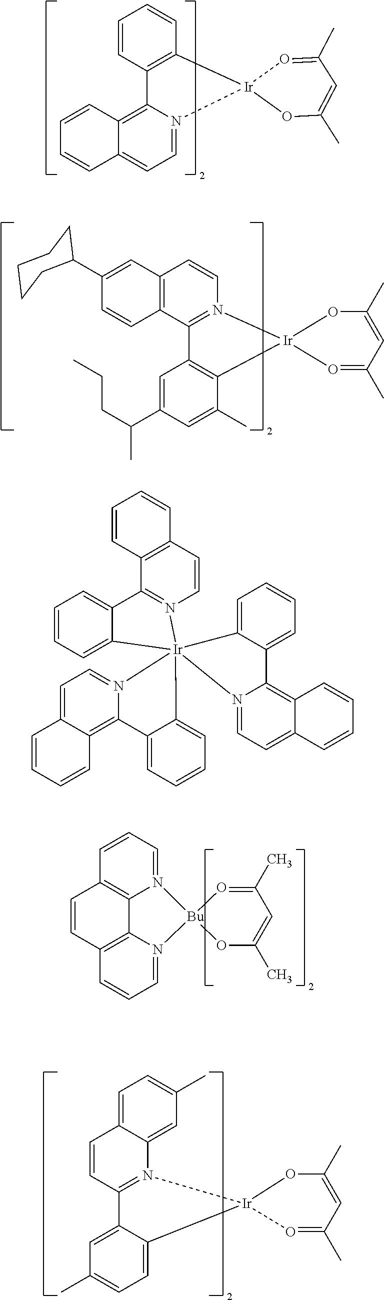

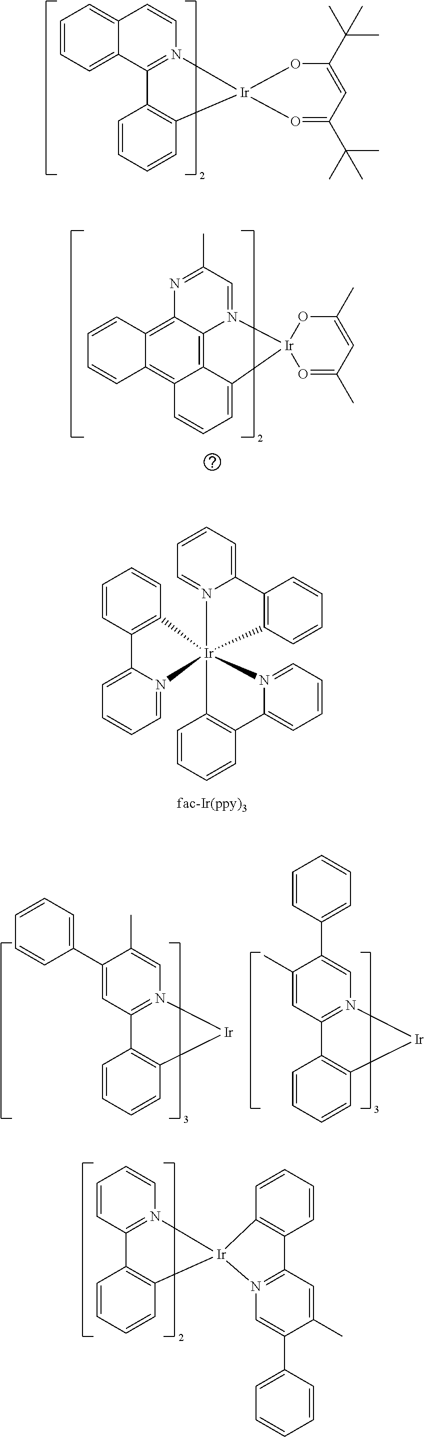

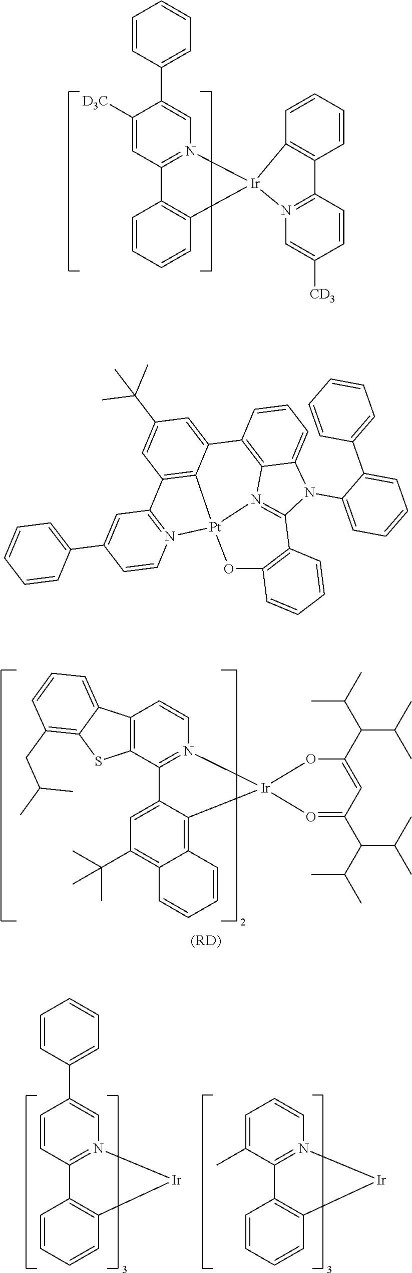

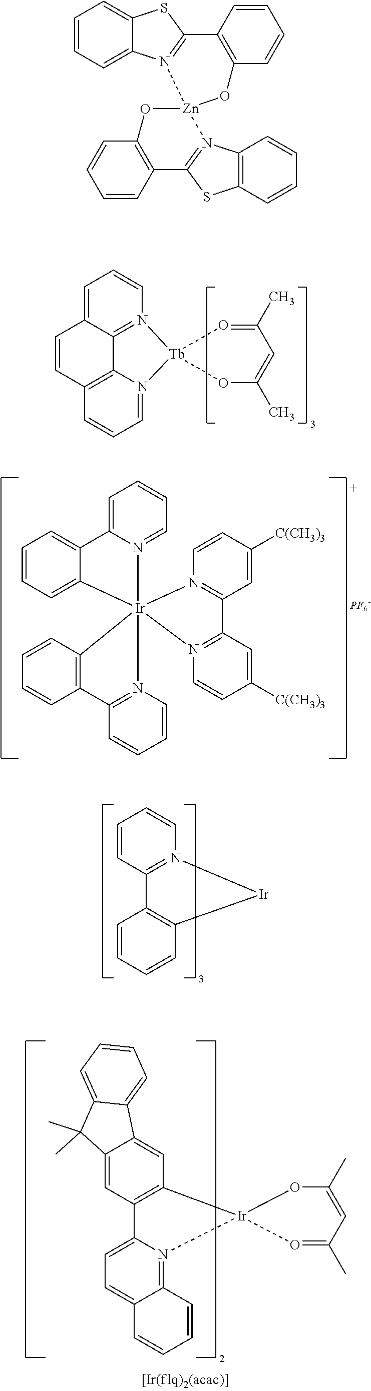

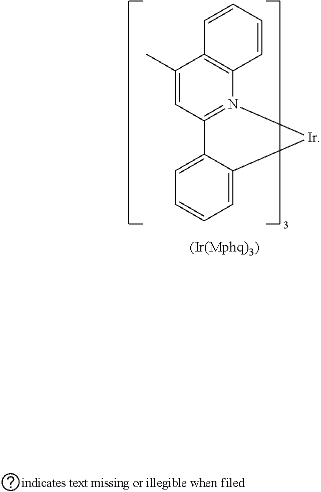

In one embodiment of the present application, the organic electroluminescent device is a red organic electroluminescent device. In a more specific embodiment, the host material of the organic light-emitting layer 330 comprises RH—N

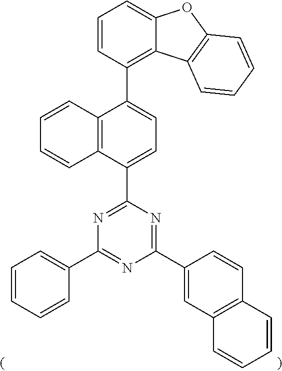 [SMILES: c1ccc(-c2nc(-c3ccc4ccccc4c3)nc(-c3ccc(-c4cccc5oc6ccccc6c45)c4ccccc34)n2)cc1]

and the compound of the present application. The guest material may be, for example, RD.

The electron transport layer 340 may be a single-layer structure or a multi-layer structure, and may comprise one or more electron transport materials. The electron transport materials may be selected from, but are not limited to, LiQ, benzimidazole derivatives, oxadiazole derivatives, quinoxaline derivatives, and other electron transport materials, and it is not particularly limited in the present application. The material of the electron transport layer 340 includes but is not limited to the following compounds:

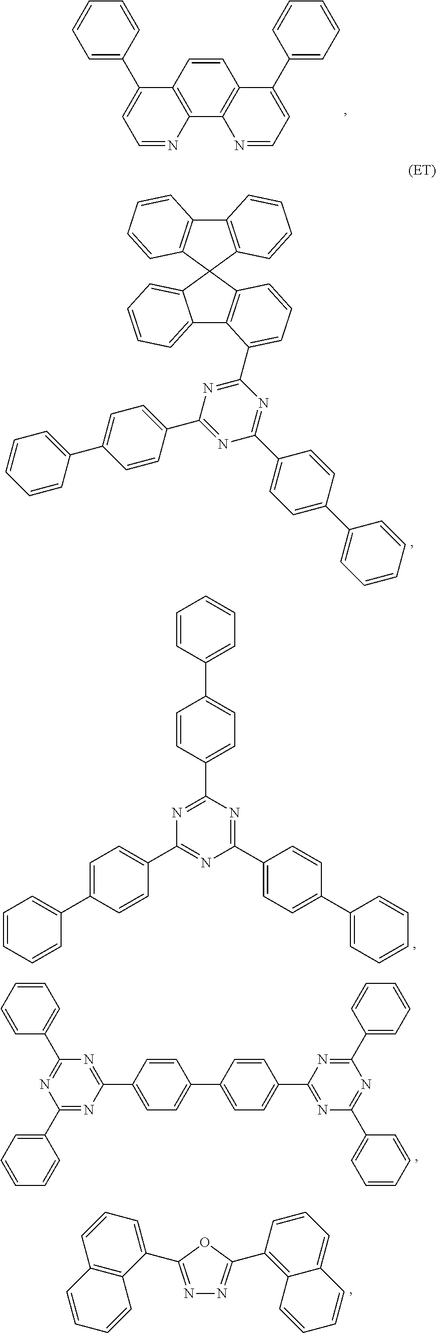

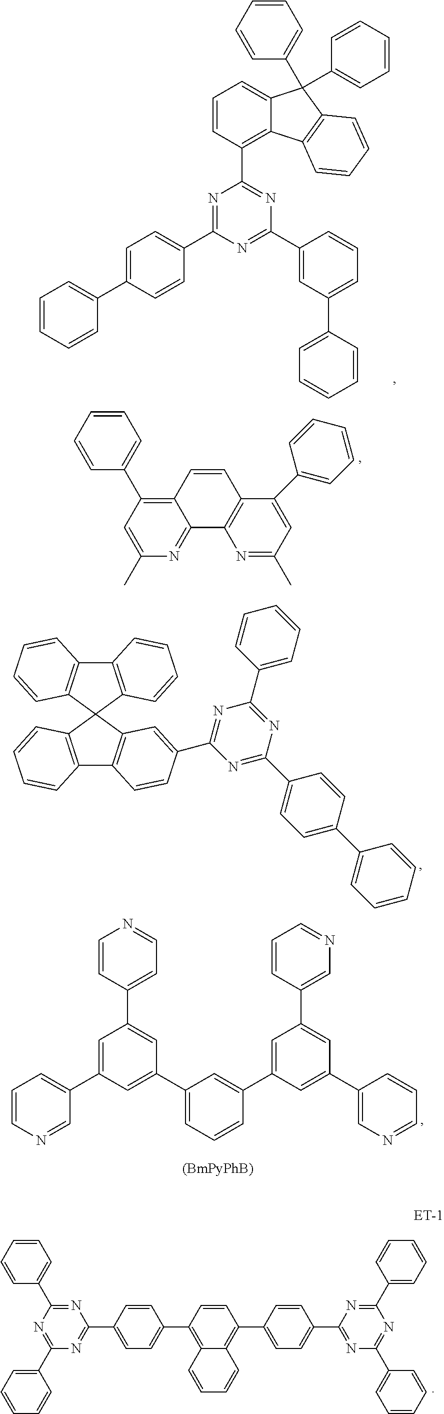

In one embodiment of the present application, the electron transport layer 340 is composed of ET and LiQ.

In the present application, the cathode 200 comprises a cathode material, which is a low work function material contributing to injection of electrons into the functional layer. Specific examples of the cathode material include, but are not limited to, metals such as magnesium, calcium, sodium, potassium, titanium, indium, yttrium, lithium, gadolinium, aluminum, silver, tin, and lead, or alloys thereof; or multilayer materials such as LiF/Al, Liq/Al, LiO₂/Al, LiF/Ca, LiF/Al, and BaF₂/Ca. Alternatively, a metal electrode comprising magnesium and silver as the cathode is included.

Alternatively, an electron injection layer 350 may be further provided between the cathode 200 and the electron transport layer 340 to enhance the ability to inject electrons into the electron transport layer 340. The electron injection layer 350 may comprise an inorganic material such as an alkali metal sulfide and an alkali metal halide or may comprise a complex of an alkali metal and an organic compound. In one embodiment of the present application, the electron injection layer 350 comprises ytterbium (Yb).

A third aspect of the present application, there is provided an electronic apparatus, comprising the organic electroluminescent device described in the second aspect.

According to one embodiment, as shown in FIG. 2, the electronic apparatus provided is an electronic apparatus 400, comprising the above organic electroluminescent device. The electronic apparatus 400 may be, for example, a display apparatus, a lighting apparatus, an optical communication apparatus, or other type of electronic apparatus, examples of which may include, for example but be not limited to, computer screens, mobile phone screens, televisions, electronic paper, emergency lamps, optical modules, etc.

The synthesis method of the arylamine compound in the present application will be demonstrated in detail with the following synthesis examples, but the present application is not limited in any way by this.

### Synthetic Example

Those skilled in the art will appreciate that the chemical reactions described in the present application can be used to suitably prepare many of the arylamine compounds of the present application. The other methods for preparing the compounds of the present application are also considered to be within the scope of the present application. For example, the synthesis of those non-exemplified compounds according to the present application can be successfully accomplished by those skilled in the art through routine methods for modification, such as appropriate protection of interfering group, such as appropriately protecting interfering groups, using alternative reagents known in the art in place of those explicitly described herein, or making conventional adjustments to the reaction conditions. Compounds for which synthesis methods are not mentioned in the present application may be obtained through commercial sources.

### Synthesis of Sub-a1

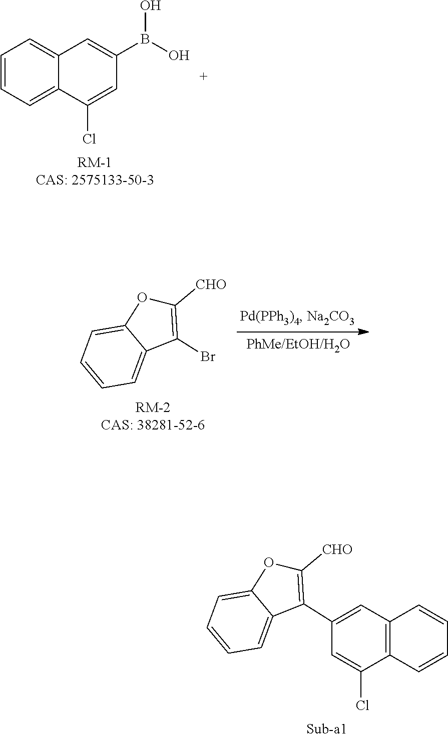 [SMILES: OB(O)c1cc(Cl)c2ccccc2c1 ; O=Cc1oc2ccccc2c1Br ; O=Cc1oc2ccccc2c1-c1cc(Cl)c2ccccc2c1]

Under a nitrogen atmosphere, to a three-necked flask were sequentially added RM-1 (11.35 g, 55 mmol), RM-2 (11.25 g, 50 mmol), tetrakis(triphenylphosphine) palladium (0.58 g, 0.5 mmol), anhydrous sodium carbonate (10.60 g, 100 mmol), toluene (120 mL), anhydrous ethanol (30 mL), and deionized water (30 mL). The reaction mixture was stirred and heated to reflux for 8 hours of reaction. After the system was cooled to room temperature, the reaction mixture was extracted with dichloromethane (100 mL×3 times). The organic phases were combined and dried over anhydrous magnesium sulfate, followed by filtering, removing the solvent by distillation under reduced pressure to yield a crude product. The crude product was purified by silica gel column chromatography using a mixed solvent of dichloromethane and n-heptane as the mobile phase, to yield Sub-a1 as a white solid (10.27 g, yield 67%).

### Synthesis of Sub-a2

Intermediate Sub-a2 was synthesized following the synthesis method of Sub-a1, except that Reactant A was used instead of RM-2, as indicated in Table 1.

TABLE 1
Synthesis of Sub-a2

| Sub-a No. | Reactant A | Sub-a structure | Yield (%) |
|---|---|---|---|
| Sub-a2 | 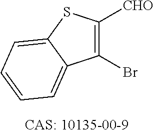 [SMILES: O=Cc1sc2ccccc2c1Br] | 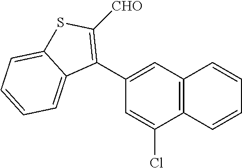 [SMILES: O=Cc1sc2ccccc2c1-c1cc(Cl)c2ccccc2c1] | 63 |

### Synthesis of Sub-b1

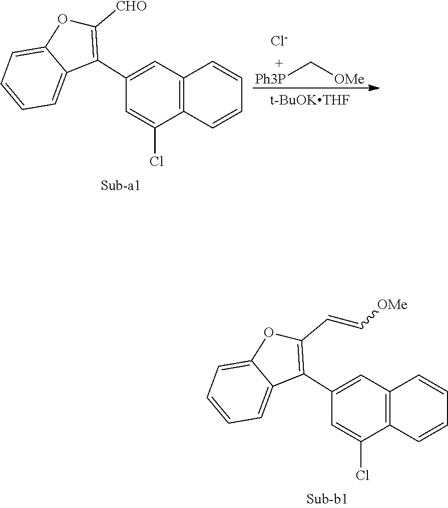

Under a nitrogen atmosphere, to a 1000 mL three-necked flask were sequentially added Sub-b1 (39.87 g, 130 mmol), (methoxymethyl)triphenylphosphonium chloride (74.38 g, 217 mmol) and anhydrous tetrahydrofuran (500 mL). The reaction system was cooled to 0° C. using an ice-water bath. A solution of Potassium tert-butanoate in anhydrous tetrahydrofuran (1 M, 220 mL) was added slowly and dropwise into the system. After the addition was completed, the system was allowed to warm slowly to room temperature and the reaction continued with stirring for 6 hours. The reaction solution was poured into 1000 mL of deionized water and extracted with ethyl acetate (250 mL×3 times). The organic phases were combined and dried over anhydrous magnesium sulfate, followed by filtering, removing the solvent by distillation under reduced pressure to yield a crude product. The crude product was purified via silica gel column chromatography using n-heptane as the mobile phase, to yield Sub-b1 as a solid (33.95 g, yield: 78%).

### Synthesis of Sub-b2

Intermediate Sub-b2 was synthesized following the synthesis method of Sub-b1, except that Reactant B was used instead of Sub-a1, as indicated in Table 2.

TABLE 2
Synthesis of Sub-b2

| Sub-b No. | Reactant B | Sub-b structure | Yield (%) |
|---|---|---|---|
| Sub-b2 | 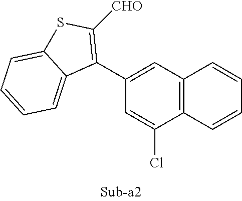 [SMILES: O=Cc1sc2ccccc2c1-c1cc(Cl)c2ccccc2c1] | 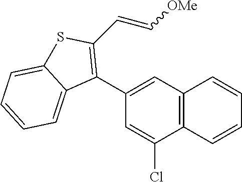 [SMILES: COC=Cc1sc2ccccc2c1-c1cc(Cl)c2ccccc2c1] | 75 |

### Synthesis of Sub-c1

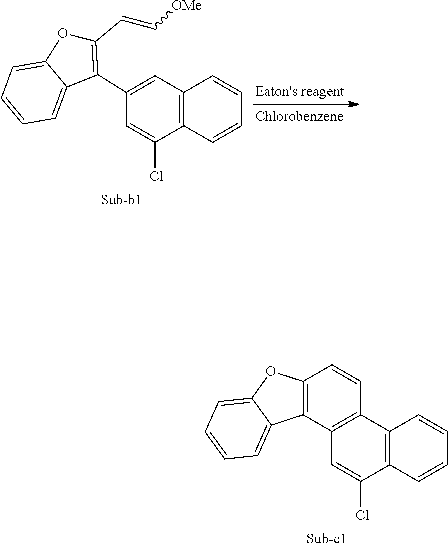 [SMILES: COC=Cc1oc2ccccc2c1-c1cc(Cl)c2ccccc2c1 ; Clc1cc2c(ccc3oc4ccccc4c32)c2ccccc12]

Under a nitrogen atmosphere, to a 1000 mL three-necked flask were added sequentially Sub-b1 (39.84 g, 119 mmol), Eaton's reagent (4.5 mL) and chlorobenzene (400 mL). The reaction mixture was stirred and heated to reflux for 4 hours of reaction. After the system was cooled to room temperature, the reaction solution was poured into 1000 mL of deionized water and neutralized with saturated sodium hydroxide solution. The mixture was then extracted with dichloromethane (250 mL×3 times). The organic phases were combined and dried over anhydrous magnesium sulfate, followed by filtering, removing the solvent by distillation under reduced pressure to yield a crude product. The crude product was purified by silica gel column chromatography using a mixed solvent of dichloromethane and n-heptane as the mobile phase, to yield Sub-c1 as a white solid (24.14 g, yield 67%).

### Synthesis of Sub-c2

Intermediate Sub-c2 was synthesized following the synthesis method of Sub-c1, except that Reactant C was used instead of Sub-b1, as indicated in Table 3.

TABLE 3
Synthesis of Sub-c2

| Sub-c No. | Reactant C | Sub-c structure | Yield (%) |
|---|---|---|---|
| Sub-c2 | 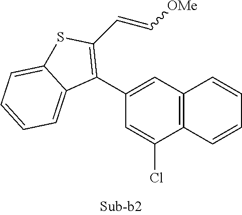 [SMILES: COC=Cc1sc2ccccc2c1-c1cc(Cl)c2ccccc2c1] | 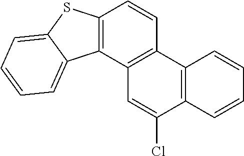 [SMILES: Clc1cc2c(ccc3sc4ccccc4c32)c2ccccc12] | 64 |

### Synthesis of Sub-d1

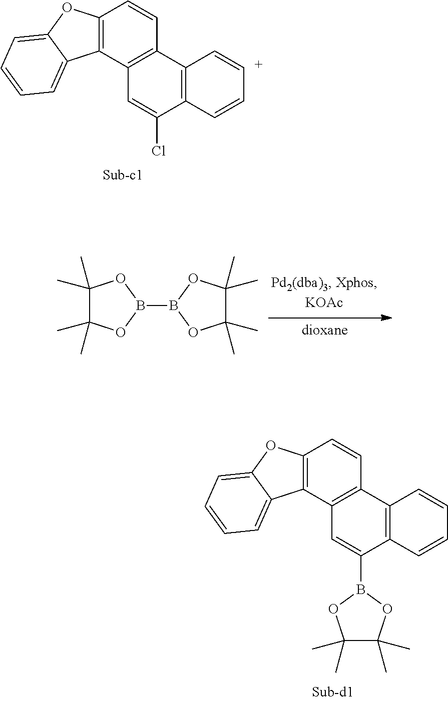

Under a nitrogen atmosphere, to a 500 mL three-necked flask were sequentially added Sub-c1 (15.14 g, 50 mmol), bis(pinacolato)diboron (14.0 g, 55 mmol), potassium acetate (10.8 g, 110 mmol), and 1,4-dioxane (150 mL). The reaction mixture was stirred and heated and when the temperature of system reached 40° C., tris (dibenzylideneacetone) dipalladium (Pd₂(dba) 3, 0.46 g, 0.50 mmol) and 2-dicyclohexylphosphino-2′,4′,6′-triisopropylbiphenyl (XPhos, 0.48 g, 1.0 mmol) were rapidly added. The reaction mixture was heated to reflux and stirred overnight. After the reaction system was cooled to room temperature, 200 mL of water was added to the system. The mixture was stirred thoroughly for 30 minutes and filtered under reduced pressure. The filter cake was washed with deionized water until neutral, and then dissolved in 100 mL of dichloromethane, and dried over anhydrous sodium sulfate. After filtration, the organic phase was distillated under reduced pressure to remove the solvent, to obtain a crude product. The crude product was dissolved in 200 mL of toluene and passed through the silica gel column to remove catalyst, and then concentrated to obtain Sub-d1 as a white solid (13.20 g, yield 67%).

### Synthesis of Sub-d2

Intermediate Sub-d2 was synthesized following the synthesis method of Sub-d1, except that Reactant D was used instead of Sub-c1, as indicated in Table 4.

TABLE 4
Synthesis of Sub-d2

| Sub-d No. | Reactant D | Sub-d structure | Yield (%) |
|---|---|---|---|
| Sub-d2 | 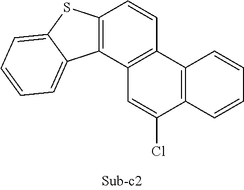 [SMILES: Clc1cc2c(ccc3sc4ccccc4c32)c2ccccc12] | 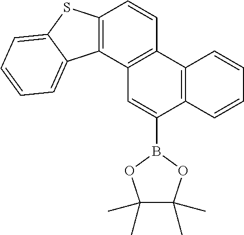 [SMILES: CC1(C)OB(c2cc3c(ccc4sc5ccccc5c43)c3ccccc23)OC1(C)C] | 63 |

### Synthesis of Sub-e1

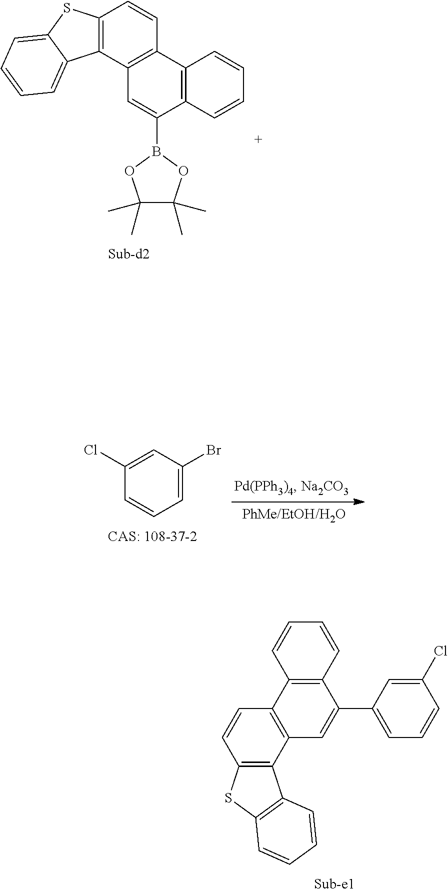 [SMILES: CC1(C)OB(c2cc3c(ccc4sc5ccccc5c43)c3ccccc23)OC1(C)C ; Clc1cccc(Br)c1 ; Clc1cccc(-c2cc3c(ccc4sc5ccccc5c43)c3ccccc23)c1]

Under a nitrogen atmosphere, to a 1000 mL three-necked flask were sequentially added m-bromochlorobenzene (9.57 g, 50 mmol), Sub-d2 (22.57 g, 55 mmol), tetrakis(triphenylphosphine) palladium (0.58 g, 0.5 mmol), anhydrous sodium carbonate (10.60 g, 100 mmol), toluene (220 mL), anhydrous ethanol (55 mL), and deionized water (55 mL). The reaction mixture was stirred and heated to reflux for 8 hours of reaction. After the system was cooled to room temperature, the reaction mixture was extracted with dichloromethane (100 mL×3 times). The organic phases were combined and dried over anhydrous magnesium sulfate, followed by filtering, removing the solvent by distillation under reduced pressure to yield a crude product. The crude product was purified by silica gel column chromatography using a mixed solvent of dichloromethane and n-heptane as the mobile phase, to yield Sub-e1 as a white solid (16.58 g, yield 84%).

### Synthesis of Sub-e2 to Sub-e8:

Sub-e2 to Sub-e8 were synthesized following the synthesis method of Sub-e1, except that Reactant E was used instead of m-bromochlorobenzene and Reactant F was used instead of Sub-d2, as indicated in Table 5.

TABLE 5
Synthesis of Sub-e2 to Sub-e8

| Sub-e No. | Reactant E | Reactant F | Sub-e structure | Yield % |
|---|---|---|---|---|
| Sub-e2 | 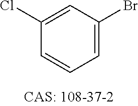 | 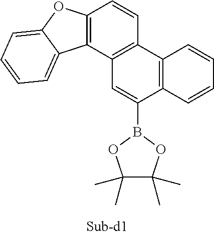 [SMILES: CC1(C)OB(c2cc3c(ccc4oc5ccccc5c43)c3ccccc23)OC1(C)C] | 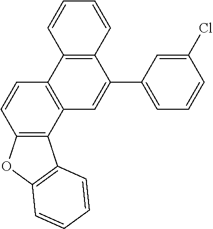 [SMILES: Clc1cccc(-c2cc3c(ccc4oc5ccccc5c43)c3ccccc23)c1] | 86 |
| Sub-e3 | 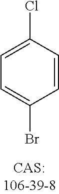 | 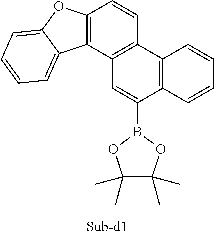 [SMILES: CC1(C)OB(c2cc3c(ccc4oc5ccccc5c43)c3ccccc23)OC1(C)C] | 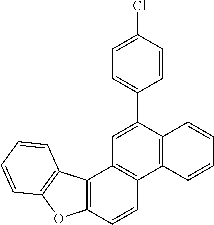 [SMILES: Clc1ccc(-c2cc3c(ccc4oc5ccccc5c43)c3ccccc23)cc1] | 82 |
| Sub-e4 | 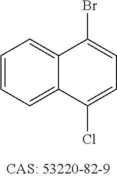 [SMILES: Clc1ccc(Br)c2ccccc12] | 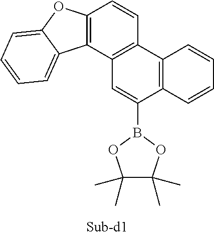 [SMILES: CC1(C)OB(c2cc3c(ccc4oc5ccccc5c43)c3ccccc23)OC1(C)C] | 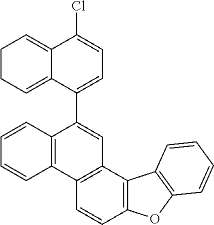 [SMILES: Clc1ccc(-c2cc3c(ccc4oc5ccccc5c43)c3ccccc23)c2c1=CCCC=2] | 75 |
| Sub-e5 | 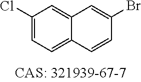 [SMILES: Clc1ccc2ccc(Br)cc2c1] | 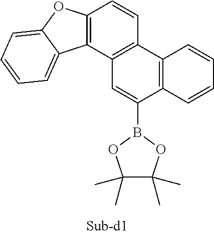 [SMILES: CC1(C)OB(c2cc3c(ccc4oc5ccccc5c43)c3ccccc23)OC1(C)C] | 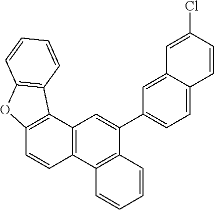 [SMILES: Clc1ccc2ccc(-c3cc4c(ccc5oc6ccccc6c54)c4ccccc34)cc2c1] | 77 |
| Sub-e6 | 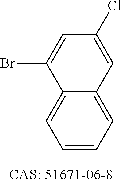 [SMILES: Clc1cc(Br)c2ccccc2c1] | 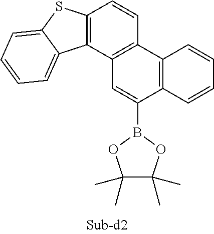 [SMILES: CC1(C)OB(c2cc3c(ccc4sc5ccccc5c43)c3ccccc23)OC1(C)C] | 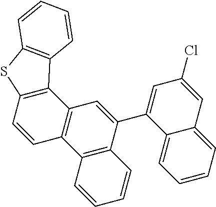 [SMILES: Clc1cc(-c2cc3c(ccc4sc5ccccc5c43)c3ccccc23)c2ccccc2c1] | 74 |
| Sub-e7 | 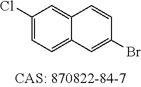 [SMILES: Clc1ccc2cc(Br)ccc2c1] | 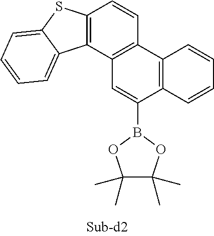 [SMILES: CC1(C)OB(c2cc3c(ccc4sc5ccccc5c43)c3ccccc23)OC1(C)C] | 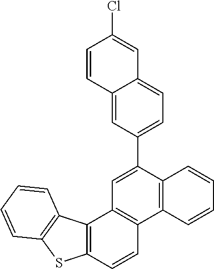 [SMILES: Clc1ccc2cc(-c3cc4c(ccc5sc6ccccc6c54)c4ccccc34)ccc2c1] | 78 |
| Sub-e8 | 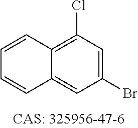 [SMILES: Clc1cc(Br)cc2ccccc12] | 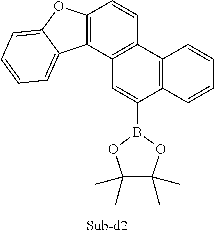 [SMILES: CC1(C)OB(c2cc3c(ccc4oc5ccccc5c43)c3ccccc23)OC1(C)C] | 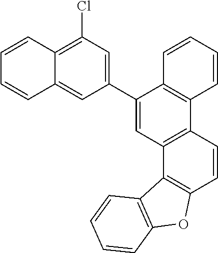 [SMILES: Clc1cc(-c2cc3c(ccc4oc5ccccc5c43)c3ccccc23)cc2ccccc12] | 77 |

### Synthesis of Compound 4:

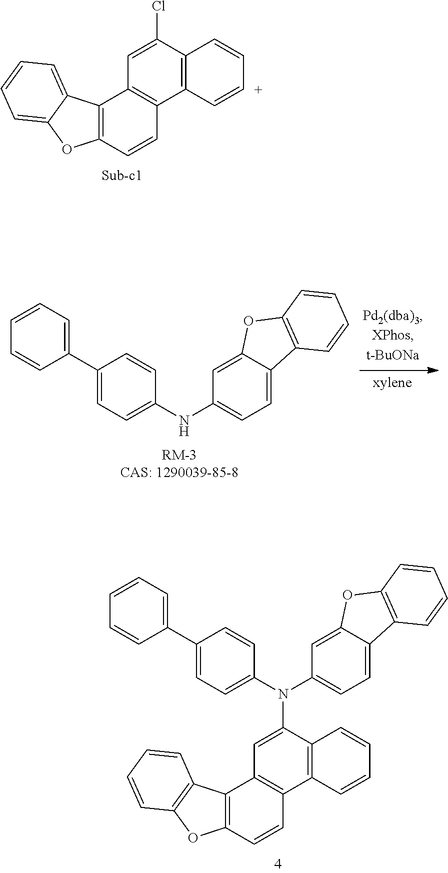 [SMILES: Clc1cc2c(ccc3oc4ccccc4c32)c2c1C=C[C+]=C2 ; c1ccc(-c2ccc(Nc3ccc4c(c3)oc3ccccc34)cc2)cc1 ; c1ccc(-c2ccc(N(c3ccc4c(c3)oc3ccccc34)c3cc4c(ccc5oc6ccccc6c54)c4ccccc34)cc2)cc1]

Under a nitrogen atmosphere, to a 250 mL three-necked flask were sequentially added Sub-c1 (11.31 g, 25 mmol), RM-3 (6.56 g, 27.5 mmol), tris(dibenzylideneacetone) dipalladium (0.916 g, 0.5 mmol), (2-dicyclohexylphosphino-2′,4′,6′triisopropylbiphenyl) (0.95 g, 1 mmol), sodium tert-butoxide (9.61 g, 50 mmol) and xylene (120 mL). The reaction mixture was heated to reflux and stirred overnight. After the reaction system was cooled to room temperature, the reaction solution was extracted with dichloromethane (100 mL×3 times). The organic phases were combined, dried over anhydrous sodium sulfate. After filtration, the filtrate was distillated under reduced pressure to remove the solvent, to obtain a crude product. The crude product was purified by silica gel column chromatography using a mixed solvent of n-heptane and dichloromethane as the mobile phase, to yield compound 4 as a white solid (11.58 g, yield 77%, m/z=602.21 [M+H]⁺).

The compounds in Table 6 were synthesized following the synthesis of Compound 4, except that Reactant G was used instead of Sub-c1 and Reactant H was used instead of RM-3, as indicated in Table 6.

TABLE 6
Synthesis of the compounds of the present application

| | | | m/z | Yield |
|---|---|---|---|---|
| Reactant G | Reactant H | Compound structure and No. | ([M + H]⁺) | % |
| 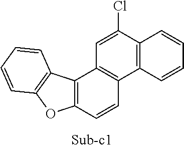 [SMILES: Clc1cc2c(ccc3oc4ccccc4c32)c2ccccc12] | 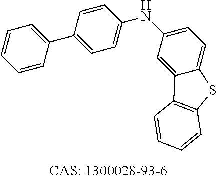 [SMILES: c1ccc(-c2ccc(Nc3ccc4sc5ccccc5c4c3)cc2)cc1] | 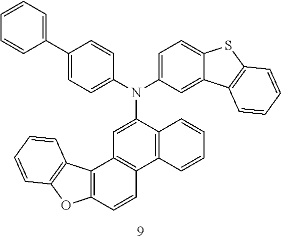 [SMILES: c1ccc(-c2ccc(N(c3ccc4sc5ccccc5c4c3)c3cc4c(ccc5oc6ccccc6c54)c4ccccc34)cc2)cc1] | 618.19 | 80 |
| 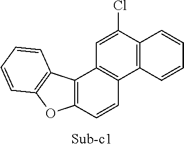 [SMILES: Clc1cc2c(ccc3oc4ccccc4c32)c2ccccc12] | 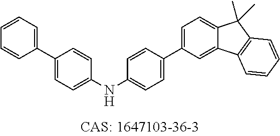 [SMILES: CC1(C)c2ccccc2-c2cc(-c3ccc(Nc4ccc(-c5ccccc5)cc4)cc3)ccc21] | 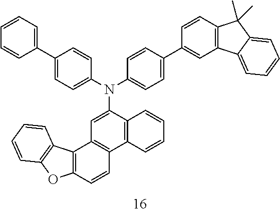 [SMILES: CC1(C)c2ccccc2-c2cc(-c3ccc(N(c4ccc(-c5ccccc5)cc4)c4cc5c(ccc6oc7ccccc7c65)c5ccccc45)cc3)ccc21] | 704.29 | 72 |
| 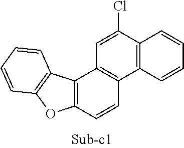 [SMILES: Clc1cc2c(ccc3oc4ccccc4c32)c2ccccc12] | 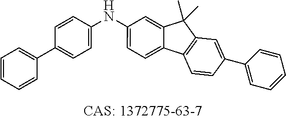 [SMILES: CC1(C)c2cc(Nc3ccc(-c4ccccc4)cc3)ccc2-c2ccc(-c3ccccc3)cc21] | 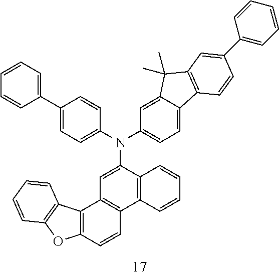 [SMILES: CC1(C)c2cc(-c3ccccc3)ccc2-c2ccc(N(c3ccc(-c4ccccc4)cc3)c3cc4c(ccc5oc6ccccc6c54)c4ccccc34)cc21] | 704.29 | 78 |
| 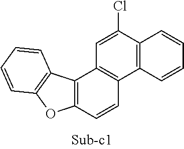 [SMILES: Clc1cc2c(ccc3oc4ccccc4c32)c2ccccc12] | 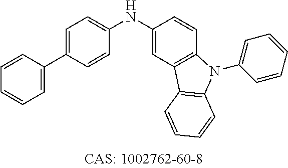 [SMILES: c1ccc(-c2ccc(Nc3ccc4c(c3)c3ccccc3n4-c3ccccc3)cc2)cc1] | 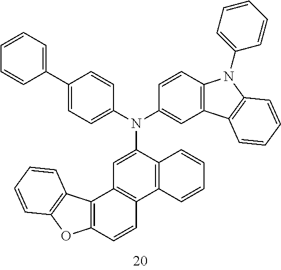 [SMILES: c1ccc(-c2ccc(N(c3ccc4c(c3)c3ccccc3n4-c3ccccc3)c3cc4c(ccc5oc6ccccc6c54)c4ccccc34)cc2)cc1] | 677.26 | 72 |
| 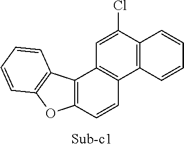 [SMILES: Clc1cc2c(ccc3oc4ccccc4c32)c2ccccc12] | 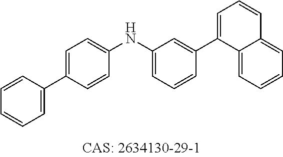 [SMILES: c1ccc(-c2ccc(Nc3cccc(-c4cccc5ccccc45)c3)cc2)cc1] | 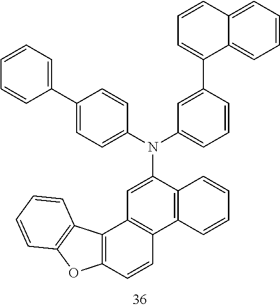 [SMILES: c1ccc(-c2ccc(N(c3cccc(-c4cccc5ccccc45)c3)c3cc4c(ccc5oc6ccccc6c54)c4ccccc34)cc2)cc1] | 638.24 | 80 |
| 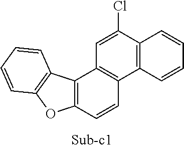 [SMILES: Clc1cc2c(ccc3oc4ccccc4c32)c2ccccc12] | 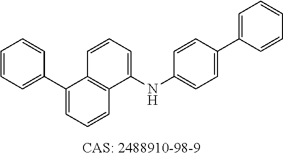 [SMILES: c1ccc(-c2ccc(Nc3cccc4c(-c5ccccc5)cccc34)cc2)cc1] | 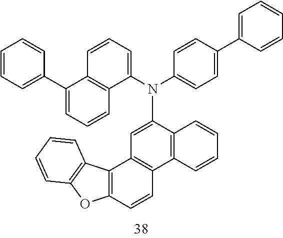 [SMILES: c1ccc(-c2ccc(N(c3cccc4c(-c5ccccc5)cccc34)c3cc4c(ccc5oc6ccccc6c54)c4ccccc34)cc2)cc1] | 638.24 | 70 |
| 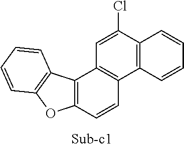 [SMILES: Clc1cc2c(ccc3oc4ccccc4c32)c2ccccc12] | 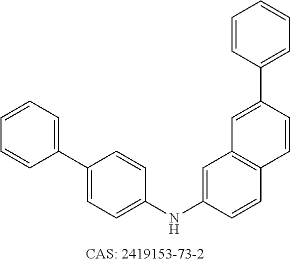 [SMILES: c1ccc(-c2ccc(Nc3ccc4ccc(-c5ccccc5)cc4c3)cc2)cc1] | 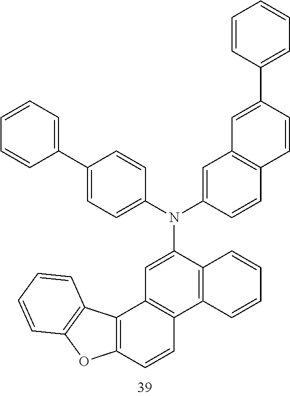 [SMILES: c1ccc(-c2ccc(N(c3ccc4ccc(-c5ccccc5)cc4c3)c3cc4c(ccc5oc6ccccc6c54)c4ccccc34)cc2)cc1] | 638.24 | 81 |
| 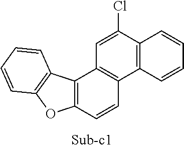 [SMILES: Clc1cc2c(ccc3oc4ccccc4c32)c2ccccc12] | 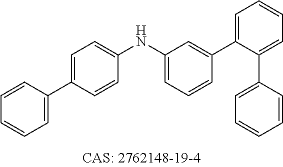 [SMILES: c1ccc(-c2ccc(Nc3cccc(-c4ccccc4-c4ccccc4)c3)cc2)cc1] | 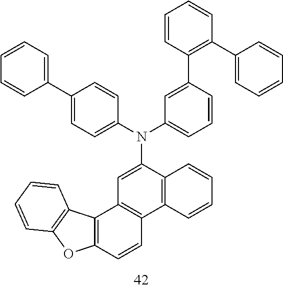 [SMILES: c1ccc(-c2ccc(N(c3cccc(-c4ccccc4-c4ccccc4)c3)c3cc4c(ccc5oc6ccccc6c54)c4ccccc34)cc2)cc1] | 664.26 | 80 |
| 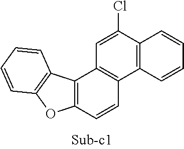 [SMILES: Clc1cc2c(ccc3oc4ccccc4c32)c2ccccc12] | 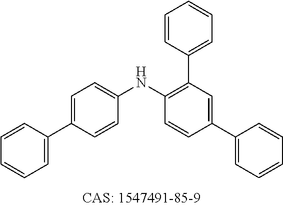 [SMILES: c1ccc(-c2ccc(Nc3ccc(-c4ccccc4)cc3-c3ccccc3)cc2)cc1] | 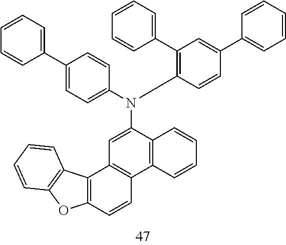 [SMILES: c1ccc(-c2ccc(N(c3ccc(-c4ccccc4)cc3-c3ccccc3)c3cc4c(ccc5oc6ccccc6c54)c4ccccc34)cc2)cc1] | 664.26 | 62 |
| 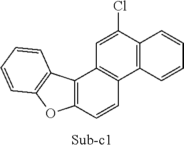 [SMILES: Clc1cc2c(ccc3oc4ccccc4c32)c2ccccc12] | 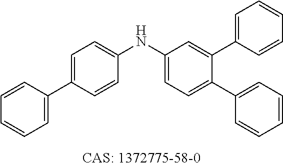 [SMILES: c1ccc(-c2ccc(Nc3ccc(-c4ccccc4)c(-c4ccccc4)c3)cc2)cc1] | 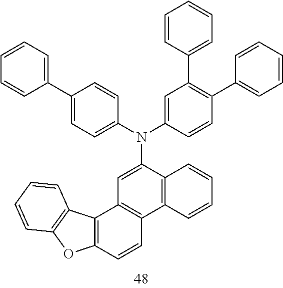 [SMILES: c1ccc(-c2ccc(N(c3ccc(-c4ccccc4)c(-c4ccccc4)c3)c3cc4c(ccc5oc6ccccc6c54)c4ccccc34)cc2)cc1] | 664.26 | 79 |
| 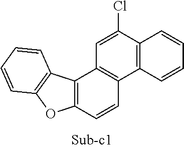 [SMILES: Clc1cc2c(ccc3oc4ccccc4c32)c2ccccc12] | 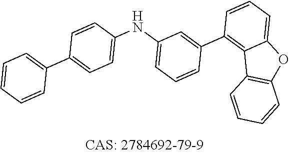 [SMILES: c1ccc(-c2ccc(Nc3cccc(-c4cccc5oc6ccccc6c45)c3)cc2)cc1] | 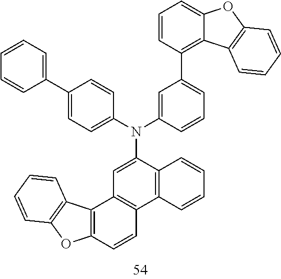 [SMILES: c1ccc(-c2ccc(N(c3cccc(-c4cccc5oc6ccccc6c45)c3)c3cc4c(ccc5oc6ccccc6c54)c4ccccc34)cc2)cc1] | 678.24 | 74 |
| 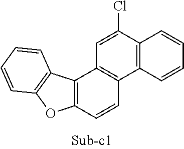 [SMILES: Clc1cc2c(ccc3oc4ccccc4c32)c2ccccc12] | 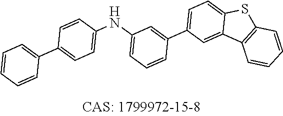 [SMILES: c1ccc(-c2ccc(Nc3cccc(-c4ccc5sc6ccccc6c5c4)c3)cc2)cc1] | 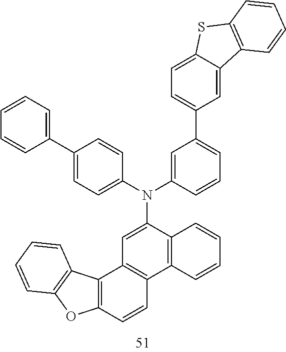 [SMILES: c1ccc(-c2ccc(N(c3cccc(-c4ccc5sc6ccccc6c5c4)c3)c3cc4c(ccc5oc6ccccc6c54)c4ccccc34)cc2)cc1] | 694.22 | 75 |
| 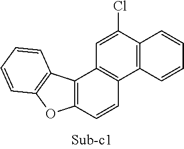 [SMILES: Clc1cc2c(ccc3oc4ccccc4c32)c2ccccc12] | 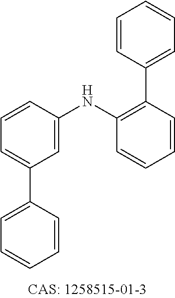 [SMILES: c1ccc(-c2cccc(Nc3ccccc3-c3ccccc3)c2)cc1] | 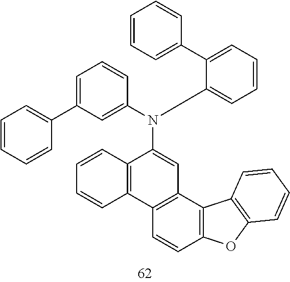 [SMILES: c1ccc(-c2cccc(N(c3ccccc3-c3ccccc3)c3cc4c(ccc5oc6ccccc6c54)c4ccccc34)c2)cc1] | 588.23 | 61 |
| 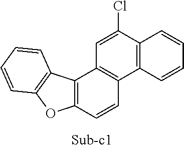 [SMILES: Clc1cc2c(ccc3oc4ccccc4c32)c2ccccc12] | 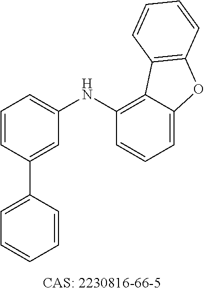 [SMILES: c1ccc(-c2cccc(Nc3cccc4oc5ccccc5c34)c2)cc1] | 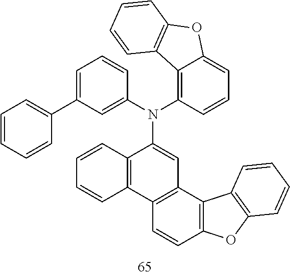 [SMILES: c1ccc(-c2cccc(N(c3cc4c(ccc5oc6ccccc6c54)c4ccccc34)c3cccc4oc5ccccc5c34)c2)cc1] | 602.21 | 65 |
| 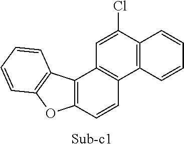 [SMILES: Clc1cc2c(ccc3oc4ccccc4c32)c2ccccc12] | 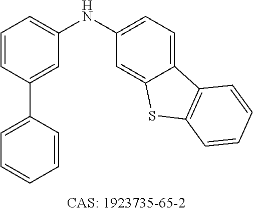 [SMILES: c1ccc(-c2cccc(Nc3ccc4c(c3)sc3ccccc34)c2)cc1] | 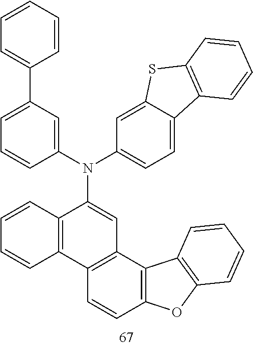 [SMILES: c1ccc(-c2cccc(N(c3ccc4c(c3)sc3ccccc34)c3cc4c(ccc5oc6ccccc6c54)c4ccccc34)c2)cc1] | 618.19 | 74 |
| 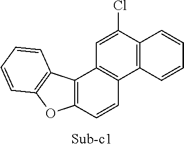 [SMILES: Clc1cc2c(ccc3oc4ccccc4c32)c2ccccc12] | 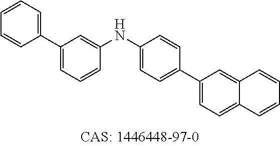 [SMILES: c1ccc(-c2cccc(Nc3ccc(-c4ccc5ccccc5c4)cc3)c2)cc1] | 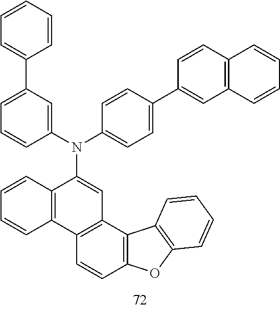 [SMILES: c1ccc(-c2cccc(N(c3ccc(-c4ccc5ccccc5c4)cc3)c3cc4c(ccc5oc6ccccc6c54)c4ccccc34)c2)cc1] | 638.24 | 79 |
| 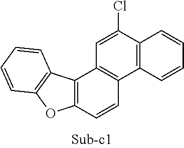 [SMILES: Clc1cc2c(ccc3oc4ccccc4c32)c2ccccc12] | 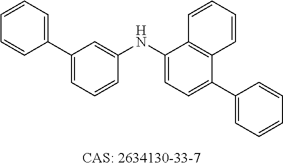 [SMILES: c1ccc(-c2cccc(Nc3ccc(-c4ccccc4)c4ccccc34)c2)cc1] | 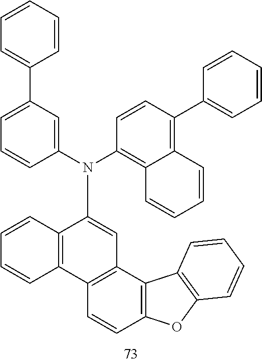 [SMILES: c1ccc(-c2cccc(N(c3ccc(-c4ccccc4)c4ccccc34)c3cc4c(ccc5oc6ccccc6c54)c4ccccc34)c2)cc1] | 638.24 | 80 |
| 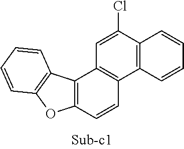 [SMILES: Clc1cc2c(ccc3oc4ccccc4c32)c2ccccc12] | 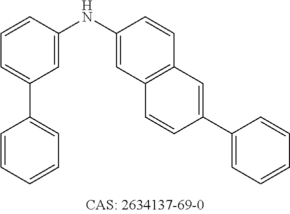 [SMILES: c1ccc(-c2cccc(Nc3ccc4cc(-c5ccccc5)ccc4c3)c2)cc1] | 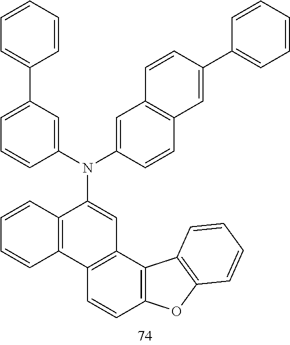 [SMILES: c1ccc(-c2cccc(N(c3ccc4cc(-c5ccccc5)ccc4c3)c3cc4c(ccc5oc6ccccc6c54)c4ccccc34)c2)cc1] | 638.24 | 79 |
| 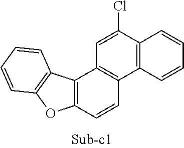 [SMILES: Clc1cc2c(ccc3oc4ccccc4c32)c2ccccc12] | 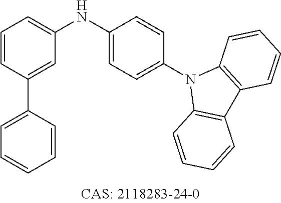 [SMILES: c1ccc(-c2cccc(Nc3ccc(-n4c5ccccc5c5ccccc54)cc3)c2)cc1] | 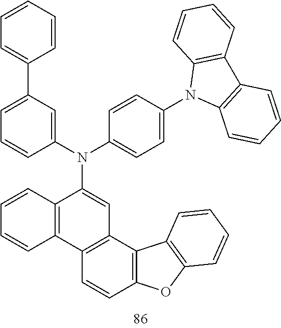 [SMILES: c1ccc(-c2cccc(N(c3ccc(-n4c5ccccc5c5ccccc54)cc3)c3cc4c(ccc5oc6ccccc6c54)c4ccccc34)c2)cc1] | 677.26 | 73 |
| 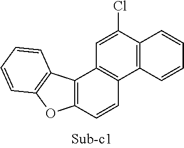 [SMILES: Clc1cc2c(ccc3oc4ccccc4c32)c2ccccc12] | 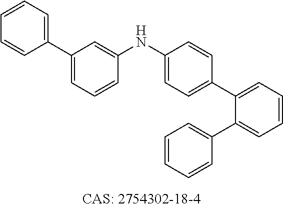 [SMILES: c1ccc(-c2cccc(Nc3ccc(-c4ccccc4-c4ccccc4)cc3)c2)cc1] | 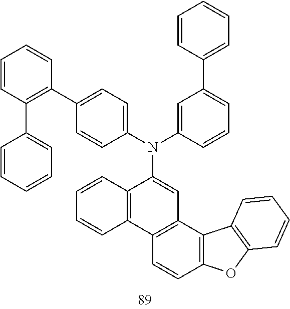 [SMILES: c1ccc(-c2cccc(N(c3ccc(-c4ccccc4-c4ccccc4)cc3)c3cc4c(ccc5oc6ccccc6c54)c4ccccc34)c2)cc1] | 664.26 | 76 |
| 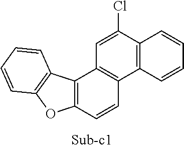 [SMILES: Clc1cc2c(ccc3oc4ccccc4c32)c2ccccc12] | 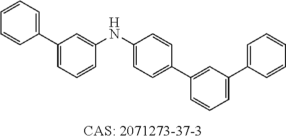 [SMILES: c1ccc(-c2cccc(Nc3ccc(-c4cccc(-c5ccccc5)c4)cc3)c2)cc1] | 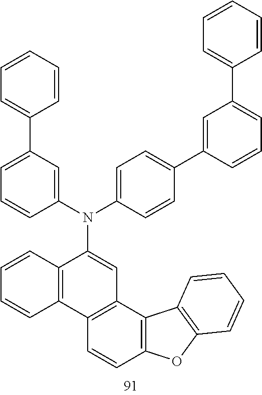 [SMILES: c1ccc(-c2cccc(-c3ccc(N(c4cccc(-c5ccccc5)c4)c4cc5c(ccc6oc7ccccc7c65)c5ccccc45)cc3)c2)cc1] | 664.26 | 74 |
| 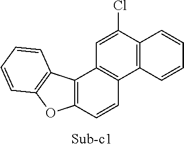 [SMILES: Clc1cc2c(ccc3oc4ccccc4c32)c2ccccc12] | 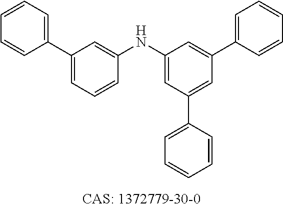 [SMILES: c1ccc(-c2cccc(Nc3cc(-c4ccccc4)cc(-c4ccccc4)c3)c2)cc1] | 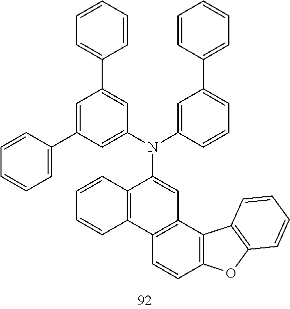 [SMILES: c1ccc(-c2cccc(N(c3cc(-c4ccccc4)cc(-c4ccccc4)c3)c3cc4c(ccc5oc6ccccc6c54)c4ccccc34)c2)cc1] | 664.26 | 70 |
| 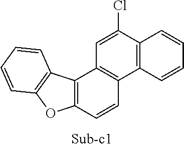 [SMILES: Clc1cc2c(ccc3oc4ccccc4c32)c2ccccc12] | 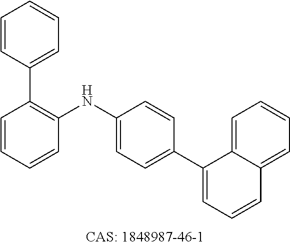 [SMILES: c1ccc(-c2ccccc2Nc2ccc(-c3cccc4ccccc34)cc2)cc1] | 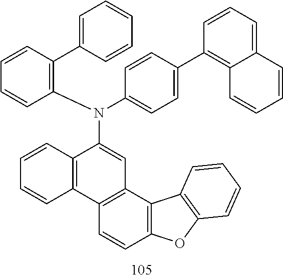 [SMILES: c1ccc(-c2ccccc2N(c2ccc(-c3cccc4ccccc34)cc2)c2cc3c(ccc4oc5ccccc5c43)c3ccccc23)cc1] | 638.24 | 65 |
| 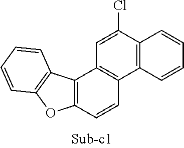 [SMILES: Clc1cc2c(ccc3oc4ccccc4c32)c2ccccc12] | 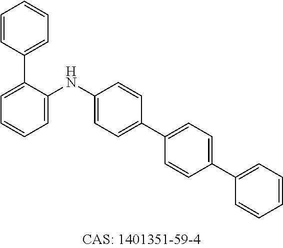 [SMILES: c1ccc(-c2ccc(-c3ccc(Nc4ccccc4-c4ccccc4)cc3)cc2)cc1] | 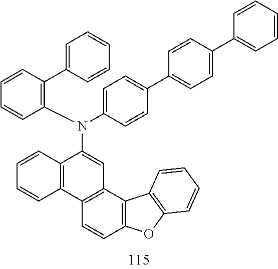 [SMILES: c1ccc(-c2ccc(-c3ccc(N(c4ccccc4-c4ccccc4)c4cc5c(ccc6oc7ccccc7c65)c5ccccc45)cc3)cc2)cc1] | 664.26 | 67 |
| 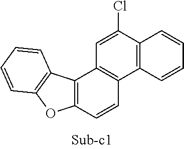 [SMILES: Clc1cc2c(ccc3oc4ccccc4c32)c2ccccc12] | 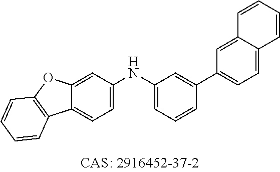 [SMILES: c1cc(Nc2ccc3c(c2)oc2ccccc23)cc(-c2ccc3ccccc3c2)c1] | 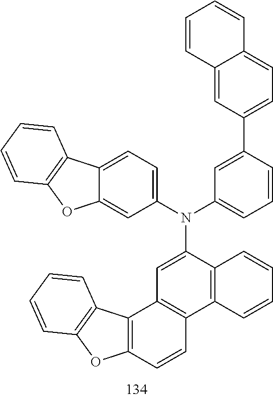 [SMILES: c1cc(-c2ccc3ccccc3c2)cc(N(c2ccc3c(c2)oc2ccccc23)c2cc3c(ccc4oc5ccccc5c43)c3ccccc23)c1] | 652.22 | 75 |
| 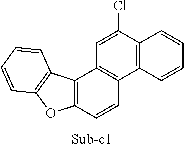 [SMILES: Clc1cc2c(ccc3oc4ccccc4c32)c2ccccc12] | 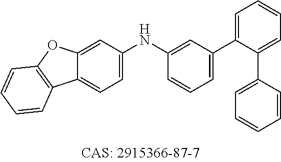 [SMILES: c1ccc(-c2ccccc2-c2cccc(Nc3ccc4c(c3)oc3ccccc34)c2)cc1] | 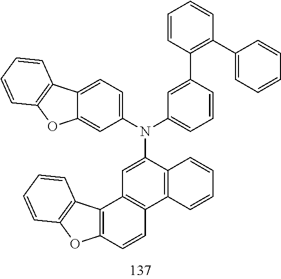 [SMILES: c1ccc(-c2ccccc2-c2cccc(N(c3ccc4c(c3)oc3ccccc34)c3cc4c(ccc5oc6ccccc6c54)c4ccccc34)c2)cc1] | 678.24 | 78 |
| 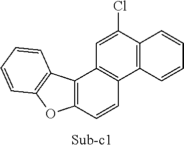 [SMILES: Clc1cc2c(ccc3oc4ccccc4c32)c2ccccc12] | 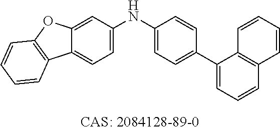 [SMILES: c1ccc2c(-c3ccc(Nc4ccc5c(c4)oc4ccccc45)cc3)cccc2c1] | 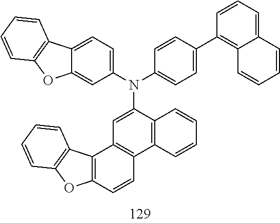 [SMILES: c1ccc2c(-c3ccc(N(c4ccc5c(c4)oc4ccccc45)c4cc5c(ccc6oc7ccccc7c65)c5ccccc45)cc3)cccc2c1] | 652.22 | 76 |
| 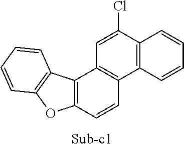 [SMILES: Clc1cc2c(ccc3oc4ccccc4c32)c2ccccc12] | 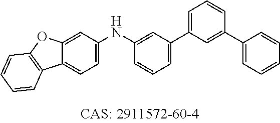 [SMILES: c1ccc(-c2cccc(-c3cccc(Nc4ccc5c(c4)oc4ccccc45)c3)c2)cc1] | 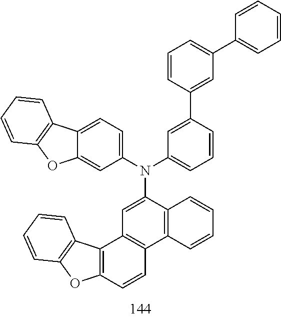 [SMILES: c1ccc(-c2cccc(-c3cccc(N(c4ccc5c(c4)oc4ccccc45)c4cc5c(ccc6oc7ccccc7c65)c5ccccc45)c3)c2)cc1] | 678.24 | 71 |
| 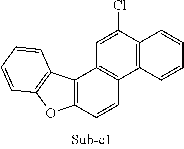 [SMILES: Clc1cc2c(ccc3oc4ccccc4c32)c2ccccc12] | 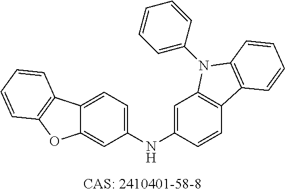 [SMILES: c1ccc(-n2c3ccccc3c3ccc(Nc4ccc5c(c4)oc4ccccc45)cc32)cc1] | 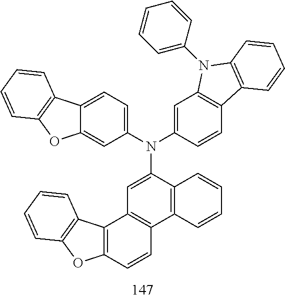 [SMILES: c1ccc(-n2c3ccccc3c3ccc(N(c4ccc5c(c4)oc4ccccc45)c4cc5c(ccc6oc7ccccc7c65)c5ccccc45)cc32)cc1] | 691.23 | 77 |
| 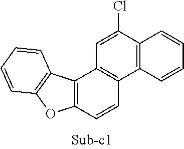 [SMILES: Clc1cc2c(ccc3oc4ccccc4c32)c2ccccc12] | 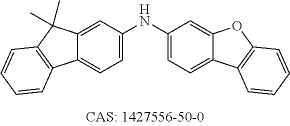 [SMILES: CC1(C)c2ccccc2-c2ccc(Nc3ccc4c(c3)oc3ccccc34)cc21] | 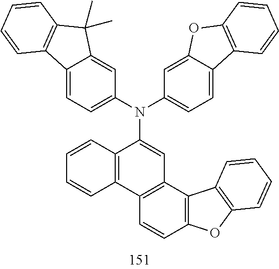 [SMILES: CC1(C)c2ccccc2-c2ccc(N(c3ccc4c(c3)oc3ccccc34)c3cc4c(ccc5oc6ccccc6c54)c4ccccc34)cc21] | 642.24 | 77 |
| 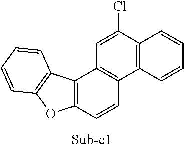 [SMILES: Clc1cc2c(ccc3oc4ccccc4c32)c2ccccc12] | 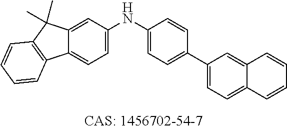 [SMILES: CC1(C)c2ccccc2-c2ccc(Nc3ccc(-c4ccc5ccccc5c4)cc3)cc21] | 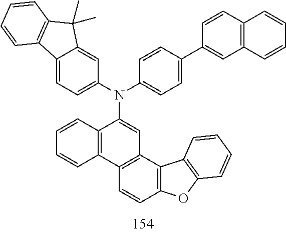 [SMILES: CC1(C)c2ccccc2-c2ccc(N(c3ccc(-c4ccc5ccccc5c4)cc3)c3cc4c(ccc5oc6ccccc6c54)c4ccccc34)cc21] | 678.28 | 73 |
| 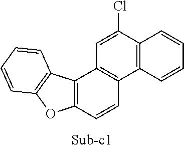 [SMILES: Clc1cc2c(ccc3oc4ccccc4c32)c2ccccc12] | 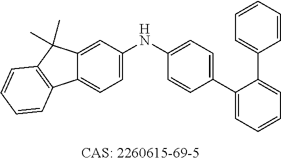 [SMILES: CC1(C)c2ccccc2-c2ccc(Nc3ccc(-c4ccccc4-c4ccccc4)cc3)cc21] | 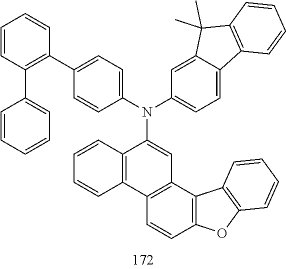 [SMILES: CC1(C)c2ccccc2-c2ccc(N(c3ccc(-c4ccccc4-c4ccccc4)cc3)c3cc4c(ccc5oc6ccccc6c54)c4ccccc34)cc21] | 704.29 | 78 |
| 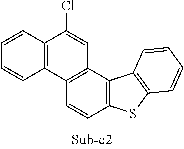 [SMILES: Clc1cc2c(ccc3sc4ccccc4c32)c2ccccc12] | 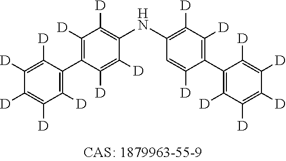 [SMILES: [2H]c1cc(Nc2c([2H])c([2H])c(-c3c([2H])c([2H])c([2H])c([2H])c3[2H])c([2H])c2[2H])c([2H])c([2H])c1-c1c([2H])c([2H])c([2H])c([2H])c1[2H]] | 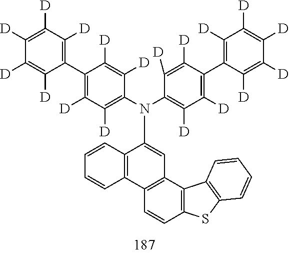 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3cc4c(ccc5sc6ccccc6c54)c4ccccc34)c([2H])c2[2H])c([2H])c1[2H]] | 622.32 | 71 |
| 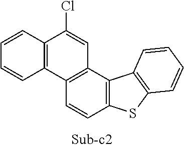 [SMILES: Clc1cc2c(ccc3sc4ccccc4c32)c2ccccc12] | 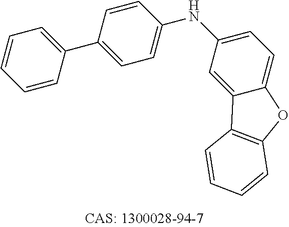 [SMILES: c1ccc(-c2ccc(Nc3ccc4oc5ccccc5c4c3)cc2)cc1] | 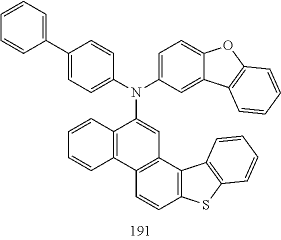 [SMILES: c1ccc(-c2ccc(N(c3ccc4oc5ccccc5c4c3)c3cc4c(ccc5sc6ccccc6c54)c4ccccc34)cc2)cc1] | 618.19 | 75 |
| 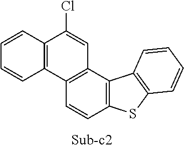 [SMILES: Clc1cc2c(ccc3sc4ccccc4c32)c2ccccc12] | 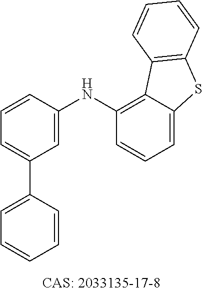 [SMILES: c1ccc(-c2cccc(Nc3cccc4sc5ccccc5c34)c2)cc1] | 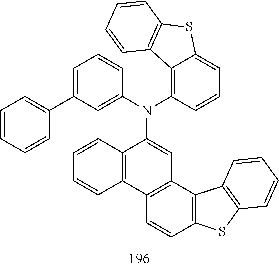 [SMILES: c1ccc(-c2cccc(N(c3cc4c(ccc5sc6ccccc6c54)c4ccccc34)c3cccc4sc5ccccc5c34)c2)cc1] | 634.16 | 63 |
| 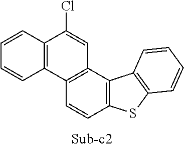 [SMILES: Clc1cc2c(ccc3sc4ccccc4c32)c2ccccc12] | 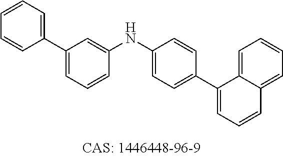 [SMILES: c1ccc(-c2cccc(Nc3ccc(-c4cccc5ccccc45)cc3)c2)cc1] | 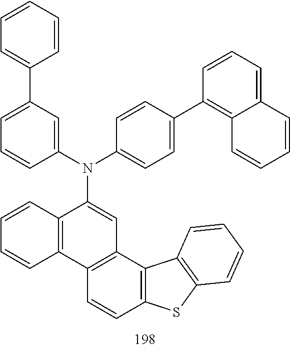 [SMILES: c1ccc(-c2cccc(N(c3ccc(-c4cccc5ccccc45)cc3)c3cc4c(ccc5sc6ccccc6c54)c4ccccc34)c2)cc1] | 654.22 | 77 |
| 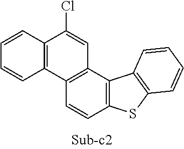 [SMILES: Clc1cc2c(ccc3sc4ccccc4c32)c2ccccc12] | 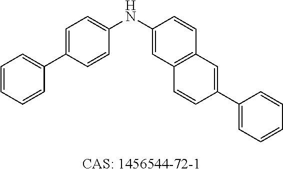 [SMILES: c1ccc(-c2ccc(Nc3ccc4cc(-c5ccccc5)ccc4c3)cc2)cc1] | 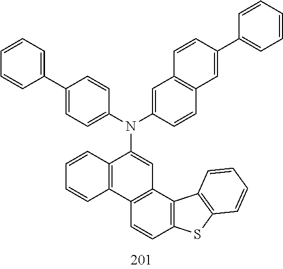 [SMILES: c1ccc(-c2ccc(N(c3ccc4cc(-c5ccccc5)ccc4c3)c3cc4c(ccc5sc6ccccc6c54)c4ccccc34)cc2)cc1] | 654.22 | 79 |
| 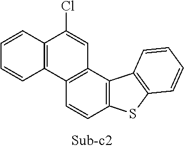 [SMILES: Clc1cc2c(ccc3sc4ccccc4c32)c2ccccc12] | 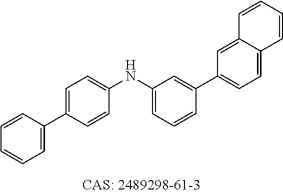 [SMILES: c1ccc(-c2ccc(Nc3cccc(-c4ccc5ccccc5c4)c3)cc2)cc1] | 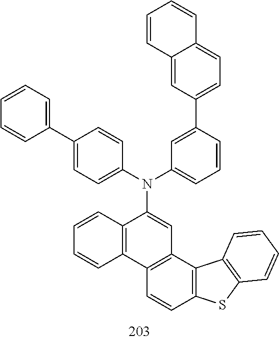 [SMILES: c1ccc(-c2ccc(N(c3cccc(-c4ccc5ccccc5c4)c3)c3cc4c(ccc5sc6ccccc6c54)c4ccccc34)cc2)cc1] | 654.22 | 80 |
| 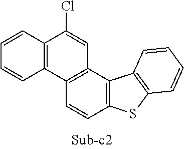 [SMILES: Clc1cc2c(ccc3sc4ccccc4c32)c2ccccc12] | 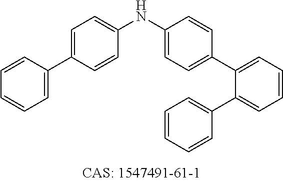 [SMILES: c1ccc(-c2ccc(Nc3ccc(-c4ccccc4-c4ccccc4)cc3)cc2)cc1] | 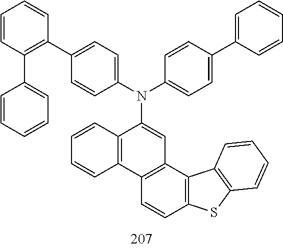 [SMILES: c1ccc(-c2ccc(N(c3ccc(-c4ccccc4-c4ccccc4)cc3)c3cc4c(ccc5sc6ccccc6c54)c4ccccc34)cc2)cc1] | 680.24 | 79 |
| 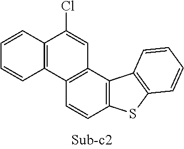 [SMILES: Clc1cc2c(ccc3sc4ccccc4c32)c2ccccc12] | 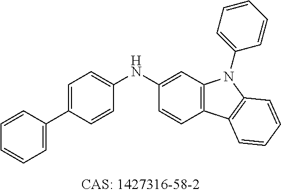 [SMILES: c1ccc(-c2ccc(Nc3ccc4c5ccccc5n(-c5ccccc5)c4c3)cc2)cc1] | 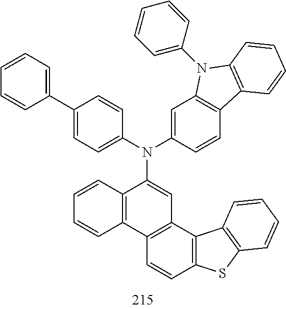 [SMILES: c1ccc(-c2ccc(N(c3ccc4c5ccccc5n(-c5ccccc5)c4c3)c3cc4c(ccc5sc6ccccc6c54)c4ccccc34)cc2)cc1] | 693.23 | 77 |
| 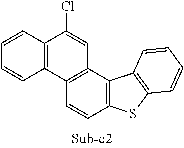 [SMILES: Clc1cc2c(ccc3sc4ccccc4c32)c2ccccc12] | 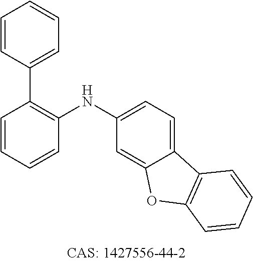 [SMILES: c1ccc(-c2ccccc2Nc2ccc3c(c2)oc2ccccc23)cc1] | 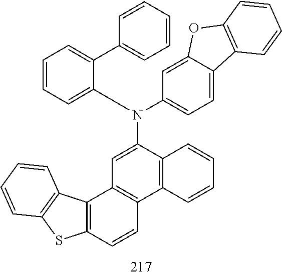 [SMILES: c1ccc(-c2ccccc2N(c2ccc3c(c2)oc2ccccc23)c2cc3c(ccc4sc5ccccc5c43)c3ccccc23)cc1] | 618.19 | 66 |
| 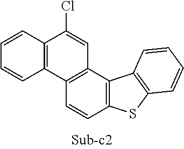 [SMILES: Clc1cc2c(ccc3sc4ccccc4c32)c2ccccc12] | 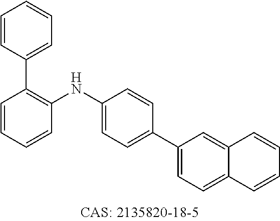 [SMILES: c1ccc(-c2ccccc2Nc2ccc(-c3ccc4ccccc4c3)cc2)cc1] | 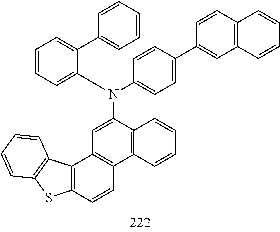 [SMILES: c1ccc(-c2ccccc2N(c2ccc(-c3ccc4ccccc4c3)cc2)c2cc3c(ccc4sc5ccccc5c43)c3ccccc23)cc1] | 654.22 | 70 |
| 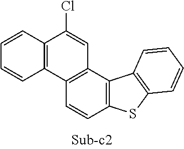 [SMILES: Clc1cc2c(ccc3sc4ccccc4c32)c2ccccc12] | 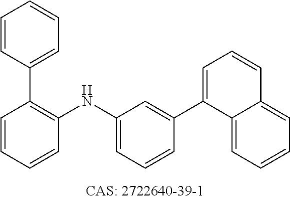 [SMILES: c1ccc(-c2ccccc2Nc2cccc(-c3cccc4ccccc34)c2)cc1] | 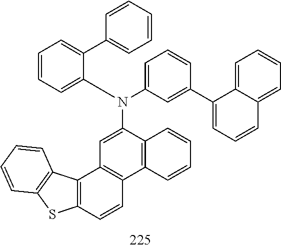 [SMILES: c1ccc(-c2ccccc2N(c2cccc(-c3cccc4ccccc34)c2)c2cc3c(ccc4sc5ccccc5c43)c3ccccc23)cc1] | 654.22 | 74 |
| 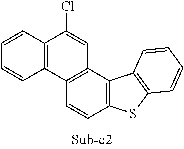 [SMILES: Clc1cc2c(ccc3sc4ccccc4c32)c2ccccc12] | 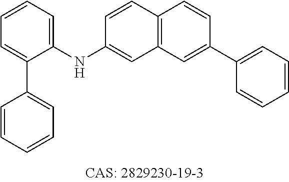 [SMILES: c1ccc(-c2ccc3ccc(Nc4ccccc4-c4ccccc4)cc3c2)cc1] | 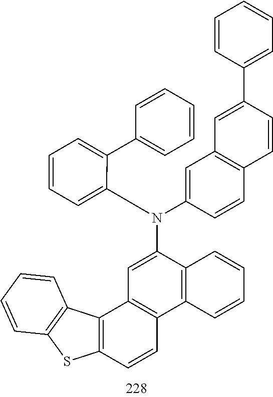 [SMILES: c1ccc(-c2ccc3ccc(N(c4ccccc4-c4ccccc4)c4cc5c(ccc6sc7ccccc7c65)c5ccccc45)cc3c2)cc1] | 654.22 | 71 |
| 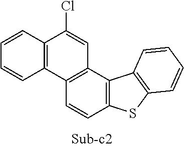 [SMILES: Clc1cc2c(ccc3sc4ccccc4c32)c2ccccc12] | 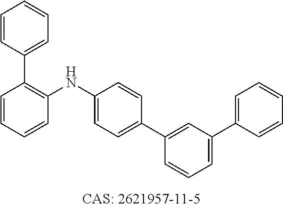 [SMILES: c1ccc(-c2cccc(-c3ccc(Nc4ccccc4-c4ccccc4)cc3)c2)cc1] | 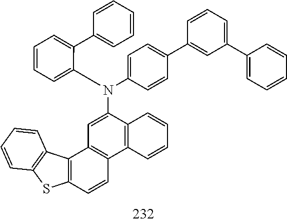 [SMILES: c1ccc(-c2cccc(-c3ccc(N(c4ccccc4-c4ccccc4)c4cc5c(ccc6sc7ccccc7c65)c5ccccc45)cc3)c2)cc1] | 680.24 | 65 |
| 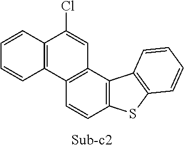 [SMILES: Clc1cc2c(ccc3sc4ccccc4c32)c2ccccc12] | 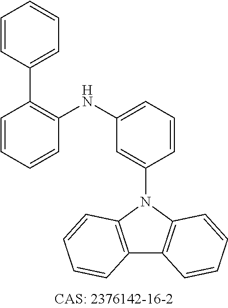 [SMILES: c1ccc(-c2ccccc2Nc2cccc(-n3c4ccccc4c4ccccc43)c2)cc1] | 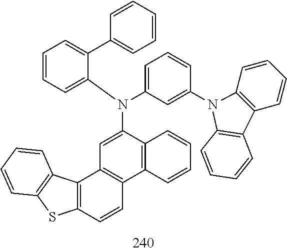 [SMILES: c1ccc(-c2ccccc2N(c2cccc(-n3c4ccccc4c4ccccc43)c2)c2cc3c(ccc4sc5ccccc5c43)c3ccccc23)cc1] | 693.23 | 66 |
| 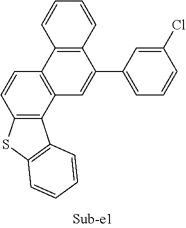 [SMILES: Clc1cccc(-c2cc3c(ccc4sc5ccccc5c43)c3ccccc23)c1] | 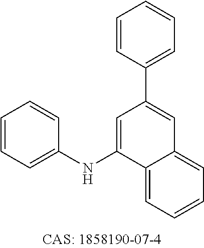 [SMILES: c1ccc(Nc2cc(-c3ccccc3)cc3ccccc23)cc1] | 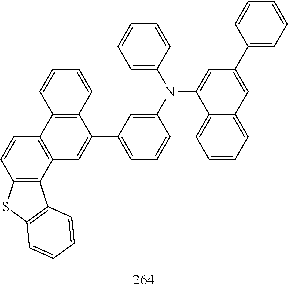 [SMILES: c1ccc(-c2cc(N(c3ccccc3)c3cccc(-c4cc5c(ccc6sc7ccccc7c65)c5ccccc45)c3)c3ccccc3c2)cc1] | 654.22 | 78 |
| 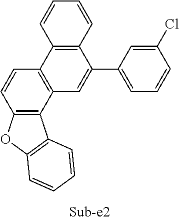 [SMILES: Clc1cccc(-c2cc3c(ccc4oc5ccccc5c43)c3ccccc23)c1] | 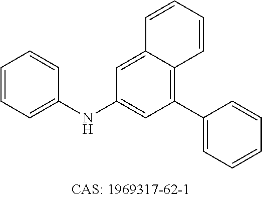 [SMILES: c1ccc(Nc2cc(-c3ccccc3)c3ccccc3c2)cc1] | 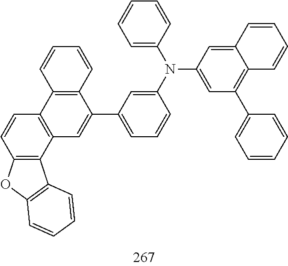 [SMILES: c1ccc(-c2cc(N(c3ccccc3)c3cccc(-c4cc5c(ccc6oc7ccccc7c65)c5ccccc45)c3)cc3ccccc23)cc1] | 638.24 | 79 |
| 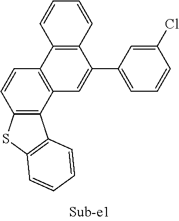 [SMILES: Clc1cccc(-c2cc3c(ccc4sc5ccccc5c43)c3ccccc23)c1] | 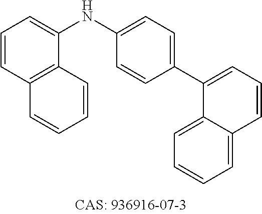 [SMILES: c1ccc2c(Nc3ccc(-c4cccc5ccccc45)cc3)cccc2c1] | 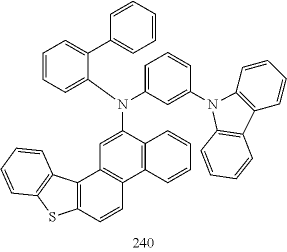 [SMILES: c1ccc(-c2ccccc2N(c2cccc(-n3c4ccccc4c4ccccc43)c2)c2cc3c(ccc4sc5ccccc5c43)c3ccccc23)cc1] | 704.24 | 79 |
| 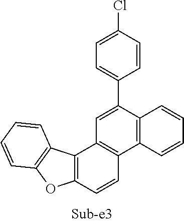 [SMILES: Clc1ccc(-c2cc3c(ccc4oc5ccccc5c43)c3ccccc23)cc1] | 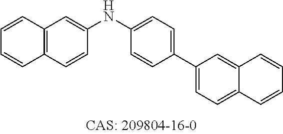 [SMILES: c1ccc2cc(Nc3ccc(-c4ccc5ccccc5c4)cc3)ccc2c1] | 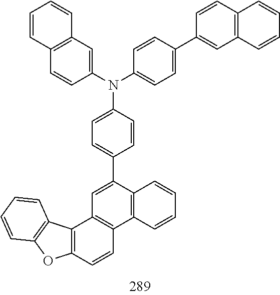 [SMILES: c1ccc2cc(-c3ccc(N(c4ccc(-c5cc6c(ccc7oc8ccccc8c76)c6ccccc56)cc4)c4ccc5ccccc5c4)cc3)ccc2c1] | 688.26 | 77 |
| 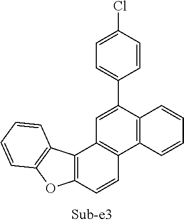 [SMILES: Clc1ccc(-c2cc3c(ccc4oc5ccccc5c43)c3ccccc23)cc1] | 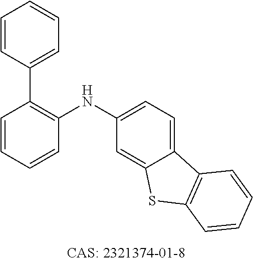 [SMILES: c1ccc(-c2ccccc2Nc2ccc3c(c2)sc2ccccc23)cc1] | 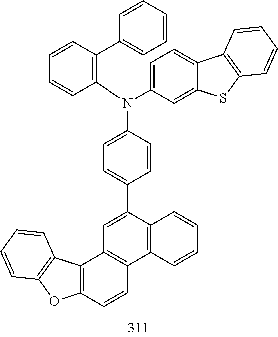 [SMILES: c1ccc(-c2ccccc2N(c2ccc(-c3cc4c(ccc5oc6ccccc6c54)c4ccccc34)cc2)c2ccc3c(c2)sc2ccccc23)cc1] | 694.22 | 80 |
| 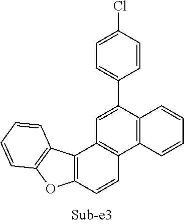 [SMILES: Clc1ccc(-c2cc3c(ccc4oc5ccccc5c43)c3ccccc23)cc1] | 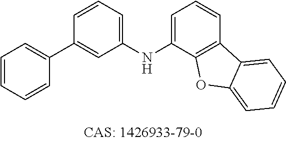 [SMILES: c1ccc(-c2cccc(Nc3cccc4c3oc3ccccc34)c2)cc1] | 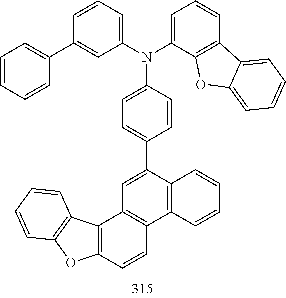 [SMILES: c1ccc(-c2cccc(N(c3ccc(-c4cc5c(ccc6oc7ccccc7c65)c5ccccc45)cc3)c3cccc4c3oc3ccccc34)c2)cc1] | 678.24 | 76 |
| 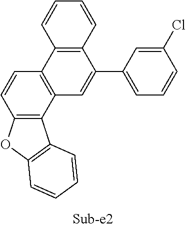 [SMILES: Clc1cccc(-c2cc3c(ccc4oc5ccccc5c43)c3ccccc23)c1] | 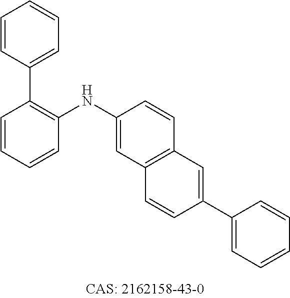 [SMILES: c1ccc(-c2ccc3cc(Nc4ccccc4-c4ccccc4)ccc3c2)cc1] | 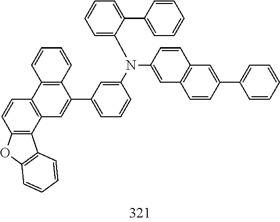 [SMILES: c1ccc(-c2ccc3cc(N(c4cccc(-c5cc6c(ccc7oc8ccccc8c76)c6ccccc56)c4)c4ccccc4-c4ccccc4)ccc3c2)cc1] | 714.28 | 74 |
| 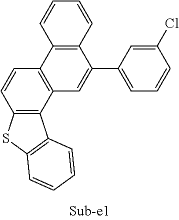 [SMILES: Clc1cccc(-c2cc3c(ccc4sc5ccccc5c43)c3ccccc23)c1] | 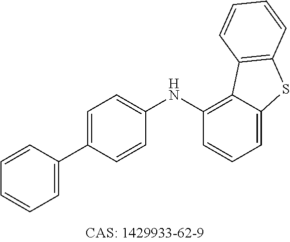 [SMILES: c1ccc(-c2ccc(Nc3cccc4sc5ccccc5c34)cc2)cc1] | 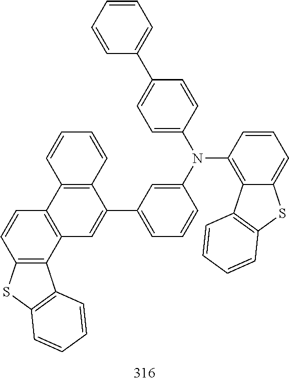 [SMILES: c1ccc(-c2ccc(N(c3cccc(-c4cc5c(ccc6sc7ccccc7c65)c5ccccc45)c3)c3cccc4sc5ccccc5c34)cc2)cc1] | 710.19 | 74 |
| 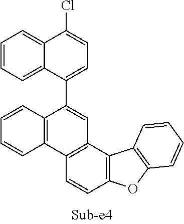 [SMILES: Clc1ccc(-c2cc3c(ccc4oc5ccccc5c43)c3ccccc23)c2ccccc12] | 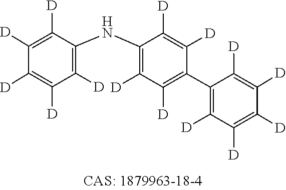 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] | 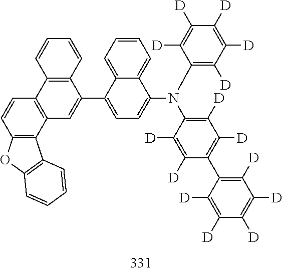 [SMILES: [2H]c1c([2H])c([2H])c(-c2c([2H])c([2H])c(N(c3c([2H])c([2H])c([2H])c([2H])c3[2H])c3ccc(-c4cc5c(ccc6oc7ccccc7c65)c5ccccc45)c4ccccc34)c([2H])c2[2H])c([2H])c1[2H]] | 652.33 | 71 |
| 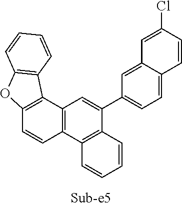 [SMILES: Clc1ccc2ccc(-c3cc4c(ccc5oc6ccccc6c54)c4ccccc34)cc2c1] | 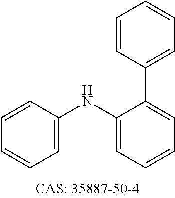 [SMILES: c1ccc(Nc2ccccc2-c2ccccc2)cc1] | 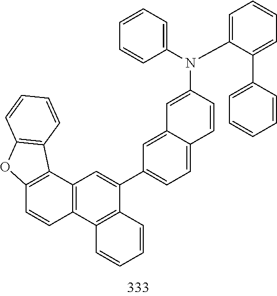 [SMILES: c1ccc(-c2ccccc2N(c2ccccc2)c2ccc3ccc(-c4cc5c(ccc6oc7ccccc7c65)c5ccccc45)cc3c2)cc1] | 638.24 | 78 |
| 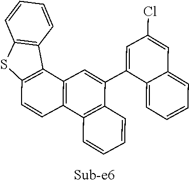 [SMILES: Clc1cc(-c2cc3c(ccc4sc5ccccc5c43)c3ccccc23)c2ccccc2c1] | 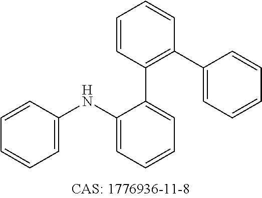 [SMILES: c1ccc(Nc2ccccc2-c2ccccc2-c2ccccc2)cc1] | 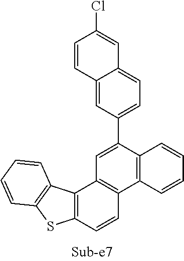 [SMILES: Clc1ccc2cc(-c3cc4c(ccc5sc6ccccc6c54)c4ccccc34)ccc2c1] | 730.25 | 61 |
| 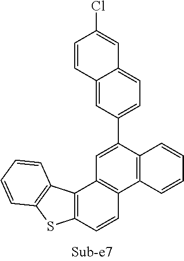 [SMILES: Clc1ccc2cc(-c3cc4c(ccc5sc6ccccc6c54)c4ccccc34)ccc2c1] | 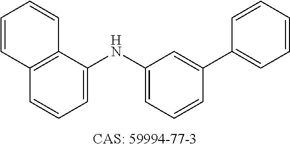 [SMILES: c1ccc(-c2cccc(Nc3cccc4ccccc34)c2)cc1] | 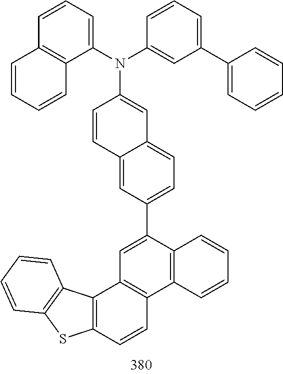 [SMILES: c1ccc(-c2cccc(N(c3ccc4cc(-c5cc6c(ccc7sc8ccccc8c76)c6ccccc56)ccc4c3)c3cccc4ccccc34)c2)cc1] | 704.24 | 71 |
| 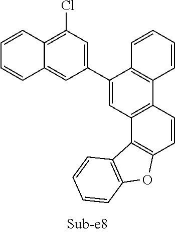 [SMILES: Clc1cc(-c2cc3c(ccc4oc5ccccc5c43)c3ccccc23)cc2ccccc12] | 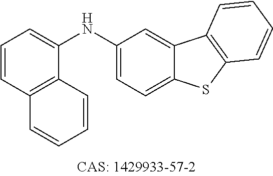 [SMILES: c1ccc2c(Nc3ccc4sc5ccccc5c4c3)cccc2c1] | 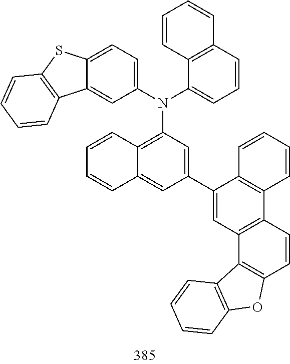 [SMILES: c1ccc2c(N(c3ccc4sc5ccccc5c4c3)c3cc(-c4cc5c(ccc6oc7ccccc7c65)c5ccccc45)cc4ccccc34)cccc2c1] | 718.22 | 79 |

### NMR Data of Some Compounds:

Compound 137: 1H-NMR (400 MHz, CD₂Cl₂) δ ppm: 8.28 (d, 1H), 8.19 (d, 1H), 7.99-7.97 (m, 2H), 7.88 (d, 1H), 7.81 (d, 1H), 7.67-7.26 (m, 17H), 7.21-7.12 (m, 4H), 7.06 (s, 1H), 6.93 (s, 1H), 6.87 (d, 1H), 6.55 (d, 1H).

### Fabrication and Evaluation of Organic Electroluminescent Device:

The present application also provides an organic electroluminescent device, comprising an anode, a cathode, and an organic layer disposed between the anode and the cathode; wherein the organic layer comprises the above compound of the present invention. The following examples provide a detailed description of the organic electroluminescent devices of the present invention. However, the following examples are merely exemplary of the present invention and are not intended to limit the scope of the invention.

### Example 1: Red Organic Electroluminescent Device

First, an anode pretreatment was performed by the following processes: the surface of ITO/Ag/ITO substrate with a thickness of 100 Å, 1000 Å, and 100 Å in sequence was performed using ultraviolet ozone and O₂:N₂ plasma to increase the work function of the anode. The surface of the ITO substrate may also be cleaned with an organic solvent to remove impurities and oil stains.

On the test substrate (anode), the PD:HT-1 was collectively evaporated at an evaporation rate ratio of 2%:98% to form a hole injection layer (HIL) with a thickness of 100 Å, and then on the hole injection layer, HT-1 was vacuum evaporated to form a hole transport layer with a thickness of 1065 Å. On the hole transport, Compound HT-2 was vacuum evaporated to form a light-emitting adjustment layer with a thickness of 840 Å.

Next, on the light-emitting adjustment layer, Compound 1:RH-N:RD were collectively evaporated at an evaporation rate ratio of 49%:49%:2% to form a red light-emitting layer (EML) with a thickness of 420 Å.

On the red light-emitting layer, Compound ET and LiQ were collectively evaporated at an evaporation rate ratio of 1:1 to form an electron transport layer (ETL) with a thickness of 350 Å. Yb was evaporated on the electron transport layer to form an electron injection layer (EIL) with a thickness of 10 Å. Then, magnesium (Mg) and silver (Ag) were evaporated on the electron injection layer at an evaporation rate ratio of 1:9 to form a cathode with a thickness of 130 Å.

In addition, CP with a thickness of 760 Å was vacuum evaporated on the above cathode as a cladding layer, thus completing the fabrication of the red organic electroluminescent device.

### Examples 2 to 60

Organic electroluminescent devices were fabricated by the same method as used in Example 1, except that Compound X in Table 7 below were used respectively instead of the Compound 1 in Example 1 when forming the light-emitting layer.

### Comparative Examples 1~4

Organic electroluminescent devices were fabricated by the same method as used in Example 1, except that Compound A, Compound B, Compound C and Compound D were used respectively instead of the Compound 1 in Example 1 when forming the light-emitting layer.

Among them, the structures of the materials, utilized in the preparation of the organic electroluminescent device n comparative examples and examples, are as follows:

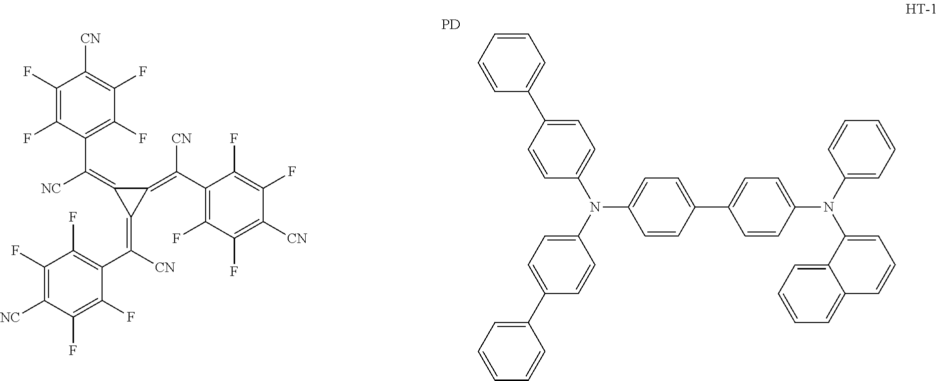 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(N(c3ccc(-c4ccccc4)cc3)c3ccc(-c4ccc(N(c5ccccc5)c5cccc6ccccc56)cc4)cc3)cc2)cc1]

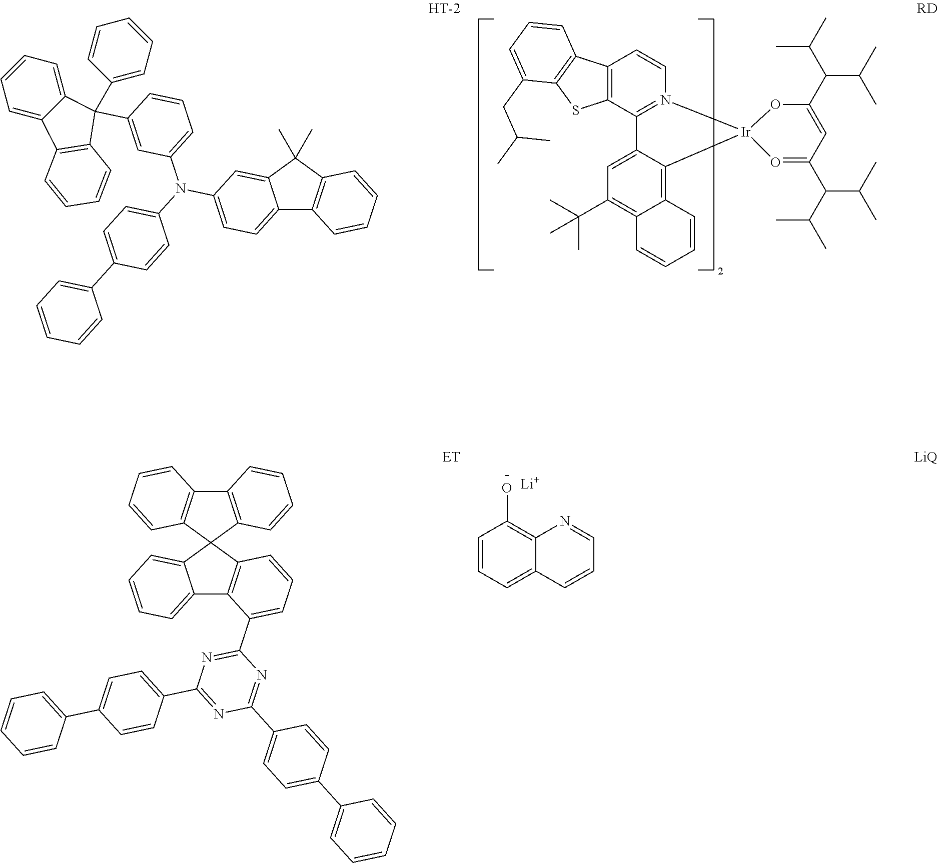

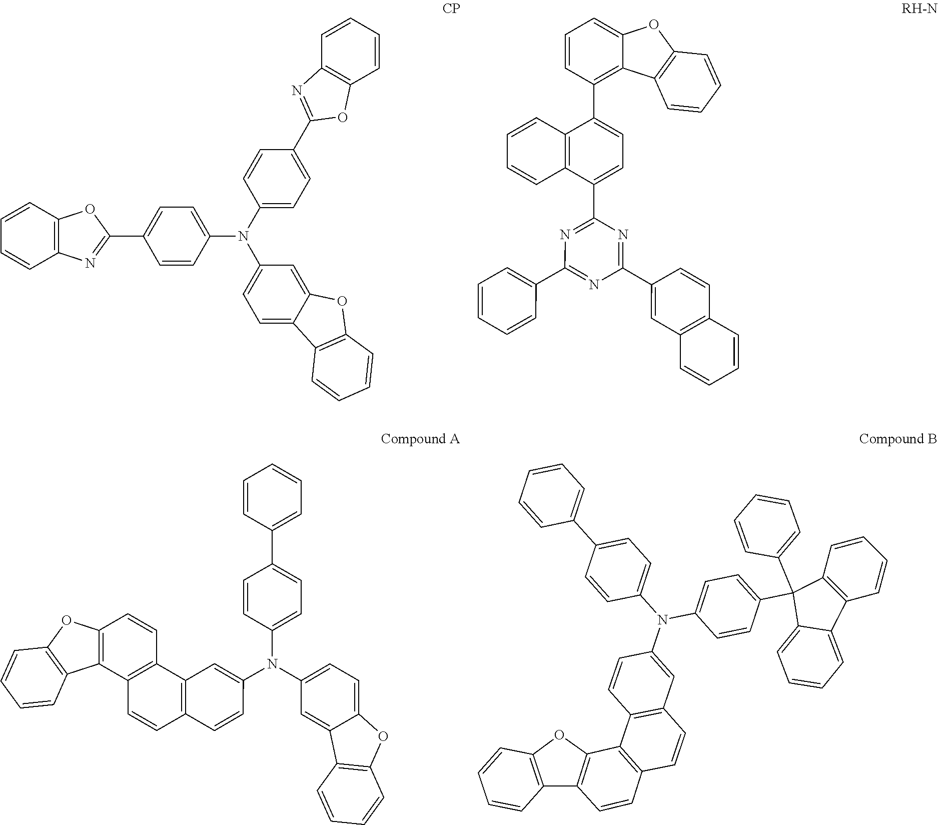 [SMILES: c1ccc2oc(-c3ccc(N(c4ccc(-c5nc6ccccc6o5)cc4)c4ccc5c(c4)oc4ccccc45)cc3)nc2c1 ; c1ccc(-c2nc(-c3ccc4ccccc4c3)nc(-c3ccc(-c4cccc5oc6ccccc6c45)c4ccccc34)n2)cc1 ; c1ccc(-c2ccc(N(c3ccc4ccc5c(ccc6oc7ccccc7c65)c4c3)c3ccc4oc5ccccc5c4c3)cc2)cc1 ; c1ccc(-c2ccc(N(c3ccc(C4(c5ccccc5)c5ccccc5-c5ccccc54)cc3)c3ccc4c(ccc5ccc6c7ccccc7oc6c54)c3)cc2)cc1]

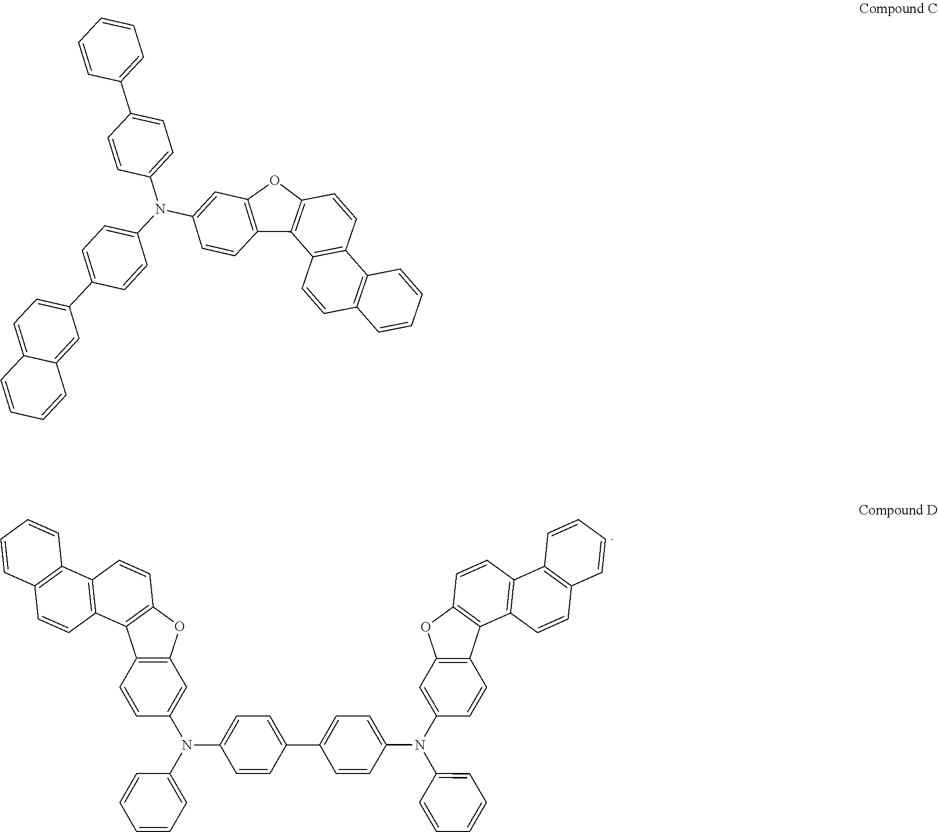 [SMILES: c1ccc(-c2ccc(N(c3ccc(-c4ccc5ccccc5c4)cc3)c3ccc4c(c3)oc3ccc5c6ccccc6ccc5c34)cc2)cc1 ; c1ccc(N(c2ccc(-c3ccc(N(c4ccccc4)c4ccc5c(c4)oc4ccc6c7ccccc7ccc6c45)cc3)cc2)c2ccc3c(c2)oc2ccc4c5ccccc5ccc4c23)cc1]

The performances of the organic electroluminescent devices prepared in Examples 1-60 and Comparative Examples 1~4 were tested. Specifically, the IVL characteristics of the devices were tested under a condition of 10 mA/cm², and the T95 device lifetime was tested under a condition of 20 mA/cm². The test results are presented in Table 7.

TABLE 7

| | Compound X | | | | | T95 |
|---|---|---|---|---|---|---|
| | in the light- | Volt | | | | (hrs) @20 |
| No. | emitting layer | (V) | Cd/A | CIEx | CIEy | mA/cm² |
| Example 1 | Compound 4 | 3.47 | 60.0 | 0.684 | 0.316 | 522 |
| Example 2 | Compound 9 | 3.48 | 59.3 | 0.684 | 0.316 | 527 |
| Example 3 | Compound 16 | 3.48 | 59.9 | 0.684 | 0.316 | 535 |
| Example 4 | Compound 17 | 3.45 | 60.4 | 0.684 | 0.316 | 536 |
| Example 5 | Compound 20 | 3.48 | 60.4 | 0.684 | 0.316 | 518 |
| Example 6 | Compound 36 | 3.50 | 58.9 | 0.684 | 0.316 | 535 |
| Example 7 | Compound 38 | 3.48 | 58.7 | 0.684 | 0.316 | 533 |
| Example 8 | Compound 39 | 3.51 | 59.9 | 0.684 | 0.316 | 539 |
| Example 9 | Compound 42 | 3.47 | 59.0 | 0.684 | 0.316 | 523 |
| Example 10 | Compound 47 | 3.52 | 58.6 | 0.684 | 0.316 | 522 |
| Example 11 | Compound 48 | 3.52 | 60.2 | 0.684 | 0.316 | 523 |
| Example 12 | Compound 54 | 3.48 | 59.3 | 0.684 | 0.316 | 537 |
| Example 13 | Compound 51 | 3.46 | 59.2 | 0.684 | 0.316 | 529 |
| Example 14 | Compound 62 | 3.45 | 59.7 | 0.684 | 0.316 | 526 |
| Example 15 | Compound 65 | 3.51 | 60.2 | 0.684 | 0.316 | 534 |
| Example 16 | Compound 67 | 3.52 | 59.8 | 0.684 | 0.316 | 527 |
| Example 17 | Compound 72 | 3.45 | 59.6 | 0.684 | 0.316 | 520 |
| Example 18 | Compound 73 | 3.46 | 60.1 | 0.684 | 0.316 | 521 |
| Example 19 | Compound 74 | 3.45 | 60.0 | 0.684 | 0.316 | 528 |
| Example 20 | Compound 86 | 3.52 | 60.3 | 0.684 | 0.316 | 524 |
| Example 21 | Compound 89 | 3.51 | 59.6 | 0.684 | 0.316 | 521 |
| Example 22 | Compound 91 | 3.51 | 60.1 | 0.684 | 0.316 | 536 |
| Example 23 | Compound 92 | 3.48 | 59.9 | 0.684 | 0.316 | 534 |
| Example 24 | Compound 105 | 3.50 | 59.9 | 0.684 | 0.316 | 538 |
| Example 25 | Compound 115 | 3.46 | 59.7 | 0.684 | 0.316 | 522 |
| Example 26 | Compound 134 | 3.46 | 59.5 | 0.684 | 0.316 | 527 |
| Example 27 | Compound 137 | 3.50 | 60.8 | 0.684 | 0.316 | 524 |
| Example 28 | Compound 129 | 3.45 | 59.0 | 0.684 | 0.316 | 525 |
| Example 29 | Compound 144 | 3.51 | 59.8 | 0.684 | 0.316 | 530 |
| Example 30 | Compound 147 | 3.52 | 59.1 | 0.684 | 0.316 | 536 |
| Example 31 | Compound 151 | 3.51 | 598 | 0.684 | 0.316 | 538 |
| Example 32 | Compound 154 | 3.46 | 60.0 | 0.684 | 0.316 | 540 |
| Example 33 | Compound 172 | 3.52 | 59.3 | 0.684 | 0.316 | 523 |
| Example 34 | Compound 187 | 3.51 | 59.6 | 0.684 | 0.316 | 481 |
| Example 35 | Compound 191 | 3.50 | 59.1 | 0.684 | 0.316 | 490 |
| Example 36 | Compound 196 | 3.48 | 59.7 | 0.684 | 0.316 | 482 |
| Example 37 | Compound 198 | 3.50 | 59.8 | 0.684 | 0.316 | 494 |
| Example 38 | Compound 201 | 3.48 | 60.5 | 0.684 | 0.316 | 480 |
| Example 39 | Compound 203 | 3.49 | 59.1 | 0.684 | 0.316 | 484 |
| Example 40 | Compound 207 | 3.48 | 59.4 | 0.684 | 0.316 | 478 |
| Example 41 | Compound 215 | 3.47 | 60.5 | 0.684 | 0.316 | 481 |
| Example 42 | Compound 222 | 3.47 | 60.4 | 0.684 | 0.316 | 489 |
| Example 43 | Compound 217 | 3.51 | 60.5 | 0.684 | 0.316 | 492 |
| Example 44 | Compound 225 | 3.47 | 58.7 | 0.684 | 0.316 | 479 |
| Example 45 | Compound 228 | 3.48 | 59.6 | 0.684 | 0.316 | 488 |
| Example 46 | Compound 232 | 3.47 | 59.4 | 0.684 | 0.316 | 495 |
| Example 47 | Compound 240 | 3.46 | 59.5 | 0.684 | 0.316 | 490 |
| Example 48 | Compound 264 | 3.52 | 64.3 | 0.684 | 0.316 | 491 |
| Example 49 | Compound 267 | 3.46 | 64.3 | 0.684 | 0.316 | 526 |
| Example 50 | Compound 288 | 3.50 | 64.5 | 0.684 | 0.316 | 485 |
| Example 51 | Compound 289 | 3.50 | 64.2 | 0.684 | 0.316 | 529 |
| Example 52 | Compound 311 | 3.52 | 65.3 | 0.684 | 0.316 | 530 |
| Example 53 | Compound 315 | 3.52 | 64.7 | 0.684 | 0.316 | 525 |
| Example 54 | Compound 321 | 3.50 | 64.5 | 0.684 | 0.316 | 524 |
| Example 55 | Compound 316 | 3.45 | 65.3 | 0.684 | 0.316 | 484 |
| Example 56 | Compound 331 | 3.48 | 65.8 | 0.684 | 0.316 | 531 |
| Example 57 | Compound 333 | 3.47 | 64.7 | 0.684 | 0.316 | 528 |
| Example 58 | Compound 354 | 3.48 | 65.2 | 0.684 | 0.316 | 489 |
| Example 59 | Compound 380 | 3.51 | 65.8 | 0.684 | 0.316 | 483 |
| Example 60 | Compound 385 | 3.46 | 65.9 | 0.684 | 0.316 | 493 |
| Comparative | Compound A | 3.53 | 49.4 | 0.684 | 0.316 | 411 |
| Example 1 | | | | | | |
| Comparative | Compound B | 3.56 | 50.8 | 0.684 | 0.316 | 422 |
| Example 2 | | | | | | |
| Comparative | Compound C | 3.55 | 51.0 | 0.684 | 0.316 | 403 |
| Example 3 | | | | | | |
| Comparative | Compound D | 3.53 | 52.3 | 0.684 | 0.316 | 388 |
| Example 4 | | | | | | |

As can be seen from the above Table 7, when the arylamine compounds of the present invention were used as the host material of the light-emitting layer for red organic electroluminescent devices, the light-emitting efficiency (Cd/A) was increased by at least 12.0% and the lifetime of T95 was prolonged by at least 13.3% in devices of Examples 1 to 60, compared to Comparative Examples 1 to 4.

The preferred embodiments of the present invention are described in detail above in conjunction with the accompanying drawings. However, the present invention is not limited to the specific details of the above embodiments. Within the scope of the technical concept of the present invention, various simple modifications can be made to the technical solutions of the present invention, and all of these simple modifications fall within the protection scope of the present invention.

1. An arylamine compound, having the structure represented by Formula 1 as follows: 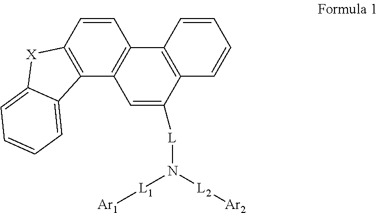
wherein, X is selected from O or S;
L, L₁ and L₂ are each independently selected from a single bond, a substituted or unsubstituted arylene having 6 to 30 carbon atoms, and a substituted or unsubstituted heteroarylene having 3 to 20 carbon atoms;
Ar₁ and Ar₂ are the same or different, and are each independently selected from a substituted or unsubstituted aryl having 6 to 30 carbon atoms, and a substituted or unsubstituted heteroaryl having 3 to 20 carbon atoms;
the substituents in L, L₁, L₂, Ar₁, and Ar₂ are the same or different, and are each independently selected from deuterium, cyano, halogen, alkyl having 1-10 carbon atoms, haloalkyl having 1-10 carbon atoms, deuterated alkyl having 1-10 carbon atoms, alkoxy having 1-10 carbon atoms, alkylthio having 1-10 carbon atoms, trialkylsilyl having 3-12 carbon atoms, aryl having 6-15 carbon atoms, heteroaryl having 3-12 carbon atoms, and cycloalkyl having 3-10 carbon atoms; optionally, any two adjacent substituents form a saturated or unsaturated 3-15 membered ring.

2. The arylamine compound according to claim 1, wherein L, L₁, and L₂ are the same or different and are each independently selected from a single bond, a substituted or unsubstituted arylene having 6 to 15 carbon atoms, and a substituted or unsubstituted heteroarylene having 5 to 18 carbon atoms.

3. The arylamine compound according to claim 1, wherein L, L₁ and L₂ are each independently selected from a single bond, a substituted or unsubstituted phenylene, a substituted or unsubstituted naphthylene, a substituted or unsubstituted biphenylene, a substituted or unsubstituted fluorenylene, a substituted or unsubstituted phenanthrylene, a substituted or unsubstituted dibenzothienylene, a substituted or unsubstituted dibenzofuranylene, and a substituted or unsubstituted carbazolylene.

4. The arylamine compound according to claim 1, wherein L₁ and L₂ are each independently selected from the group consisting of a single bond and the following groups:

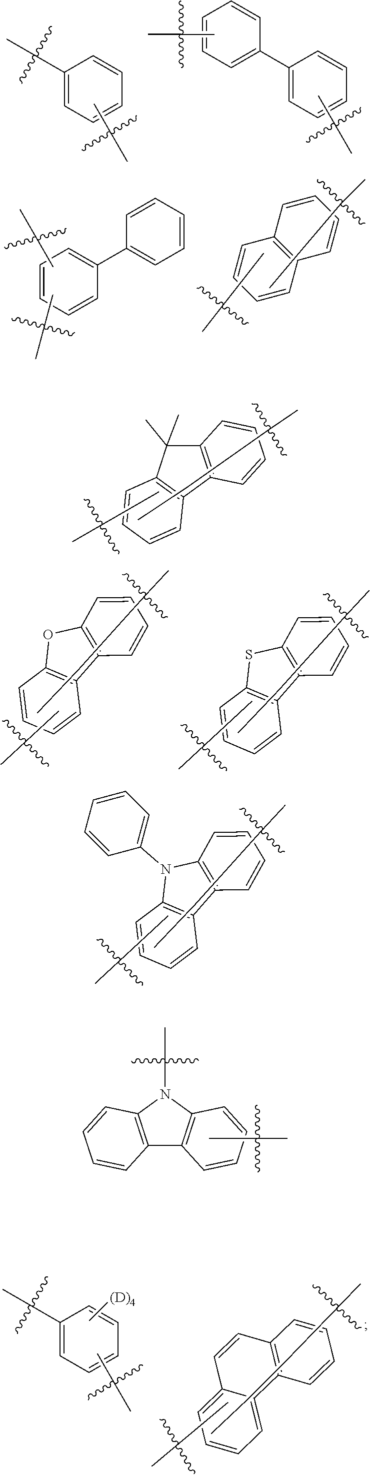

5. The arylamine compound according to claim 1, wherein Ar₁ and Ar₂ are each independently selected from a substituted or unsubstituted aryl having 6 to 25 carbon atoms, and a substituted or unsubstituted heteroaryl having 12 to 18 carbon atoms.

6. The arylamine compound according to claim 1, wherein Ar₁ and Ar₂ are the same or different, and are each independently selected from a substituted or unsubstituted phenyl, a substituted or unsubstituted naphthyl, a substituted or unsubstituted biphenyl, a substituted or unsubstituted terphenyl, a substituted or unsubstituted fluorenyl, a substituted or unsubstituted phenanthryl, a substituted or unsubstituted anthracenyl, a substituted or unsubstituted pyrenyl, a substituted or unsubstituted triphenylene, a substituted or unsubstituted spirobifluorenyl, a substituted or unsubstituted dibenzothienyl, a substituted or unsubstituted dibenzofuranyl, and a substituted or unsubstituted carbazolyl.

7. The arylamine compound according to claim 1, wherein Ar₁ and Ar₂ are the same or different, and are each independently selected from the group consisting of the following groups:

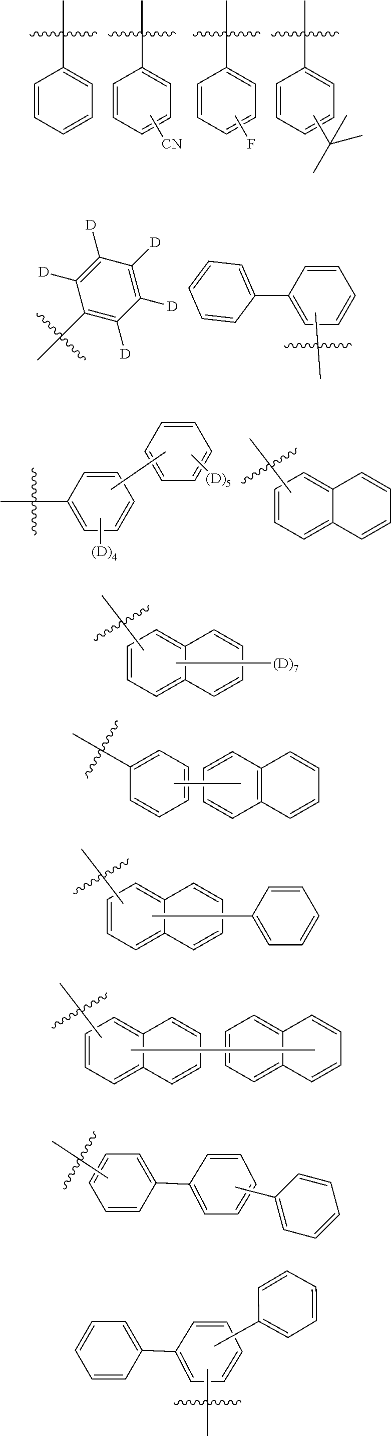

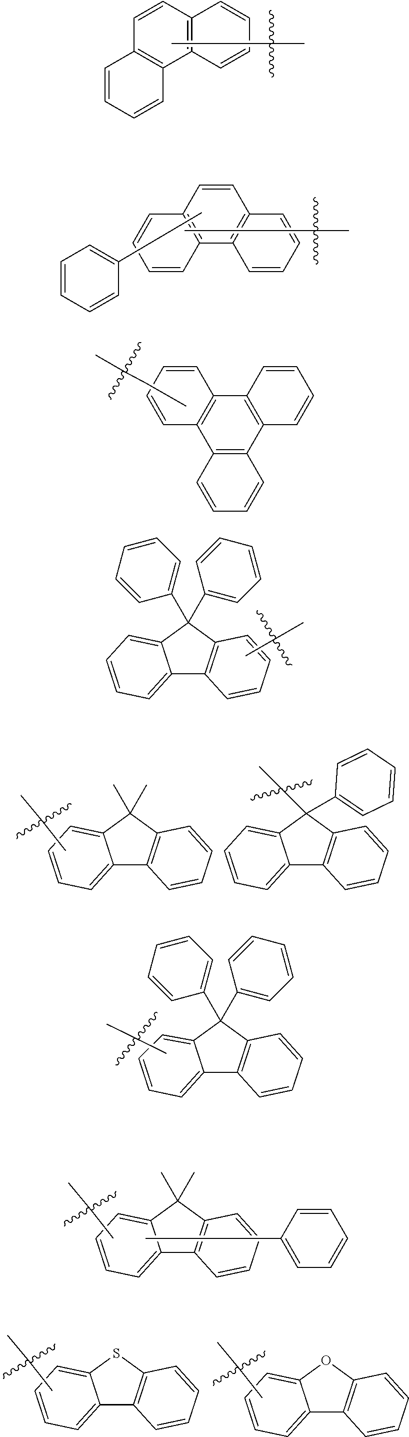

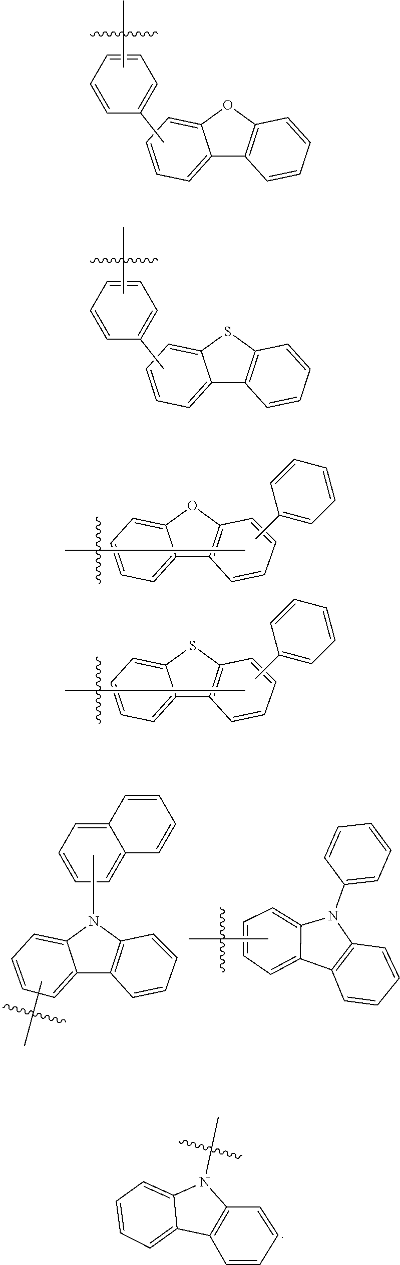

8. The arylamine compound according to claim 1, wherein 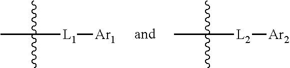
are the same or different, and are each independently selected from the group consisting of the following groups:

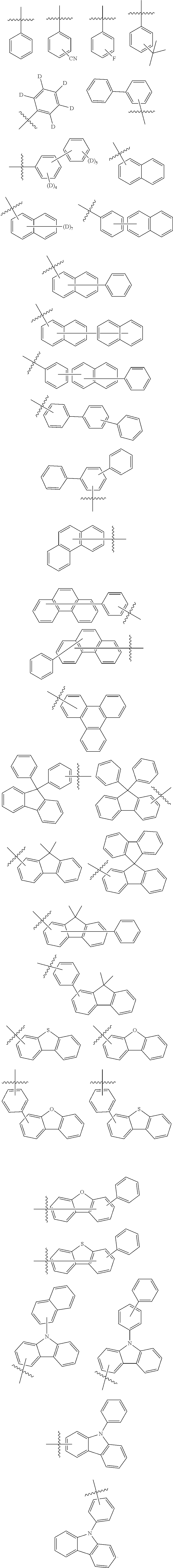

9. The arylamine compound according to claim 1, wherein 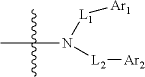
in Formula 1 is selected from the group consisting of the following groups,

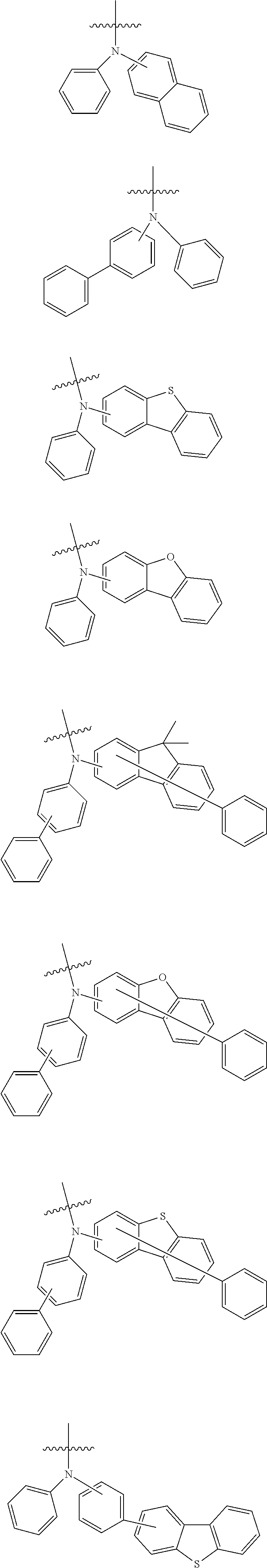

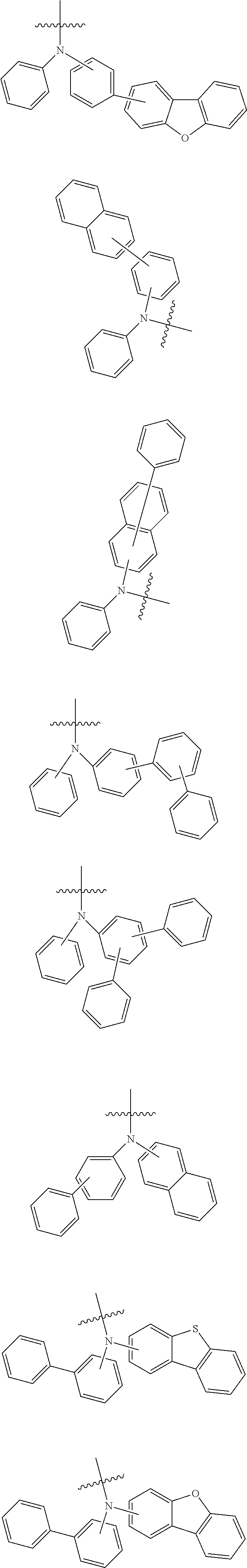

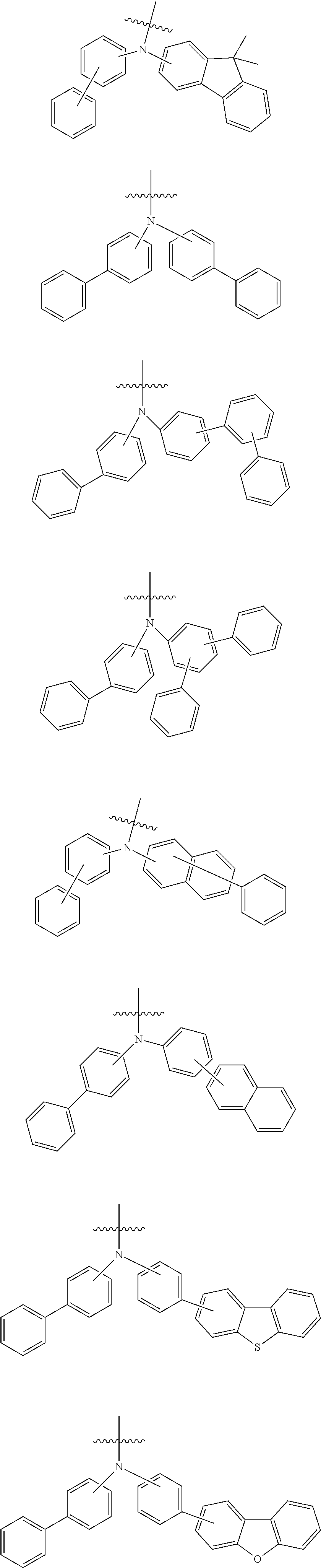

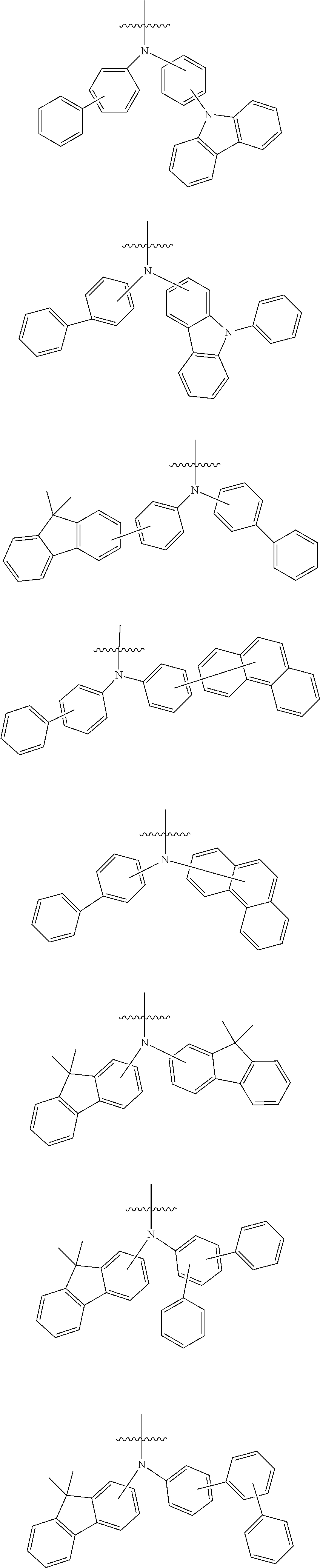

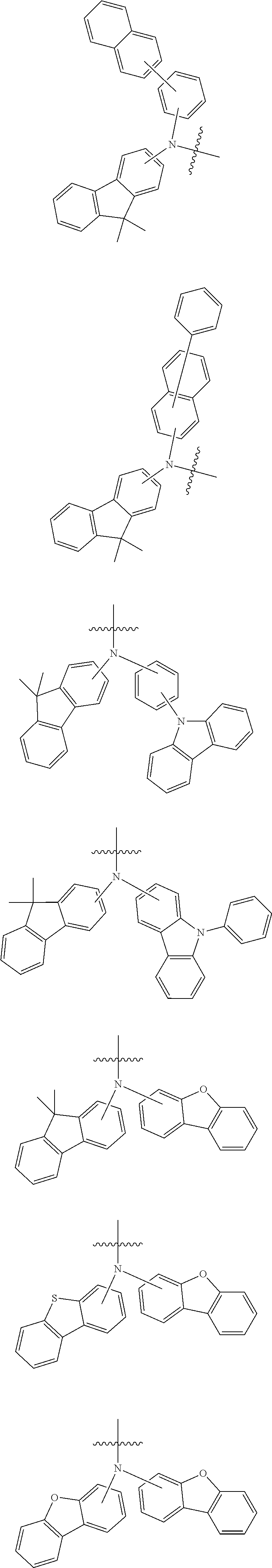

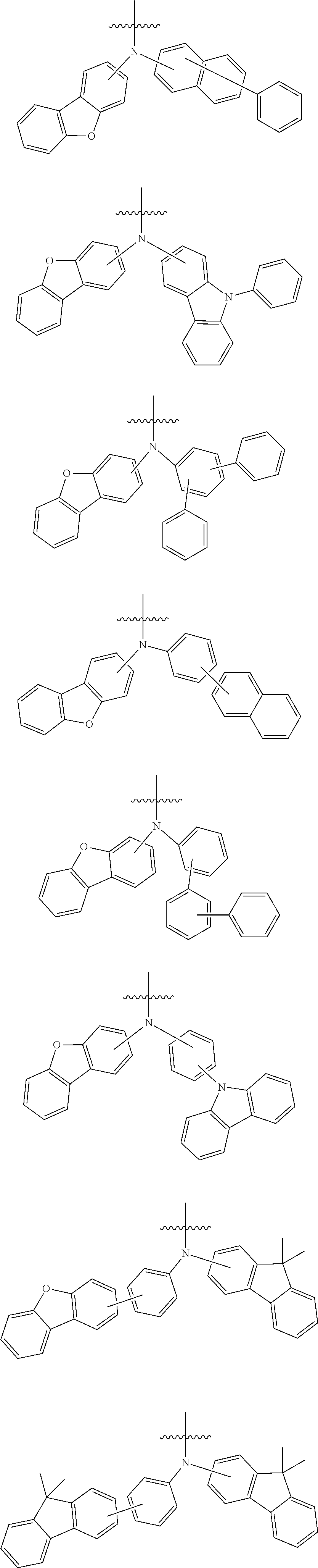

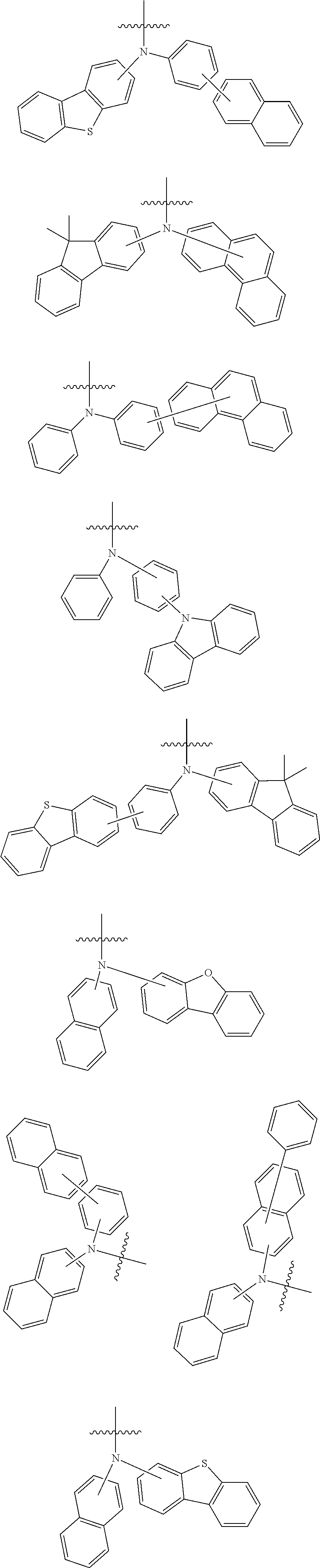

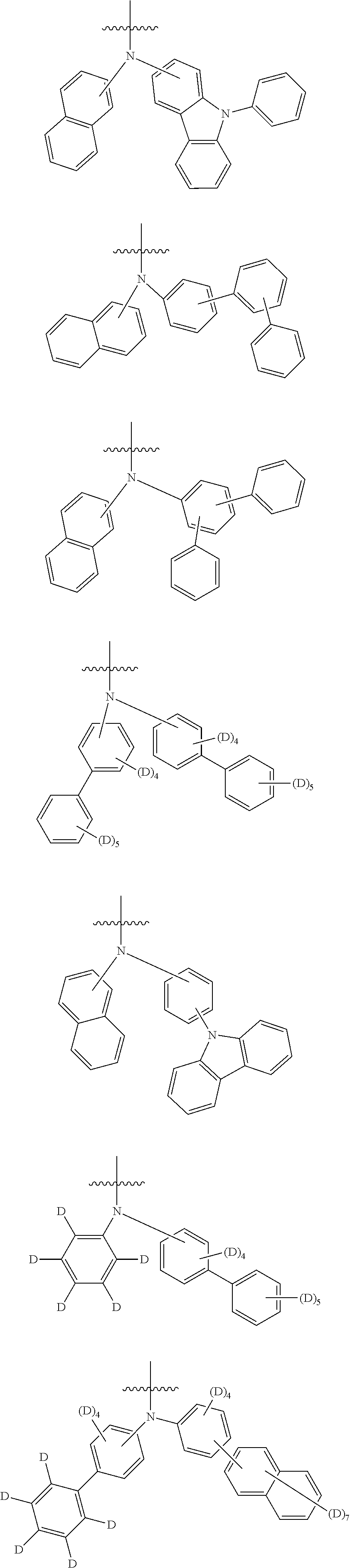

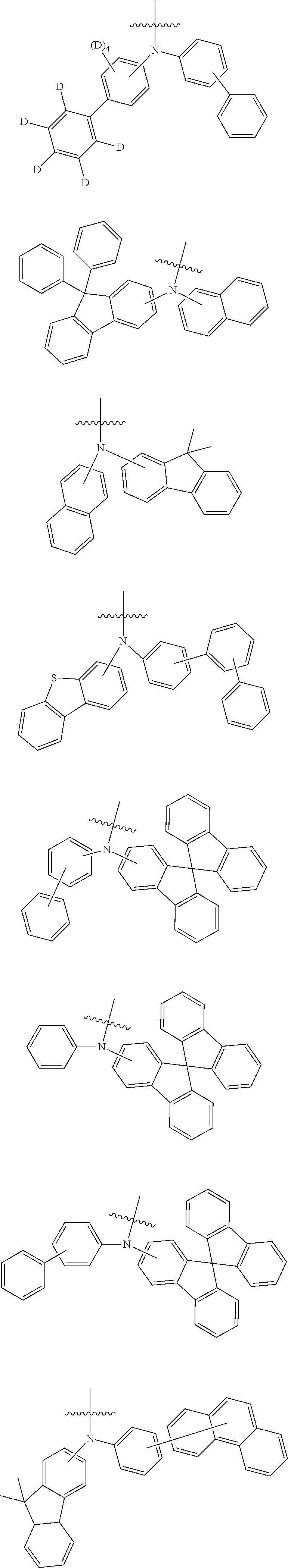

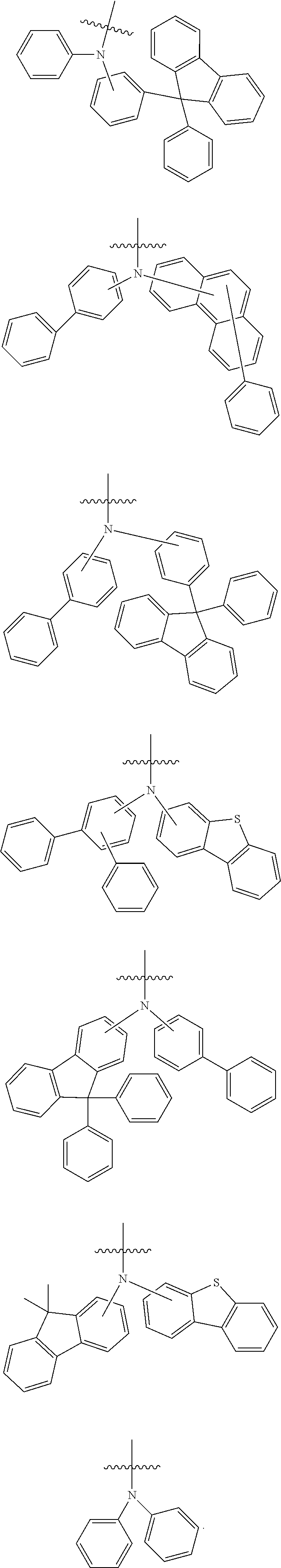

10. The arylamine compound according to claim 1, wherein the arylamine compound is selected from the group consisting of the following groups:

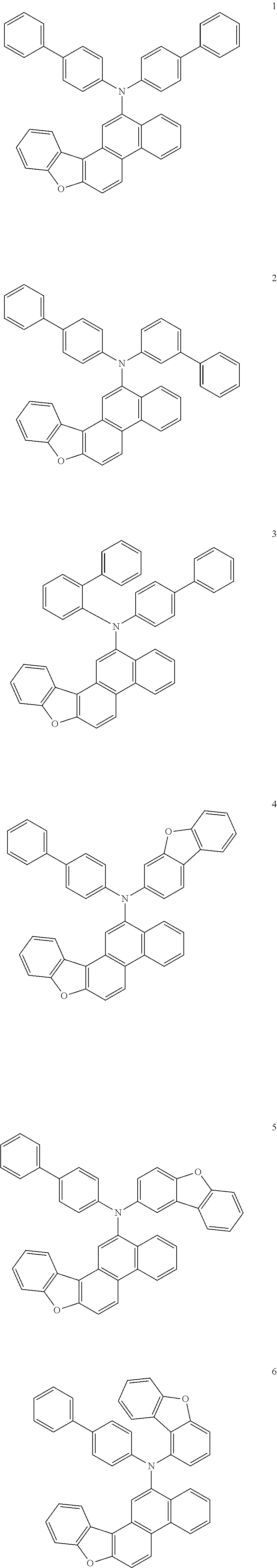 [SMILES: c1ccc(-c2ccc(N(c3ccc(-c4ccccc4)cc3)c3cc4c(ccc5oc6ccccc6c54)c4ccccc34)cc2)cc1 ; c1ccc(-c2ccc(N(c3cccc(-c4ccccc4)c3)c3cc4c(ccc5oc6ccccc6c54)c4ccccc34)cc2)cc1 ; c1ccc(-c2ccc(N(c3ccccc3-c3ccccc3)c3cc4c(ccc5oc6ccccc6c54)c4ccccc34)cc2)cc1 ; c1ccc(-c2ccc(N(c3ccc4c(c3)oc3ccccc34)c3cc4c(ccc5oc6ccccc6c54)c4ccccc34)cc2)cc1 ; c1ccc(-c2ccc(N(c3ccc4oc5ccccc5c4c3)c3cc4c(ccc5oc6ccccc6c54)c4ccccc34)cc2)cc1 ; c1ccc(-c2ccc(N(c3cc4c(ccc5oc6ccccc6c54)c4ccccc34)c3cccc4oc5ccccc5c34)cc2)cc1]

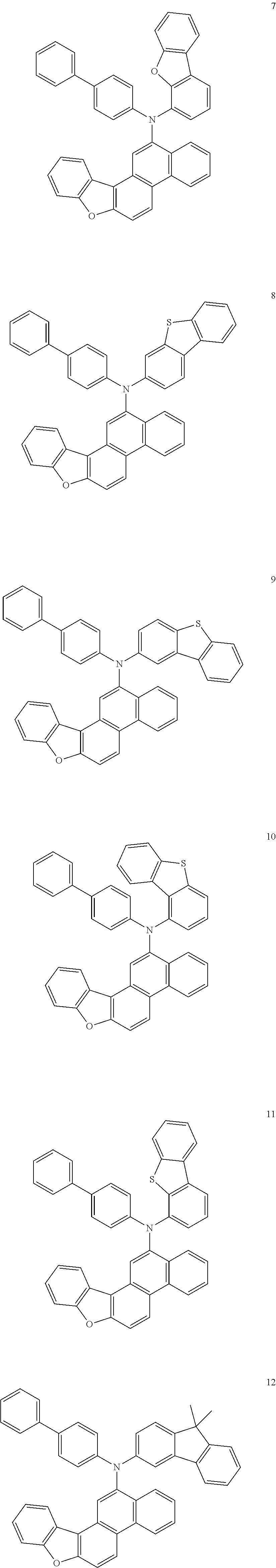 [SMILES: c1ccc(-c2ccc(N(c3cc4c(ccc5oc6ccccc6c54)c4ccccc34)c3cccc4c3oc3ccccc34)cc2)cc1 ; c1ccc(-c2ccc(N(c3ccc4c(c3)sc3ccccc34)c3cc4c(ccc5oc6ccccc6c54)c4ccccc34)cc2)cc1 ; c1ccc(-c2ccc(N(c3ccc4sc5ccccc5c4c3)c3cc4c(ccc5oc6ccccc6c54)c4ccccc34)cc2)cc1 ; c1ccc(-c2ccc(N(c3cc4c(ccc5oc6ccccc6c54)c4ccccc34)c3cccc4sc5ccccc5c34)cc2)cc1 ; c1ccc(-c2ccc(N(c3cc4c(ccc5oc6ccccc6c54)c4ccccc34)c3cccc4c3sc3ccccc34)cc2)cc1 ; CC1(C)c2ccccc2-c2cc(N(c3ccc(-c4ccccc4)cc3)c3cc4c(ccc5oc6ccccc6c54)c4ccccc34)ccc21]

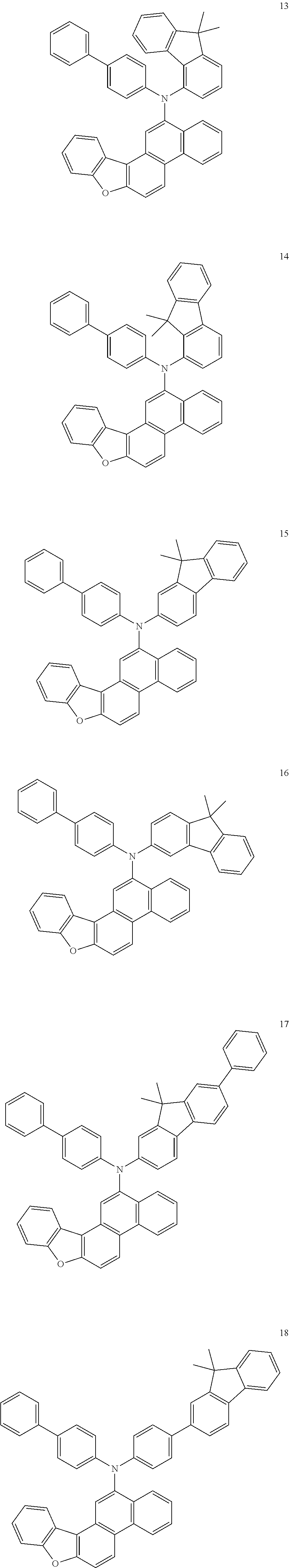

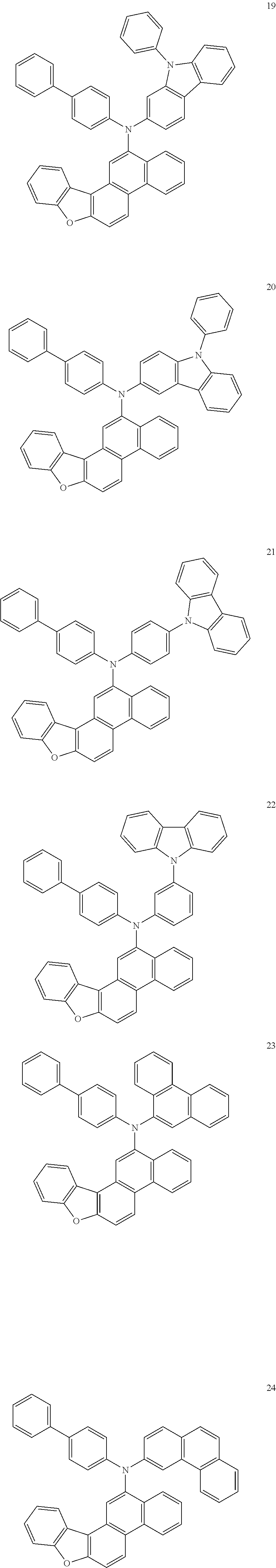

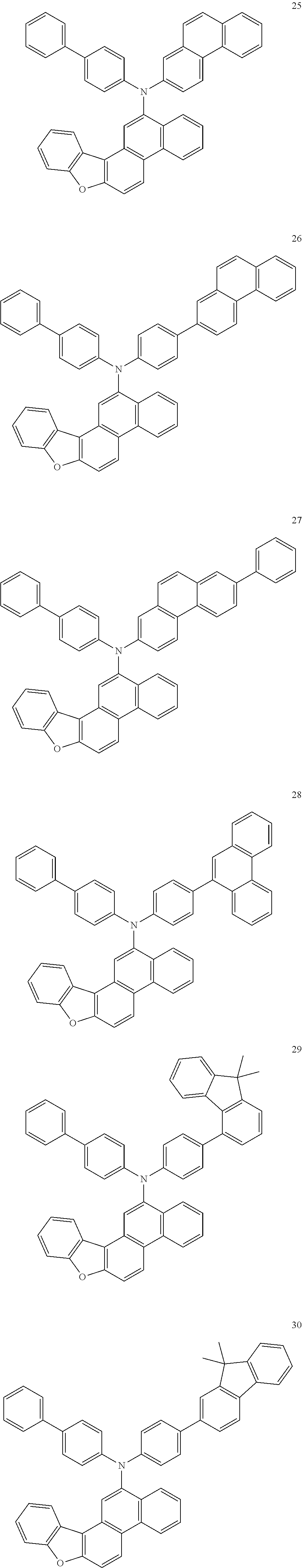

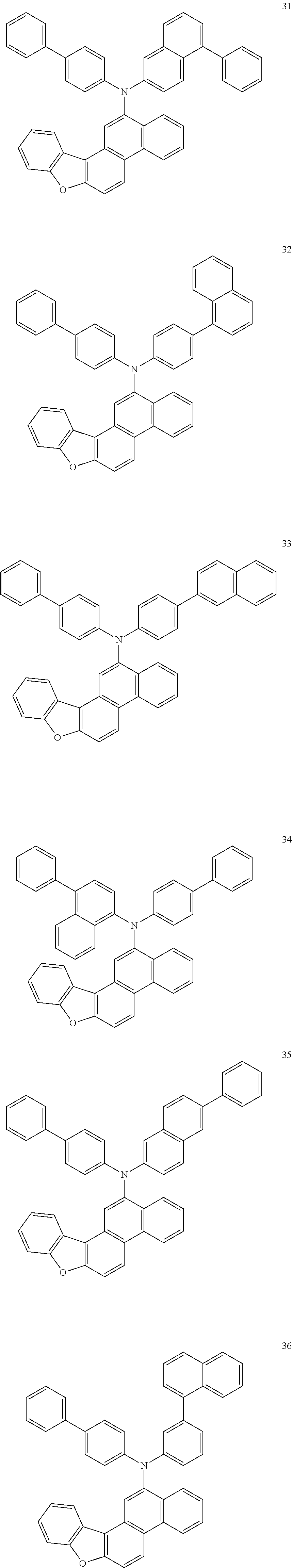

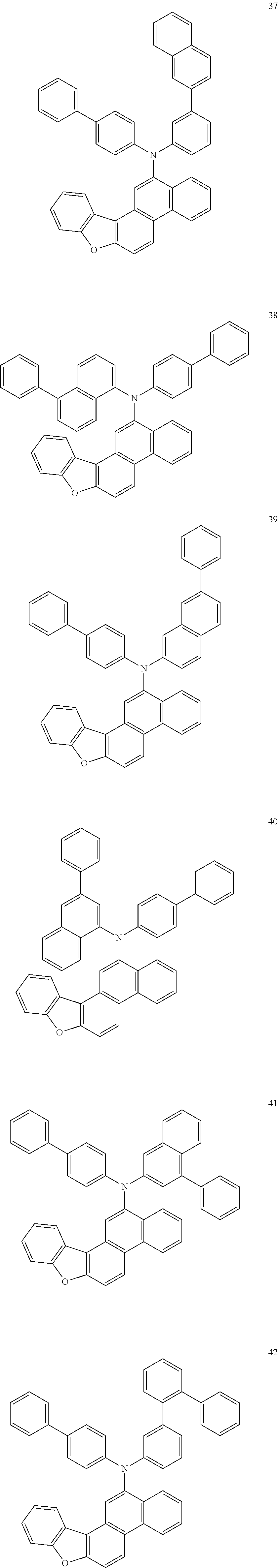

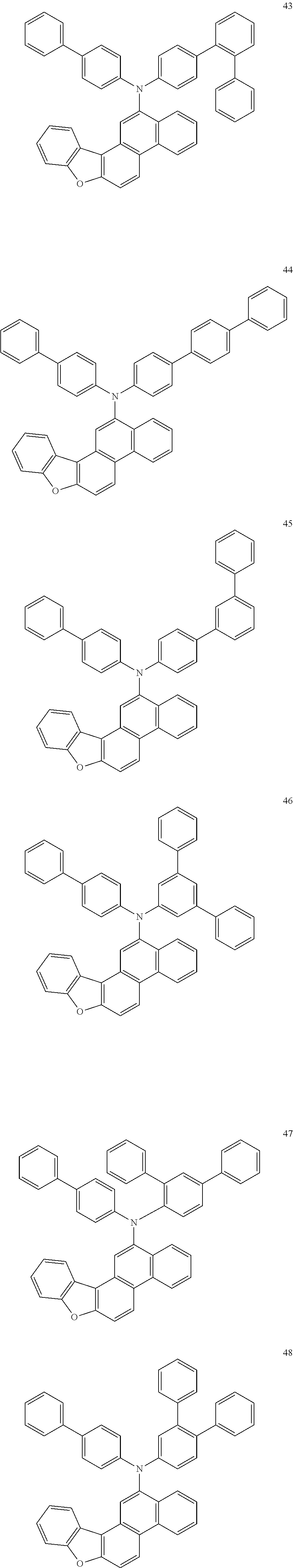

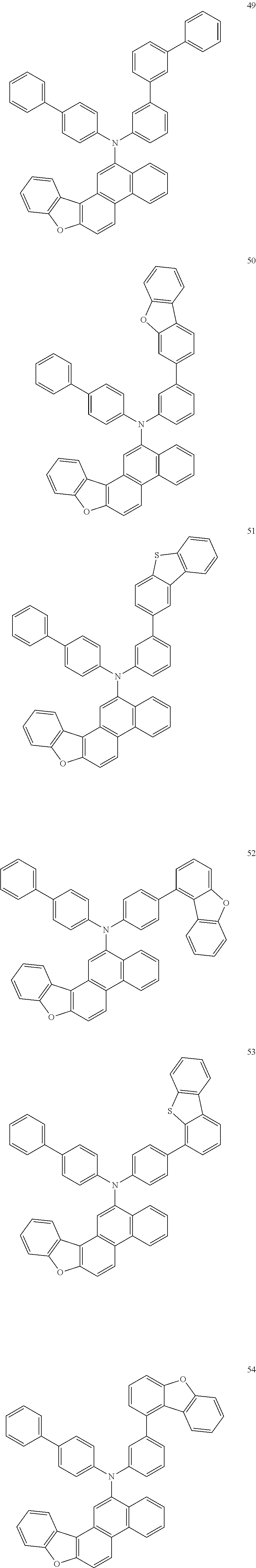

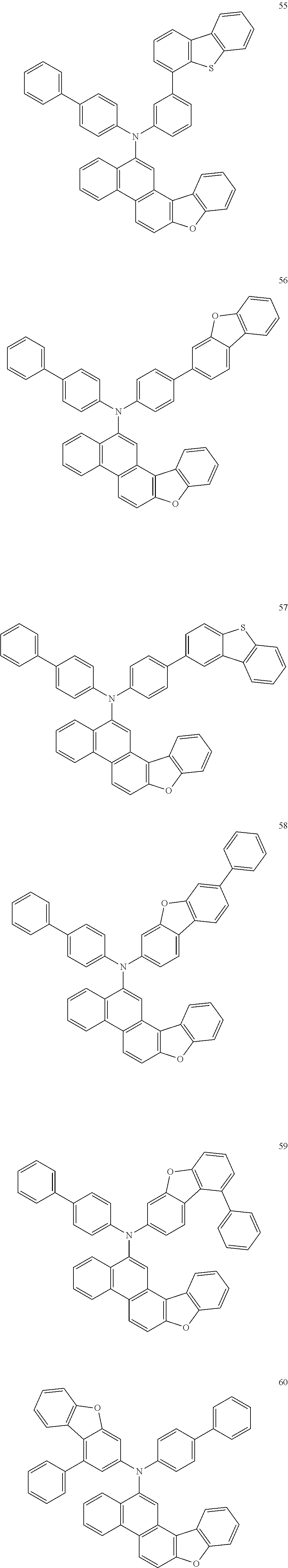

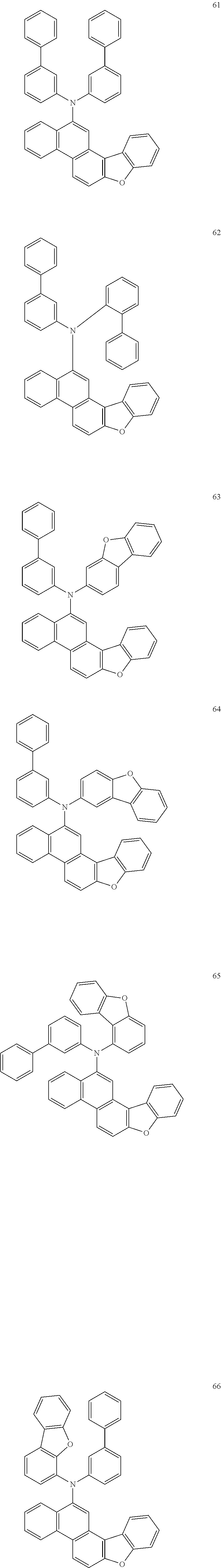 [SMILES: c1ccc(-c2cccc(N(c3cccc(-c4ccccc4)c3)c3cc4c(ccc5oc6ccccc6c54)c4ccccc34)c2)cc1 ; c1ccc(-c2cccc(N(c3ccccc3-c3ccccc3)c3cc4c(ccc5oc6ccccc6c54)c4ccccc34)c2)cc1 ; c1ccc(-c2cccc(N(c3ccc4c(c3)oc3ccccc34)c3cc4c(ccc5oc6ccccc6c54)c4ccccc34)c2)cc1 ; c1ccc(-c2cccc(N(c3ccc4oc5ccccc5c4c3)c3cc4c(ccc5oc6ccccc6c54)c4ccccc34)c2)cc1 ; c1ccc(-c2cccc(N(c3cc4c(ccc5oc6ccccc6c54)c4ccccc34)c3cccc4oc5ccccc5c34)c2)cc1 ; c1ccc(-c2cccc(N(c3cc4c(ccc5oc6ccccc6c54)c4ccccc34)c3cccc4c3oc3ccccc34)c2)cc1]

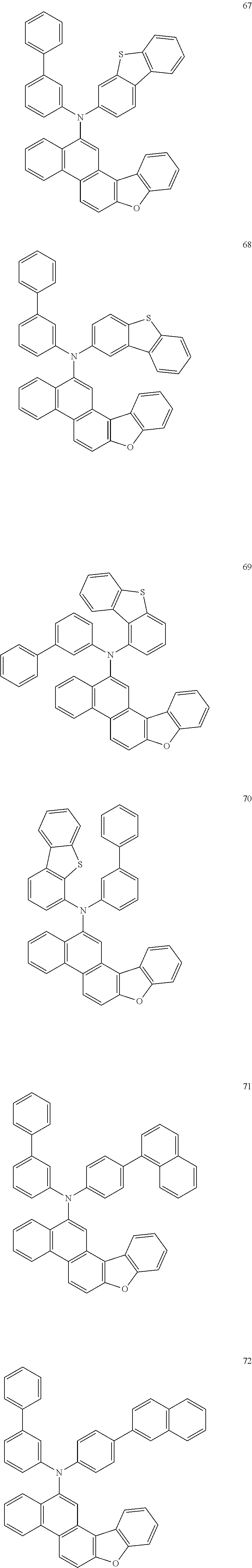 [SMILES: c1ccc(-c2cccc(N(c3ccc4c(c3)sc3ccccc34)c3cc4c(ccc5oc6ccccc6c54)c4ccccc34)c2)cc1 ; c1ccc(-c2cccc(N(c3ccc4sc5ccccc5c4c3)c3cc4c(ccc5oc6ccccc6c54)c4ccccc34)c2)cc1 ; c1ccc(-c2cccc(N(c3cc4c(ccc5oc6ccccc6c54)c4ccccc34)c3cccc4sc5ccccc5c34)c2)cc1 ; c1ccc(-c2cccc(N(c3cc4c(ccc5oc6ccccc6c54)c4ccccc34)c3cccc4c3sc3ccccc34)c2)cc1 ; c1ccc(-c2cccc(N(c3ccc(-c4cccc5ccccc45)cc3)c3cc4c(ccc5oc6ccccc6c54)c4ccccc34)c2)cc1 ; c1ccc(-c2cccc(N(c3ccc(-c4ccc5ccccc5c4)cc3)c3cc4c(ccc5oc6ccccc6c54)c4ccccc34)c2)cc1]

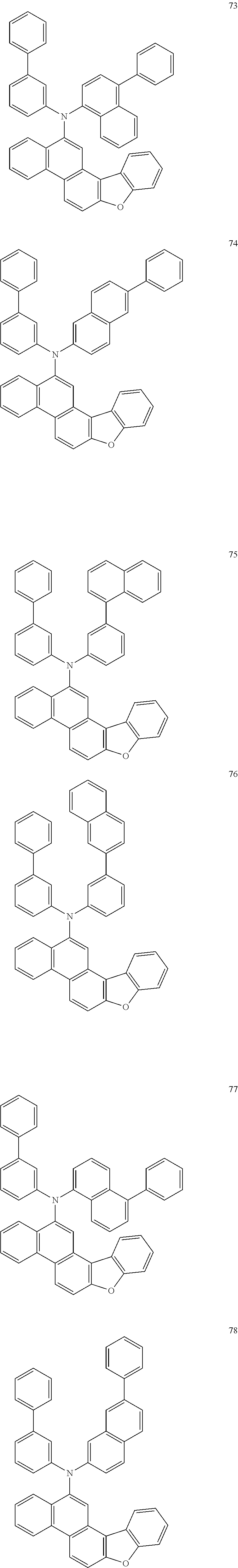

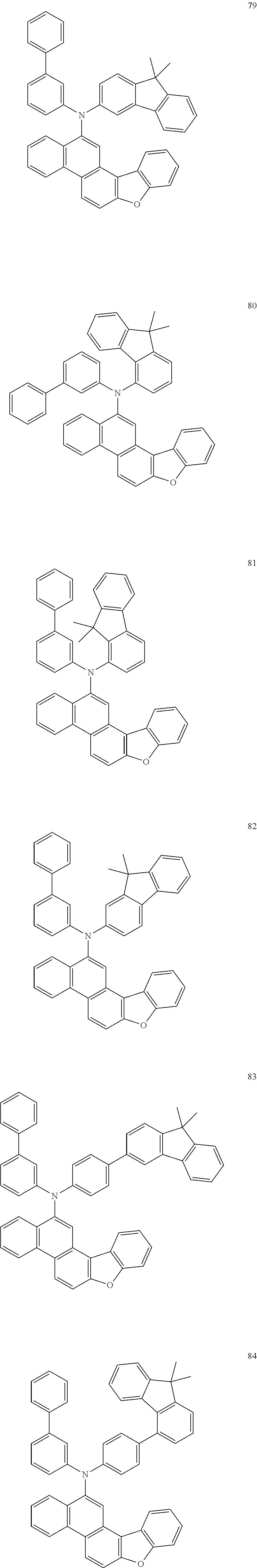

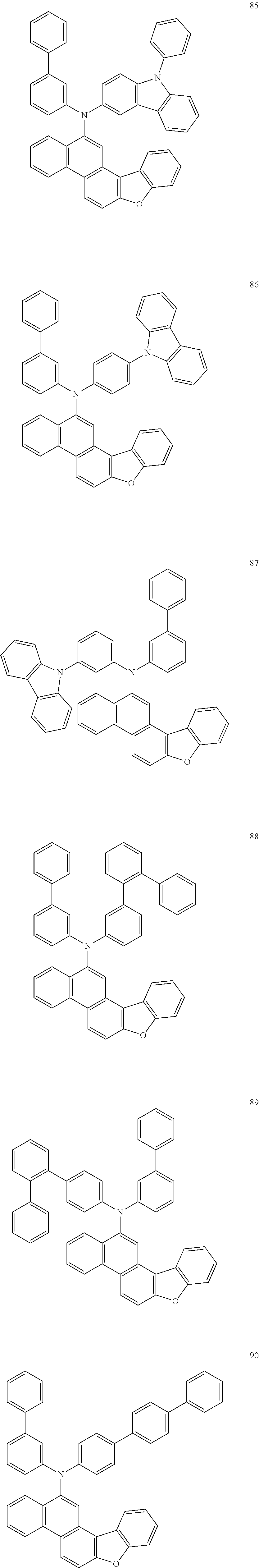

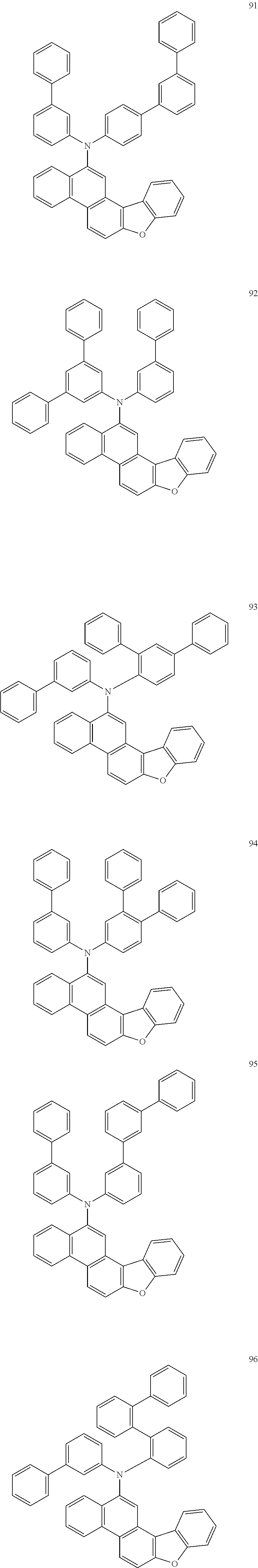

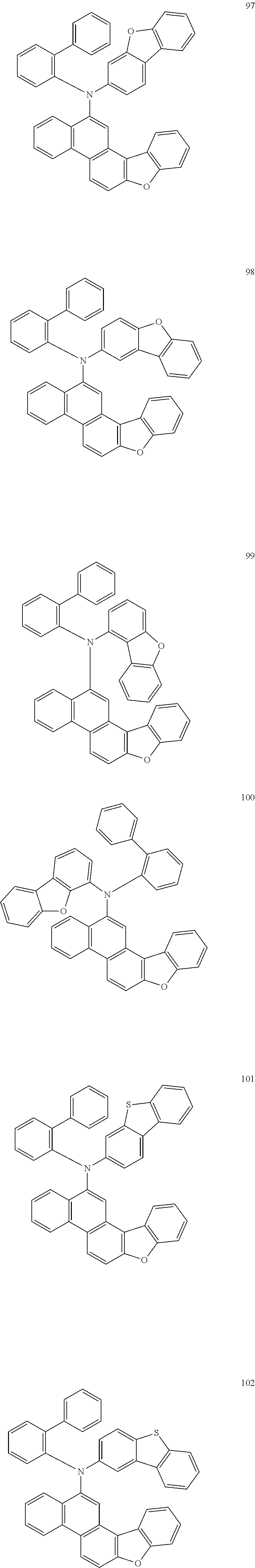 [SMILES: c1ccc(-c2ccccc2N(c2ccc3c(c2)oc2ccccc23)c2cc3c(ccc4oc5ccccc5c43)c3ccccc23)cc1 ; c1ccc(-c2ccccc2N(c2ccc3oc4ccccc4c3c2)c2cc3c(ccc4oc5ccccc5c43)c3ccccc23)cc1 ; c1ccc(-c2ccccc2N(c2cc3c(ccc4oc5ccccc5c43)c3ccccc23)c2cccc3oc4ccccc4c23)cc1 ; c1ccc(-c2ccccc2N(c2cc3c(ccc4oc5ccccc5c43)c3ccccc23)c2cccc3c2oc2ccccc23)cc1 ; c1ccc(-c2ccccc2N(c2ccc3c(c2)sc2ccccc23)c2cc3c(ccc4oc5ccccc5c43)c3ccccc23)cc1 ; c1ccc(-c2ccccc2N(c2ccc3sc4ccccc4c3c2)c2cc3c(ccc4oc5ccccc5c43)c3ccccc23)cc1]

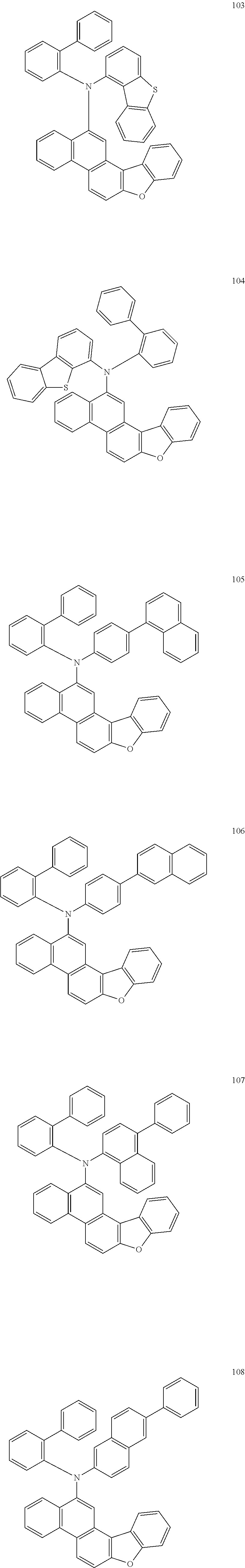 [SMILES: c1ccc(-c2ccccc2N(c2cc3c(ccc4oc5ccccc5c43)c3ccccc23)c2cccc3sc4ccccc4c23)cc1 ; c1ccc(-c2ccccc2N(c2cc3c(ccc4oc5ccccc5c43)c3ccccc23)c2cccc3c2sc2ccccc23)cc1 ; c1ccc(-c2ccccc2N(c2ccc(-c3cccc4ccccc34)cc2)c2cc3c(ccc4oc5ccccc5c43)c3ccccc23)cc1 ; c1ccc(-c2ccccc2N(c2ccc(-c3ccc4ccccc4c3)cc2)c2cc3c(ccc4oc5ccccc5c43)c3ccccc23)cc1 ; c1ccc(-c2ccccc2N(c2ccc(-c3ccccc3)c3ccccc23)c2cc3c(ccc4oc5ccccc5c43)c3ccccc23)cc1 ; c1ccc(-c2ccc3cc(N(c4ccccc4-c4ccccc4)c4cc5c(ccc6oc7ccccc7c65)c5ccccc45)ccc3c2)cc1]

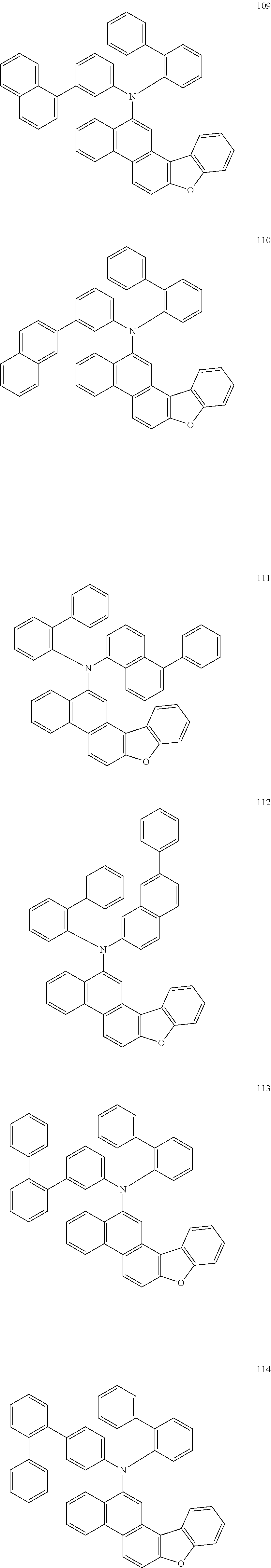

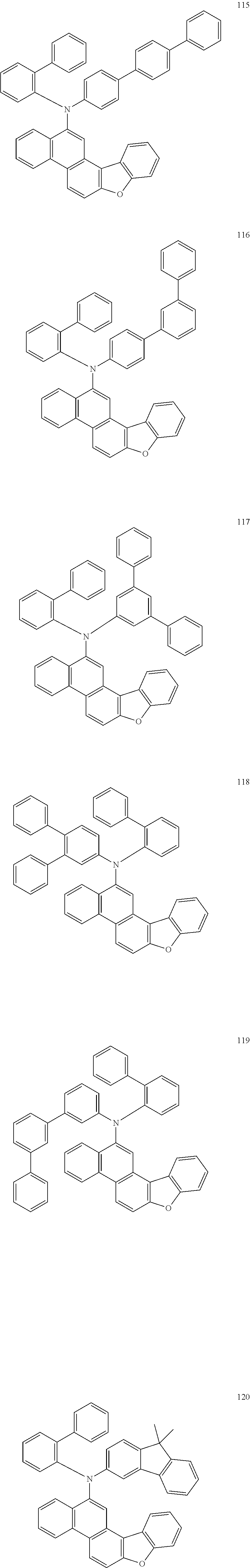

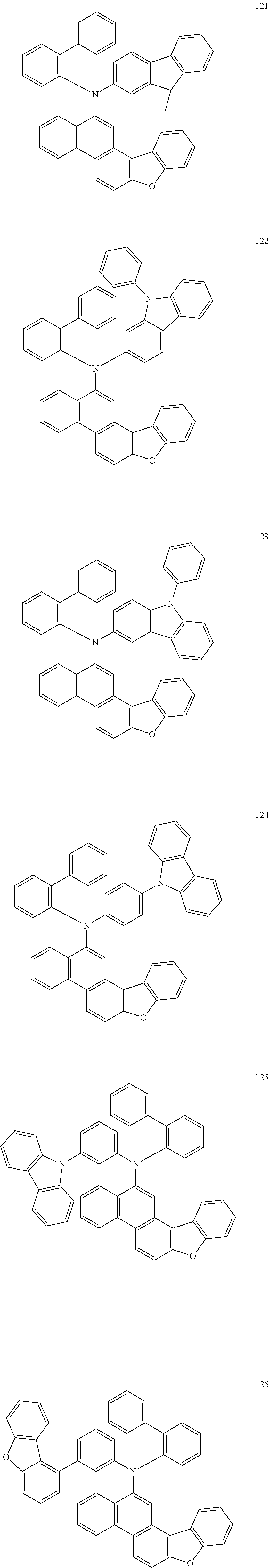

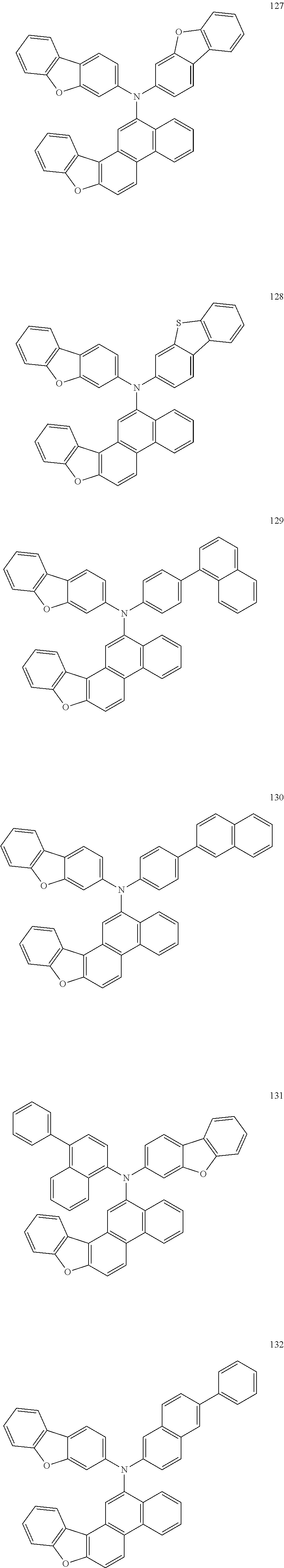

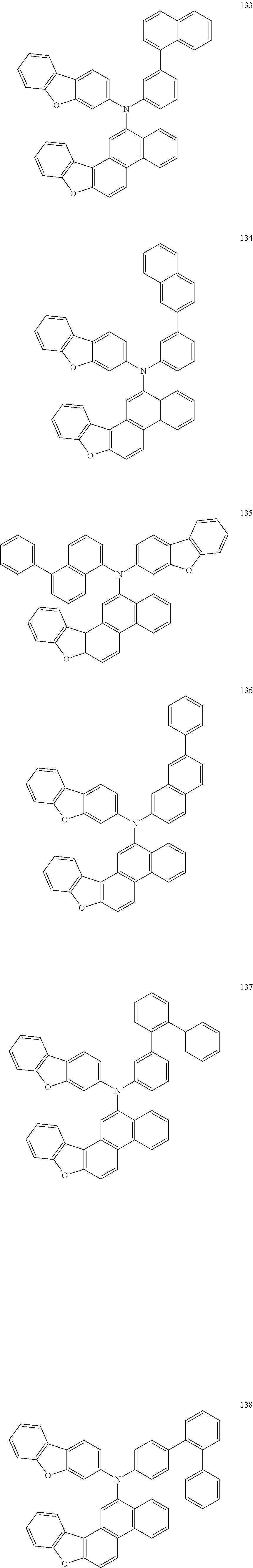

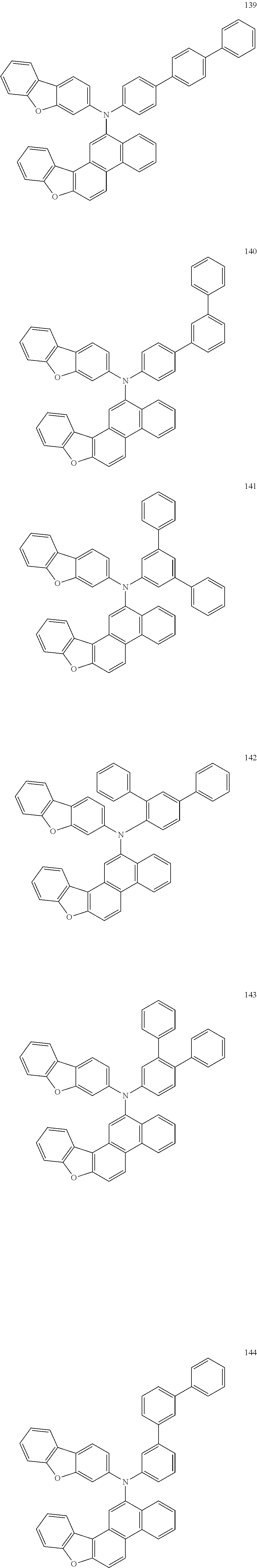

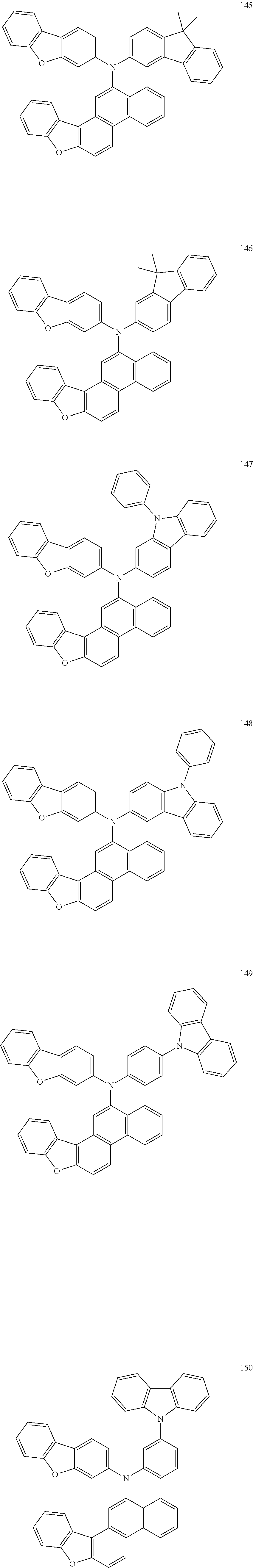

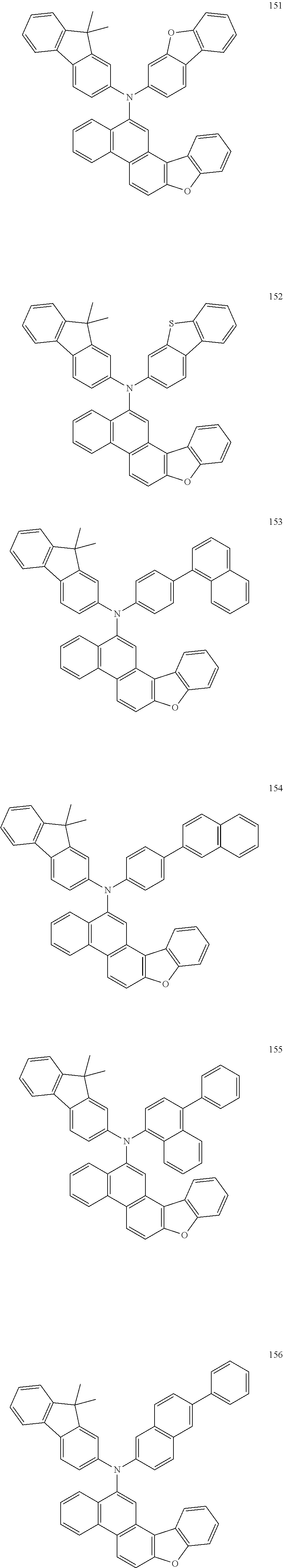

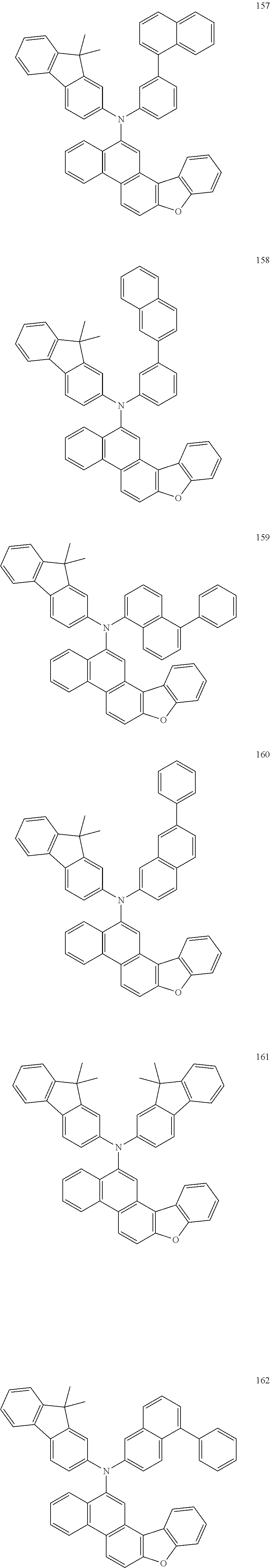

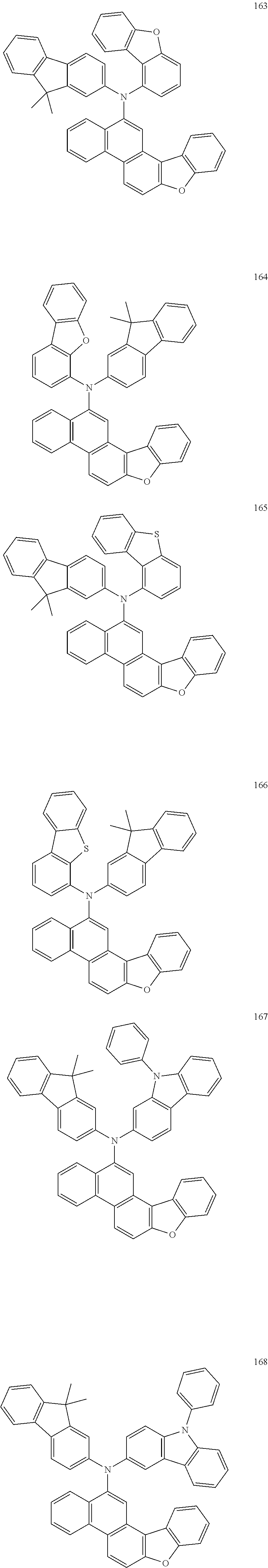

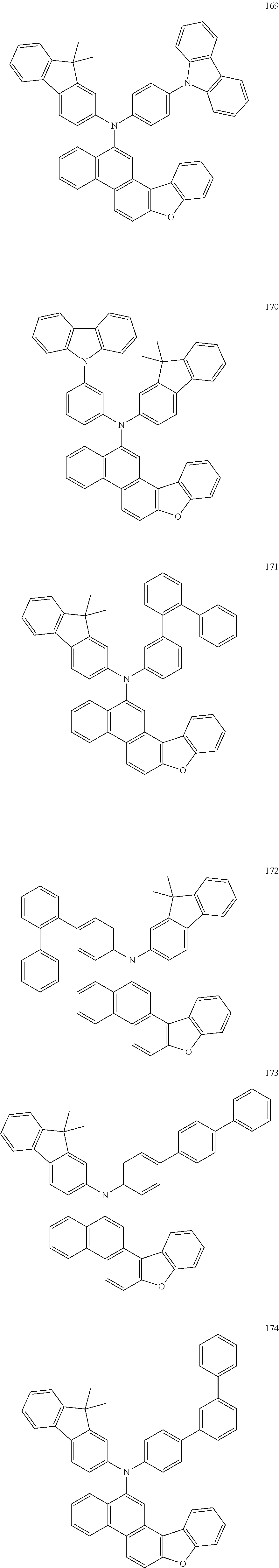

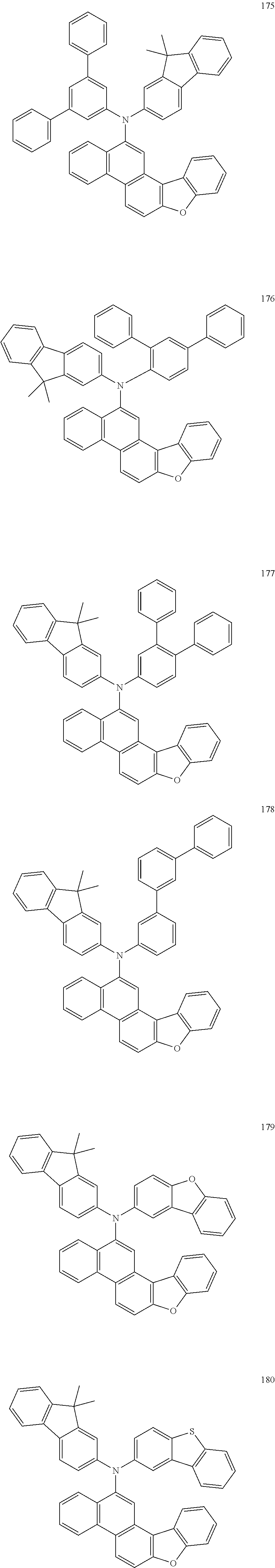

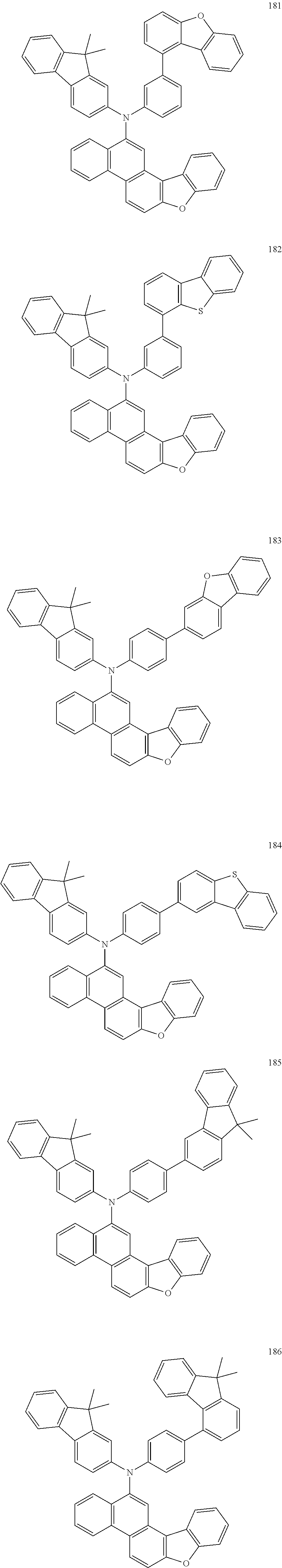

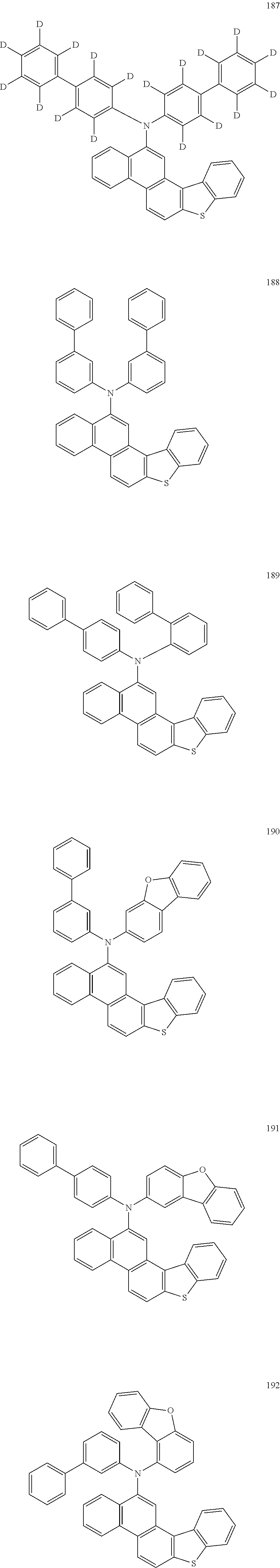

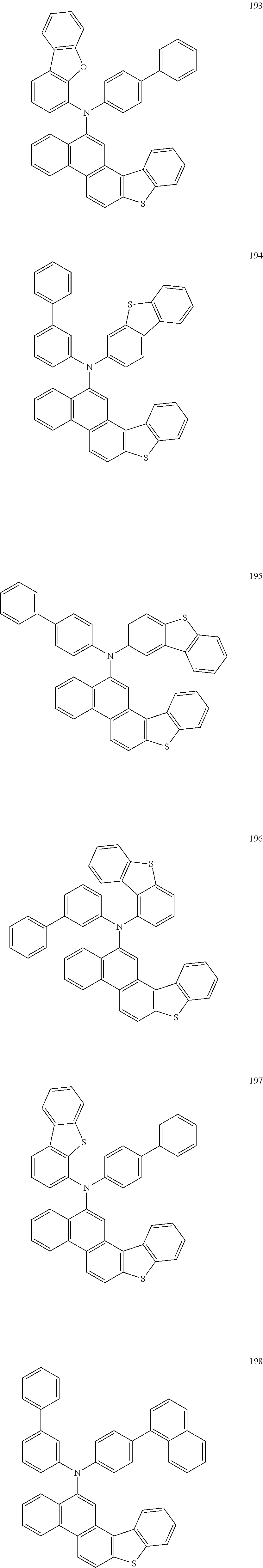 [SMILES: c1ccc(-c2ccc(N(c3cc4c(ccc5sc6ccccc6c54)c4ccccc34)c3cccc4c3oc3ccccc34)cc2)cc1 ; c1ccc(-c2cccc(N(c3ccc4c(c3)sc3ccccc34)c3cc4c(ccc5sc6ccccc6c54)c4ccccc34)c2)cc1 ; c1ccc(-c2ccc(N(c3ccc4sc5ccccc5c4c3)c3cc4c(ccc5sc6ccccc6c54)c4ccccc34)cc2)cc1 ; c1ccc(-c2cccc(N(c3cc4c(ccc5sc6ccccc6c54)c4ccccc34)c3cccc4sc5ccccc5c34)c2)cc1 ; c1ccc(-c2ccc(N(c3cc4c(ccc5sc6ccccc6c54)c4ccccc34)c3cccc4c3sc3ccccc34)cc2)cc1 ; c1ccc(-c2cccc(N(c3ccc(-c4cccc5ccccc45)cc3)c3cc4c(ccc5sc6ccccc6c54)c4ccccc34)c2)cc1]

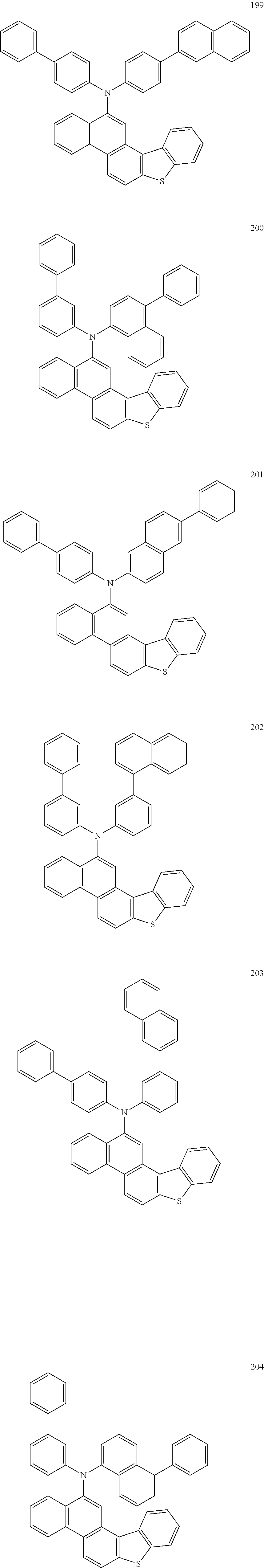

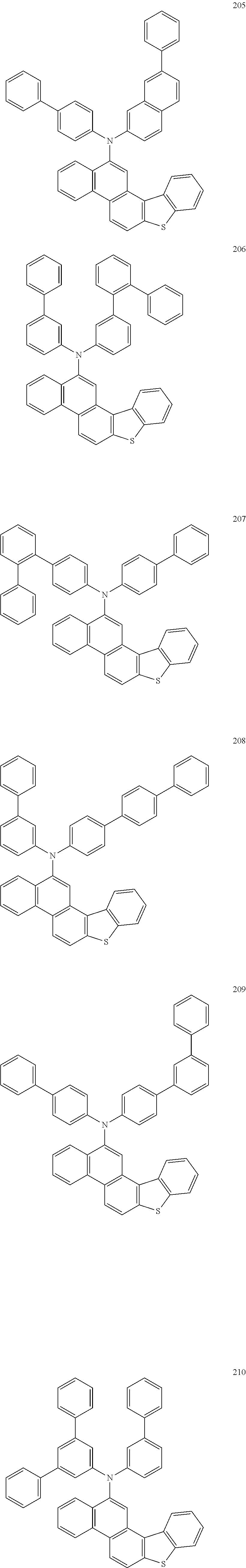

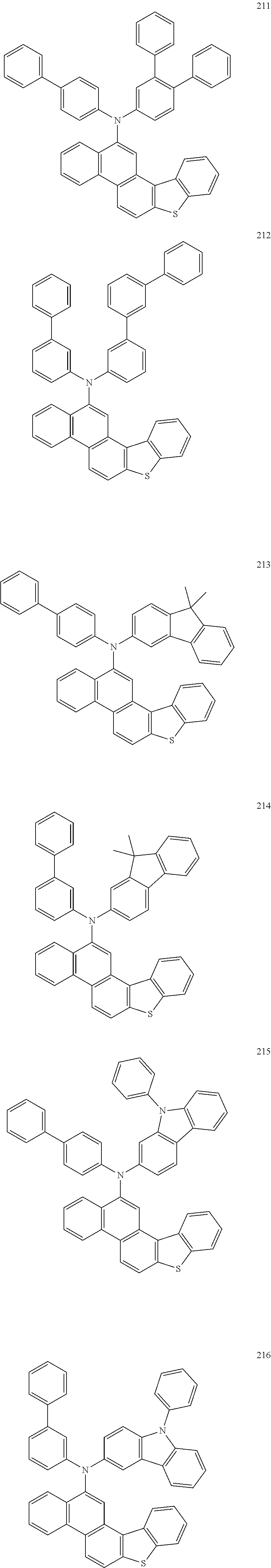

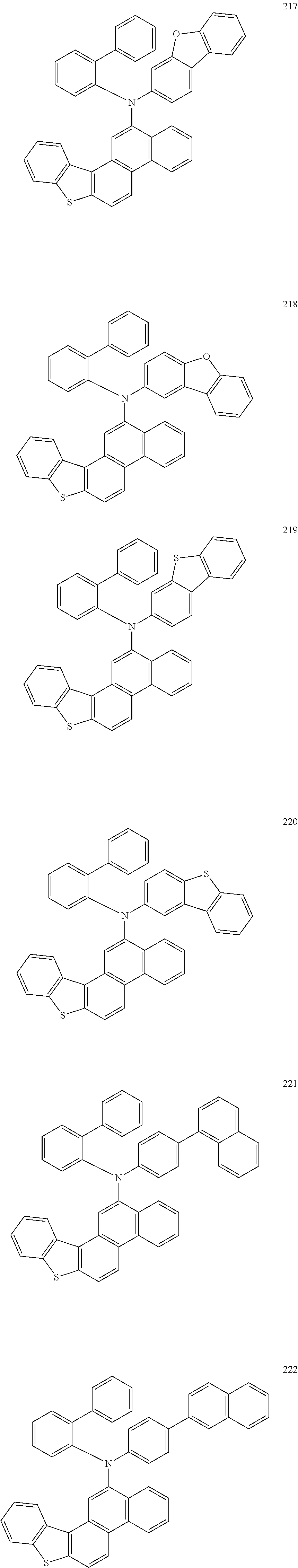 [SMILES: c1ccc(-c2ccccc2N(c2ccc3c(c2)oc2ccccc23)c2cc3c(ccc4sc5ccccc5c43)c3ccccc23)cc1 ; c1ccc(-c2ccccc2N(c2ccc3oc4ccccc4c3c2)c2cc3c(ccc4sc5ccccc5c43)c3ccccc23)cc1 ; c1ccc(-c2ccccc2N(c2ccc3c(c2)sc2ccccc23)c2cc3c(ccc4sc5ccccc5c43)c3ccccc23)cc1 ; c1ccc(-c2ccccc2N(c2ccc3sc4ccccc4c3c2)c2cc3c(ccc4sc5ccccc5c43)c3ccccc23)cc1 ; c1ccc(-c2ccccc2N(c2ccc(-c3cccc4ccccc34)cc2)c2cc3c(ccc4sc5ccccc5c43)c3ccccc23)cc1 ; c1ccc(-c2ccccc2N(c2ccc(-c3ccc4ccccc4c3)cc2)c2cc3c(ccc4sc5ccccc5c43)c3ccccc23)cc1]

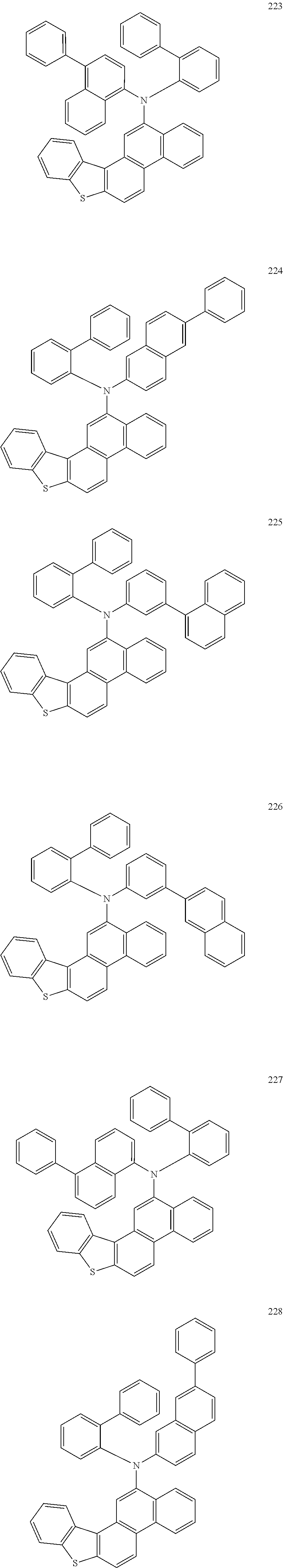 [SMILES: c1ccc(-c2ccccc2N(c2ccc(-c3ccccc3)c3ccccc23)c2cc3c(ccc4sc5ccccc5c43)c3ccccc23)cc1 ; c1ccc(-c2ccc3cc(N(c4ccccc4-c4ccccc4)c4cc5c(ccc6sc7ccccc7c65)c5ccccc45)ccc3c2)cc1 ; c1ccc(-c2ccccc2N(c2cccc(-c3cccc4ccccc34)c2)c2cc3c(ccc4sc5ccccc5c43)c3ccccc23)cc1 ; c1ccc(-c2ccccc2N(c2cccc(-c3ccc4ccccc4c3)c2)c2cc3c(ccc4sc5ccccc5c43)c3ccccc23)cc1 ; c1ccc(-c2ccccc2N(c2cccc3c(-c4ccccc4)cccc23)c2cc3c(ccc4sc5ccccc5c43)c3ccccc23)cc1 ; c1ccc(-c2ccc3ccc(N(c4ccccc4-c4ccccc4)c4cc5c(ccc6sc7ccccc7c65)c5ccccc45)cc3c2)cc1]

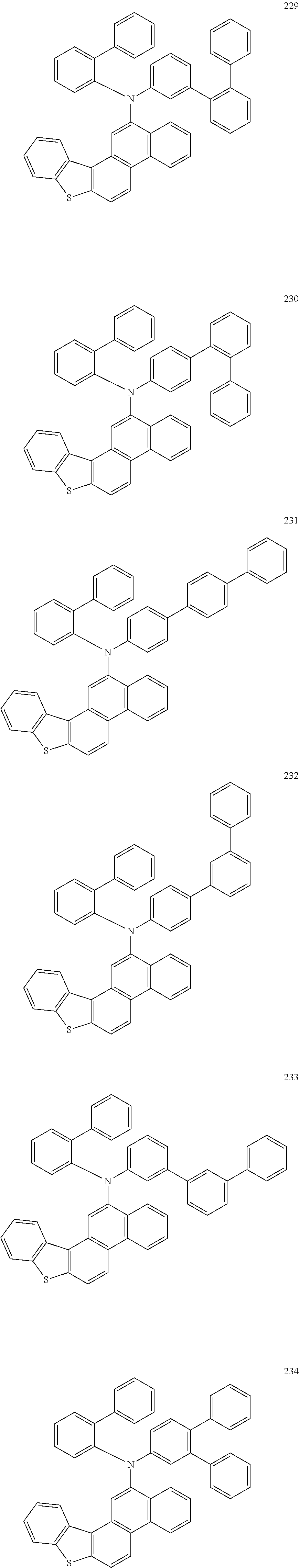

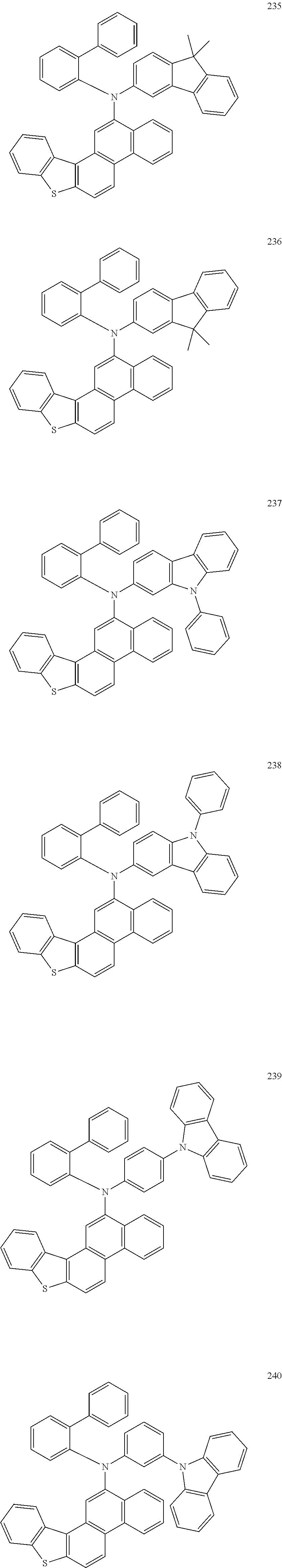

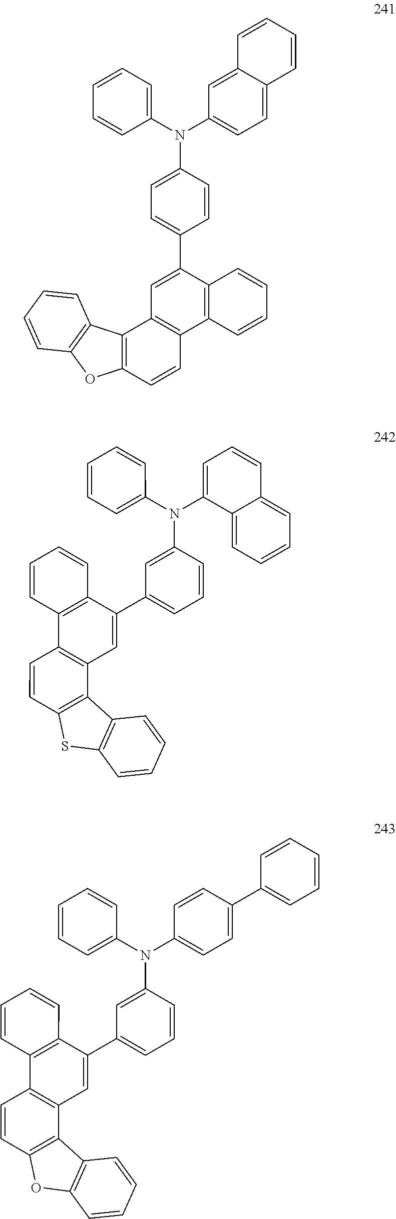 [SMILES: c1ccc(N(c2ccc(-c3cc4c(ccc5oc6ccccc6c54)c4ccccc34)cc2)c2ccc3ccccc3c2)cc1 ; c1ccc(N(c2cccc(-c3cc4c(ccc5sc6ccccc6c54)c4ccccc34)c2)c2cccc3ccccc23)cc1 ; c1ccc(-c2ccc(N(c3ccccc3)c3cccc(-c4cc5c(ccc6oc7ccccc7c65)c5ccccc45)c3)cc2)cc1]

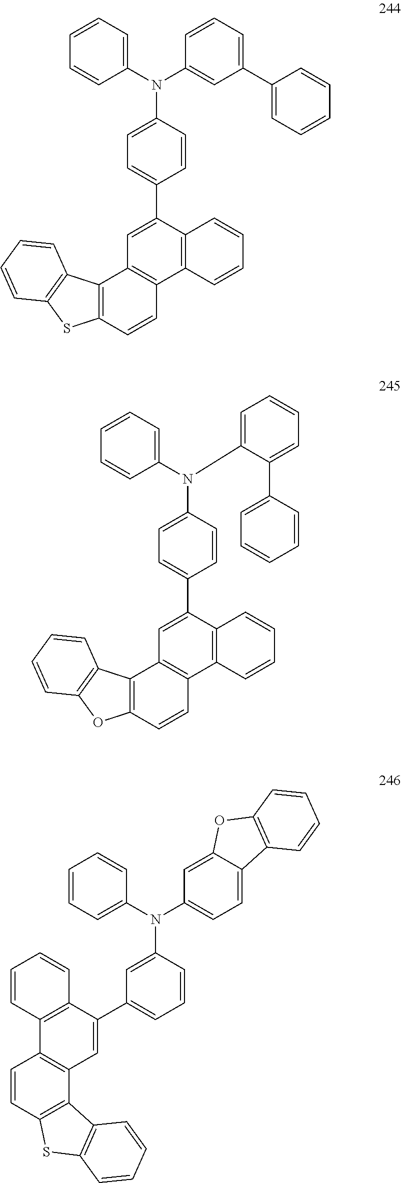 [SMILES: c1ccc(-c2cccc(N(c3ccccc3)c3ccc(-c4cc5c(ccc6sc7ccccc7c65)c5ccccc45)cc3)c2)cc1 ; c1ccc(-c2ccccc2N(c2ccccc2)c2ccc(-c3cc4c(ccc5oc6ccccc6c54)c4ccccc34)cc2)cc1 ; c1ccc(N(c2cccc(-c3cc4c(ccc5sc6ccccc6c54)c4ccccc34)c2)c2ccc3c(c2)oc2ccccc23)cc1]

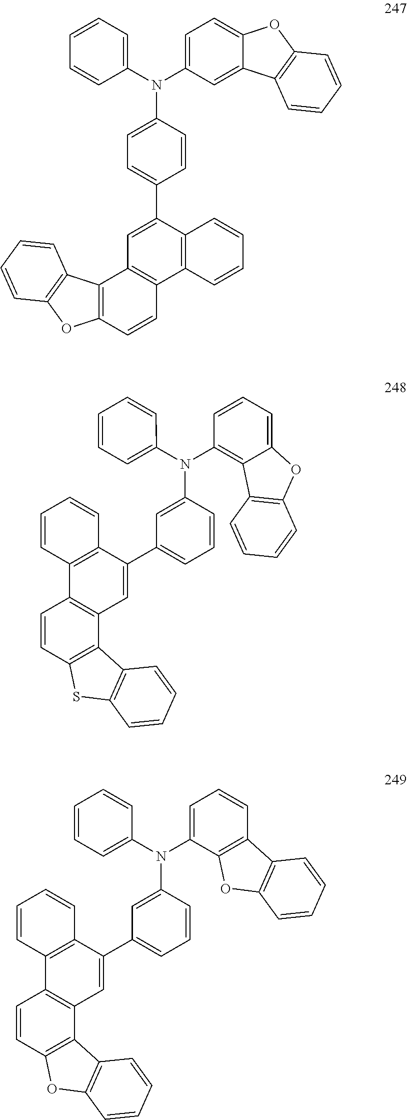 [SMILES: c1ccc(N(c2ccc(-c3cc4c(ccc5oc6ccccc6c54)c4ccccc34)cc2)c2ccc3oc4ccccc4c3c2)cc1 ; c1ccc(N(c2cccc(-c3cc4c(ccc5sc6ccccc6c54)c4ccccc34)c2)c2cccc3oc4ccccc4c23)cc1 ; c1ccc(N(c2cccc(-c3cc4c(ccc5oc6ccccc6c54)c4ccccc34)c2)c2cccc3c2oc2ccccc23)cc1]

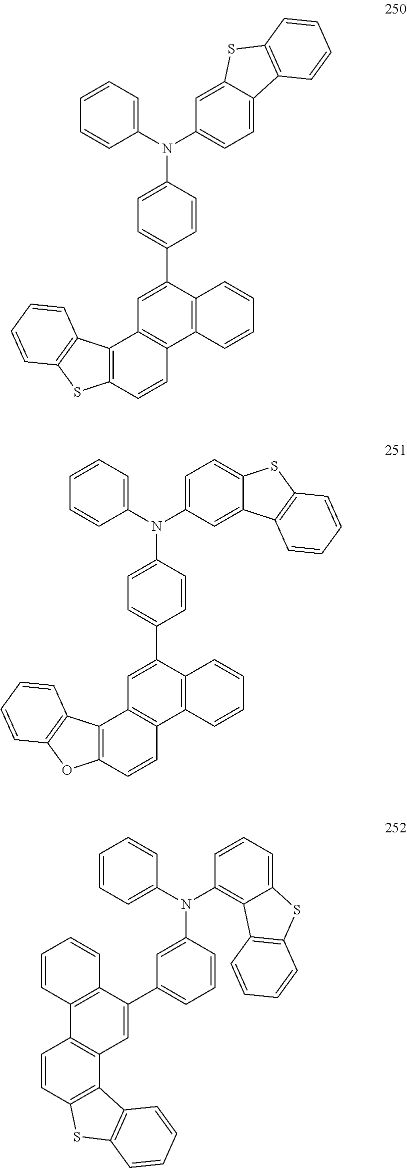 [SMILES: c1ccc(N(c2ccc(-c3cc4c(ccc5sc6ccccc6c54)c4ccccc34)cc2)c2ccc3c(c2)sc2ccccc23)cc1 ; c1ccc(N(c2ccc(-c3cc4c(ccc5oc6ccccc6c54)c4ccccc34)cc2)c2ccc3sc4ccccc4c3c2)cc1 ; c1ccc(N(c2cccc(-c3cc4c(ccc5sc6ccccc6c54)c4ccccc34)c2)c2cccc3sc4ccccc4c23)cc1]

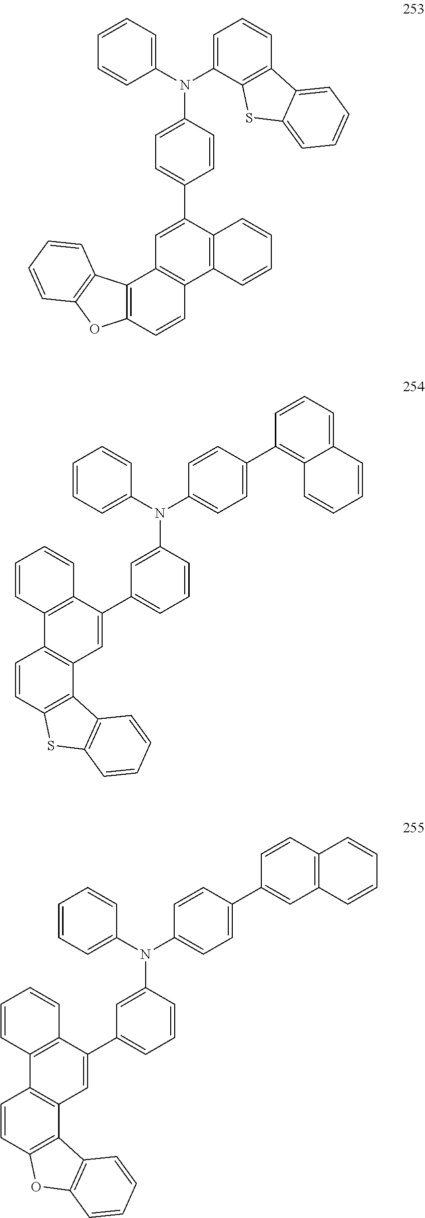 [SMILES: c1ccc(N(c2ccc(-c3cc4c(ccc5oc6ccccc6c54)c4ccccc34)cc2)c2cccc3c2sc2ccccc23)cc1 ; c1ccc(N(c2ccc(-c3cccc4ccccc34)cc2)c2cccc(-c3cc4c(ccc5sc6ccccc6c54)c4ccccc34)c2)cc1 ; c1ccc(N(c2ccc(-c3ccc4ccccc4c3)cc2)c2cccc(-c3cc4c(ccc5oc6ccccc6c54)c4ccccc34)c2)cc1]

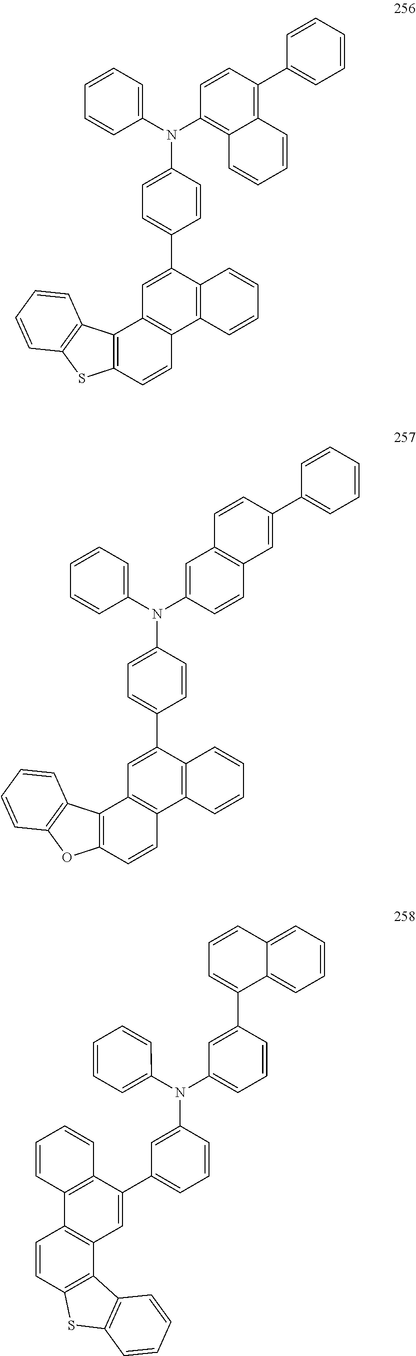 [SMILES: c1ccc(-c2ccc(N(c3ccccc3)c3ccc(-c4cc5c(ccc6sc7ccccc7c65)c5ccccc45)cc3)c3ccccc23)cc1 ; c1ccc(-c2ccc3cc(N(c4ccccc4)c4ccc(-c5cc6c(ccc7oc8ccccc8c76)c6ccccc56)cc4)ccc3c2)cc1 ; c1ccc(N(c2cccc(-c3cccc4ccccc34)c2)c2cccc(-c3cc4c(ccc5sc6ccccc6c54)c4ccccc34)c2)cc1]

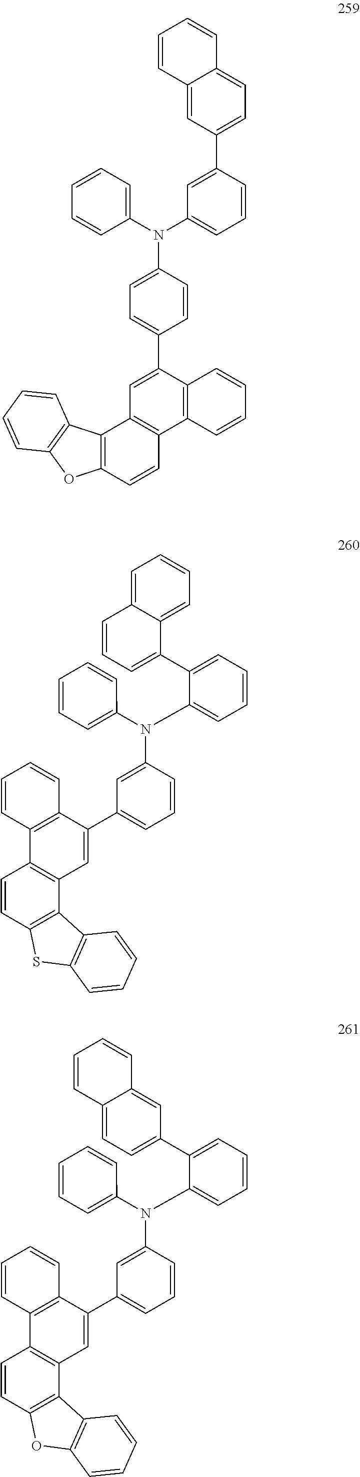 [SMILES: c1ccc(N(c2ccc(-c3cc4c(ccc5oc6ccccc6c54)c4ccccc34)cc2)c2cccc(-c3ccc4ccccc4c3)c2)cc1 ; c1ccc(N(c2cccc(-c3cc4c(ccc5sc6ccccc6c54)c4ccccc34)c2)c2ccccc2-c2cccc3ccccc23)cc1 ; c1ccc(N(c2cccc(-c3cc4c(ccc5oc6ccccc6c54)c4ccccc34)c2)c2ccccc2-c2ccc3ccccc3c2)cc1]

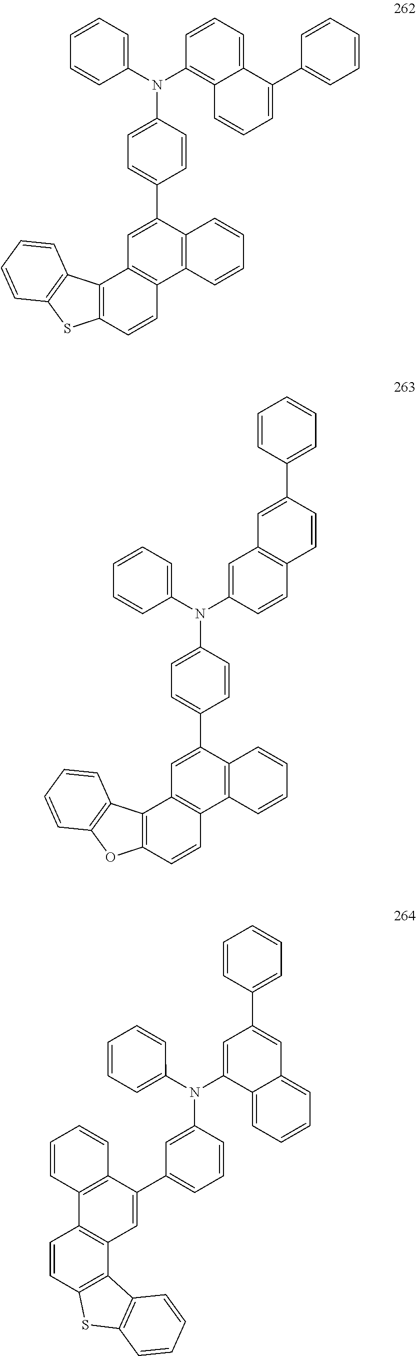 [SMILES: c1ccc(-c2cccc3c(N(c4ccccc4)c4ccc(-c5cc6c(ccc7sc8ccccc8c76)c6ccccc56)cc4)cccc23)cc1 ; c1ccc(-c2ccc3ccc(N(c4ccccc4)c4ccc(-c5cc6c(ccc7oc8ccccc8c76)c6ccccc56)cc4)cc3c2)cc1 ; c1ccc(-c2cc(N(c3ccccc3)c3cccc(-c4cc5c(ccc6sc7ccccc7c65)c5ccccc45)c3)c3ccccc3c2)cc1]

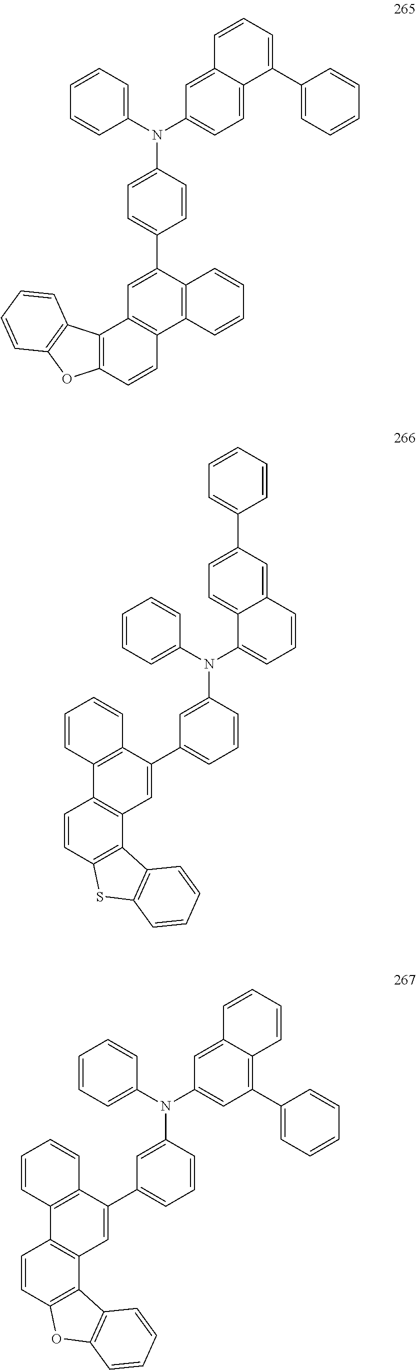 [SMILES: c1ccc(-c2cccc3cc(N(c4ccccc4)c4ccc(-c5cc6c(ccc7oc8ccccc8c76)c6ccccc56)cc4)ccc23)cc1 ; c1ccc(-c2ccc3c(N(c4ccccc4)c4cccc(-c5cc6c(ccc7sc8ccccc8c76)c6ccccc56)c4)cccc3c2)cc1 ; c1ccc(-c2cc(N(c3ccccc3)c3cccc(-c4cc5c(ccc6oc7ccccc7c65)c5ccccc45)c3)cc3ccccc23)cc1]

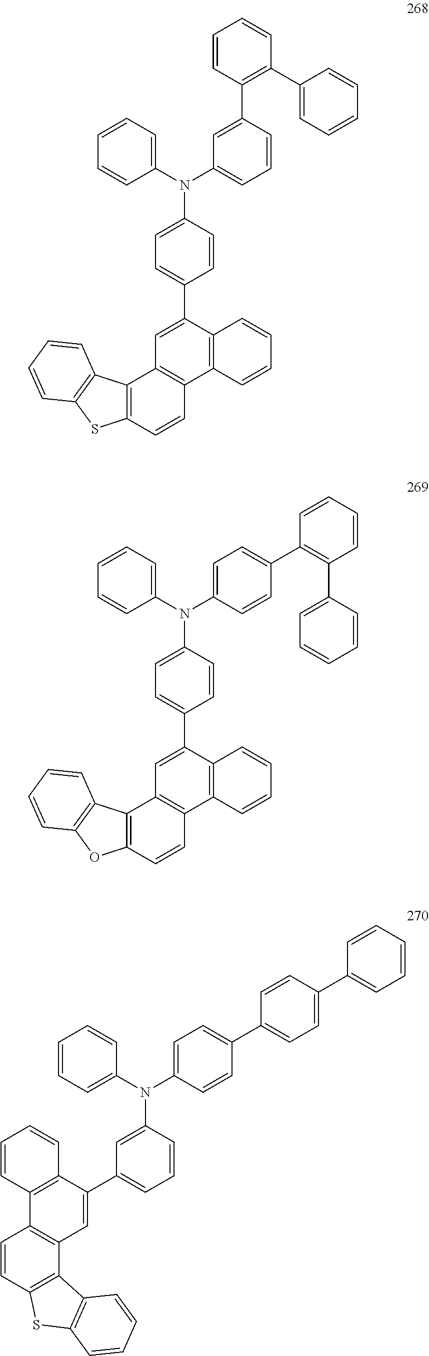 [SMILES: c1ccc(-c2ccccc2-c2cccc(N(c3ccccc3)c3ccc(-c4cc5c(ccc6sc7ccccc7c65)c5ccccc45)cc3)c2)cc1 ; c1ccc(-c2ccccc2-c2ccc(N(c3ccccc3)c3ccc(-c4cc5c(ccc6oc7ccccc7c65)c5ccccc45)cc3)cc2)cc1 ; c1ccc(-c2ccc(-c3ccc(N(c4ccccc4)c4cccc(-c5cc6c(ccc7sc8ccccc8c76)c6ccccc56)c4)cc3)cc2)cc1]

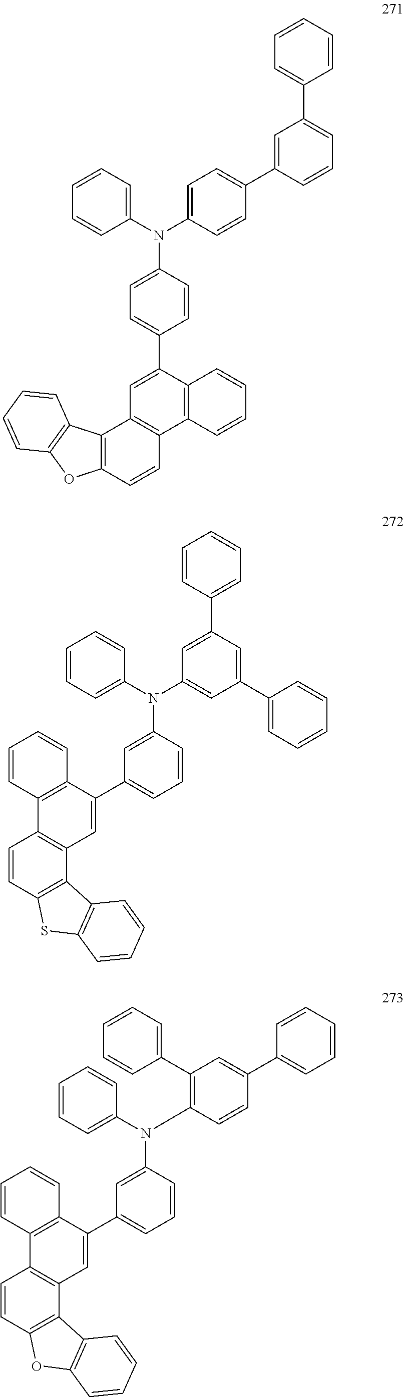 [SMILES: c1ccc(-c2cccc(-c3ccc(N(c4ccccc4)c4ccc(-c5cc6c(ccc7oc8ccccc8c76)c6ccccc56)cc4)cc3)c2)cc1 ; c1ccc(-c2cc(-c3ccccc3)cc(N(c3ccccc3)c3cccc(-c4cc5c(ccc6sc7ccccc7c65)c5ccccc45)c3)c2)cc1 ; c1ccc(-c2ccc(N(c3ccccc3)c3cccc(-c4cc5c(ccc6oc7ccccc7c65)c5ccccc45)c3)c(-c3ccccc3)c2)cc1]

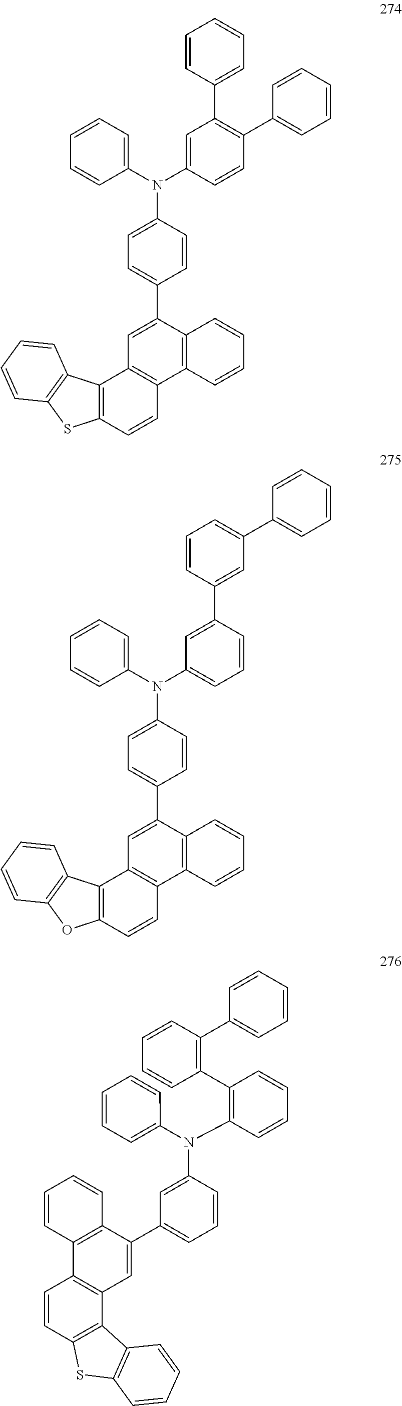 [SMILES: c1ccc(-c2ccc(N(c3ccccc3)c3ccc(-c4cc5c(ccc6sc7ccccc7c65)c5ccccc45)cc3)cc2-c2ccccc2)cc1 ; c1ccc(-c2cccc(-c3cccc(N(c4ccccc4)c4ccc(-c5cc6c(ccc7oc8ccccc8c76)c6ccccc56)cc4)c3)c2)cc1 ; c1ccc(-c2ccccc2-c2ccccc2N(c2ccccc2)c2cccc(-c3cc4c(ccc5sc6ccccc6c54)c4ccccc34)c2)cc1]

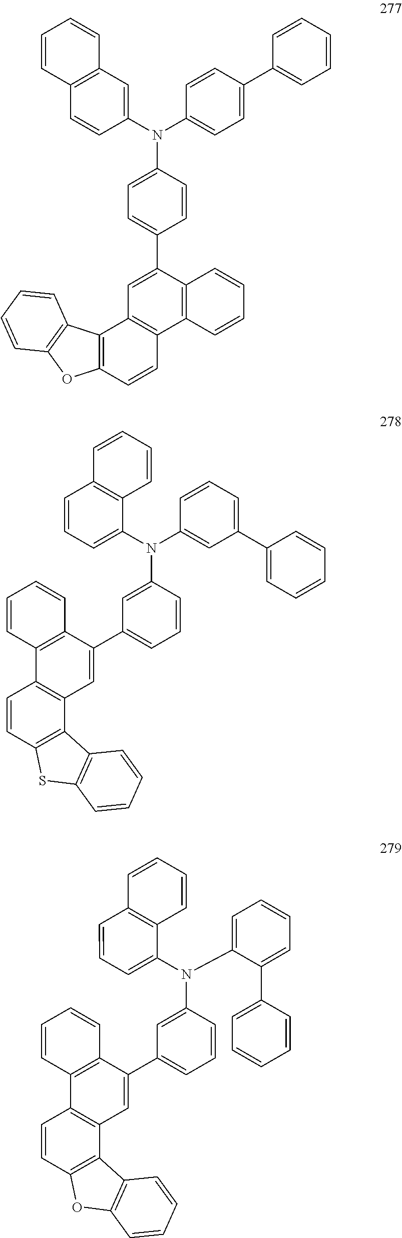

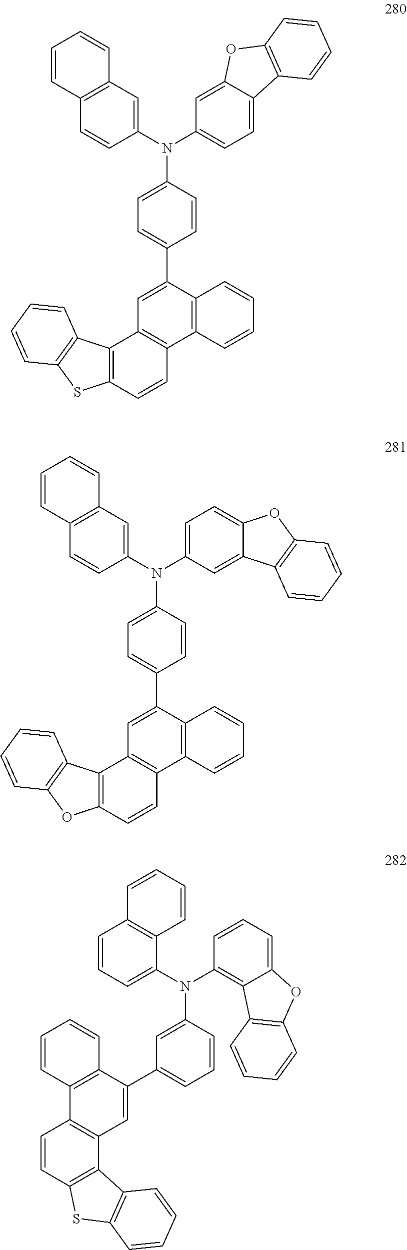 [SMILES: c1ccc2cc(N(c3ccc(-c4cc5c(ccc6sc7ccccc7c65)c5ccccc45)cc3)c3ccc4c(c3)oc3ccccc34)ccc2c1 ; c1ccc2cc(N(c3ccc(-c4cc5c(ccc6oc7ccccc7c65)c5ccccc45)cc3)c3ccc4oc5ccccc5c4c3)ccc2c1 ; c1cc(-c2cc3c(ccc4sc5ccccc5c43)c3ccccc23)cc(N(c2cccc3ccccc23)c2cccc3oc4ccccc4c23)c1]

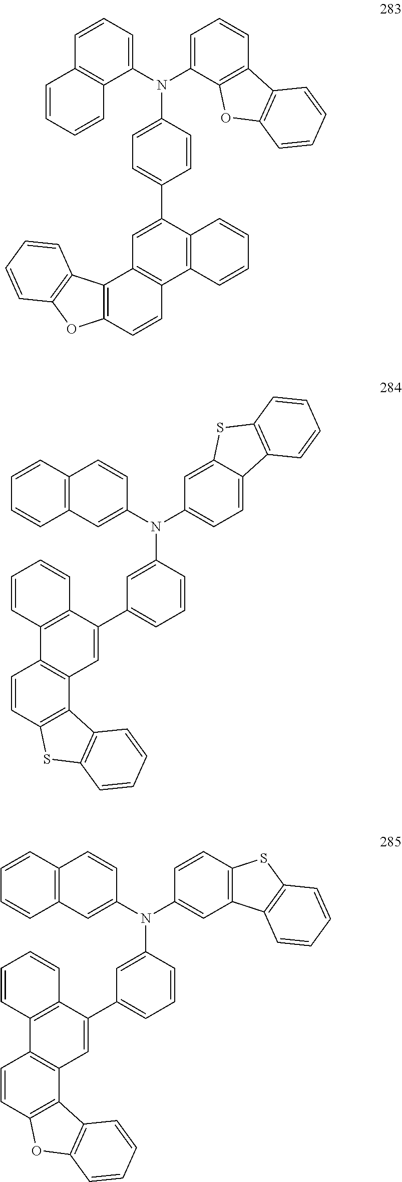 [SMILES: c1ccc2c(N(c3ccc(-c4cc5c(ccc6oc7ccccc7c65)c5ccccc45)cc3)c3cccc4c3oc3ccccc34)cccc2c1 ; c1cc(-c2cc3c(ccc4sc5ccccc5c43)c3ccccc23)cc(N(c2ccc3ccccc3c2)c2ccc3c(c2)sc2ccccc23)c1 ; c1cc(-c2cc3c(ccc4oc5ccccc5c43)c3ccccc23)cc(N(c2ccc3ccccc3c2)c2ccc3sc4ccccc4c3c2)c1]

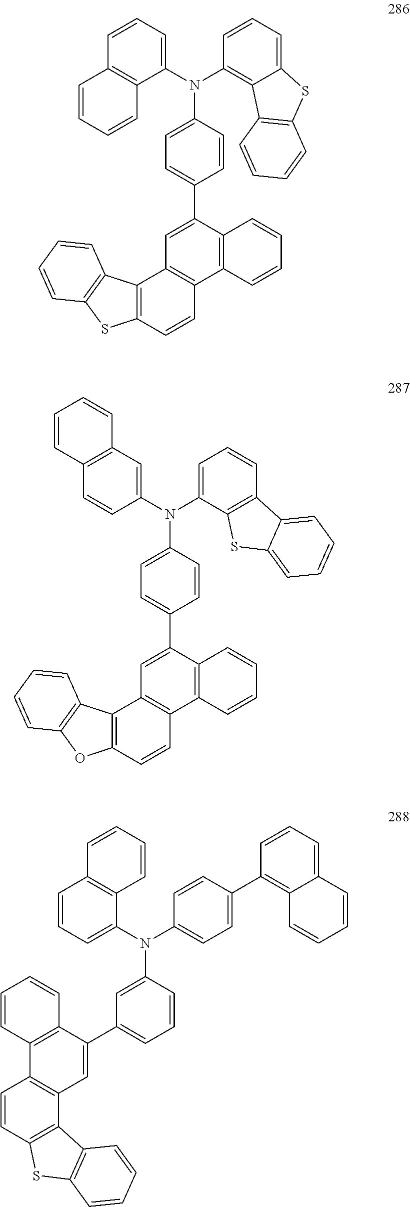 [SMILES: c1ccc2c(N(c3ccc(-c4cc5c(ccc6sc7ccccc7c65)c5ccccc45)cc3)c3cccc4sc5ccccc5c34)cccc2c1 ; c1ccc2cc(N(c3ccc(-c4cc5c(ccc6oc7ccccc7c65)c5ccccc45)cc3)c3cccc4c3sc3ccccc34)ccc2c1 ; c1cc(-c2cc3c(ccc4sc5ccccc5c43)c3ccccc23)cc(N(c2ccc(-c3cccc4ccccc34)cc2)c2cccc3ccccc23)c1]

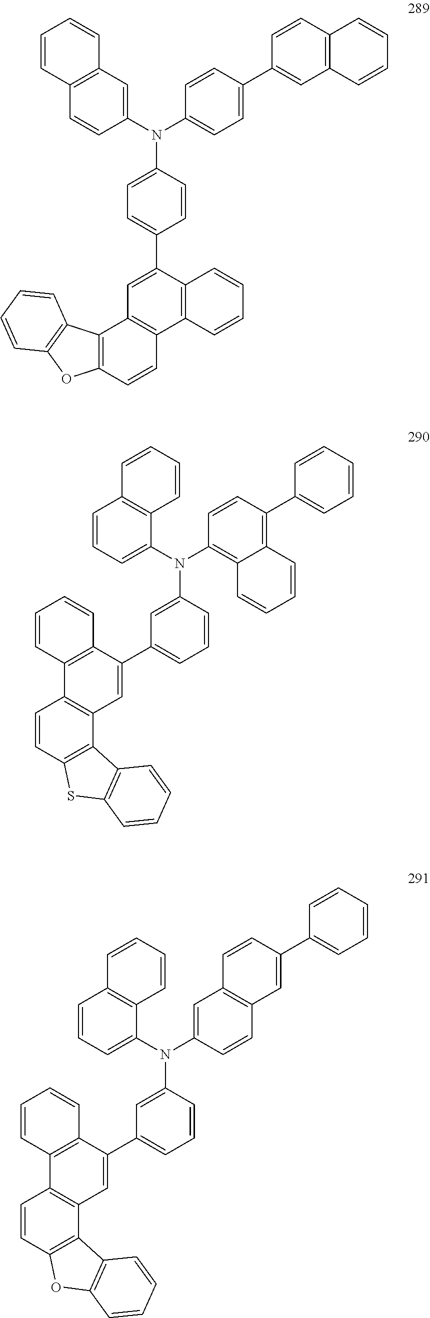 [SMILES: c1ccc2cc(-c3ccc(N(c4ccc(-c5cc6c(ccc7oc8ccccc8c76)c6ccccc56)cc4)c4ccc5ccccc5c4)cc3)ccc2c1 ; c1ccc(-c2ccc(N(c3cccc(-c4cc5c(ccc6sc7ccccc7c65)c5ccccc45)c3)c3cccc4ccccc34)c3ccccc23)cc1 ; c1ccc(-c2ccc3cc(N(c4cccc(-c5cc6c(ccc7oc8ccccc8c76)c6ccccc56)c4)c4cccc5ccccc45)ccc3c2)cc1]

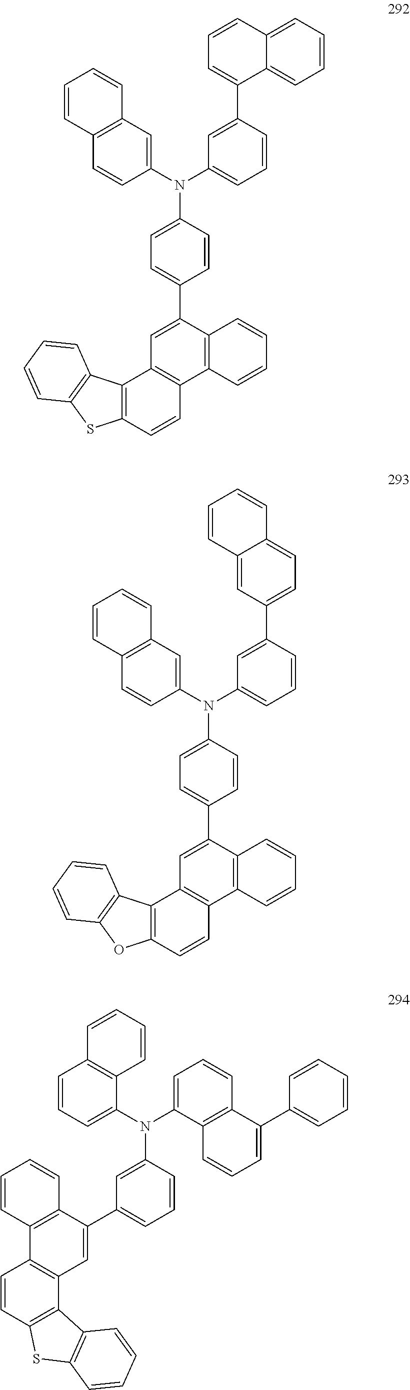 [SMILES: c1cc(-c2cccc3ccccc23)cc(N(c2ccc(-c3cc4c(ccc5sc6ccccc6c54)c4ccccc34)cc2)c2ccc3ccccc3c2)c1 ; c1cc(-c2ccc3ccccc3c2)cc(N(c2ccc(-c3cc4c(ccc5oc6ccccc6c54)c4ccccc34)cc2)c2ccc3ccccc3c2)c1 ; c1ccc(-c2cccc3c(N(c4cccc(-c5cc6c(ccc7sc8ccccc8c76)c6ccccc56)c4)c4cccc5ccccc45)cccc23)cc1]

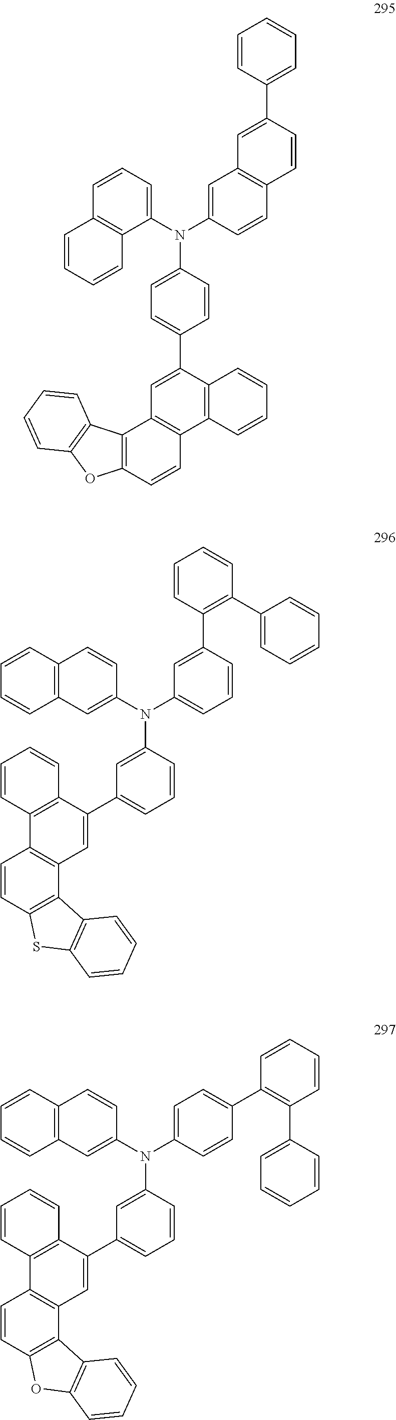 [SMILES: c1ccc(-c2ccc3ccc(N(c4ccc(-c5cc6c(ccc7oc8ccccc8c76)c6ccccc56)cc4)c4cccc5ccccc45)cc3c2)cc1 ; c1ccc(-c2ccccc2-c2cccc(N(c3cccc(-c4cc5c(ccc6sc7ccccc7c65)c5ccccc45)c3)c3ccc4ccccc4c3)c2)cc1 ; c1ccc(-c2ccccc2-c2ccc(N(c3cccc(-c4cc5c(ccc6oc7ccccc7c65)c5ccccc45)c3)c3ccc4ccccc4c3)cc2)cc1]

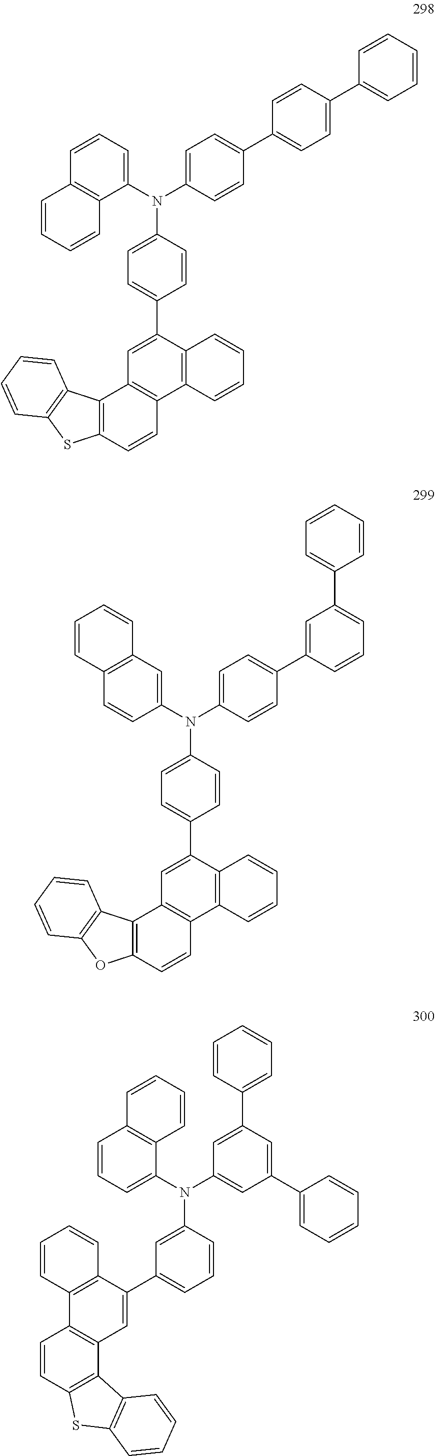 [SMILES: c1ccc(-c2ccc(-c3ccc(N(c4ccc(-c5cc6c(ccc7sc8ccccc8c76)c6ccccc56)cc4)c4cccc5ccccc45)cc3)cc2)cc1 ; c1ccc(-c2cccc(-c3ccc(N(c4ccc(-c5cc6c(ccc7oc8ccccc8c76)c6ccccc56)cc4)c4ccc5ccccc5c4)cc3)c2)cc1 ; c1ccc(-c2cc(-c3ccccc3)cc(N(c3cccc(-c4cc5c(ccc6sc7ccccc7c65)c5ccccc45)c3)c3cccc4ccccc34)c2)cc1]

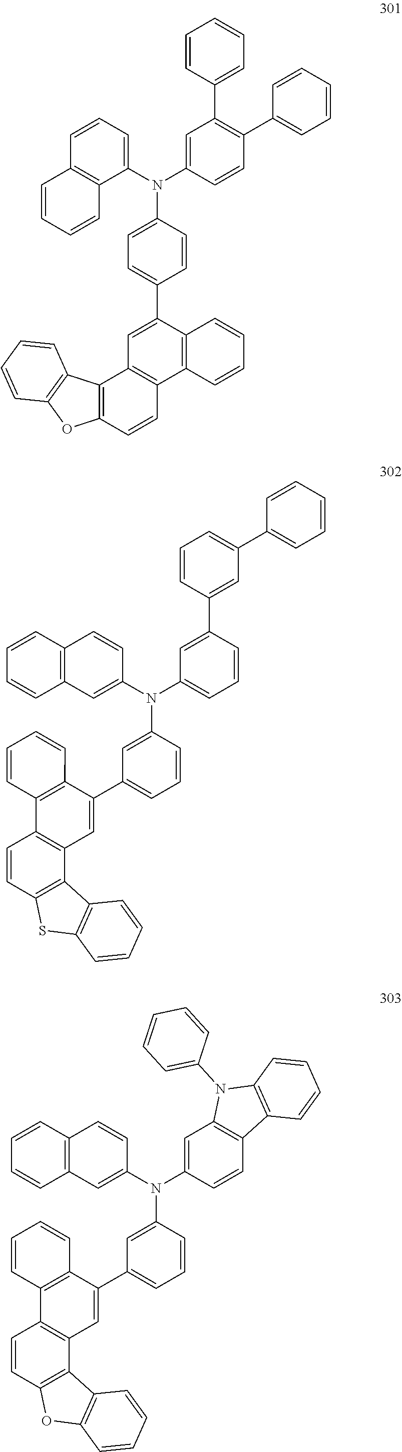 [SMILES: c1ccc(-c2ccc(N(c3ccc(-c4cc5c(ccc6oc7ccccc7c65)c5ccccc45)cc3)c3cccc4ccccc34)cc2-c2ccccc2)cc1 ; c1ccc(-c2cccc(-c3cccc(N(c4cccc(-c5cc6c(ccc7sc8ccccc8c76)c6ccccc56)c4)c4ccc5ccccc5c4)c3)c2)cc1 ; c1ccc(-n2c3ccccc3c3ccc(N(c4cccc(-c5cc6c(ccc7oc8ccccc8c76)c6ccccc56)c4)c4ccc5ccccc5c4)cc32)cc1]

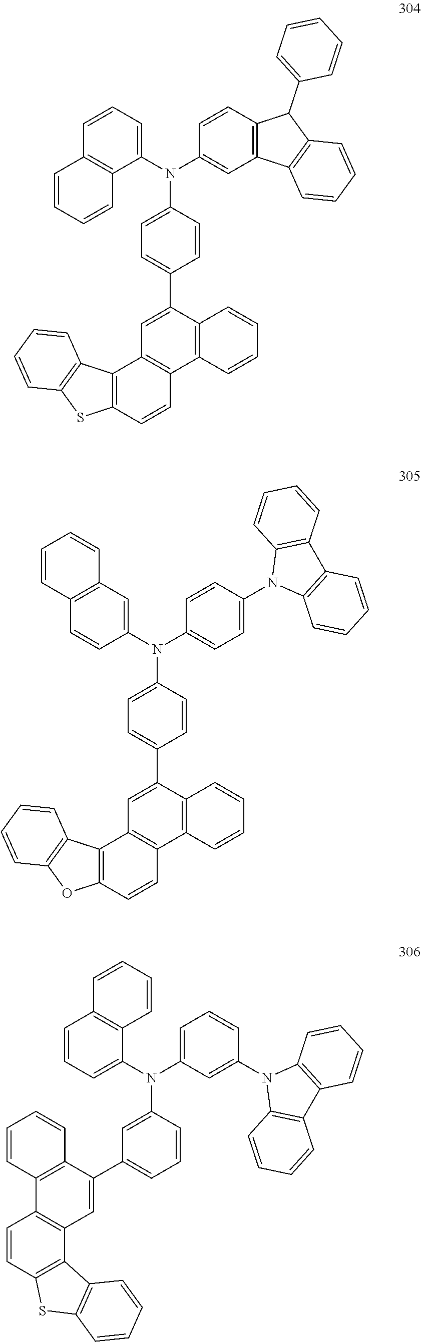 [SMILES: c1ccc(C2c3ccccc3-c3cc(N(c4ccc(-c5cc6c(ccc7sc8ccccc8c76)c6ccccc56)cc4)c4cccc5ccccc45)ccc32)cc1 ; c1ccc2cc(N(c3ccc(-c4cc5c(ccc6oc7ccccc7c65)c5ccccc45)cc3)c3ccc(-n4c5ccccc5c5ccccc54)cc3)ccc2c1 ; c1cc(-c2cc3c(ccc4sc5ccccc5c43)c3ccccc23)cc(N(c2cccc(-n3c4ccccc4c4ccccc43)c2)c2cccc3ccccc23)c1]

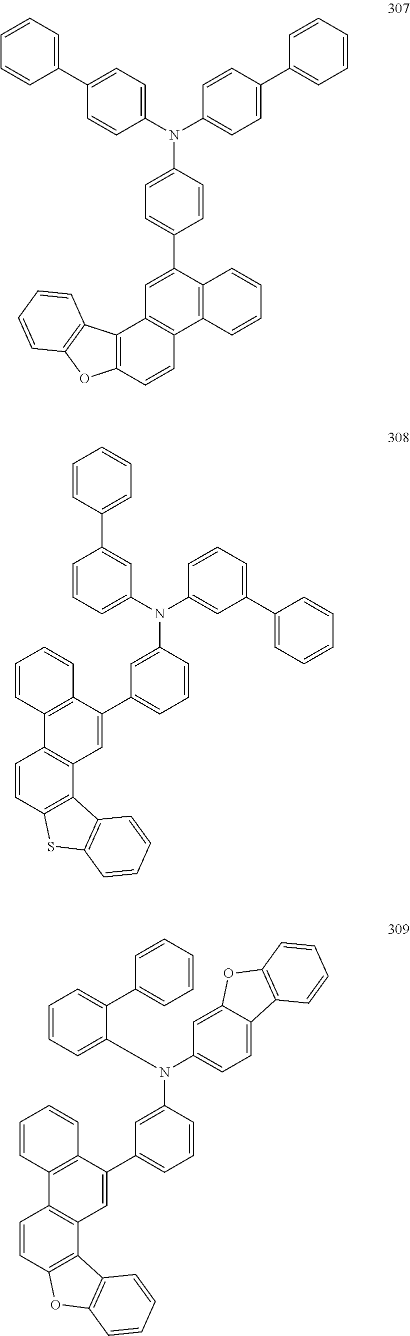 [SMILES: c1ccc(-c2ccc(N(c3ccc(-c4ccccc4)cc3)c3ccc(-c4cc5c(ccc6oc7ccccc7c65)c5ccccc45)cc3)cc2)cc1 ; c1ccc(-c2cccc(N(c3cccc(-c4ccccc4)c3)c3cccc(-c4cc5c(ccc6sc7ccccc7c65)c5ccccc45)c3)c2)cc1 ; c1ccc(-c2ccccc2N(c2cccc(-c3cc4c(ccc5oc6ccccc6c54)c4ccccc34)c2)c2ccc3c(c2)oc2ccccc23)cc1]

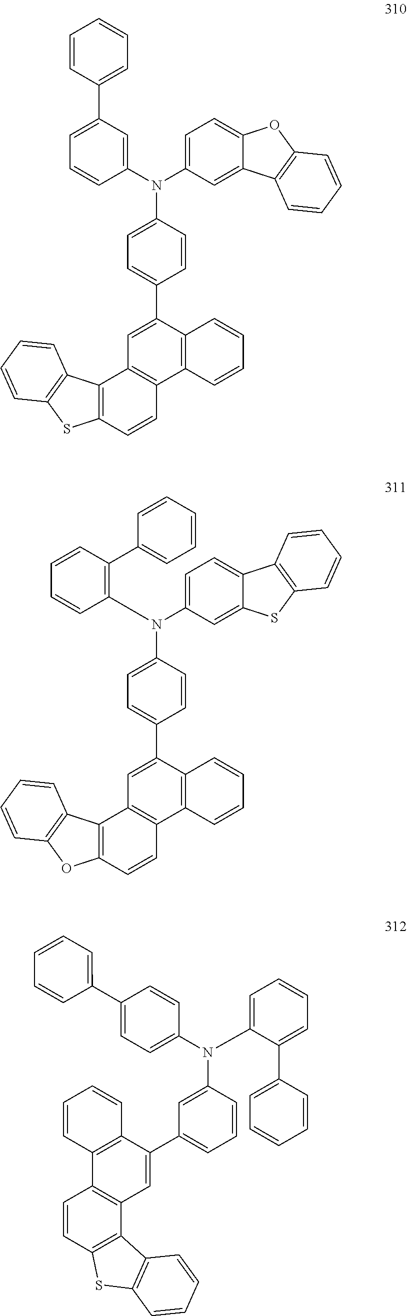 [SMILES: c1ccc(-c2cccc(N(c3ccc(-c4cc5c(ccc6sc7ccccc7c65)c5ccccc45)cc3)c3ccc4oc5ccccc5c4c3)c2)cc1 ; c1ccc(-c2ccccc2N(c2ccc(-c3cc4c(ccc5oc6ccccc6c54)c4ccccc34)cc2)c2ccc3c(c2)sc2ccccc23)cc1 ; c1ccc(-c2ccc(N(c3cccc(-c4cc5c(ccc6sc7ccccc7c65)c5ccccc45)c3)c3ccccc3-c3ccccc3)cc2)cc1]

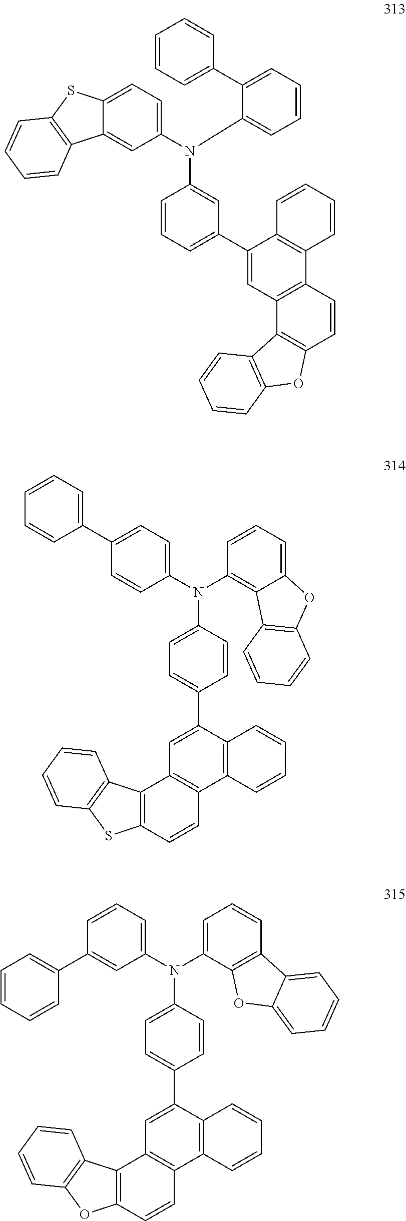 [SMILES: c1ccc(-c2ccccc2N(c2cccc(-c3cc4c(ccc5oc6ccccc6c54)c4ccccc34)c2)c2ccc3sc4ccccc4c3c2)cc1 ; c1ccc(-c2ccc(N(c3ccc(-c4cc5c(ccc6sc7ccccc7c65)c5ccccc45)cc3)c3cccc4oc5ccccc5c34)cc2)cc1 ; c1ccc(-c2cccc(N(c3ccc(-c4cc5c(ccc6oc7ccccc7c65)c5ccccc45)cc3)c3cccc4c3oc3ccccc34)c2)cc1]

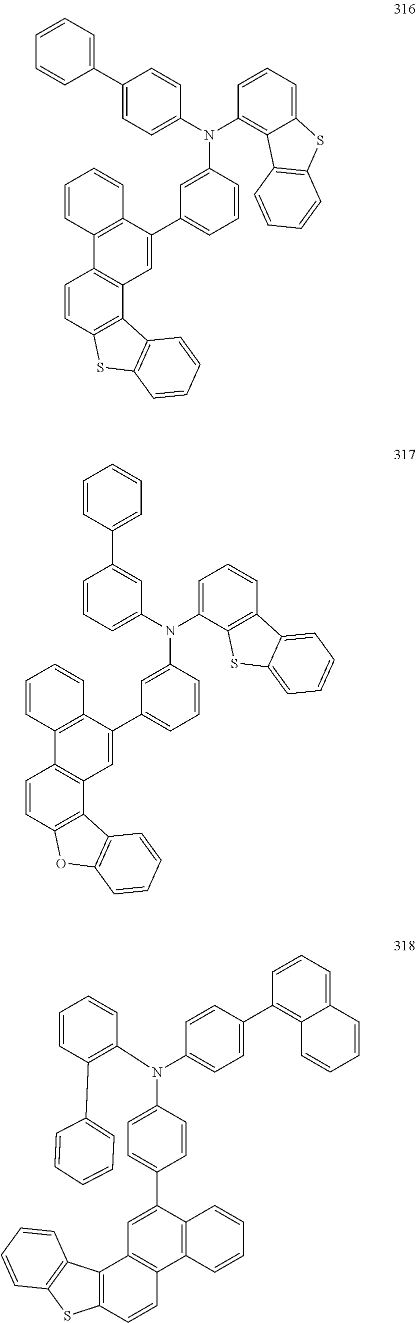 [SMILES: c1ccc(-c2ccc(N(c3cccc(-c4cc5c(ccc6sc7ccccc7c65)c5ccccc45)c3)c3cccc4sc5ccccc5c34)cc2)cc1 ; c1ccc(-c2cccc(N(c3cccc(-c4cc5c(ccc6oc7ccccc7c65)c5ccccc45)c3)c3cccc4c3sc3ccccc34)c2)cc1 ; c1ccc(-c2ccccc2N(c2ccc(-c3cccc4ccccc34)cc2)c2ccc(-c3cc4c(ccc5sc6ccccc6c54)c4ccccc34)cc2)cc1]

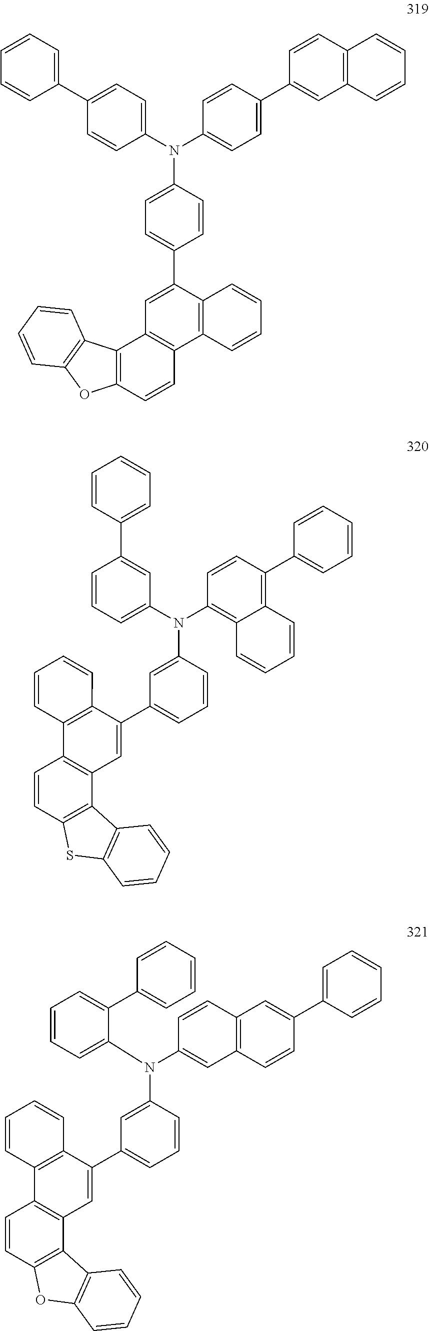 [SMILES: c1ccc(-c2ccc(N(c3ccc(-c4ccc5ccccc5c4)cc3)c3ccc(-c4cc5c(ccc6oc7ccccc7c65)c5ccccc45)cc3)cc2)cc1 ; c1ccc(-c2cccc(N(c3cccc(-c4cc5c(ccc6sc7ccccc7c65)c5ccccc45)c3)c3ccc(-c4ccccc4)c4ccccc34)c2)cc1 ; c1ccc(-c2ccc3cc(N(c4cccc(-c5cc6c(ccc7oc8ccccc8c76)c6ccccc56)c4)c4ccccc4-c4ccccc4)ccc3c2)cc1]

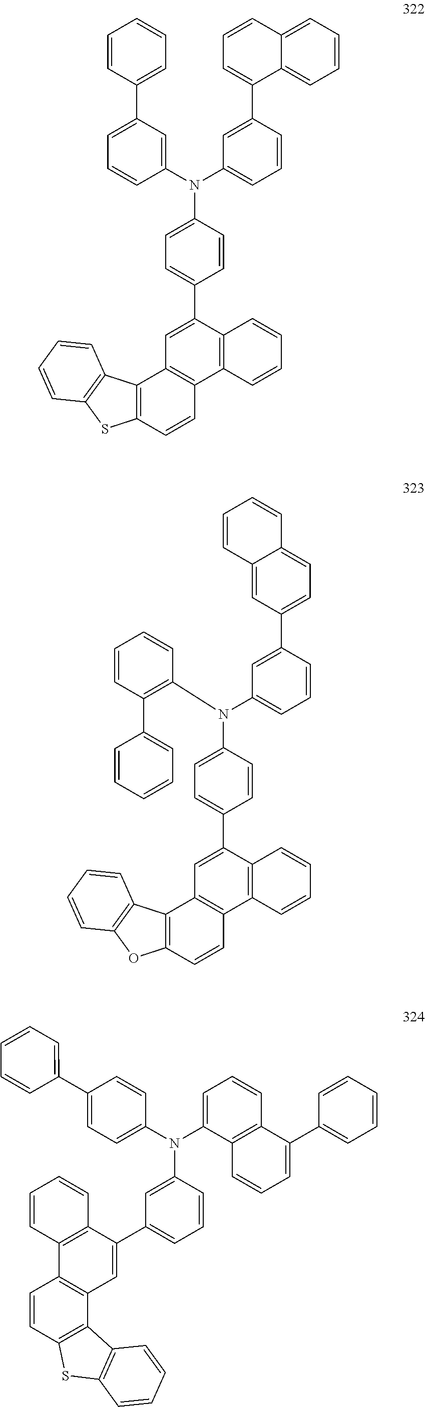 [SMILES: c1ccc(-c2cccc(N(c3ccc(-c4cc5c(ccc6sc7ccccc7c65)c5ccccc45)cc3)c3cccc(-c4cccc5ccccc45)c3)c2)cc1 ; c1ccc(-c2ccccc2N(c2ccc(-c3cc4c(ccc5oc6ccccc6c54)c4ccccc34)cc2)c2cccc(-c3ccc4ccccc4c3)c2)cc1 ; c1ccc(-c2ccc(N(c3cccc(-c4cc5c(ccc6sc7ccccc7c65)c5ccccc45)c3)c3cccc4c(-c5ccccc5)cccc34)cc2)cc1]

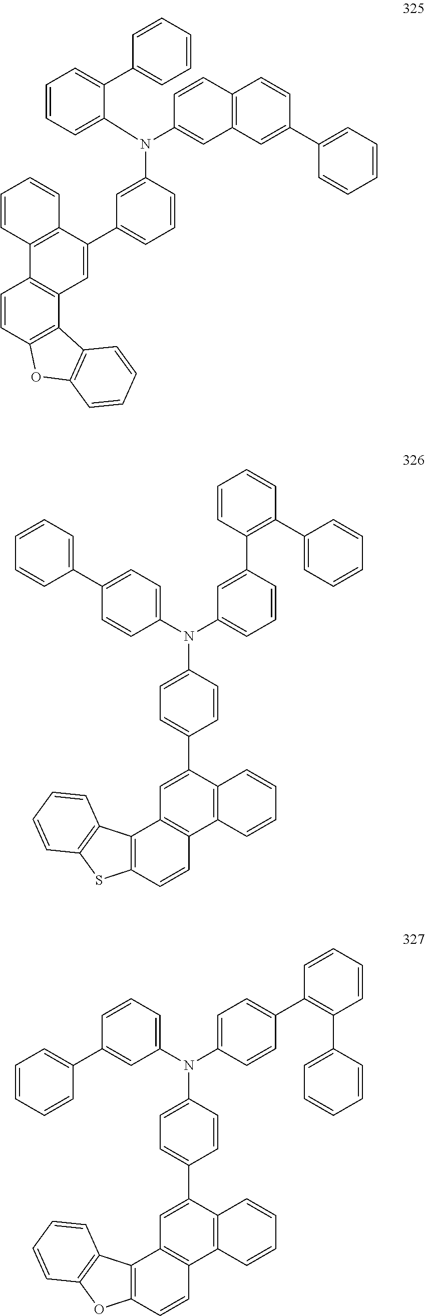 [SMILES: c1ccc(-c2ccc3ccc(N(c4cccc(-c5cc6c(ccc7oc8ccccc8c76)c6ccccc56)c4)c4ccccc4-c4ccccc4)cc3c2)cc1 ; c1ccc(-c2ccc(N(c3ccc(-c4cc5c(ccc6sc7ccccc7c65)c5ccccc45)cc3)c3cccc(-c4ccccc4-c4ccccc4)c3)cc2)cc1 ; c1ccc(-c2cccc(N(c3ccc(-c4ccccc4-c4ccccc4)cc3)c3ccc(-c4cc5c(ccc6oc7ccccc7c65)c5ccccc45)cc3)c2)cc1]

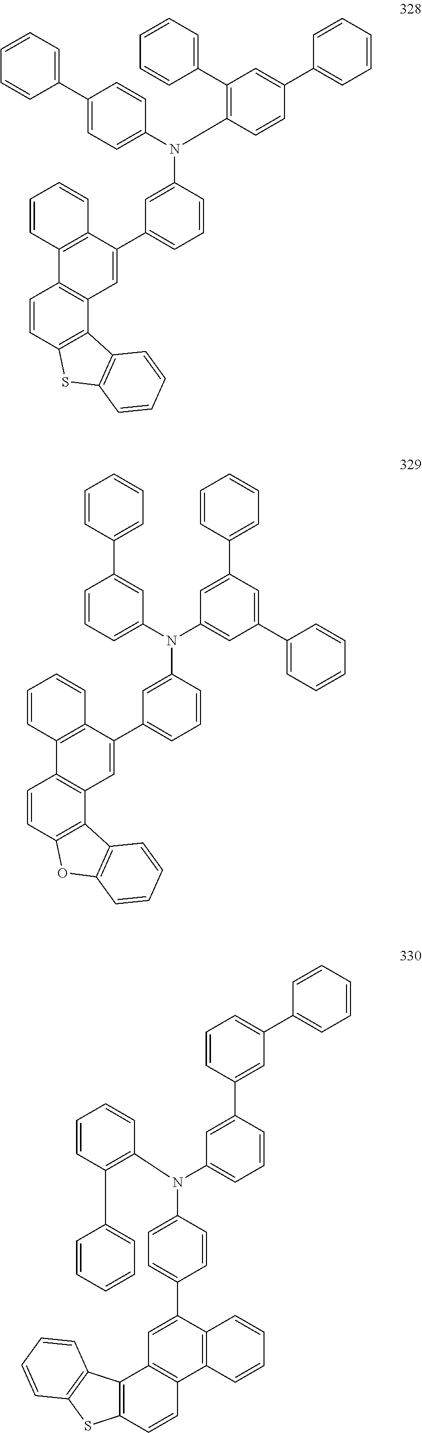 [SMILES: c1ccc(-c2ccc(N(c3cccc(-c4cc5c(ccc6sc7ccccc7c65)c5ccccc45)c3)c3ccc(-c4ccccc4)cc3-c3ccccc3)cc2)cc1 ; c1ccc(-c2cccc(N(c3cc(-c4ccccc4)cc(-c4ccccc4)c3)c3cccc(-c4cc5c(ccc6oc7ccccc7c65)c5ccccc45)c3)c2)cc1 ; c1ccc(-c2cccc(-c3cccc(N(c4ccc(-c5cc6c(ccc7sc8ccccc8c76)c6ccccc56)cc4)c4ccccc4-c4ccccc4)c3)c2)cc1]

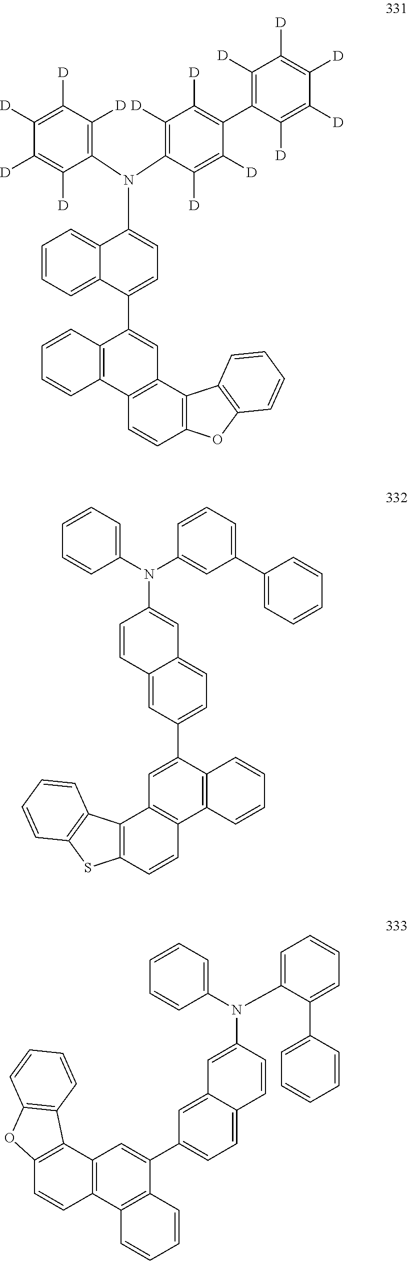 [SMILES: [2H]c1c([2H])c([2H])c(-c2c([2H])c([2H])c(N(c3c([2H])c([2H])c([2H])c([2H])c3[2H])c3ccc(-c4cc5c(ccc6oc7ccccc7c65)c5ccccc45)c4ccccc34)c([2H])c2[2H])c([2H])c1[2H] ; c1ccc(-c2cccc(N(c3ccccc3)c3ccc4cc(-c5cc6c(ccc7sc8ccccc8c76)c6ccccc56)ccc4c3)c2)cc1 ; c1ccc(-c2ccccc2N(c2ccccc2)c2ccc3ccc(-c4cc5c(ccc6oc7ccccc7c65)c5ccccc45)cc3c2)cc1]

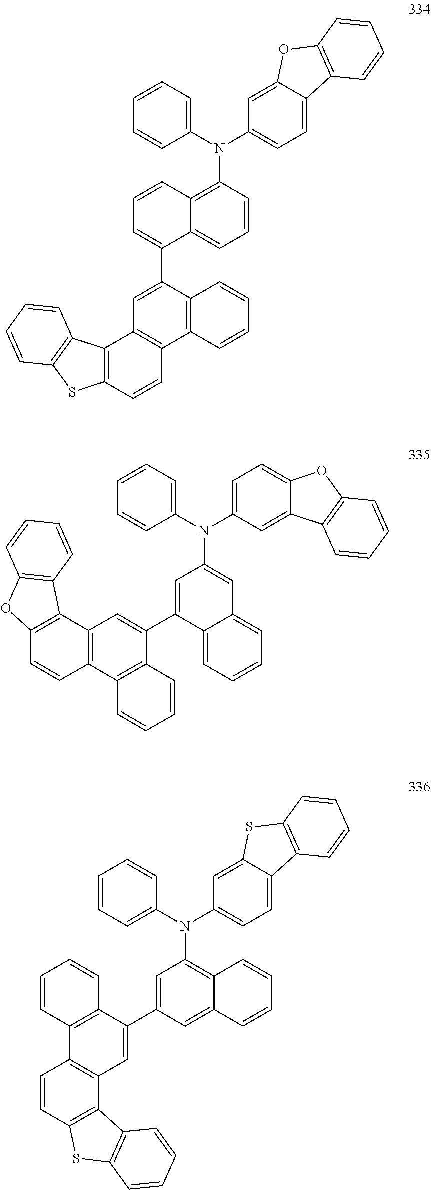 [SMILES: c1ccc(N(c2ccc3c(c2)oc2ccccc23)c2cccc3c(-c4cc5c(ccc6sc7ccccc7c65)c5ccccc45)cccc23)cc1 ; c1ccc(N(c2cc(-c3cc4c(ccc5oc6ccccc6c54)c4ccccc34)c3ccccc3c2)c2ccc3oc4ccccc4c3c2)cc1 ; c1ccc(N(c2ccc3c(c2)sc2ccccc23)c2cc(-c3cc4c(ccc5sc6ccccc6c54)c4ccccc34)cc3ccccc23)cc1]

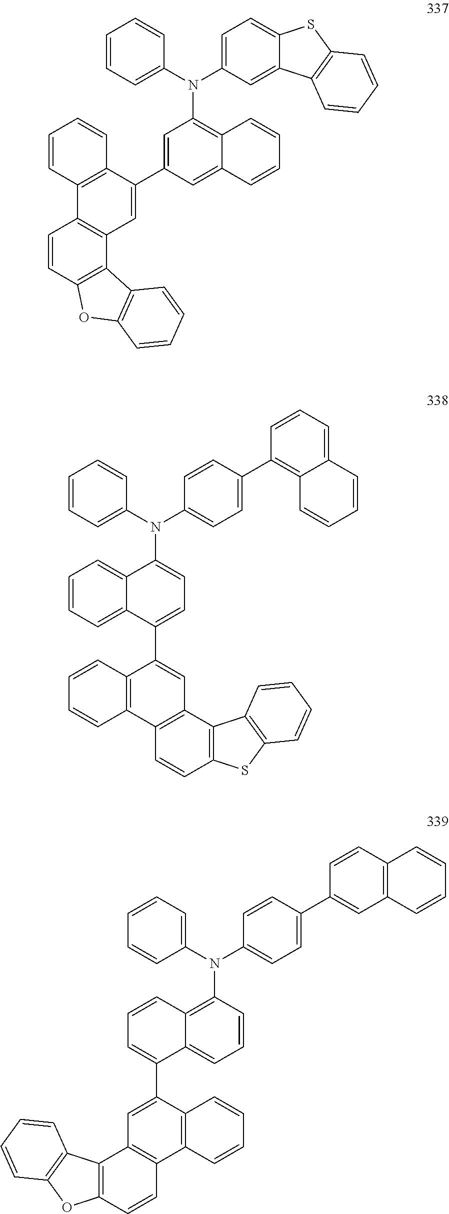 [SMILES: c1ccc(N(c2ccc3sc4ccccc4c3c2)c2cc(-c3cc4c(ccc5oc6ccccc6c54)c4ccccc34)cc3ccccc23)cc1 ; c1ccc(N(c2ccc(-c3cccc4ccccc34)cc2)c2ccc(-c3cc4c(ccc5sc6ccccc6c54)c4ccccc34)c3ccccc23)cc1 ; c1ccc(N(c2ccc(-c3ccc4ccccc4c3)cc2)c2cccc3c(-c4cc5c(ccc6oc7ccccc7c65)c5ccccc45)cccc23)cc1]

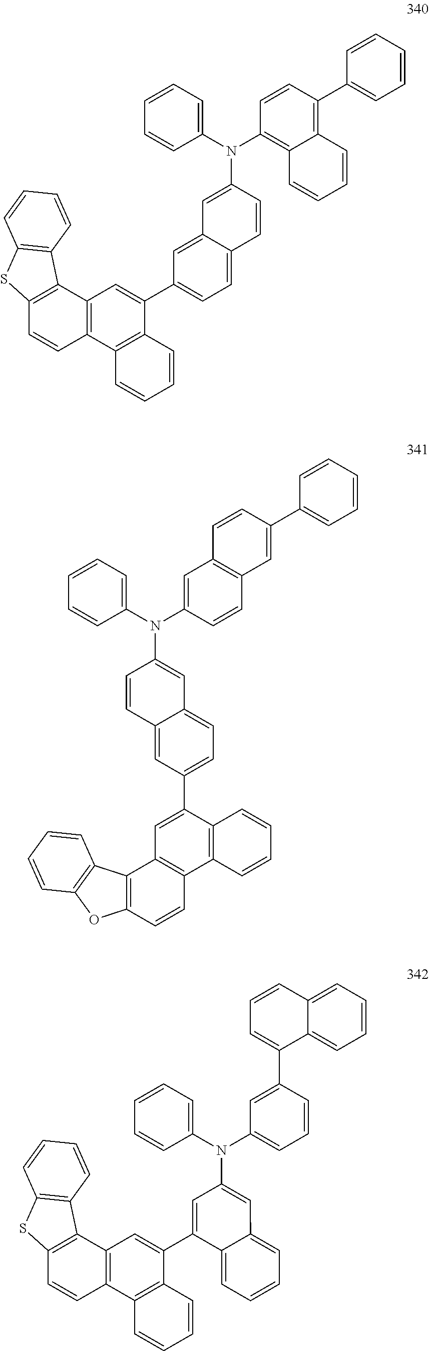 [SMILES: c1ccc(-c2ccc(N(c3ccccc3)c3ccc4ccc(-c5cc6c(ccc7sc8ccccc8c76)c6ccccc56)cc4c3)c3ccccc23)cc1 ; c1ccc(-c2ccc3cc(N(c4ccccc4)c4ccc5cc(-c6cc7c(ccc8oc9ccccc9c87)c7ccccc67)ccc5c4)ccc3c2)cc1 ; c1ccc(N(c2cccc(-c3cccc4ccccc34)c2)c2cc(-c3cc4c(ccc5sc6ccccc6c54)c4ccccc34)c3ccccc3c2)cc1]

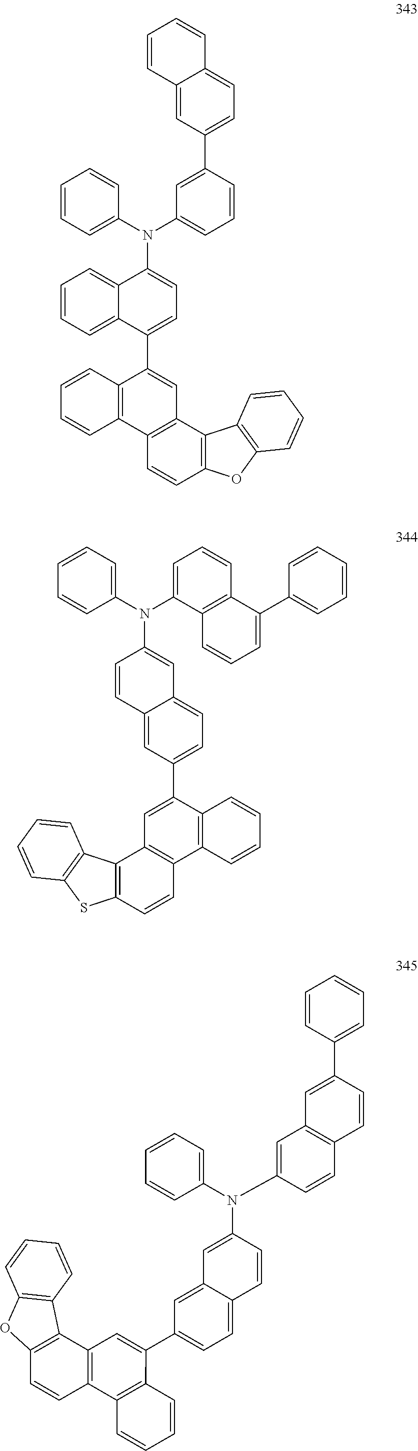 [SMILES: c1ccc(N(c2cccc(-c3ccc4ccccc4c3)c2)c2ccc(-c3cc4c(ccc5oc6ccccc6c54)c4ccccc34)c3ccccc23)cc1 ; c1ccc(-c2cccc3c(N(c4ccccc4)c4ccc5cc(-c6cc7c(ccc8sc9ccccc9c87)c7ccccc67)ccc5c4)cccc23)cc1 ; c1ccc(-c2ccc3ccc(N(c4ccccc4)c4ccc5ccc(-c6cc7c(ccc8oc9ccccc9c87)c7ccccc67)cc5c4)cc3c2)cc1]

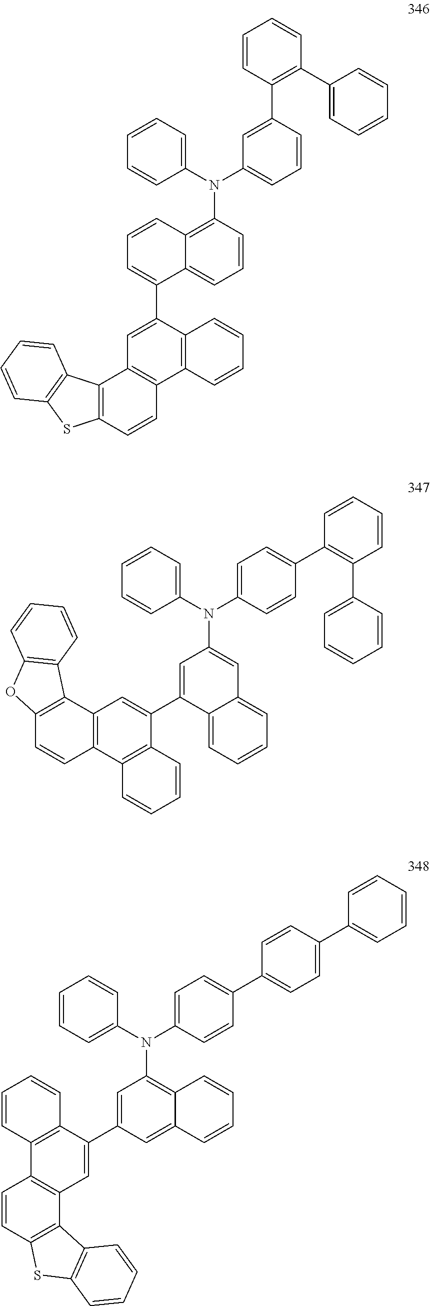 [SMILES: c1ccc(-c2ccccc2-c2cccc(N(c3ccccc3)c3cccc4c(-c5cc6c(ccc7sc8ccccc8c76)c6ccccc56)cccc34)c2)cc1 ; c1ccc(-c2ccccc2-c2ccc(N(c3ccccc3)c3cc(-c4cc5c(ccc6oc7ccccc7c65)c5ccccc45)c4ccccc4c3)cc2)cc1 ; c1ccc(-c2ccc(-c3ccc(N(c4ccccc4)c4cc(-c5cc6c(ccc7sc8ccccc8c76)c6ccccc56)cc5ccccc45)cc3)cc2)cc1]

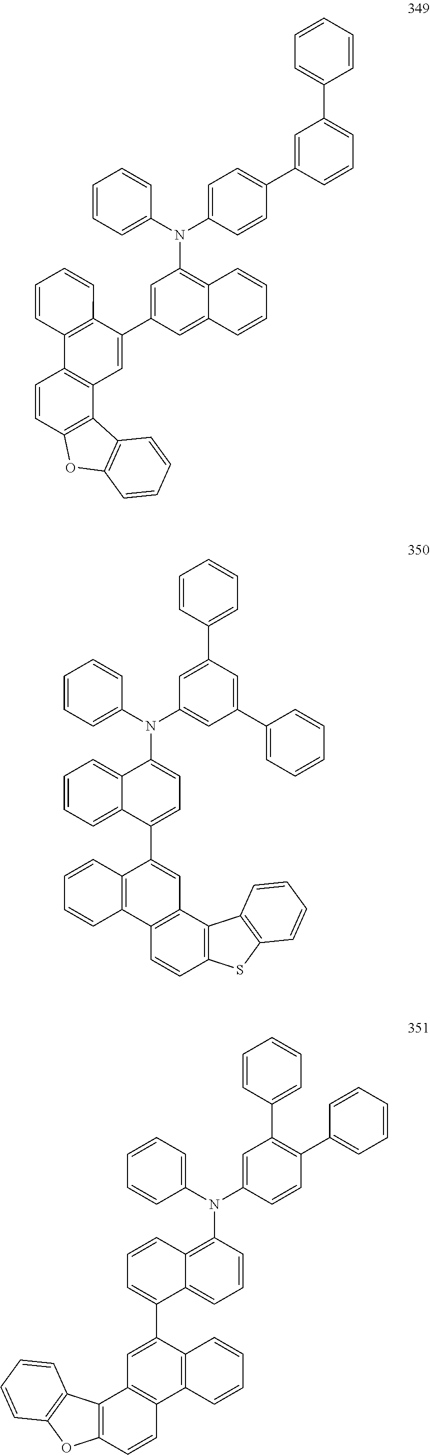 [SMILES: c1ccc(-c2cccc(-c3ccc(N(c4ccccc4)c4cc(-c5cc6c(ccc7oc8ccccc8c76)c6ccccc56)cc5ccccc45)cc3)c2)cc1 ; c1ccc(-c2cc(-c3ccccc3)cc(N(c3ccccc3)c3ccc(-c4cc5c(ccc6sc7ccccc7c65)c5ccccc45)c4ccccc34)c2)cc1 ; c1ccc(-c2ccc(N(c3ccccc3)c3cccc4c(-c5cc6c(ccc7oc8ccccc8c76)c6ccccc56)cccc34)cc2-c2ccccc2)cc1]

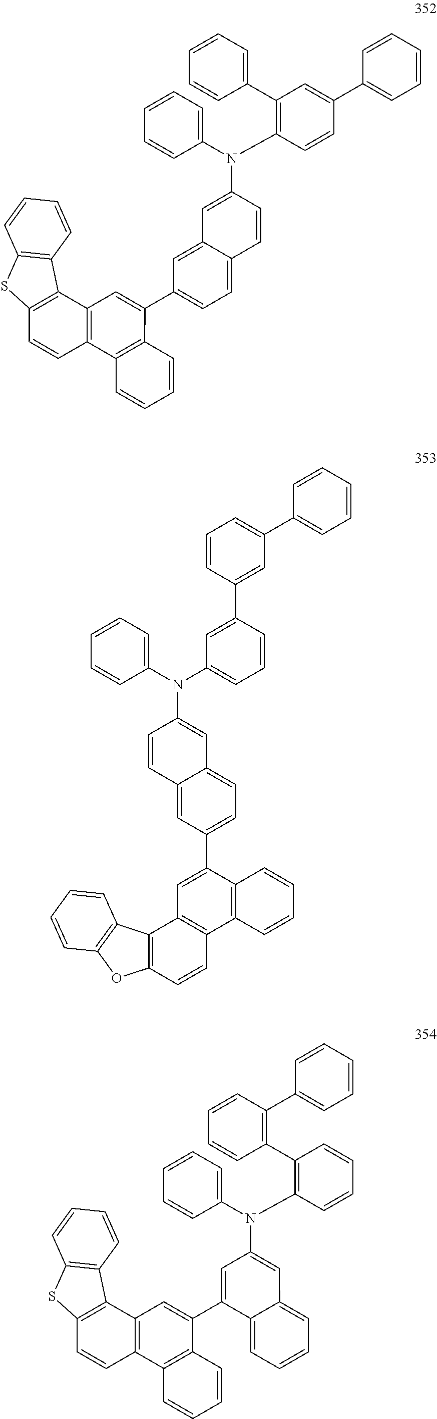 [SMILES: c1ccc(-c2ccc(N(c3ccccc3)c3ccc4ccc(-c5cc6c(ccc7sc8ccccc8c76)c6ccccc56)cc4c3)c(-c3ccccc3)c2)cc1 ; c1ccc(-c2cccc(-c3cccc(N(c4ccccc4)c4ccc5cc(-c6cc7c(ccc8oc9ccccc9c87)c7ccccc67)ccc5c4)c3)c2)cc1 ; c1ccc(-c2ccccc2-c2ccccc2N(c2ccccc2)c2cc(-c3cc4c(ccc5sc6ccccc6c54)c4ccccc34)c3ccccc3c2)cc1]

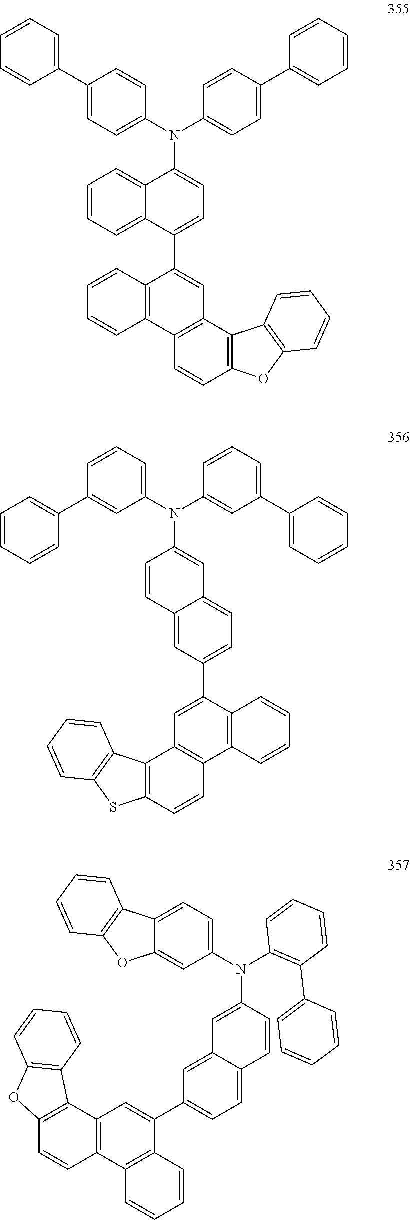 [SMILES: c1ccc(-c2ccc(N(c3ccc(-c4ccccc4)cc3)c3ccc(-c4cc5c(ccc6oc7ccccc7c65)c5ccccc45)c4ccccc34)cc2)cc1 ; c1ccc(-c2cccc(N(c3cccc(-c4ccccc4)c3)c3ccc4cc(-c5cc6c(ccc7sc8ccccc8c76)c6ccccc56)ccc4c3)c2)cc1 ; c1ccc(-c2ccccc2N(c2ccc3ccc(-c4cc5c(ccc6oc7ccccc7c65)c5ccccc45)cc3c2)c2ccc3c(c2)oc2ccccc23)cc1]

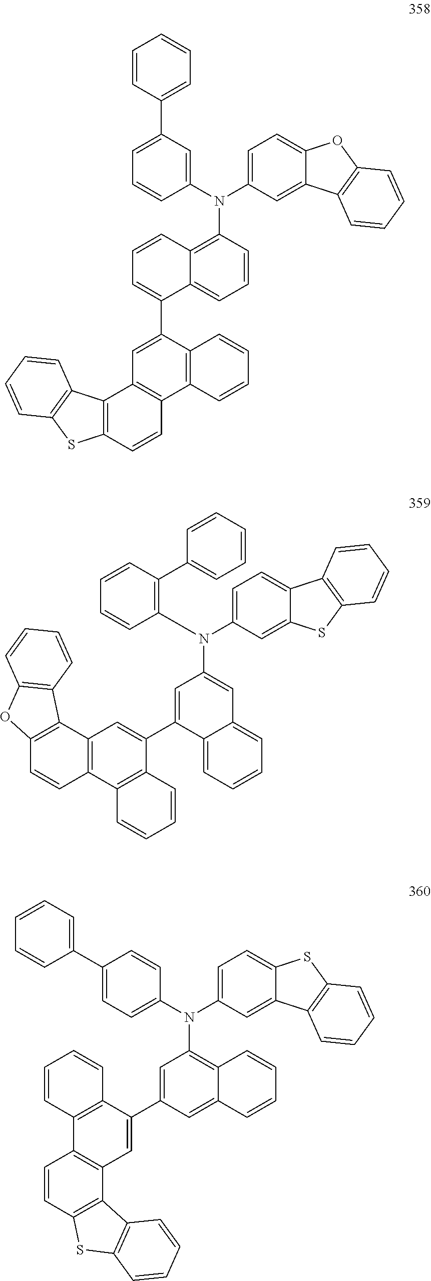 [SMILES: c1ccc(-c2cccc(N(c3ccc4oc5ccccc5c4c3)c3cccc4c(-c5cc6c(ccc7sc8ccccc8c76)c6ccccc56)cccc34)c2)cc1 ; c1ccc(-c2ccccc2N(c2cc(-c3cc4c(ccc5oc6ccccc6c54)c4ccccc34)c3ccccc3c2)c2ccc3c(c2)sc2ccccc23)cc1 ; c1ccc(-c2ccc(N(c3ccc4sc5ccccc5c4c3)c3cc(-c4cc5c(ccc6sc7ccccc7c65)c5ccccc45)cc4ccccc34)cc2)cc1]

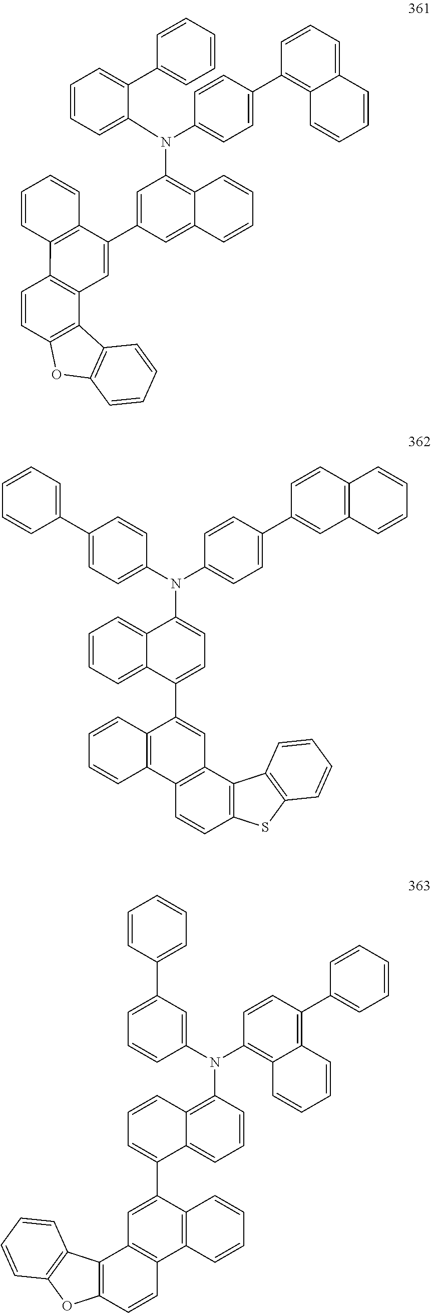 [SMILES: c1ccc(-c2ccccc2N(c2ccc(-c3cccc4ccccc34)cc2)c2cc(-c3cc4c(ccc5oc6ccccc6c54)c4ccccc34)cc3ccccc23)cc1 ; c1ccc(-c2ccc(N(c3ccc(-c4ccc5ccccc5c4)cc3)c3ccc(-c4cc5c(ccc6sc7ccccc7c65)c5ccccc45)c4ccccc34)cc2)cc1 ; c1ccc(-c2cccc(N(c3ccc(-c4ccccc4)c4ccccc34)c3cccc4c(-c5cc6c(ccc7oc8ccccc8c76)c6ccccc56)cccc34)c2)cc1]

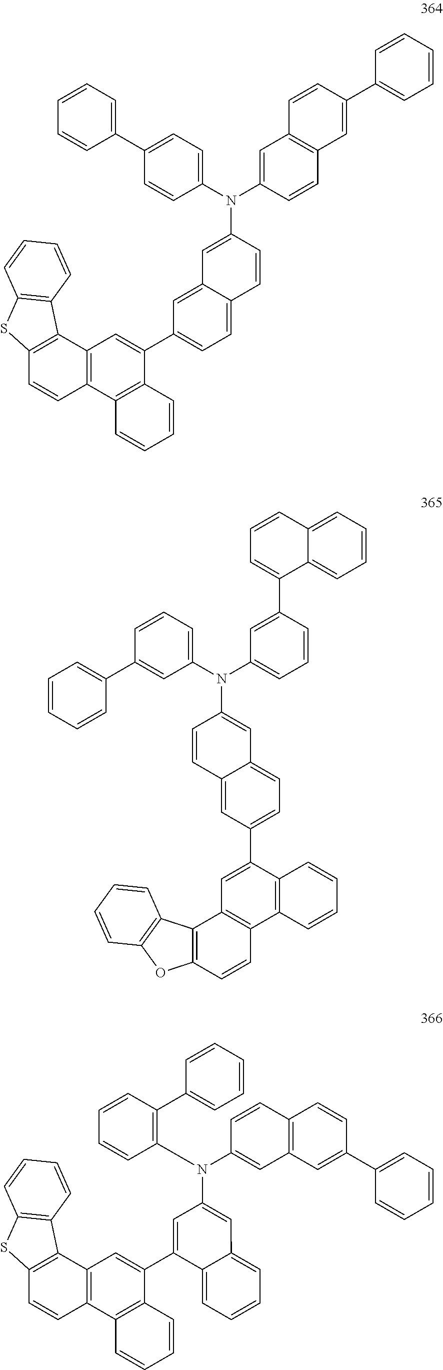 [SMILES: c1ccc(-c2ccc(N(c3ccc4cc(-c5ccccc5)ccc4c3)c3ccc4ccc(-c5cc6c(ccc7sc8ccccc8c76)c6ccccc56)cc4c3)cc2)cc1 ; c1ccc(-c2cccc(N(c3cccc(-c4cccc5ccccc45)c3)c3ccc4cc(-c5cc6c(ccc7oc8ccccc8c76)c6ccccc56)ccc4c3)c2)cc1 ; c1ccc(-c2ccc3ccc(N(c4cc(-c5cc6c(ccc7sc8ccccc8c76)c6ccccc56)c5ccccc5c4)c4ccccc4-c4ccccc4)cc3c2)cc1]

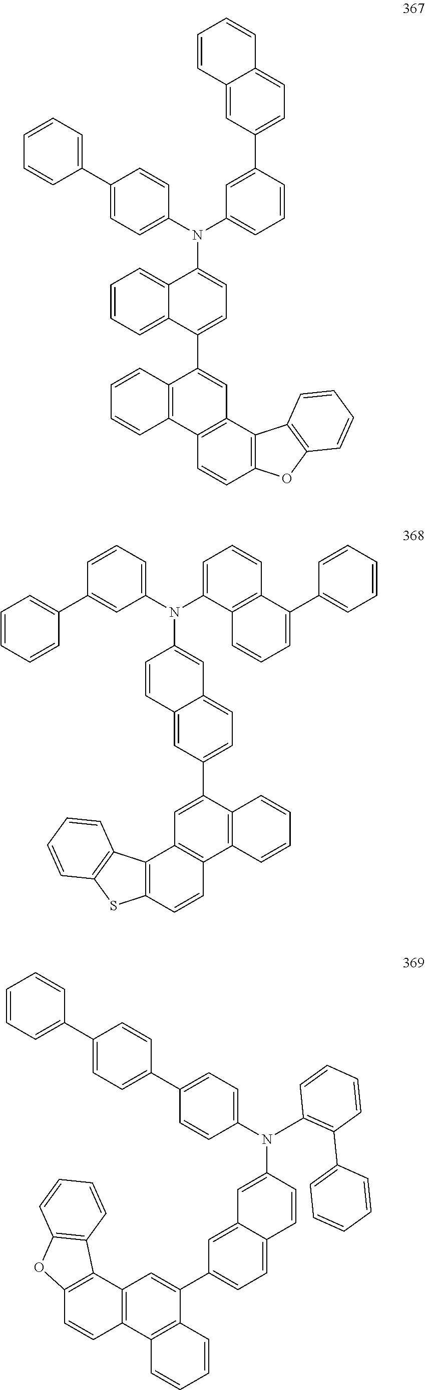 [SMILES: c1ccc(-c2ccc(N(c3cccc(-c4ccc5ccccc5c4)c3)c3ccc(-c4cc5c(ccc6oc7ccccc7c65)c5ccccc45)c4ccccc34)cc2)cc1 ; c1ccc(-c2cccc(N(c3ccc4cc(-c5cc6c(ccc7sc8ccccc8c76)c6ccccc56)ccc4c3)c3cccc4c(-c5ccccc5)cccc34)c2)cc1 ; c1ccc(-c2ccc(-c3ccc(N(c4ccc5ccc(-c6cc7c(ccc8oc9ccccc9c87)c7ccccc67)cc5c4)c4ccccc4-c4ccccc4)cc3)cc2)cc1]

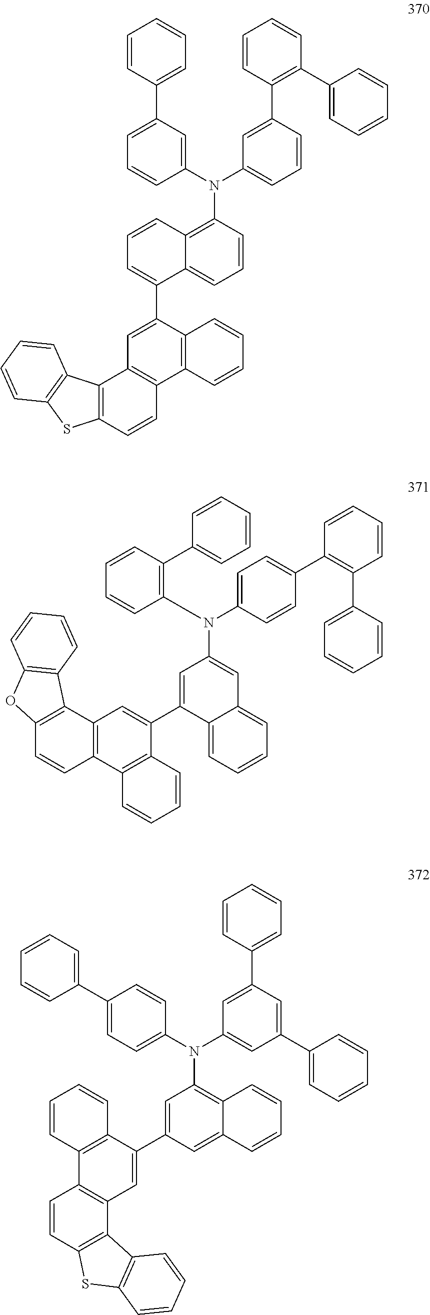 [SMILES: c1ccc(-c2cccc(N(c3cccc(-c4ccccc4-c4ccccc4)c3)c3cccc4c(-c5cc6c(ccc7sc8ccccc8c76)c6ccccc56)cccc34)c2)cc1 ; c1ccc(-c2ccccc2-c2ccc(N(c3cc(-c4cc5c(ccc6oc7ccccc7c65)c5ccccc45)c4ccccc4c3)c3ccccc3-c3ccccc3)cc2)cc1 ; c1ccc(-c2ccc(N(c3cc(-c4ccccc4)cc(-c4ccccc4)c3)c3cc(-c4cc5c(ccc6sc7ccccc7c65)c5ccccc45)cc4ccccc34)cc2)cc1]

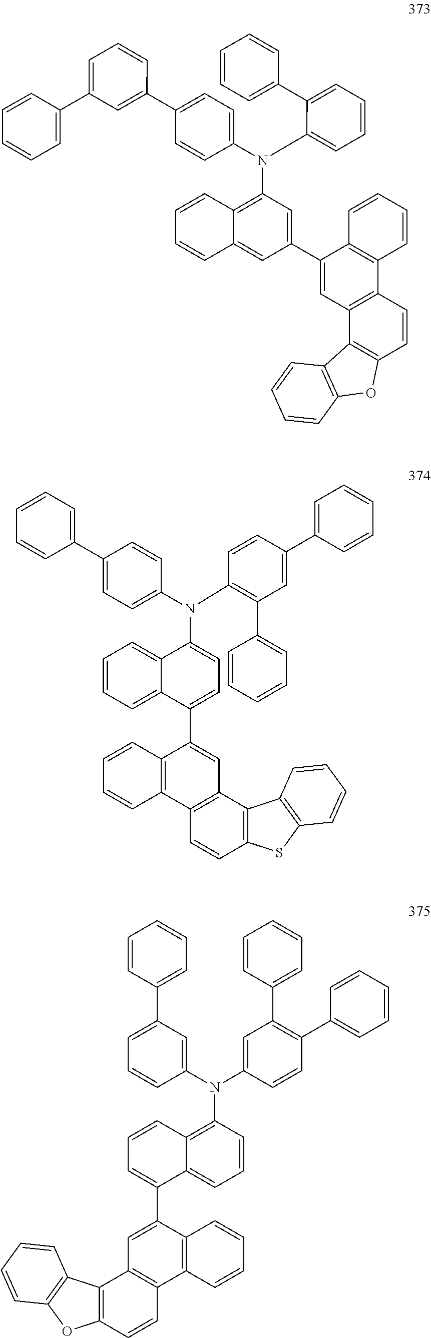 [SMILES: c1ccc(-c2cccc(-c3ccc(N(c4ccccc4-c4ccccc4)c4cc(-c5cc6c(ccc7oc8ccccc8c76)c6ccccc56)cc5ccccc45)cc3)c2)cc1 ; c1ccc(-c2ccc(N(c3ccc(-c4ccccc4)cc3-c3ccccc3)c3ccc(-c4cc5c(ccc6sc7ccccc7c65)c5ccccc45)c4ccccc34)cc2)cc1 ; c1ccc(-c2cccc(N(c3ccc(-c4ccccc4)c(-c4ccccc4)c3)c3cccc4c(-c5cc6c(ccc7oc8ccccc8c76)c6ccccc56)cccc34)c2)cc1]

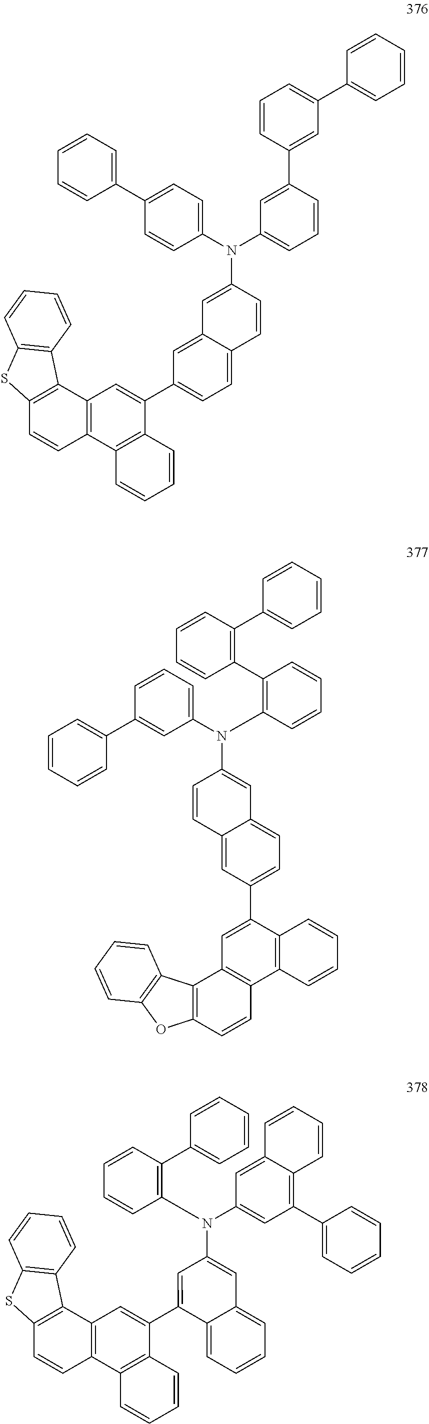 [SMILES: c1ccc(-c2ccc(N(c3cccc(-c4cccc(-c5ccccc5)c4)c3)c3ccc4ccc(-c5cc6c(ccc7sc8ccccc8c76)c6ccccc56)cc4c3)cc2)cc1 ; c1ccc(-c2cccc(N(c3ccc4cc(-c5cc6c(ccc7oc8ccccc8c76)c6ccccc56)ccc4c3)c3ccccc3-c3ccccc3-c3ccccc3)c2)cc1 ; c1ccc(-c2ccccc2N(c2cc(-c3ccccc3)c3ccccc3c2)c2cc(-c3cc4c(ccc5sc6ccccc6c54)c4ccccc34)c3ccccc3c2)cc1]

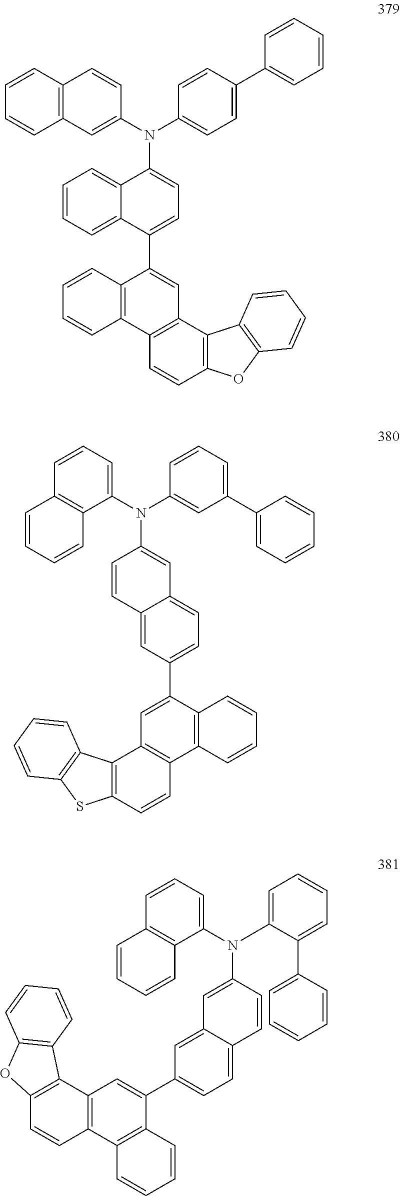 [SMILES: c1ccc(-c2ccc(N(c3ccc4ccccc4c3)c3ccc(-c4cc5c(ccc6oc7ccccc7c65)c5ccccc45)c4ccccc34)cc2)cc1 ; c1ccc(-c2cccc(N(c3ccc4cc(-c5cc6c(ccc7sc8ccccc8c76)c6ccccc56)ccc4c3)c3cccc4ccccc34)c2)cc1 ; c1ccc(-c2ccccc2N(c2ccc3ccc(-c4cc5c(ccc6oc7ccccc7c65)c5ccccc45)cc3c2)c2cccc3ccccc23)cc1]

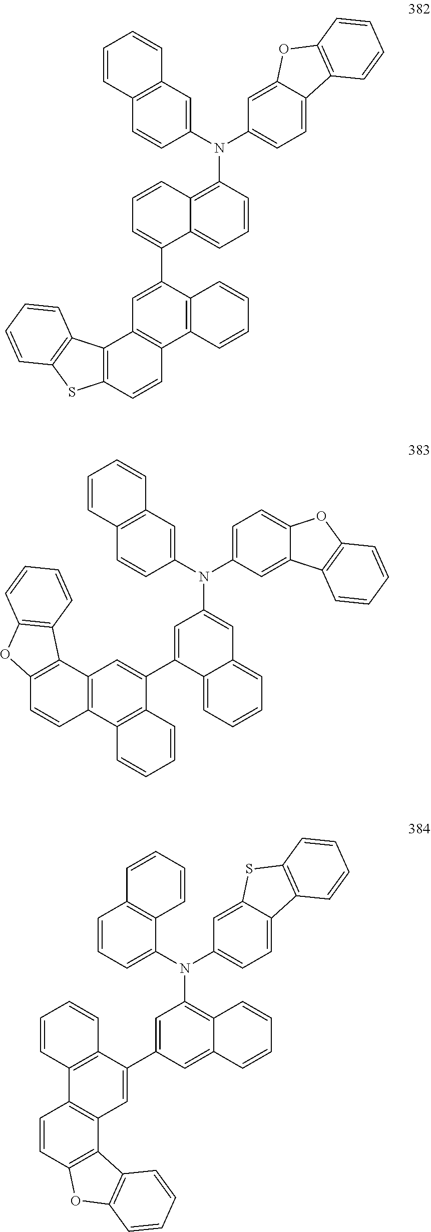 [SMILES: c1ccc2cc(N(c3ccc4c(c3)oc3ccccc34)c3cccc4c(-c5cc6c(ccc7sc8ccccc8c76)c6ccccc56)cccc34)ccc2c1 ; c1ccc2cc(N(c3cc(-c4cc5c(ccc6oc7ccccc7c65)c5ccccc45)c4ccccc4c3)c3ccc4oc5ccccc5c4c3)ccc2c1 ; c1ccc2c(N(c3ccc4c(c3)sc3ccccc34)c3cc(-c4cc5c(ccc6oc7ccccc7c65)c5ccccc45)cc4ccccc34)cccc2c1]

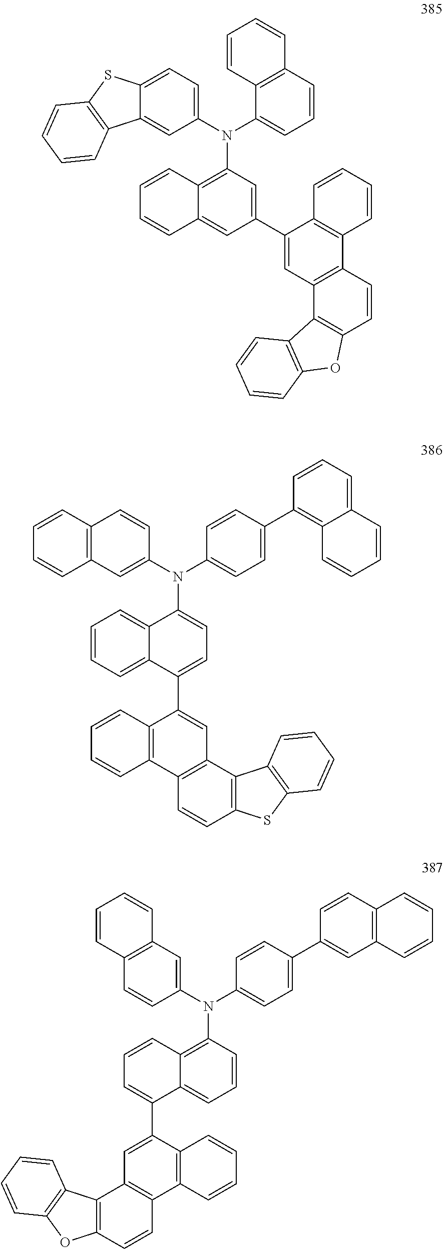 [SMILES: c1ccc2c(N(c3ccc4sc5ccccc5c4c3)c3cc(-c4cc5c(ccc6oc7ccccc7c65)c5ccccc45)cc4ccccc34)cccc2c1 ; c1ccc2cc(N(c3ccc(-c4cccc5ccccc45)cc3)c3ccc(-c4cc5c(ccc6sc7ccccc7c65)c5ccccc45)c4ccccc34)ccc2c1 ; c1ccc2cc(-c3ccc(N(c4ccc5ccccc5c4)c4cccc5c(-c6cc7c(ccc8oc9ccccc9c87)c7ccccc67)cccc45)cc3)ccc2c1]

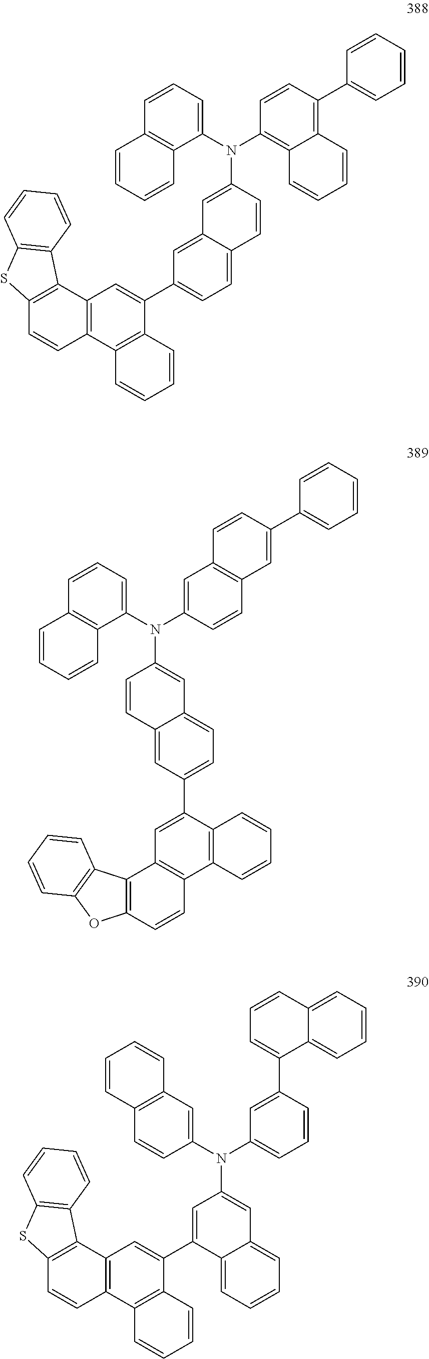 [SMILES: c1ccc(-c2ccc(N(c3ccc4ccc(-c5cc6c(ccc7sc8ccccc8c76)c6ccccc56)cc4c3)c3cccc4ccccc34)c3ccccc23)cc1 ; c1ccc(-c2ccc3cc(N(c4ccc5cc(-c6cc7c(ccc8oc9ccccc9c87)c7ccccc67)ccc5c4)c4cccc5ccccc45)ccc3c2)cc1 ; c1cc(-c2cccc3ccccc23)cc(N(c2ccc3ccccc3c2)c2cc(-c3cc4c(ccc5sc6ccccc6c54)c4ccccc34)c3ccccc3c2)c1]

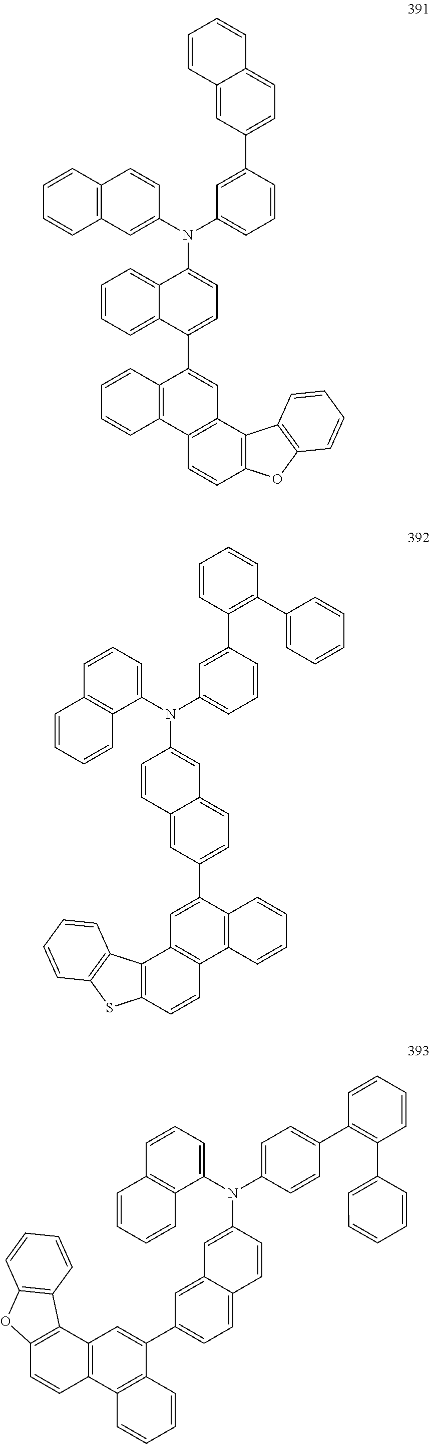 [SMILES: c1cc(-c2ccc3ccccc3c2)cc(N(c2ccc3ccccc3c2)c2ccc(-c3cc4c(ccc5oc6ccccc6c54)c4ccccc34)c3ccccc23)c1 ; c1ccc(-c2ccccc2-c2cccc(N(c3ccc4cc(-c5cc6c(ccc7sc8ccccc8c76)c6ccccc56)ccc4c3)c3cccc4ccccc34)c2)cc1 ; c1ccc(-c2ccccc2-c2ccc(N(c3ccc4ccc(-c5cc6c(ccc7oc8ccccc8c76)c6ccccc56)cc4c3)c3cccc4ccccc34)cc2)cc1]

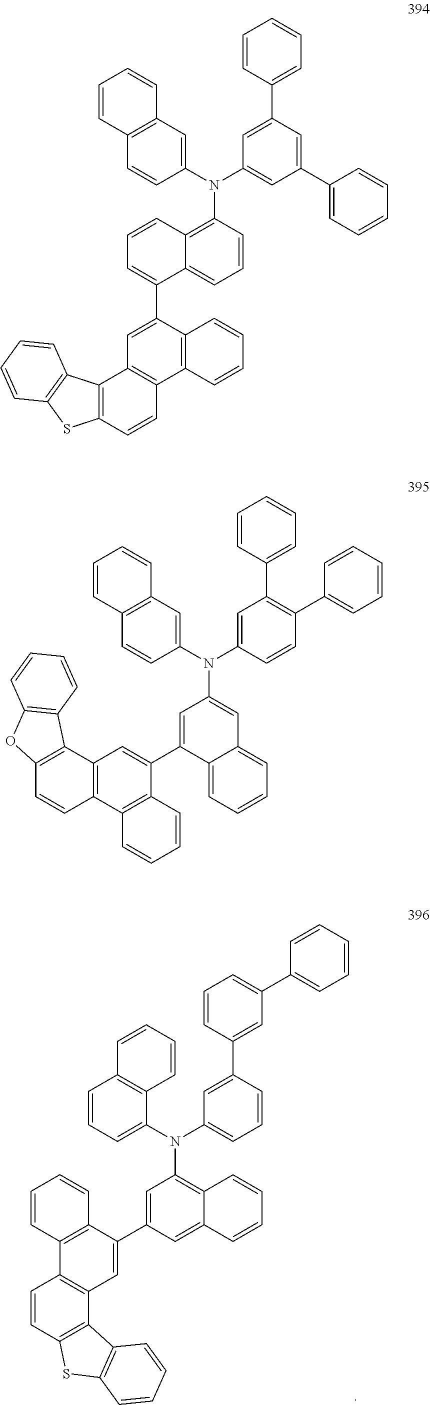 [SMILES: c1ccc(-c2cc(-c3ccccc3)cc(N(c3ccc4ccccc4c3)c3cccc4c(-c5cc6c(ccc7sc8ccccc8c76)c6ccccc56)cccc34)c2)cc1 ; c1ccc(-c2ccc(N(c3ccc4ccccc4c3)c3cc(-c4cc5c(ccc6oc7ccccc7c65)c5ccccc45)c4ccccc4c3)cc2-c2ccccc2)cc1 ; c1ccc(-c2cccc(-c3cccc(N(c4cccc5ccccc45)c4cc(-c5cc6c(ccc7sc8ccccc8c76)c6ccccc56)cc5ccccc45)c3)c2)cc1]

11. An organic electroluminescent device, comprising an anode and a cathode disposed opposite each other, and a functional layer disposed between the anode and the cathode; wherein the functional layer comprises the arylamine compound according to claim 1.

12. The organic electroluminescent device according to claim 11, wherein the functional layer comprises a light-emitting layer, and the organic light-emitting layer contains a host material and a guest material; and the host material comprises the arylamine compound.

13. An electronic apparatus, comprising the organic electroluminescent device according to claim 11.

14. The arylamine compound according to claim 2, wherein the substituents in L, L₁ and L₂ are the same or different, and are each independently selected from deuterium, fluorine, cyano, alkyl having 1-4 carbon atoms, haloalkyl having 1-4 carbon atoms, deuterated alkyl having 1-4 carbon atoms, trialkylsilyl having 3-7 carbon atoms, phenyl and deuterophenyl.

15. The arylamine compound according to claim 3, wherein the substituents in L, L₁ and L₂ are the same or different, and are each independently selected from deuterium, fluorine, cyano, methyl, ethyl, isopropyl, tert-butyl, trifluoromethyl, trideuteromethyl, trimethylsilyl, pentadeuterophenyl, and phenyl.

16. The arylamine compound according to claim 4, wherein L is selected from the group consisting of a single bond, and the following groups:

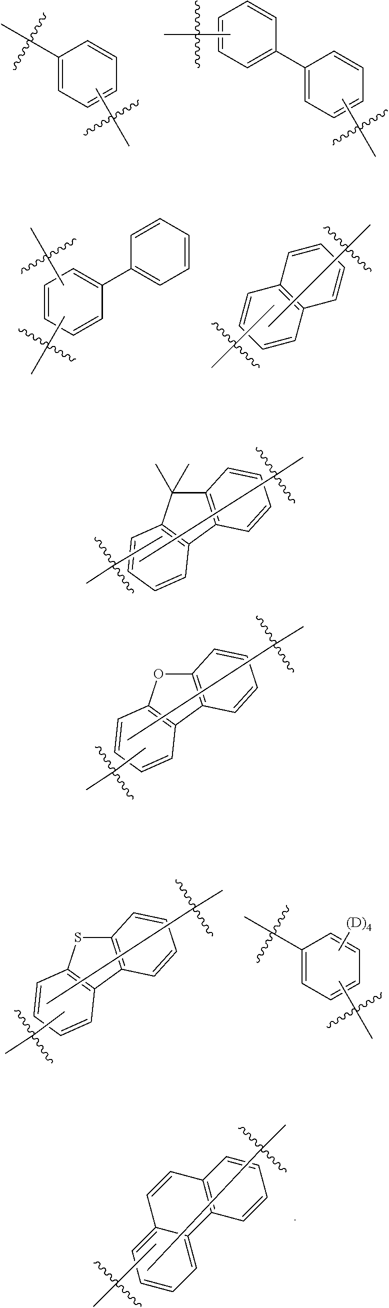

17. The arylamine compound according to claim 5, wherein the substituents in Ar₁ and Ar₂ are each independently selected from deuterium, halogen, cyano, haloalkyl having 1-4 carbon atoms, deuterated alkyl having 1-4 carbon atoms, alkyl having 1-4 carbon atoms, cycloalkyl having 5-10 carbon atoms, aryl having 6-15 carbon atoms, heteroaryl having 5-12 carbon atoms, trialkylsilyl having 3-7 carbon atoms, and a deuterated aryl having 6-15 carbon atoms; optionally, any two adjacent substituents form a benzene ring or a fluorene ring.

18. The arylamine compound according to claim 6, wherein the substituents in Ar₁ and Ar₂ are the same or different, and are each independently selected from deuterium, fluorine, cyano, methyl, ethyl, isopropyl, tert-butyl, trifluoromethyl, trideuteromethyl, trimethylsilyl, pentadeuterophenyl, phenyl, and naphthyl.
